(19) United States
(12) Patent Application Publication
(10) Pub. No.: US 2009/0169503 A1
(43) Pub. Date: Jul. 2, 2009

(54) DNA-BASED VACCINATION OF RETROVIRAL-INFECTED INDIVIDUALS UNDERGOING TREATMENT

(75) Inventors: Barbara K. Felber, Rockville, MD (US); George N. Pavlakis, Rockville, MD (US)

Correspondence Address: TOWNSEND AND TOWNSEND AND CREW, LLP, TWO EMBARCADERO CENTER, 8TH FLOOR, SAN FRANCISCO, CA, 94111, US

(73) Assignee: THE GOVERNMENT OF THE UNITED STATES, AS REPRESENTED BY THE SECRETARY OF DEPT OF H&HS, Rockville, MD (US)

(21) Appl. No.: 11/571,879

(22) PCT Filed: Jul. 11, 2005
(86) PCT No.: PCT/US2005/024498

§ 371 Date: Dec. 1, 2008

Related U.S. Application Data
(60) Provisional application No. 60/586,539, filed on Jul. 9, 2004.

(51) Int. Cl. A61K 38/20; A61K 39/00; A61P 31/00

(52) U.S. Cl. 424/85.2; 424/207.1; 424/208.1

(57) ABSTRACT

This invention provides DNA vaccines for the treatment of patients undergoing retroviral therapy. The vaccines are surprisingly effective at controlling viremia.

### CROSS-REFERENCE TO RELATED APPLICATIONS

This application claims benefit of U.S. provisional application No. 60/586,539, filed Jul. 9, 2004, which application is incorporated by reference herein.

### BACKGROUND OF THE INVENTION

Antiretroviral therapy (ART) to treat HIV has changed the outlook of HIV infection, since well-managed patients can remain free of symptoms for long periods. However, chronic use of the drugs leads to toxicities and virus resistance. Therapy must be continued indefinitely, since HIV (or SIV in macaques) remaining in pharmacological sanctuaries, rebounds rapidly upon treatment interruption

The administration of nucleic acid-based vaccines, including both naked DNA and viral-based vaccines, to individuals that have undergone ART has been suggested (see, e.g., WO01/08702, WO04/041997). Further, the administration of DNA vaccines in prime boost protocols has been suggested (see, e.g., US application no. 2004/033237; Hel et al., J. Immunol. 169:4778-4787, 2002; Barnett et al., AIDS Res. and Human Retroviruses Volume 14, Supplement 3, 1998, pp. S-299-S-309 and Girard et al., C R Acad. Sci. III 322:959-966, 1999 for reviews). DNA immunization, when used in a boosting protocol with modified vaccinia virus Ankara (MVA) or with a recombinant fowl pox virus (rFPV) in the macaque model, has been shown to induce CTL responses and antibody responses (see, e.g., Hanke et al, J. Virol. 73:7524-7532, 1999; Hanke et al., Immunol. Letters 66:177-181; Robinson et al., Nat. Med. 5:526-534, 1999), but no protection from a viral challenge was achieved in the immunized animals.

DNA immunization followed by administration of another highly attenuated poxvirus has also been tested for the ability to elicit IgG responses, but the interpretation of the results is hampered by the fact that serial challenges were performed (see, e.g., Fuller et al., Vaccine 15:924-926, 1997; Barnett et al., supra). In contrast, in a murine model of malaria, DNA vaccination used in conjunction with a recombinant vaccinia virus was promising in protecting from malaria infection (see, e.g., Sedegah et al., Proc. Natl. Acad. Sci. USA 95:7648-7653, 1998; Schneider et al., Nat. Med. 4:397-402, 1998).

Other prime boost strategies for the treatment of HIV infection are described in WO01/82964, WO04/041997. In these methods, immunogenicity of a recombinant poxvirus-based vaccine is enhanced by administering a nucleic acid, e.g., a DNA plasmid vaccine, to stimulate an immune response to the HIV antigens provided in the poxvirus vaccine, and thereby increase the ability of the recombinant pox virus, e.g., NYVAC or ALVAC, to expand a population of immune cells. Individuals who are treated with such a vaccine regimen may be at risk for infection with the virus or may have already been infected. Such protocols can control viremia for a period of time. However, these protocols rely on the use of DNA plasmid vaccines in conjunction with poxvirus vaccines. DNA plasmid vaccines by themselves have not been previously shown to have the ability to control viremia.

In contrast to intervention during early infection, results have been mixed in chronic infection, and most reports suggest that immune therapy during chronic infection was transiently effective, if at all, in controlling virus load and boosting immune response (see, e.g., Lori, et al., Science 290:1591-1593, 2000; Markowitz, et al., J Infect Dis 186:634-643, 2002; Tryniszewska, et al., J Immunol 169:5347-5357, 2002). Perhaps the most successful protocol reported is the therapeutic dendritic cell vaccination. Treatment of macaque and human APCs in vitro with immunogen and re-infusion in the absence of antiretroviral therapy (see, e.g., Lu, et al., Nat Med 9:27-32, 2003) resulted in long-lasting decrease in virus load. Several indications from the reported immunotherapy studies suggest that restoration of the immune system and perhaps more efficient immunization procedures may improve virus control.

DNA immunization plasmids have been developed that encode fusion proteins that contain a destabilizing amino acid sequence attached to a polypeptide sequence of interest; or that encode secreted fusion proteins, e.g., containing a secretory peptide attached to a polypeptide of interest (see. e.g., WO02/36806). Both of these types of plasmids exhibit increased immunogenicity of the polypeptide of interest that is comprised in the two types of fusion proteins. However, these DNA immunization plasmids have not been tested for their ability to control viremia in subjects that have undergone ART. It is highly desirable that additional methods of virus control and immune restoration are developed. This invention addresses this need.

### BRIEF SUMMARY OF THE INVENTION

The invention is based on the discovery of DNA vaccines for the treatment of retrovirus infection that are surprisingly effective at controlling viremia in primates that are receiving or will receive antiretroviral therapy (ART), either alone or in conjunction with other therapeutic vaccines. This vaccination can induce long-lasting virus-specific immune responses, and control viremia post-ART. DNA therapeutic vaccination appears surprisingly effective and, further, shows evidence of triggering a Th1 response with more prominent induction of cellular immune responses.

The invention thus provides a method of treating an individual, preferably a human, infected with a retrovirus, the method comprising: administering a DNA vaccine comprising an expression vector selected from the group consisting of a) an expression vector encoding a fusion protein comprising a degradation polypeptide linked to an immunogenic retrovirus polypeptide or b) an expression vector encoding a secreted fusion protein comprising a secretory polypeptide linked to an immunogenic retrovirus polypeptide; and administering antiretroviral therapy (ART); wherein administration of the DNA vaccine results in control of viremia upon cessation of ART. In preferred embodiments, the DNA vaccine is administered to an individual who is undergoing ART.

In some embodiments, an expression vector encoding a secreted polypeptide is administered in conjunction with an expression vector encoding a fusion polypeptide comprising a destabilizing sequence. In such an embodiment, the antigenic retroviral polypeptide in the secreted polypeptide is often a different antigen than the antigenic polypeptide that is linked to the destabilizing sequence.

In particular embodiments, the destabilizing sequence in the fusion polypeptides that are administered in vaccines can be selected from the group consisting of c-Mos aa1-35, cyclin B aa 10-95, β-catenin aa 19-44, and β-catenin aa 18-47. Often, the destabilizing sequence is β-catenin aa 18-47.

In some embodiments, the secretory polypeptide is MCP-3.

The antigenic polypeptides that can be incorporated into the fusion proteins can be from any retrovirus, e.g., HIV-1, HIV-2, HTLV, SIV, but are often from HIV-1. Most often, the immunogenic retrovirus polypeptide is from an HIV antigen, such as Gag, Env, Pol, Nef, Vpr, Vpu, Vif, Tat, or Rev. In some embodiments, the HIV antigen comprises linked epitopes from HIV antigens, e.g., HIV Gag, Pol, Tat, Rev, or Nef, linked in any order; or linked epitopes of HIV antigens, e.g., Tat, Rev, Env, or Nef, linked in any order. One or more of the HIV genes, e.g., Gag, Env, Pol, Nef, Vpr, Vpu, Vif, Tat, or Rev, is often engineered so that an inactive protein is produced. In some embodiments, the linked epitopes are fusion proteins, such as Gag/Pol fusion proteins. The HIV antigens can be administered in one or more expression vectors, For example, a Gag/Pol fusion protein can be encoded in one expression vector and an Env protein on another expression vector.

The vaccines of the invention can also be administered with a nucleic acid sequence encoding a co-stimulatory molecule, i.e., an adjuvant, such as IL-12 or IL-15. The nucleic acid sequence encoding the co-stimulatory molecule is most often administered at the same time as one or more of the expression vectors of the invention and at the same site. However, this need not necessarily be the case. The vectors may be administered at different sites and/or at different times.

In some embodiments, the expression vector is administered by intramuscular injection. The vaccine can be administered at a single site or multiple sites. Further, combinations of expression vectors can be administered. In some embodiments, an expression vector encoding a secreted fusion protein is administered at a site that is different from the site of administration of an expression vector encoding an antigenic fusion protein comprising a destabilizing polypeptide sequence.

In other embodiments, the method of the invention further comprises at least a second administration of the expression plasmid. Thus, multiple administrations of the same or different expression plasmids is contemplated in the invention.

The invention also provides a method of treating an individual undergoing antiretroviral therapy, the method comprising administering to the individual a DNA vaccine comprising an expression vector selected from the group consisting of a) an expression vector encoding a fusion protein comprising a degradation polypeptide linked to an immunogenic retrovirus polypeptide and/or b) an expression vector encoding a secreted fusion protein comprising a secretory polypeptide linked to an immunogenic retrovirus polypeptide; wherein administration of the DNA vaccine results in lower levels of viremia compared to viremia prior to ART administration upon cessation of ART. The vectors often comprise mutated retroviral genes, e.g., mutated HIV genes that express inactive proteins. For example, gag, pol, nef, tat, may be mutated to inactivate protein function. Such vectors can also be administered with vectors that encode native antigens (or native antigen epitopes) without modifications.

The nucleic acid constructs of the invention for treatment of retroviral infection, e.g., HIV, can be used in conjunction with other therapeutic treatments, including other nucleic acid-based vaccines, such as virus vectors, e.g., poxvirus vectors, retroviral vectors, e.g., lentiviral vectors, adenoviral vectors, adeno-associated viral vectors and the like. Further, other immunogenic formulations can be administered in conjunction with the constructs, including purified protein antigens or inactivated virus particles.

### BRIEF DESCRIPTION OF THE DRAWINGS

FIG. 1 provides a schematic of immunotherapy of Rhesus macaques chronically infected by SIVmac251. Animals received 3-4 immunizations during therapy and were observed for several months after ART termination.

FIG. 2A and FIG. 2B provide exemplary data showing virus load in plasma of all macaques in the study from infection to end of follow-up period. Thick gray bars indicate the period under ART. (A) 12 animals treated with ART+DNA vaccination (B) control group treated only with ART

FIG. 3 provides exemplary data showing a comparison of virus load before and after ART: (Left) Comparison of average virus load over fixed periods of the 10 weeks preceding and the 13 weeks following ART therapy. Average viremia before and after therapy is shown for ART group (top) and ART+DNA vaccine group (bottom). (Right) Comparison of average virus load for the entire chronic period before therapy, versus the entire period after ART release.

FIG. 4A-FIG. 4C provide exemplary data showing elispot analysis of vaccine-treated and control animals. Elispot analysis for 10 ART+DNA vaccination animals (A, B) and 3 ART only controls (C). Gray and open stacked bars represent Elispot values (right scale) for gag and gp120env, respectively, for the indicated dates. Dotted line indicates virus load (left scale).

FIG. 5 provides exemplary data showing immunological analysis of treated animals. This analysis showed induction of cellular and humoral immune responses after DNA vaccination. FIG. 5A shows the ELISPOT response to gag and env for 10 vaccinated animals, shown as median and quartiles, divided into 4 periods, chronic phase, ART before vaccination, ART and DNA vaccination, and follow-up after drug termination. Antibodies against SIV proteins were measured by Elisa (FIG. 5B). The animals had high antibody levels against SIV. Ab levels were slightly decreased during ART and were not increased during vaccination, whereas after ART termination the antibody levels were increased to higher levels.

FIG. 6 shows exemplary modifications to Vif.

FIG. 7 shows exemplary modifications to Tat.

FIG. 8 shows exemplary modifications to Nef.

FIG. 9 shows exemplary modifications to Pol.

FIG. 10 is a schematic for expression of an exemplary HIV-1 Gag-pol in-frame for a vaccine vector.

FIG. 11 provides a schematic showing the generation of an exemplary Nef-tat-vif-(NTV) fusion protein lacking nef/tat/vif function for use in the vaccine constructs of the invention.

FIG. 12 shows a comparison of wt vs modified SIV pol. The modified SIV pol lacks function.

### DETAILED DESCRIPTION OF THE INVENTION

### Definitions

A “nucleic acid vaccine” or “DNA vaccine” refers to a vaccine that includes one or more expression vectors, preferably administered as purified DNA, which enters the cells in the body, and is expressed.

A “destabilizing amino acid sequence” or “destabilization sequence” as used herein refers to a sequence that targets a protein for degradation. Such sequences are well known in the art. Typically, the destabilizing sequence targets the protein to the ubiquitin proteosomal degradation pathway. Such sequences are well known in the art. Exemplary sequences are described, e.g., in WO 02/36806.

A “secretory polypeptide” as used herein refers to a polypeptide that comprises a secretion signal that is typically secreted. Typically, a “secretory polypeptide” that is comprised by a fusion protein is an immunostimulatory molecule such as a chemokine or cytokine.

“Viral load” is the amount of virus present in the blood of a patient. Viral load is also referred to as viral titer or viremia. Viral load can be measured in variety of standard ways. In preferred embodiments, the administration of the DNA constructs controls viremia and leads to a greater reduction in viral load.

### Introduction

A recurring problem in anti-retroviral therapy is the rebound in viremia when therapy ceases. This invention is based on the discovery that vectors that produce either secreted or intracellularly degraded antigens are surprisingly effective at controlling viremia when administered to ART-treated subjects. These vectors can be used for the treatment of retroviral infection, e.g., for the treatment of HIV infection.

### Expression Vectors Encoding Fusion Polypeptides Comprising a Degradation Signal

The nucleic acid vaccines of the invention are typically administered as “naked” DNA, i.e., as plasmid-based vectors. Since the antigens expressed by these DNA vectors are also well expressed in other expression systems, such as recombinant virus vectors, other expression vector systems may also be used either alternatively, or in combination with DNA vectors. These include viral vector systems such as cytomegalovirus, herpes virus, adenovirus, and the like. Such viral vector systems are well known in the art. The constructs of the invention can thus also be administered in viral vectors where the retroviral antigens, e.g., the HIV antigens, are incorporated into the viral genetic material.

Expression vectors encoding a fusion protein comprising a destabilization sequence linked to the immunogenic protein are used in the invention. Such vectors are described, e.g., in WO02/36806. A variety of sequence elements have been found to confer short lifetime on cellular proteins. For example, the amino acid residues present in the N-terminus may destabilize a protein sequence. Another example of destabilizing sequences are so-called PEST sequences, which are abundant in the amino acids Pro, Asp, Glu, Ser, Thr (they need not be in a particular order), and can occur in internal positions in a protein sequence. A number of proteins reported to have PEST sequence elements are rapidly targeted to the 26S proteasome. A PEST sequence typically correlates with a) predicted surface exposed loops or turns and b) serine phosphorylation sites, e.g. the motif S/TP is the target site for cyclin dependent kinases.

Additional destabilization sequences relate to sequences present in the n-terminal region. In particular the rate of ubiquitination, which targets proteins for degradation by the 26S proteasome can be influence by the identity of the N-terminal residue of the protein. Thus, destabilization sequences can also comprise such N-terminal residues, “N-end rule” targeting (see, e.g., Tobery et al., J. Exp. Med. 185:909-920.)

Destabilizing sequences present in particular proteins are well known in the art. Exemplary destabilization sequences include c-myc aa 2-120; cyclin A aa 13-91; Cyclin B aa 13-91; IkBα aa 20-45; β-Catenin aa 9-44; β-Catenin aa 18-447, c-Jun aa1-67; and c-Mos aa1-35; and fragments and variants, of those segments that mediate destabilization. Such fragments can be identified using methodology well known in the art. For example, polypeptide half-life can be determined by a pulse-chase assay that detects the amount of polypeptide that is present over a time course using an antibody to the polypeptide, or to a tag linked to the polypeptide. Exemplary assays are described, e.g., in WO02/36806.

Expression Vectors that Encode Secreted Fusion Proteins

The vaccines of the invention (naked DNA or viral vector-based nucleic acid vaccines) can also encode fusion proteins that include a secretory polypeptide. In some embodiments, the secretory polypeptide is an immunostimulation molecule, such as a chemokine, cytokine, or lymphokine. Exemplary secretory polypeptides include immunostimulatory chemokines such as MCP-3 or IP-10, or cytokines such as GM-CSF, IL-4, or IL-2. Often, secretory fusion proteins employed in the methods here contain MCP-3 amino acid sequences to tissue plasminogen activator sequences. Constructs encoding secretory fusion proteins are disclosed, e.g., in WO02/36806.

### Selection of Epitopes

Antigenic polypeptide sequences for provoking an immune response selective for a specific retroviral pathogen are known. With minor exceptions, the following discussion of HIV epitopes/immunogenic polypeptides is applicable to other retroviruses, e.g., SIV, except for the differences in sizes of the respective viral proteins. HIV antigens for a multitude of HIV-1 and HIV-2 isolates, including members of the various genetic subtypes of HIV, are known and reported (see, e.g., Myers et al., Los Alamos Database, Los Alamos National Laboratory, Los Alamos, N. Mex. (1992); the updated version of this data base is online and is incorporated herein by reference (http://hiv-web.lanl.gov/content/index)) and antigens derived from any of these isolates cam be used in the methods of this invention. Immunogenic proteins can be derived from any of the various HIV isolates, including any of the various envelope proteins such as gp120, gp160 and gp41; gag antigens such as p24gag and p55gag, as well as proteins derived from pol, tat, vif, rev, nef, vpr, vpu.

The expression constructs may also contain Rev-independent fragments of genes that retain the desired function (e.g., for antigenicity of Gag or Pol, particle formation (Gag) or enzymatic activity (Pol)), or may also contain Rev-independent variants that have been mutated such the encoded protein loses function. For example, the gene may be modified to mutate an active site of reverse transcriptase or integrase proteins. Rev-independent fragments of gag and env are described, for example, in WO01/46408 and U.S. Pat. Nos. 5,972,596 and 5,965,726. Typically, rev-independent HIV sequences that are modified to eliminate all enzymatic activities of the encoded proteins are used in the constructs of the invention.

A DNA vaccine of the invention can be administered as one or more constructs. For example, a vaccine can comprises an HIV antigen fusion protein where multiple HIV polypeptides, structural and/or regulatory polypeptides or immunogenic epitopes thereof, are administered in a single expression vectors. In other embodiments, the vaccines are administered as multiple expression vectors, or as one or more expression vectors encoding multiple expression units, e.g., discistronic expression vectors.

### Anti-Retroviral Therapy

The vaccines are administered to retrovirus-infected individuals, typically HIV-1-infected humans, who are undergoing or have undergone ART therapy.

Antiviral retroviral treatment typically involves the use of two broad categories of therapeutics. They are reverse transcriptase inhibitors and protease inhibitors. There are two type of reverse transcriptase inhibitors: nucleoside analog reverse transcriptase inhibitors and non-nucleoside reverse transcriptase inhibitors. Both types of inhibitors block infection by blocking the activity of the HIV reverse transcriptase, the viral enzyme that translates HIV RNA into DNA which can later be incorporated into the host cell chromosomes.

Nucleoside and nucleotide analogs mimic natural nucleotides, molecules that act as the building blocks of DNA and RNA. Both nucleoside and nucleotide analogs must undergo phosphorylation by cellular enzymes to become active; however, a nucleotide analog is already partially phosphorylated and is one step closer to activation when it enters a cell. Following phosphorylation, the compounds compete with the natural nucleotides for incorporation by HIV's reverse transcriptase enzyme into newly synthesized viral DNA chains, resulting in chain termination.

Examples of anti-retroviral nucleoside analogs are: AZT, ddI, ddC, d4T, and 3TC. Combinations of different nucleoside analogs are also available, for example 3TC in combination with in combination withAZT and (Combivir).

Nonnucleoside reverse transcriptase inhibitors (NNRTIs) are a structurally and chemically dissimilar group of antiretroviral compounds. They are highly selective inhibitors of HIV-1 reverse transcriptase. At present these compounds do not affect other retroviral reverse transcriptase enzymes such as hepatitis viruses, herpes viruses, HIV-2, and mammalian enzyme systems. They are used effectively in triple-therapy regimes. Examples of NNRTIs are Delavirdine and Nevirapine which have been approved for clinical use in combination with nucleoside analogs for treatment of HIV-infected adults who experience clinical or immunologic deterioration. A detailed review can be found in “Nonnucleoside Reverse Transcriptase Inhibitors” AIDS Clinical Care (October 1997) Vol. 9, No. 10, p. 75.

Protease inhibitors are compositions that inhibit HIV protease, which is virally encoded and necessary for the infection process to proceed. Clinicians in the United States have a number of clinically effective proteases to use for treating HIV-infected persons. These include: SAQUINAVIR (Invirase); INDINAVIR (Crixivan); and RITONAVIR (Norvir).

### Preparation of Vaccines

In the methods of the invention, the nucleic acid vaccine is directly introduced into the cells of the individual receiving the vaccine regimen. This approach is described, for instance, in Wolff et. al., Science 247:1465 (1990) as well as U.S. Pat. Nos. 5,580,859; 5,589,466; 5,804,566; 5,739,118; 5,736,524; 5,679,647; and WO 98/04720. Examples of DNA-based delivery technologies include, “naked DNA”, facilitated (bupivicaine, polymers, peptide-mediated) delivery, and cationic lipid complexes or liposomes. The nucleic acids can be administered using ballistic delivery as described, for instance, in U.S. Pat. No. 5,204,253 or pressure (see, e.g., U.S. Pat. No. 5,922,687). Using this technique, particles comprised solely of DNA are administered, or in an alternative embodiment, the DNA can be adhered to particles, such as gold particles, for administration.

As is well known in the art, a large number of factors can influence the efficiency of expression of antigen genes and/or the immunogenicity of DNA vaccines. Examples of such factors include the reproducibility of inoculation, construction of the plasmid vector, choice of the promoter used to drive antigen gene expression and stability of the inserted gene in the plasmid. In some embodiments, nucleic acid-based vaccines comprising expression vectors of the invention are viral vectors in which the retroviral antigens for vaccination are included in the viral vector genome.

Any of the conventional vectors used for expression in eukaryotic cells may be used for directly introducing DNA into tissue. Expression vectors containing regulatory elements from eukaryotic viruses are typically used in eukaryotic expression vectors, e.g., CMV, viral LTRs and the like. Typical vectors include those with a human CMV promoter, no splice sites, and a bovine growth hormone polyA site. Exemplary vectors are described in the “Examples” section.

Therapeutic quantities of plasmid DNA can be produced for example, by fermentation in E. coli, followed by purification. Aliquots from the working cell bank are used to inoculate growth medium, and grown to saturation in shaker flasks or a bioreactor according to well known techniques. Plasmid DNA can be purified using standard bioseparation technologies such as solid phase anion-exchange resins. If required, supercoiled DNA can be isolated from the open circular and linear forms using gel electrophoresis or other methods.

Purified plasmid DNA can be prepared for injection using a variety of formulations. The simplest of these is reconstitution of lyophilized DNA in sterile phosphate-buffer saline (PBS). This approach, i.e., “naked DNA,” is particularly suitable for intramuscular (IM) or intradermal (ID) administration.

### Assessment of Immunogenic Response

To assess a patient's immune system during and after treatment and to further evaluate the treatment regimen, various parameters can be measured. Measurements to evaluate vaccine response include: antibody measurements in the plasma, serum, or other body fluids; and analysis of in vitro cell proliferation in response to a specific antigen, indicating the function of CD4+ cells. Such assays are well known in the art. For example, for measuring CD4+ T cells, many laboratories measure absolute CD4+ T-cell levels in whole blood by a multi-platform, three-stage process. The CD4+ T-cell number is the product of three laboratory techniques: the white blood cell (WBC) count; the percentage of WBCs that are lymphocytes (differential); and the percentage of lymphocytes that are CD4+ T-cells. The last stage in the process of measuring the percentage of CD4+ T-lymphocytes in the whole-blood sample is referred to as “immunophenotyping by flow cytometry. Systems for measuring CD4+ cells are commercially available. For example Becton Dickenson's FACSCount System automatically measure absolutes CD4+, CD8+, and CD3+ T lymphocytes.

Other measurements of immune response include assessing CD8+ responses. These techniques are well known. CD8+ T-cell responses can be measured, for example, by using tetramer staining of fresh or cultured PBMC (see, e.g., Altman, et al., Proc. Natl. Acad. Sci. USA 90:10330, 1993; Altman, et al., Science 274:94, 1996), or γ-interferon release assays such as ELISPOT assays (see, e.g., Lalvani, et al., J. Exp. Med. 186:859, 1997; Dunbar, et al., Curr. Biol. 8:413, 1998; Murali-Krishna, et al., Immunity 8:177, 1998), or by using functional cytotoxicity assays.

### Viral Titer

Viremia is measured by assessing viral titer in a patient. There are a variety of methods of perform this. For example, plasma HIV RNA concentrations can be quantified by either target amplification methods (e.g., quantitative RT polymerase chain reaction [RT-PCR], Amplicor HIV Monitor assay, Roche Molecular Systems; or nucleic acid sequence-based amplification, [NASBA®], NucliSens™ HIV-1 QT assay, Organon Teknika) or signal amplification methods (e.g., branched DNA [bDNA], Quantiplex™ HIV RNA bDNA assay, Chiron Diagnostics). The bDNA signal amplification method amplifies the signal obtained from a captured HIV RNA target by using sequential oligonucleotide hybridization steps, whereas the RT-PCR and NASBA® assays use enzymatic methods to amplify the target HIV RNA into measurable amounts of nucleic acid product. Target HIV RNA sequences are quantitated by comparison with internal or external reference standards, depending upon the assay used.

Administration of vaccine constructs of the invention to individuals undergoing ART controls viremia, e.g., in periods when the patient may stop receiving ART. Controlling viremia refers to lowering of the plasma levels of virus to levels lower than those observed in the period of chronic infection prior to ART, usually to levels to levels one to two logs lower than the set point observed in the period of chronic infection prior to ART. Inclusion of the vaccine constructs described herein results in enhanced control of viremia in comparison to treatment protocols that do not comprise administration of optimized DNA vectors or that do not that encode fusion proteins comprising a destabilization signal/and or secreted fusion proteins.

### Administration of DNA Constructs

To maximize the immunotherapeutic effects of DNA vaccines, alternative methods for formulating purified plasmid DNA may be desirable. A variety of methods have been described, and new techniques may become available. Cationic lipids can also be used in the formulation (see, e.g., as described by WO 93/24640; Mannino & Gould-Fogerite, BioTechniques 6(7): 682 (1988); U.S. Pat. No. 5,279,833; WO 91/06309; and Felgner, et al., Proc. Nat'l Acad. Sci. USA 84:7413 (1987). In addition, glycolipids, fusogenic liposomes, peptides and compounds referred to collectively as protective, interactive, non-condensing compounds (PINC) could also be complexed to purified plasmid DNA to influence variables such as stability, intramuscular dispersion, or trafficking to specific organs or cell types.

The administration procedure for DNA is not critical. Vaccine compositions (e.g., compositions containing the DNA expression vectors) can be formulated in accordance with standard techniques well known to those skilled in the pharmaceutical art. Such compositions can be administered in dosages and by techniques well known to those skilled in the medical arts taking into consideration such factors as the age, sex, weight, and condition of the particular patient, and the route of administration.

In therapeutic applications, the vaccines are administered to a patient in an amount sufficient to elicit a therapeutic effect, e.g., a CD8⁺, CD4⁺, and/or antibody response to the HIV-1 antigens encoded by the vaccines that at least partially arrests or slows symptoms and/or complications of HIV infection. An amount adequate to accomplish this is defined as “therapeutically effective dose.” Typically, a therapeutically effective dose results in control of virema upon release from ART, i.e., lower levels of viremia after ART cessation compared to viremia observed prior to ART administration. Amounts effective for this use will depend on, e.g., the particular composition of the vaccine regimen administered, the manner of administration, the stage and severity of the disease, the general state of health of the patient, and the judgment of the prescribing physician.

Suitable quantities of DNA vaccine, e.g., plasmid or naked DNA can be about 1 μg to about 100 mg, preferably 0.1 to 10 mg, but lower levels such as 1-10 μg can be employed. For example, an HIV DNA vaccine, e.g., naked DNA or polynucleotide in an aqueous carrier, can be injected into tissue, e.g., intramuscularly or intradermally, in amounts of from 10 μl per site to about 1 ml per site. The concentration of polynucleotide in the formulation is usually from about 0.1 μg/ml to about 20 mg/ml.

The vaccine may be delivered in a physiologically compatible solution such as sterile PBS in a volume of, e.g., one ml. The vaccines may also be lyophilized prior to delivery. As well known to those in the art, the dose may be proportional to weight.

The compositions included in the vaccine regimen can be administered alone, or can be co-administered or sequentially administered with other immunological, antigenic, vaccine, or therapeutic compositions. These include adjuvants, and chemical or biological agent given in combination with, or recombinantly fused to, an antigen to enhance immunogenicity of the antigen. Such other compositions can also include purified antigens from the immunodeficiency virus or a second recombinant vector system that expresses f such antigens and is thus able to produce additional therapeutic compositions. For examples, adjuvant compositions can include expression vectors encoding IL-12 or IL-15 or other biological response modifiers (e.g., cytokines or co-stimulating molecules, further discussed below). Again, co-administration is performed by taking into consideration such known factors as the age, sex, weight, and condition of the particular patient, and, the route of administration.

Compositions that may also be administered with the vaccines include other agents to potentiate or broaden the immune response, e.g., IL-2 or CD40 ligand, which can be administered at specified intervals of time, or continuously administered. For example, IL-2 can be administered in a broad range, e.g., from 10,000 to 1,000,000 or more units. Administration can occur continuously following vaccination.

The vaccines can additionally be complexed with other components such as peptides, polypeptides and carbohydrates for delivery. For example, expression vectors, i.e., nucleic acid vectors that are not contained within a viral particle, can be complexed to particles or beads that can be administered to an individual, for example, using a vaccine gun. Nucleic acid vaccines are administered by methods well known in the art as described in Donnelly et al. (Ann. Rev. Immunol. 15:617-648 (1997)); Felgner et al. (U.S. Pat. No. 5,580,859, issued Dec. 3, 1996); Felgner (U.S. Pat. No. 5,703,055, issued Dec. 30, 1997); and Carson et al. (U.S. Pat. No. 5,679,647, issued Oct. 21, 1997), each of which is incorporated herein by reference. One skilled in the art would know that the choice of a pharmaceutically acceptable carrier, including a physiologically acceptable compound, depends, for example, on the route of administration of the expression vector.

For example, naked DNA or polynucleotide in an aqueous carrier can be injected into tissue, such as muscle, in amounts of from 10 μl per site to about 1 ml per site. The concentration of polynucleotide in the formulation is from about 0.1 μg/ml to about 2 mg/ml.

Vaccines can be delivered via a variety of routes. Typical delivery routes include parenteral administration, e.g., intradermal, intramuscular or subcutaneous routes. Other routes include oral administration, intranasal, and intravaginal routes. In such compositions the nucleic acid vector can be in admixture with a suitable carrier, diluent, or excipient such as sterile water, physiological saline, glucose or the like.

The expression vectors of use for the invention can be delivered to the interstitial spaces of tissues of a patient (see, e.g., Felgner et al., U.S. Pat. Nos. 5,580,859, and 5,703,055). Administration of expression vectors of the invention to muscle is a particularly effective method of administration, including intradermal and subcutaneous injections and transdermal administration. Transdermal administration, such as by iontophoresis, is also an effective method to deliver expression vectors of the invention to muscle. Epidermal administration of expression vectors of the invention can also be employed. Epidermal administration involves mechanically or chemically irritating the outermost layer of epidermis to stimulate an immune response to the irritant (Carson et al., U.S. Pat. No. 5,679,647).

The vaccines can also be formulated for administration via the nasal passages. Formulations suitable for nasal administration, wherein the carrier is a solid, include a coarse powder having a particle size, for example, in the range of about 10 to about 500 microns which is administered in the manner in which snuff is taken, i.e., by rapid inhalation through the nasal passage from a container of the powder held close up to the nose. Suitable formulations wherein the carrier is a liquid for administration as, for example, nasal spray, nasal drops, or by aerosol administration by nebulizer, include aqueous or oily solutions of the active ingredient. For further discussions of nasal administration of AIDS-related vaccines, references are made to the following patents, U.S. Pat. Nos. 5,846,978, 5,663,169, 5,578,597, 5,502,060, 5,476,874, 5,413,999, 5,308,854, 5,192,668, and 5,187,074.

The vaccines can be incorporated, if desired, into liposomes, microspheres or other polymer matrices (see, e.g., Felgner et al., U.S. Pat. No. 5,703,055; Gregoriadis, Liposome Technology, Vols. I to III (2nd ed. 1993). Liposomes, for example, which consist of phospholipids or other lipids, are nontoxic, physiologically acceptable and metabolizable carriers that are relatively simple to make and administer. Liposomes include emulsions, foams, micelles, insoluble monolayers, liquid crystals, phospholipid dispersions, lamellar layers and the like.

Liposome carriers can serve to target a particular tissue or infected cells, as well as increase the half-life of the vaccine. In these preparations the vaccine to be delivered is incorporated as part of a liposome, alone or in conjunction with a molecule which binds to, e.g., a receptor prevalent among lymphoid cells, such as monoclonal antibodies which bind to the CD45 antigen, or with other therapeutic or immunogenic compositions. Thus, liposomes either filled or decorated with a desired immunogen of the invention can be directed to the site of lymphoid cells, where the liposomes then deliver the immunogen(s).

Liposomes for use in the invention are formed from standard vesicle-forming lipids, which generally include neutral and negatively charged phospholipids and a sterol, such as cholesterol. The selection of lipids is generally guided by consideration of, e.g., liposome size, acid lability and stability of the liposomes in the blood stream. A variety of methods are available for preparing liposomes, as described in, e.g., Szoka, et al., Ann. Rev. Biophys. Bioeng. 9:467 (1980), U.S. Pat. Nos. 4,235,871, 4,501,728, 4,837,028, and 5,019,369.

### EXAMPLES

### Example

### Administration of DNA Vaccines to ART-Treated Macaques Controls Viremia upon Release from ART

The following example shows the ability of DNA vaccination during antiretroviral therapy to decrease virus replication in macaques chronically infected with highly pathogenic SIVmac251. In this example, animals were treated with a combination of three drugs and vaccinated with combinations of vectors expressing SIV antigens. Vaccinated animals showed a boost in cellular immune responses. After release from therapy, the virus load and immune response of the immunized animals were compared to animals treated only with ART. The mean viral load for the 10 weeks before ART was compared to the mean virus load for the 13 weeks following ART termination. Vaccinated animals showed significant drops in viremia and persistence of cellular immune responses at high levels compared to controls, indicating a benefit from DNA therapeutic vaccination. The vaccine regimen and results were performed and analyzed as follows.

Thirty one Indian rhesus macaques (Macaca mulatta) in four groups were studied. All Rhesus macaques were infected with pathogenic SIVmac251 via the mucosal route. These groups were:

Group 1 (group v1), (n=9) previously naïve, infected animals received DNA vaccine during ART.

Group 2 (group v2), (n=6) previously vaccinated, infected animals also received DNA vaccine during ART.

Group 3 (group c1), (n=12) previously naïve infected animals received ART only.

Group 4, (group c2) (n=4) previously vaccinated, infected animals received ART only.

Animals in groups 1 and 3 were previously naïve, infected with SIVmac251. Animals in groups 2 and 4 were previously vaccinated with SIV DNA vectors, infected by SIVmac251 as part of another study and recycled for this immunotherapy study. Animals had been infected for period varying from 15 to 70 weeks prior to the start of antiretroviral treatment (ART). Animals were treated with a combination of three antiretroviral drugs effective against SIVmac (PMPA, stavudine, ddI) for approximately 20 weeks. Drug dosage was as follows: PMPA, 20 mg/kg SC SID; ddI, 5 mg/kg IV SID; Stavudine, 1.2 mg/kg PO BID.

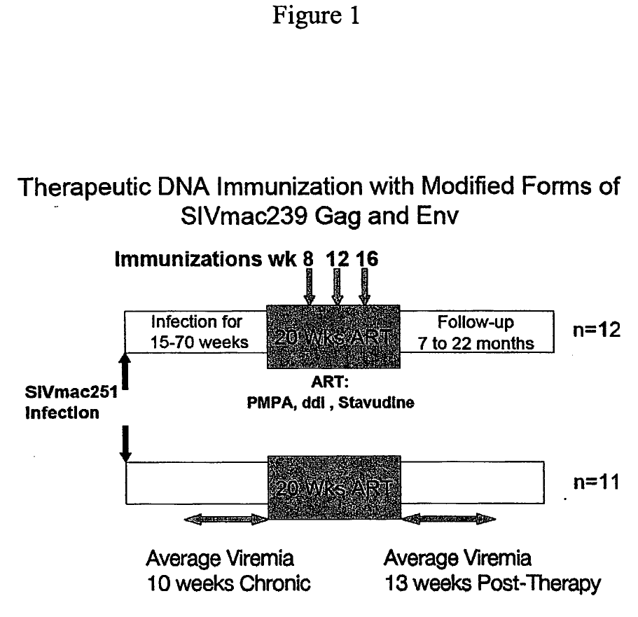

The animals in groups 1 and 2 received in addition 3 or four DNA vaccinations, usually at week 8, 12, and 16 of treatment, as indicated in FIG. 1. These vaccinations consisted of combinations of optimized expression vectors for SIV antigens, including antigens which are further modified for efficient secretion and uptake by antigen presenting cells (antigen fusions to MCP3 chemokine) or modified for more efficient intracellular degradation (antigen fusions to a Catenin peptide, CATE).

Animals were vaccinated via the intramuscular route with a total of 8 mg of plasmids. DNAs were injected separately or in groups in PBS in several different sites. Animals 56 and 57 (group 1), and 920, 922, 923, 628 (group 2) received together with the SIV DNAs 2 mg of an IL-15 producing plasmid in citrate buffer containing bupivacaine. Animals 926 and 626 (group 2) received together with the SIV DNAs 2 mg of an IL-12 producing plasmid in citrate buffer containing bupivacaine. The bioactive IL-12 or IL-15 produced by these plasmids was included as a molecular adjuvant in an effort to further enhance the effects of DNA vaccination.

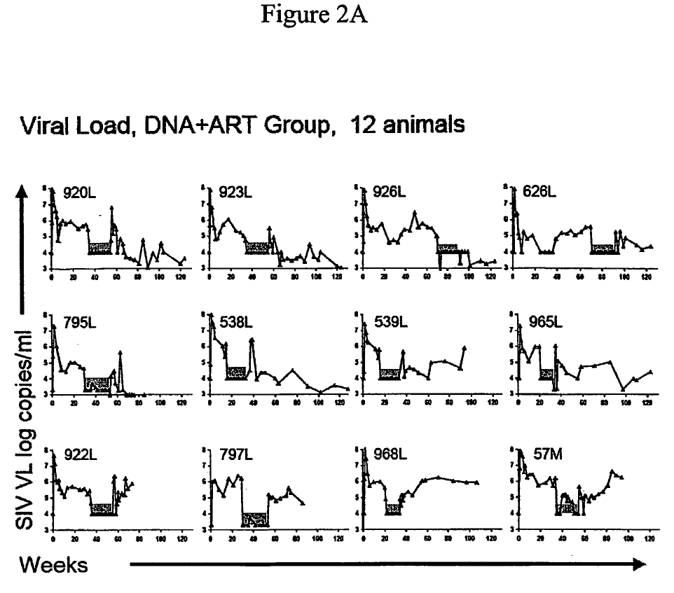

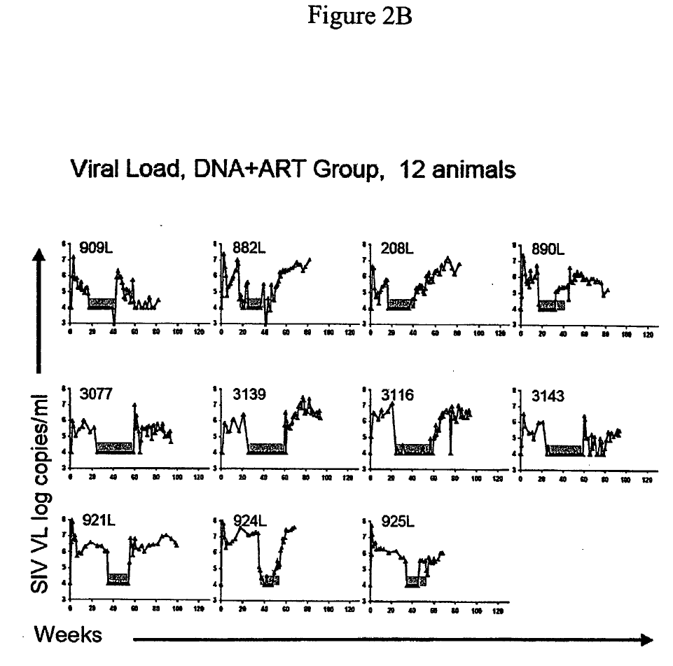

The animals were treated in smaller groups over a period of 3 years, as they became available from other studies. Of the 31 treated animals, eight were excluded from the primary statistical analysis. Five of these animals (3 in the vaccine group, 2 controls) were excluded because they did not control virus for at least ⅓ of the period during ART. The remaining three animals were excluded because they had undetectable viremia before ART initiation. The primary statistical analysis described herein was therefore performed in 23 animals, of which 12 received ART plus vaccination during therapy, and 11 received only ART and were used as the control group (Table 1, FIG. 2).

Table 1 shows a list of the animals indicating the length of time of infection (median=24 weeks), ART treatment (median=20 weeks) and post-ART follow-up period (median=40 weeks), the types and amounts of DNA used, the number of immunizations and the animal haplotypes. All animals showed a benefit during ART by decreasing virus load to below the cut-off value for the assay for at least ⅓ of the time during ART. Animals were kept in ART for at least 20 weeks, except for some animals that showed signals of drug toxicities, for which ART was terminated earlier (965, 968, 926, 626). The animals were studied during and after ART by measuring viral loads in plasma and anti-SIV responses by Elispot and antibody assays. Viral load in plasma was monitored by analysis of RNA as described (Romano, et al., J. Virol. Methods 86:61-70, 2000; Suryanarayana, et al., AIDS Res Hum Retroviruses 14:183-189, 1998).

TABLE 1
History and treatment of the animals in the immunotherapy study.

| | | | | | post- | | | total | | |
|---|---|---|---|---|---|---|---|---|---|---|
| | | prior | infection | | ART | DNA vectors | | amount of | time of | |
| group | | prophylactic | till ART, | ART, | followup | used, | Cytokine | DNA, | immunization, | |
| # | animal# | vaccination | weeks | weeks | weeks | SIVmac239 | DNA | mg/animal | weeks in ART | HAPLOTYPE |
| v1 | 795L | | 29 | 23 | 33 | gag, env | | 7.5 | 8, 10, 13, 17 | A01-A11-B017 |
| v1 | 797L | | 29 | 23 | 34 | gag, env | | 7.5 | 8, 10, 13, 17 | A01-A02-B01-w201 |
| v1 | 538L | | 15 | 20 | 93 | gag, env, RTNV | | 10 | 2, 6, 10, 14 | A01-B01 |
| v1 | 539L | | 15 | 20 | 59 | gag, env, RTNV | | 10 | 2, 6, 10, 14 | A08-B03-w201 |
| v1 | 965L | | 20 | 13 | 90 | gag, env, RTNV | | 10 | 2, 6, 10 | A11-B01 |
| v1 | 968L | | 20 | 14 | 74 | gag, env, RTNV | | 10 | 2, 6, 10, 14 | B01 |
| v1 | 57M | | 34 | 20 | 40 | gag, env, poINTV | IL-15 | 10 | 9, 13, 17 | A11-B01-B03-B17 |
| v2 | 920L | Y | 34 | 20 | 70 | gag, env, poINTV | IL-15 | 10 | 9, 13, 17 | A02-A11-w201 |
| v2 | 923L | Y | 34 | 20 | 70 | gag, env, poINTV | IL-15 | 10 | 9, 13, 17 | B03-B17-w201-0401/06 |
| v2 | 922L | Y | 34 | 20 | 19 | gag, env, poINTV | IL-15 | 10 | 9, 13, 17 | w201 |
| v2 | 926L | Y | 70 | 19 | 35 | gag, env, poINTV | IL-12 | 10.1 | 8, 12, 16 | A02-B17-w201 |
| v2 | 626 | Y | 70 | 19 | 35 | gag, env, poINTV | IL-12 | 10.1 | 8, 12, 16 | A01-A08 |
| c1 | 882L | | 16 | 25 | 41 | | | | | * |
| c1 | 890L | | 16 | 25 | 49 | | | | | * |
| c1 | 909L | | 16 | 25 | 49 | | | | | * |
| c1 | 208M | | 16 | 25 | 49 | | | | | * |
| c1 | 3077 | | 24 | 34 | 36 | | | | | * |
| c1 | 3139 | | 24 | 34 | 36 | | | | | * |
| c1 | 3116 | | 24 | 34 | 36 | | | | | * |
| c1 | 3143 | | 24 | 34 | 36 | | | | | * |
| c2 | 921L | Y | 34 | 20 | 45 | | | | | A01-0401/06 |
| c2 | 924L | Y | 34 | 20 | 14 | | | | | w201 |
| c2 | 925L | Y | 34 | 20 | 14 | | | | | neg |
| | | | 24 | 20 | 40 | (=median) | | | | |

Stars indicate animals known to be negative for MamuA*01.
neg, negative for all examined haplotypes.

FIG. 2 shows the measurements of virus loads in plasma from initial infection to the end of follow-up period for all animals. During ART, an assay with a cutoff value of 20,000 RNA copies/ml was used, and the values below the cutoff were assigned the value of 10,000. Most of the samples below cutoff during the other periods were analyzed, if available in sufficient quantity, by more sensitive assays having cutoff values of 2,000 and 100 RNA copies/ml of plasma. After release from therapy, virus rebound rapidly in the majority of the animals. The vaccinated animals (FIG. 2A) showed evidence of virus suppression, since the virus decreased dramatically few weeks after ART termination, despite initial rebound(s). Seven of the 12 vaccinated animals showed significant long-term benefit in the levels of viremia; five of these suppressed virus at levels close to or below detection level for several months. In contrast, virus loads in most of the control animals returned to levels similar to those prior to therapy (FIG. 2B). The inability of ART alone to induce long-lasting benefits in virus load seen in this study is in agreement with the experience of other investigators in macaques and also with the results in humans, where therapy termination results in general in virus rebound at levels similar to the chronic state of viremia prior to ART.

For statistical comparisons, the (log 10 transformed) average viremia during the 10 weeks immediately preceding ART and during the first 13 weeks of follow-up, available for all animals in the study, was determined. The change in average viremia was used as a measure of the effects of vaccination.

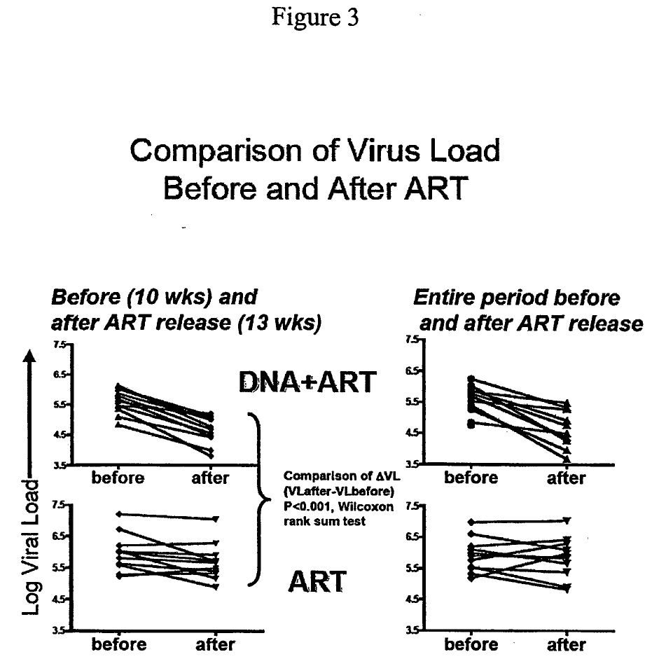

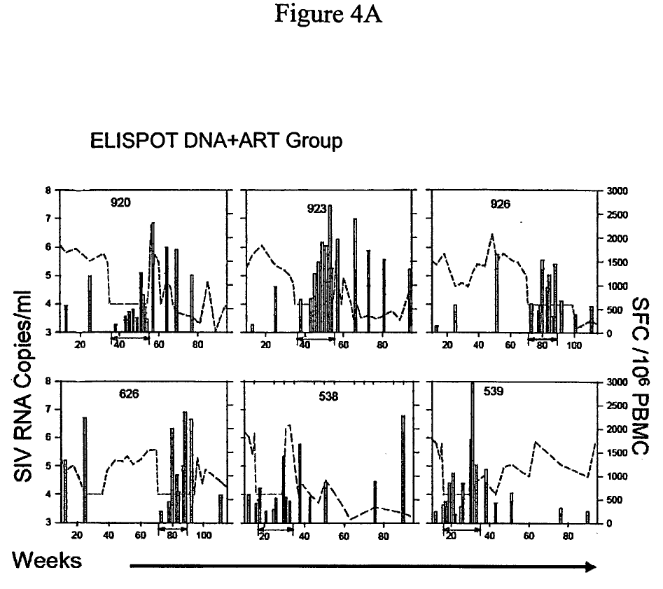

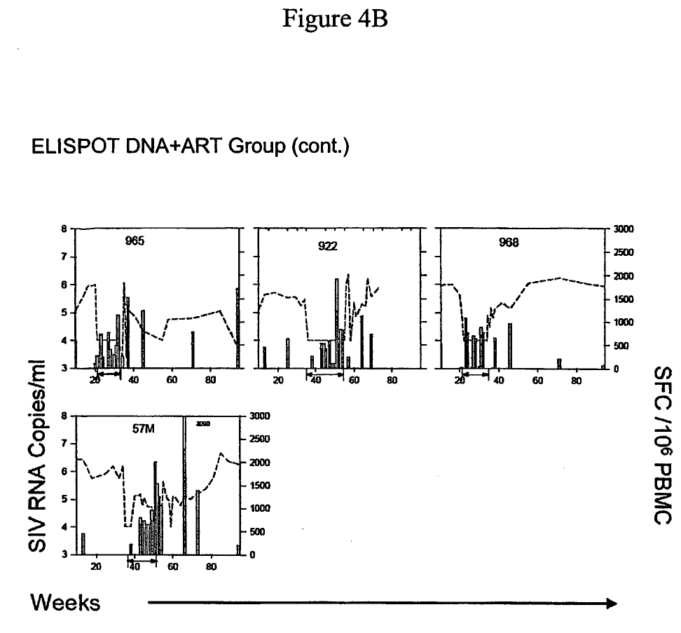

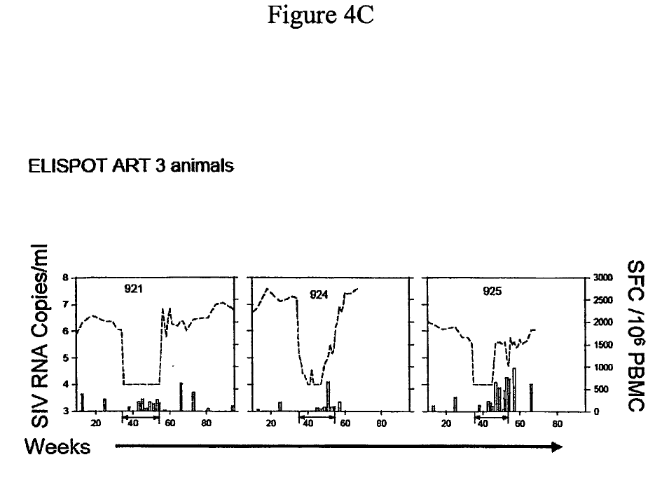

The comparison of the change in viremia for the vaccine and control groups is shown in FIG. 3. All animals in the vaccine group showed lower average viremia after ART release, compared to the chronic phase. The mean difference in the log-base 10 transformed virus load measurements for each animal (mean VL after ART minus mean VL before ART) was −0.93 for the combined vaccination group and −0.28 for the combined control group (FIG. 4). The difference was highly statistically significant across the two groups (P=0.001 with a Wilcoxon rank sum test).

Five of the animals in the vaccine group (see Table 1, animals 920, 922, 923, 926 and 626) and three in the control group (animals 921, 924 and 925) were prophylactically vaccinated with SIV gag and env DNA vectors before SIV infection, as part of previous studies. To analyze any effects of the prophylactic DNA vaccination on immunotherapy outcome, the previously vaccinated animals in the vaccine and control groups were compared to the rest of the animals in their corresponding group. An interaction between the previous vaccination and only therapeutic vaccination was test for using 2-way analysis of variance. There was no evidence for interaction (P=0.97), suggesting that the benefit derived by therapeutic vaccination is not affected by previous prophylactic vaccination. Therefore, combining the previously vaccinated animals in the two groups of therapeutically vaccinated and controls, was appropriate. In addition, if only the animals without any previous treatment or prophylactic vaccination (7 vaccines and 8 controls) are considered, the results are also significant, indicating that therapeutic vaccination provides a benefit.

It is evident from FIG. 2 that several animals had initial rebounds of virus after ART release, followed by periods of decreased viral loads. This subsequent decrease could indicate attempts of the immune system to control the virus. Therefore, the concern was that comparisons of viremia for relatively short periods of time may misrepresent the long-term effects of immunotherapy. On the other hand, some previous work has suggested that the benefits of immunotherapy may be transient. To study this, additional analyses including the longer follow-up available for these animals were performed. The differences in virus load using the entire chronic and release period on all 23 animals (FIG. 3, Right) was evaluated. In this analysis, each animal has a different follow-up time as indicated in FIG. 2. In this comparison, the mean difference in virus load was −1.05 log-base 10 for the combined vaccination group and −0.068 for the combined control group. This difference was statistically significant (P=0.0004 with a Wilcoxon rank sum test). Control of viremia for long periods of time after an initial virus rebound immediately following ART termination explains the bigger difference found upon analyzing the entire available periods of chronic SIV infection and post-ART for all animals.

### Immunological Analysis

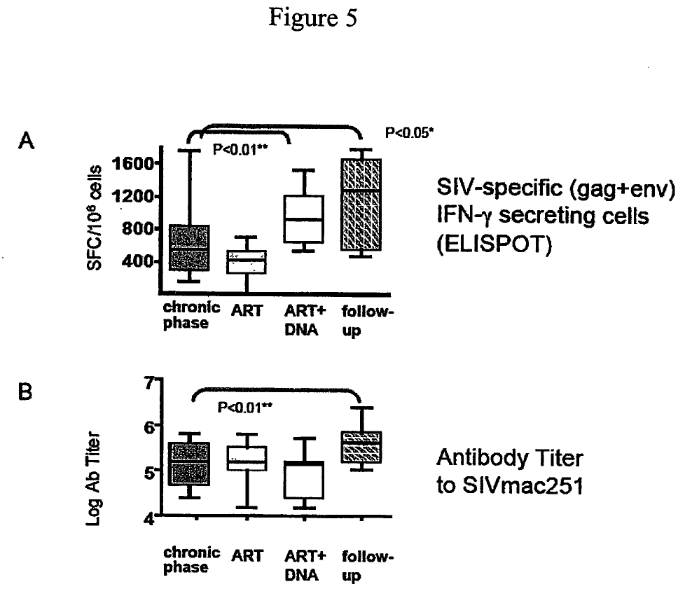

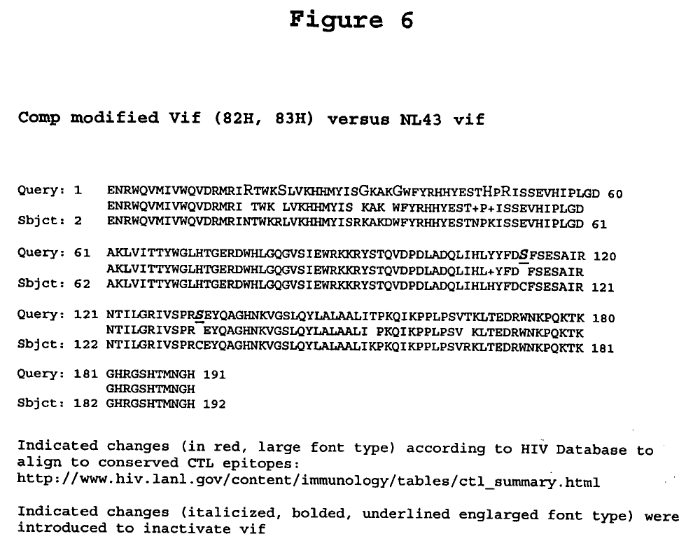

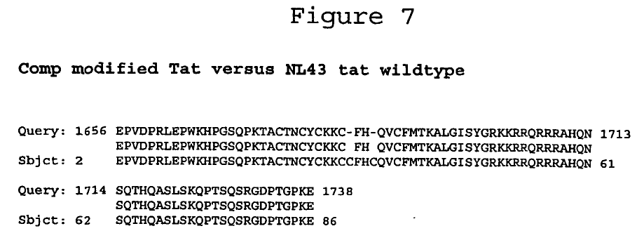

Immunological analysis was performed for 10/12 ART+DNA animals and 3/11 ART animals. This analysis showed induction of cellular and humoral immune responses after DNA vaccination. IFN-gamma production from PBMC stimulated by overlapping peptide pools (15 mers overlapping by 11) for gag and gp120env (FIG. 4) was measured. FIG. 5A shows the ELISPOT response to gag and env for 10 vaccinated animals, shown as median and quartiles, divided into 4 periods, chronic phase, ART before vaccination, ART and DNA vaccination, and follow-up after drug termination. ELISPOT numbers decrease immediately upon drug treatment, as expected from the low virus load, and immediately increase upon vaccination. Antibodies against SIV proteins were measured by Elisa. The animals had high antibody levels against SIV (reciprocal titers 10⁵-10⁶). Ab levels were not increased during vaccination, were slightly decreased during ART, whereas after ART termination the antibody levels were increased to higher levels (FIG. 5B).

The mean and peak Elispot values for gag were compared using a Wilcoxon signed rank test during the first period of ART treatment prior to, and the period during therapeutic vaccination. There was an overall increase during therapeutic vaccination (median difference=255.8, 1^{st} quartile=115.7 and 3^{rd} quartile: 479.5); P-value=0.001. Similar trends were detected using peak measurements (P=0.001).

### Animals Receiving DNA Vectors Expressing in Addition IL-12 or IL-15

As shown in Table 1, some animals in this study received DNA vectors expressing biologically active macaque IL-12 or IL-15. This showed that the DNA vectors for these cytokines were safe for animals infected with SIV, since no adverse effects were observed. This is similar to the conclusions obtained in non-SIV infected animals, including neonate macaques. The levels of Elispot responses for the animals receiving IL-15 were similar. Comparison of the decrease in viremia for the animals receiving IL-15 DNA versus the animals that did not, showed no statistical differences (P=0.64 and P=0.79 for mean and peak gag responses, respectively). Since defects in IL-12 and IL-15 have been shown in HIV infected people, inclusion of IL-12 or IL-15 can be beneficial when used in therapeutic vaccination procedures.

The differences in virus load of all 31 treated animals without excluding any animal that completed the ART period, using the entire chronic and release period, was also analyzed. As in the analysis performed with the 23 animals, supra, there is no interaction between previous vaccination and just immunotherapy, allowing the combination of animals in two groups. The mean difference for vaccine was 0.97 and for the control group 0.26. The difference between groups was highly significant (P=0.002) using Wilcoxon rank sum test (data not shown).

For the above comparisons conducted ANCOVA (analysis of covariance) was also conducted adjusting for differences in chronic viral load between the groups. For all three analyses above of the 23 as well as the 31 animals, the vaccine group was different from control after adjusting for average log transformed chronic VL levels (P<0.001 for all analyses).

To verify that vaccination previous to SIV infection and enrollment in the exemplary therapeutic vaccination protocol described in this example did not affect the outcome of the study, an additional comparison excluding all previously vaccinated animals was conducted. Even upon exclusion of all animals that were vaccinated as part of previous studies before SIV infection and comparison of the 7 remaining vaccines (mean Difference in log 10 Virus Load (DVL)=1.10) to the naïve group (mean DVL=−0.07), the results were significant (P=0.002, using Wilcoxon rank sum test, data not shown).

Therefore, we conclude that DNA vaccination during ART resulted in virus control after release from ART for prolonged periods of time (months). The majority of the animals appear to benefit from this immunization, and the average benefit is estimated between 0.65 and 1 log 10decrease in virus load compared to the control group.

A number of alternative statistical analyses were run to verify that these results are not affected by treatment variations or exclusion criteria. These included additional viral load analyses using ANCOVA: For Area Under Curve (AUC) analyses: we compared differences in the standardized AUC (log scale) between chronic and release periods. These analyses were done using complete follow-up on each animal. For 23 animal analysis, we found highly significant differences between vaccinated and non-vaccinated animals (P=0.003). Also significant differences using 31 animals (P=0.007).

### SUMMARY

In summary, all the analyses show that, relative to the SIV infection period, post-therapy viral load is substantially lower in therapeutically DNA vaccinated animals compared with un-vaccinated animals. Chronically infected animals, unable to control viremia on their own, do so upon ART and DNA vaccination. A number of animals were able to fully suppress viremia close to the detection limits of the assay. These included both previously prophylactically vaccinated as well as naïve animals. ART alone did not give any evidence of permanent virus decrease, in agreement with data from several studies on Therapy Interruption in monkeys and humans.

The animals that were studied were of diverse background as shown by the haplotype data (Table 1) and were unable to suppress virus replication prior to treatment. The data presented herein above suggested that ART alone was not able to produce a lasting decrease in chronic virus loads after release, in agreement with other studies. The decrease in virus load seen in vaccinated animals suggests that ART and vaccination had an important positive effect on the immune system. Interestingly, the virus rebounds upon termination of ART, and it is further suppressed after some weeks, presumably by the immune system. In agreement with this, the cellular immune responses measured by ELISPOT agree with the notion that virus rebound leads to increased CTL activity and elimination of the infected cells. In several animals showing low virus loads high Elispot numbers against gag and env proteins were maintained. This is in contrast to the expected decrease in the level of immune responses upon a decrease in viremia, and suggests that the immune system of the therapeutically immunized animals has reached a different steady state. This observation is reflected in the negative correlation of viral load with Elispot values seen during the release period.

Not to be bound by theory, it may be hypothesized that the previously prophylactic vaccinated animals have a healthier immune system and could respond to the therapeutic vaccination more effectively than non-vaccinated animals. The analysis described in this example failed to show any significant difference between the two groups. Analysis of the animals that did not receive any vaccination prior to SIVmac251 infection (7 vaccines and 8 controls) resulted in the same conclusion, i.e., the vaccines showed a statistically significant drop in viremia compared to the controls. Therefore, the benefit of immunotherapy did not depend on previous prophylactic vaccination.

### Exemplary Constructs of the Invention:

“Gag” refers to DNA sequences encoding the Gag protein, which generates components of the virion core; “Pro” denotes “protease”. The protease, reverse transcriptase, and integrase genes comprise the “pol” gene.

“MCP3” in these constructs denotes MCP-3 amino acids 33-109 linked to IP-10 secretory peptide (alternatively, it can be linked to its own natural secretory peptide or any other functional secretory signal, e.g., the tissue plasminogen activator (tPA) signal peptide; “CATE” denotes β-catenin aino acids 18-47.

### Construction of Vectors Encoding Fusion Proteins Comprising Destabilizing Sequences:

In order to design “Gag-destabilized” constructs, a literature search for characterized sequences able to target proteins to the ubiquitin-proteasome degradation pathway gave the following, not necessarily representative, list:

c-Myc aa 2-120

### Cyclin A aa 13-91

Cyclin B aa 13-91 (*10-95 in vectors in examples herein)
IkBα aa20-45
β-Catenin aa 19-44 (aa18-47 in vectors in examples herein)
c-Jun aa 1-67
c-Mos aa 1-35

Exemplary 30 aa of β-catenin destabilization sequence (amino acids 18-47):

RKAAVSHWQQQSYLDSGTHSGATTTAPSLS

β-catenin (18-47) added at the N terminus of HIV antigens with initiator AUG Met:

MRKAAVSHWQQQSYLDSGIHSGATTTAPSLS

In some embodiments, the gag p37 and p55 plasmids may have the same p37 and p55 gag sequences disclosed in the patents containing INS-gag sequences (see, e.g., U.S. Pat. No. 5,972,596 and U.S. Pat. No. 5,965,726).

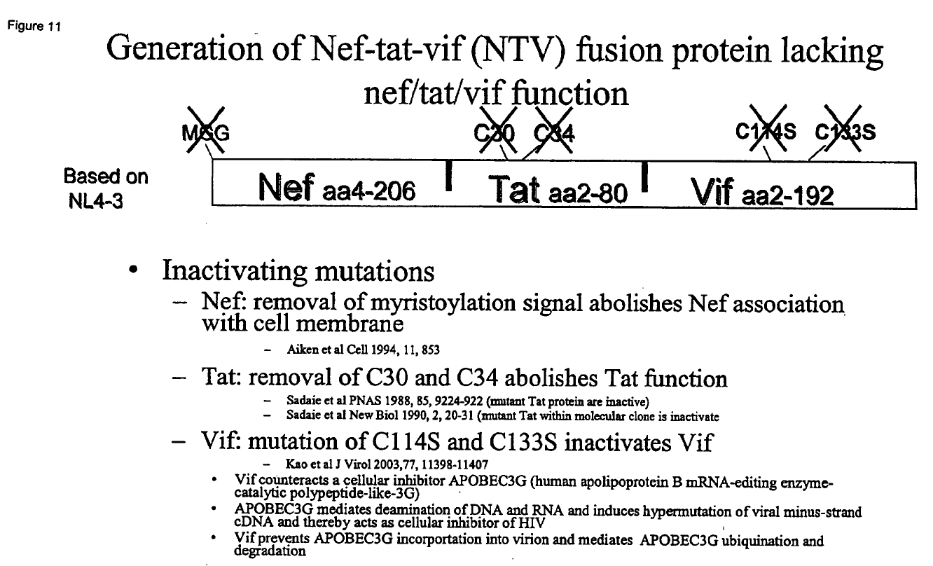

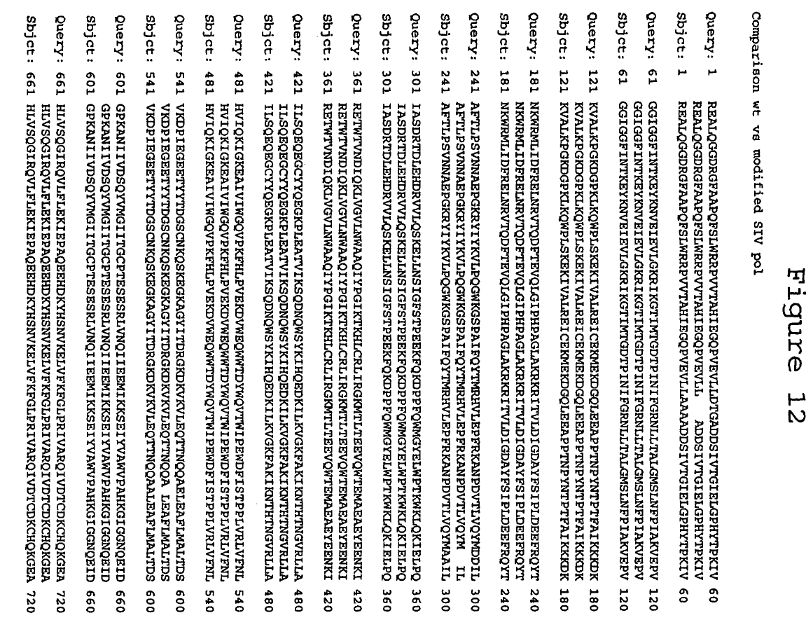

Exemplary SIV constructs are provided below. All plasmids have CMV promoter and BGH poly adenylation signal, the kan resistant gene for growth in E. coli. The pol genes (protease, RT, int) are mutated to render them inactive. SIV inactivating mutations were analagous to the mutations in HIV pol set forth in FIG. 11. A comparison of wt vs. modified SIV pol is provided in FIG. 14.

Plasmid pSIVgagDX:
lower case, underlined: CMV promoter;
italics: BGH polyadenylation signal
Gag gene: 770-2302
(1)cctggccattgcatacgttgtatccatatcataatatgtacatttatattggctcatgtcca
acattaccgccatgttgacattgattattgactagttattaatagtaatcaatacggggtcatta
gttcatagcccatatatggagttccgcgttacataacttacggtaaatggcccgcctggctgacc
gcccaacgacccccgcccattgacgtcaataatgacgtatgttcccatagtaacgccaataggga
ctttccattgacgtcaatgggtggagtatttacggtaaactgcccacttggcagtacatcaagtg
tatcatatgccaagtacgccccctattgacgtcaatgacggtaaatggcccgcctggcattatgc
ccagtacatgaccttatgggactttcctacttggcagtacatctacgtattagtcatcgctatta
ccatggtgatgcggttttggcagtacatcaatgggcgtggatagcggtttgactcacggggattt
ccfaagtccaccccattgacgtcaatgggagtttgtttggcaccaaaatcaacgggactttccaa
aatgtcgtaacaactccgccccattgacgcaaatgggcggtaggcgtgtacggtgggaggtctat
ataagcagagctcgtttagtgaaccgtcagatcgcctggagacgccatccacgctgttttgacct
ccatagaagacaccgggaccgatccagcctccgcgggcgcgCGTCGACAGAGAGATGGGCGTGAG
AAACTCCGTCTTGTCAGGGAAGAAAGCAGATGAATTAGAAAAAATTAGGCTACGACCCTAACGGA
AAGAAAAAGTACATGTTGAAGCATGTAGTATGGGCAGCAAATGAATTAGATAGATTTGGATTAGC
AGAAAGCCTGTTGGAGAACAAAGAAGGATGTCAAAAAATACTTTCGGTCTTAGCTCCATTAGTGC
CAACAGGCTCAGAAAATTTAAAAAGCCTTTATAATACTGTCTGCGTCATCTGGTGCATTCACGCA
GAAGAGAAAGTGAAACACACTGAGGAAGCAAAACAGATAGTGCAGAGACACCTAGTGGTGGATAA
CAGGAACCACCGAAACCATGCCGAAGACCTCTCGACCAACAGCACCATCTAGCGGCAGAGGAGGA
AACTACCCAGTACAGCAGATCGGTGGCAACTACGTCCACCTGCCACTGTCCCCGAGAACCCTGAA
CGCTTGGGTCAAGCTGATCGAGGAGAAGAAGTTCGGAGCAGAAGTAGTGCCAGGATTCCAGGCAC
TGTCAGAAGGTTGCACCCCCTACGACATCAACCAGATGCTGAACTGCGTTGGAGACCATCAGGCG
GCTATGCAGATCATCCGTGACATCATCAACGAGGAGGCTGCAGATTGGGACTTGCAGCACCCACA
ACCAGCTCCACAACAAGGACAACTTAGGGAGCCGTCAGGATCAGACATCGCAGGAACCACCTCCT
CAGTTGACGAACAGATCCAGTGGATGTACCGTCAGCAGAACCCGATCCCAGTAGGCAACATCTAC
CGTCGATGGATCCAGCTGGGTCTGCAGAAATGCGTCCGTATGTACAACCCGACCAACATTCTAGA
TGTAAAACAAGGGCCAAAAGAGCCATTTCAGAGCTATGTAGACAGGTTCTACAAAAGTTTAAGAG
CAGAACAGACAGATGCAGCAGTAAAGAATTGGATGACTCAAACACTGCTGATTCAAAATGCTAAC
CCAGATTGCAAGCTAGTGCTGAAGGGGCTGGGTGTGAATCCCACCCTAGAAGAAATGCTGACGGC
TTGTCAAGGAGTAGGGGGGCCGGGACAGAAGGCTAGATTAATGGCAGAAGCCCTGAAAGAGGCCC
TCGCACCAGTGCCAATCCCTTTTGCAGCAGCCCAACAGAGGGGACCAAGAAAGCCAATTAAGTGT
TGGAATTGTGGGAAAGAGGGACACTCTGCAAGGCAATGCAGAGCCCCAAGAAGACAGGGATGCTG
GAAATGTGGAAAAATGGACCATGTTATGGCCAAATGCCCAGACAGACAGGCGGGTTTTTTAGGCC
TTGGTCCATGGGGAAAGAAGCCCCGCAATTTCCCCATGGCTCAAGTGCATCAGGGGCTGATGCCA
ACTGCTCCCCCAGAGGACCCAGCTGTGGATCTGCTAAAGAACTACATGCAGTTGGGCAAGCAGCA
GAGAGAAAAGCAGAGAGAAAGCAGAGAGAAGCCTTACAAGGAGGTGACAGAGGATTTGCTGCACC
TCAATTCTCTCTTTGGAGGAGACCAGTAGGAATCGAGCTCGGTACGATCCACCCCTCCCCCGTGC
CTTCCTTGACCCTGGAAGGTGCCACTCCCACTGTCCTTTCCTAATAAAATGAGGAAATTGCATCG
CATTGTCTGAGTAGGTGTCATTCTATTCTGGGGGGTGGGGTGGGGCAGCACAGCAAGGGGGAGGA
TTGGGAAGACAATAGCAGGCATGCTGGGGATGCGGTGGGCTCTATGGGTACCCAGGTGCTGAAGA
ATTGACCCGGTTCCTCCTGGGCCAGAAAGAAGCAGGCACATCCCCTTCTCTGTGACACACCCTGT
CCACGCCCCTGGTTCTTAGTTCCAGCCCCACTCATAGGACACTCATAGCTCAGGAGGGCTCCGCC
TTCAATCCCACCCGCTAAAGTACTTGGAGCGGTCTCTCCCTCCCTCATCAGCCCACCAAACCAAA
CCTAGCCTCCAAGAGTGGGAAGAAATTAAAGCAAGATAGGCTATTAAGTGCAGAGGGAGAGAAAA
TGCCTCCAACATGTGAGGAAGTAATGAGAGAAATCATAGAATTTCTTCCGCTTCCTCGCTCACTG
ACTCGCTGCGCTCGGTCGTTCGGCTGCGGCGAGCGGTATCAGCTCACTCAAAGGCGGTAATACGG
TTATCCACAGAATCAGGGGATAACGCAGGAAAGAACATGTGAGCAAAAGGCCAGCAAAAGGCCAG
GAACCGTAAAAAGGCCGCGTTGCTGGCGTTTTTCCATAGGCTCCGCCCCCCTGACGAGCATCACA
AAAATCGACGCTCAAGTCAGAGGTGGCGAAACCCGACAGGACTATAAAGATACCAGGCGTTTCCC
CCTGGAAGCTCCCTCGTGCGCTCTCCTGTTCCGACCCTGCCGCTTACCGGATACCTGTCCGCCTT
TCTCCCTTCGGGAAGCGTGGCGCTTTCTCAATGCTCACGCTGTAGGTATCTCAGTTCGGTGTAGG
TCGTTCGCTCCAALGCTGGGCTGTGTGCACGAACCCCCCGTTCAGCCCGACCGCTGCGCCTTATC
CGGTAACTATCGTCTTGAGTCCAACCCGGTAAGACACGACTTATCGCCACTGGCAGCAGCCACTG
GTAACAGGATTAGCAGAGCGAGGTATGTAGGCGGTGCTACAGAGTTCTTGIAAGTGGTGGCCTAA
CTACGGCTACACTAGAAGGACAGTATTTGGTATCTGCGCTCTGCTGJAAGCCAGTTACCTTCGGA
AAAAGAGTTGGTAGCTCTTGATCCGGCAAACAAAAAAACCACCGCTGGTAGCGGTGGTTTTTTTG
TTTGCAAGCAGCAGATTACGCGCAGAAAJAAAGGATCTCAAAGAAGATCCTTTGATCTTTTCTAC
GGGGTCTGACGCTCAGTGGAACGAAAAACTCACGTTAAGGGATTTTGGTCATGAGATTATCAAAG
GATCTTCACCTAGATCCTTTTAAAATTAAAAATGAAGTTTTAATCAATCTAAAGTATATATGAGT
AAAACTTGGTCTGACAGTTACCAATGCTTAATCAGTGAGGCACCTATCTCAGCGATCTGTCTATT
TCGTTCATCCATAGTTGCCTGACTCCGGGGGGGGGGGGCGCTGAGGTCTGCCTCGTGAAGAAGGT
GTTGCTGACTCATACCAGGCCTGAATTAATCGCCCCATCATCCAGCCAGAAAGTGAGGGAGCCAC
GGTTGATGAGAGCTTTGTTGTAGGTGGACCAGTTGGTGATTTTGAACTTTTGCTTTGCCACGGAA
CGGTCTGCGTTGTCGGGAAGATGCGTGATCTGATCCTTCAACTCAGCAAGTTCGATTTATTCAAC
AAAAAGCCGCCGTCCCGTCAAGCTCATCGAGCATCAAATGAAACTGCAATTTATTCATATCAGGA
TTATCAATACCATATTTTTGAAAGCCGTTTCTGTAAATGAAAGGAGAAAAAACTCACCGAGGCAG
TTCCATAGGATGGCAAGATCCTGGTATCGGTCTGCGATTCCGACTCGTCCAACATCAAATACAAC
CTATTAATTTCCCCTCGTCAAAAATAAGGTTATCAAGTGAGAAAATCACCATGAGTGACGACTGA
ATCCGGTGAGAAAAAATGGCAAAAGCTTATGCATTTCTTTCCAGACTTGTTCAACAGGCCAGCCA
TTACGCTCGTCATCAATCACTCGCATCAACCAAACCGTTATTCATTCGTGATTGCGCCTGAGCGA
GACGAAAAAAATACGCGATCGCTGTTAAAAGGACAATTACAAACAGGAATCGAATGCAACCGGCG
CAGGAACACTGCCAGGTTTTCCCGGGGATCGCAGTGGTGAGTAACCATGCATCATCAGGAGTACG
GATAAATGCTTGATGGTCGGAAGAGGCATAAATTCCGTCAGCCAGTTTAGTCTGACCATCTCATC
TGTAACATCATTGGCAACGCTACCTTTGCCATGTTTCAGAAACAACTCTGGCGCATCGGGCTTCC
CATACAATCGATAGATTGTCGCACCTGATTGCCCGACATTATCGCGAGCCCATTTATACCCATAT
TCAGCATCCATGTTGGAATTTAATCGCGGCCTCGAGCAAGACGTTTCCCGTTGAATATGGCTCAT
AACACCCCTTGTATTACTGTTTATGTAAGCAGACAGTTTTATTGTTCATGATGATATATTTTTAT
CTTGTGCAATGTAACATCAGAGATTTTGAGACACAACGTGGCTTTCCCCCCCCCCCCATTATTGA
AGCATTTATCAGGGTTATTGTCTCATGAGCGGATACATATTTGAATGTATTTAGAAAAATAAACA
AATAGGGGTTCCGCGCACATTTCCCCGAAA&AAGTGCCACCTGACGTCTAAGAAACCATTATTAT
CATGACATTAACCTATAAAAATAGGCGTATCACGAGGCCCTTTCGTCTCGCGCGTTTCGGTGATG
ACGGTGAAAAAACCTCTGACACATGCAGCTCCCGGAGACGGTCACAGCTTGTCTGTAAAAGCGGA
TGCCGGGAGCAGACAAGCCCGTCAGGGCGCGTCAGCGGGTGTTGGCGGGTGTCGGGGCTGGCTTA
ACTATGCGGCATCAGAGCAGATTGTACTGAGAGTGCACCATATGCGGTGTGAAAATACCGCACAG
ATGCGTAAGGAGAAAATACCGCATCAGATTGGCTATTGG (5558)
Protein SIV p57gag
M G V R N S V L S G K K A D E L E K R L R A N G K K K Y M L K H V
V W A A N E L D R F G L A E S L L E N K E G C Q K L S V L A A L V
A T G S E N L K S L Y N T V C V W C H A E E K V K H T E E A K Q V
Q R H L V V E T G T T E T M A K T S R A T A A S S G R G G N Y A V
Q Q G G N Y V H L A L S A R T L N A W V K L E E K K F G A E V V A
G F Q A L S E G C T A Y D N Q M L N C V G D H Q A A M Q R D N E E
A A D W D L Q H A Q A A A Q Q G Q L R E A S G S D A G T T S S V D
E Q Q W M Y R Q Q N A A V G N Y R R W Q L G L Q K C V R M Y N A T
N L D V K Q G A K E A F Q S Y V D R F Y K S L R A E Q T D A A V K
N W M T Q T L L Q N A N A D C K L V L K G L G V N A T L E E M L T
A C Q G V G G A G Q K A R L M A E A L K E A L A A V A A F A A A Q
Q R G A R K A K C W N C G K E G H S A R Q C R A A R R Q G C W K C
G K M D H V M A K C A D R Q A G F L G L G A W G K K A R N F A M A
Q V H Q G L M A T A A A E D A A V D L L K N Y M Q L G K Q Q R E K
Q R E S R E K A Y K E V T E D L L H L N S L F G G D Q •
pCATESVgagDX gene: 758-2395
CCTGGCCATTGCATACGTTGTATCCATATCATAJLTATGTACATTTATATTGGCTCATGTCCAAC
ATTACCGCCATGTTGACATTGATTATTGACTAGTTATTAATAGTAATCAATTACGGGGTCATTAG
TTCATAGCCCATATATGGAGTTCCGCGTTACATAACTTACGGTAAATGGCCCGCCTGGCTGACCG
CCCAACGACCCCCGCCCATTGACGTCAATAATGACGTATGTTCCCATAGTAACGCCAATAGGGAC
TTTCCATTGACGTCAATGGGTGGAGTATTTACGGTAAACTGCCCACTTGGCAGTACATCAAGTGT
ATCATATGCCAAGTACGCCCCCTATTGACGTCAATGACGGTAAATGGCCCGCCTGGCATTATGCC
CAGTACATGACCTTATGGGACTTTCCTACTTGGCAGTACATCTACGTATTAGTCATCGCTATTAC
CATGGTGATGCGGTTTTGGCAGTACATCAATGGGCGTGGATAGCGGTTTGACTCACGGGGATTTC
CAAGTCTCCACCCCATTGACGTCAATGGGAGTTTGTTTTGGCACCAAAATCAACGGGACTTTCCA
AAATGTCGTAACAACTCCGCCCCATTGACGCAAATGGGCGGTAGGCGTGTACGGTGGGAGGTCTA
TATAAGCAGAGCTCGTTTAGTGAACCGTCAGATCGCCTGGAGACGCCATCCACGCTGTTTTGACC
TCCATAGAAGACACCGGGACCGATCCAGCCTCCGCGGGCGCGCATGAGAAAAGCGGCTGTTAGTC
ACTGGCAGCAGCAGTCTTACCTGGACTCTGGAATCCATTCTGGTGCCACTACCACAGCTCCTTCT
CTGAGTgctagcgcaggagcaGGCGTGAGAAACTCCGTCTTGTCAGGGAAGAAAGCAGATGAATT
AGAAAAAATTAGGCTACGACCCAACGGAAAGAAAAAGTACATGTTGAAGCATGTAGTATGGGCAG
CAAATGAATTAGATAGATTTGGATTAGCAGAAAGCCTGTTGGAGAACAAAGAAGGATGTCAAAAA
ATACTTTCGGTCTTAGCTCCATTAGTGCCAACAGGCTCAGAAAATTTAAAAAGCCTTTATAATAC
TGTCTGCGTCATCTGGTGCATTCACGCAGAAGAGAAAGTGAAACACACTGAGGAAGCAAAACAGA
TAGTGCAGAGACACCTAGTGGTGGAAACAGGAACCACCGAAACCATGCCGAAGACCTCTCGACCA
ACAGCACCATCTAGCGGCAGAGGAGGAAACTACCCAGTACAGCAGATCGGTGGCAACTACGTCCA
CCTGCCACTGTCCCCGAGAACCCTGAACGCTTGGGTCAAGCTGATCGAGGAGAAGAAGTTCGGAG
CAGAAGTAGTGCCAGGATTCCAGGCACTGTCAGAAGGTTGCACCCCCTACGACATCAACCAGATG
CTGAACTGCGTTGGAGACCATCAGGCGGCTATGCAGATCATCCGTGACATCATCAACGAGGAGGC
TGCAGATTGGGACTTGCAGCACCCACAACCAGCTCCACAACAAGGACAACTTAGGGAGCCGTCAG
GATCAGACATCGCAGGAACCACCTCCTCAGTTGACGAACAGATCCAGTGGATGTACCGTCAGCAG
AACCCGATCCCAGTAGGCAACATCTACCGTCGATGGATCCAGCTGGGTCTGCAGAAATGCGTCCG
TATGTACAACCCGACCAACATTCTAGATGTAAAACAAGGGCCAAAAGAGCCATTTCAGAGCTATG
TAGACAGGTTCTACAAAAGTTTAAGAGCAGAACAGACAGATGCAGCAGTAAAGAATTGGATGACT
CAAACACTGCTGATTCAAAATGCTAACCCAGATTGCAAGCTAGTGCTGAAGGGGCTGGGTGTGAA
TCCCACCCTAGAAGAAATGCTGACGGCTTGTCAAGGAGTAGGGGGGCCGGGACAGAAGGCTAGAT
TAATGGCAGAAGCCCTGAAAGAGGCCCTCGCACCAGTGCCAATCCCTTTTGCAGCAGCCCAACAG
AGGGGACCAAGAAAGCCAATTAAGTGTTGGAATTGTGGGAAAGAGGGACACTCTGCAAGGCAATG
CAGAGCCCCAAGAAGACAGGGATGCTGGAAATGTGGAAAAATGGACCATGTTATGGCCAAATGCC
CAGACAGACAGGCGGGTTTTTTAGGCCTTGGTCCATGGGGAAAGAAGCCCCGCAATTTCCCCATG
GCTCAAGTGCATCAGGGGCTGATGCCAACTGCTCCCCCAGAGGACCCAGCTGTGGATCTGCTAAA
GAACTACATGCAGTTGGGCAAGCAGCAGAGAGAAAAGCAGAGAGAAAGCAGAGAGAAGCCTTACA
AGGAGGTGACAGAGGATTTGCTGCACCTCAATTCTCTCTTTGGAGGAGACCAGTAGGAATTctga
TACGATCCAGATCTGCTGTGCCTTCTAGTTGCCAGCCATCTGTTGTTTGCCCCTCCCCCGTGCCT
TCCTTGACCCTGGAAGGTGCCACTCCCACTGTCCTTTCCTAATAAAATGAGGAAATTGCATCGCA
TTGTCTGAGTAGGTGTCATTCTATTCTGGGGGGTGGGGTGGGGCAGCACAGCAAGGGGGAGGATT
GGGAAGACAATAGCAGGCATGCTGGGGATGCGGTGGGCTCTATGGGTACCCAGGTGCTGAAGAAT
TGACCCGGTTCCTCCTGGGCCAGAAAGAAGCAGGCACATCCCCTTCTCTGTGACACACCCTGTCC
ACGCCCCTGGTTCTTAGTTCCAGCCCCACTCATAGGACACTCATAGCTCAGGAGGGCTCCGCCTT
CAATCCCACCCGCTAAAGTACTTGGAGCGGTCTCTCCCTCCCTCATCAGCCCACCAAACCAAACC
TAGCCTCCAAGAGTGGGAAGAAATTAAAGCAAGATAGGCTATTAAGTGCAGAGGGAGAGAAAATG
CCTCCAACATGTGAGGAAGTAATGAGAGAAATCATAGAATTTCTTCCGCTTCCTCGCTCACTGAC
TCGCTGCGCTCGGTCGTTCGGCTGCGGCGAGCGGTATCAGCTCACTCAAAGGCGGTAATACGGTT
ATCCACAGAATCAGGGGATAACGCAGGAAAGAACATGTGAGCAAAAGGCCAGCAAAAGGCCAGGA
ACCGTAAAAAGGCCGCGTTGCTGGCGTTTTTCCATAGGCTCCGCCCCCCTGACGAGCATCACAAA
AATCGACGCTCAAGTCAGAGGTGGCGAAACCCGACAGGACTATAAAGATACCAGGCGTTTCCCCC
TGGAAGCTCCCTCGTGCGCTCTCCTGTTCCGACCCTGCCGCTTACCGGATACCTGTCCGCCTTTC
TCCCTTCGGGAAGCGTGGCGCTTTCTCAATGCTCACGCTGTAGGTATCTCAGTTCGGTGTAGGTC
GTTCGCTCCAAGCTGGGCTGTGTGCACGAACCCCCCGTTCAGCCCGACCGCTGCGCCTTATCCGG
TAACTATCGTCTTGAGTCCAACCCGGTAAGACACGACTTATCGCCACTGGCAGCAGCCACTGGTA
ACAGGATTAGCAGAGCGAGGTATGTAGGCGGTGCTACAGAGTTCTTGAAGTGGTGGCCTAACTAC
GGCTACACTAGAAGGACAGTATTTGGTATCTGCGCTCTGCTGAAGCCAGTTACCTTCGGAAAAAG
AGTTGGTAGCTCTTGATCCGGCAAACAAACCACCGCTGGTAGCGGTGGTTTTTTTGTTTGCAAGC
AGCAGATTACGCGCAGAAAAAAAGGATCTCAAGAAGATCCTTTGATCTTTTCTACGGGGTCTGAC
GCTCAGTGGAACGAAAACTCACGTTAAGGGATTTTGGTCATGAGATTATCAAAAAGGATCTTCAC
CTAGATCCTTTTAAATTAAAAATGAAGTTTTAAATCAATCTAAAGTATATATGAGTAAACTTGGT
CTGACAGTTACCAATGCTTAATCAGTGAGGCACCTATCTCAGCGATCTGTCTATTTCGTTCATCC
ATAGTTGCCTGACTCCGGGGGGGGGGGGCGCTGAGGTCTGCCTCGTGAAGAAGGTGTTGCTGACT
CATACCAGGCCTGAATCGCCCCATCATCCAGCCAGAAAGTGAGGGAGCCACGGTTGATGAGAGCT
TTGTTGTAGGTGGACCAGTTGGTGATTTTGAACTTTTGCTTTGCCACGGAACGGTCTGCGTTGTC
GGGAAGATGCGTGATCTGATCCTTCAACTCAGCAAAAGTTCGATTTATTCAACAAAGCCGCCGTC
CCGTCAAGTCAGCGTAATGCTCTGCCAGTGTTACAACCAATTAACCAATTCTGATTAGAAAAACT
CATCGAGCATCAAATGAAACTGCAATTTATTCATATCAGGATTATCAATACCATATTTTTGAAAA
AGCCGTTTCTGTAATGAAGGAGAAAACTCACCGAGGCAGTTCCATAGGATGGCAAGATCCTGGTA
TCGGTCTGCGATTCCGACTCGTCCAACATCAATACAACCTATTAATTTCCCCTCGTCAAAAATAA
GGTTATCAAGTGAGAAATCACCATGAGTGACGACTGAATCCGGTGAGAATGGCAAAAGCTTATGC
ATTTCTTTCCAGACTTGTTCAACAGGCCAGCCATTACGCTCGTCATCAAAATCACTCGCATCAAC
CAAACCGTTATTCATTCGTGATTGCGCCTGAGCGAGACGAAATACGCGATCGCTGTTAAAAGGAC
AATTACAAACAGGAATCGAATGCAACCGGCGCAGGAACACTGCCAGCGCATCAACAATATTTTCA
CCTGAATCAGGATATTCTTCTAATACCTGGAATGCTGTTTTCCCGGGGATCGCAGTGGTGAGTAA
CCATGCATCATCAGGAGTACGGATAAAATGCTTGATGGTCGGAAGAGGCATAAATTCCGTCAGCC
AGTTTAGTCTGACCATCTCATCTGTAACATCATTGGCAACGCTACCTTTGCCATGTTTCAGAAAC
AACTCTGGCGCATCGGGCTTCCCATACAATCGATAGATTGTCGCACCTGATTGCCCGACATTATC
GCGAGCCCATTTATACCCATATAAAATCAGCATCCATGTTGGAATTTAATCGCGGCCTCGAGCAA
GACGTTTCCCGTTGAATATGGCTCATAACACCCCTTGTATTACTGTTTATGTAAGCAGACAGTTT
TATTGTTCATGATGATATATTTTTATCTTGTGCAATGTAACATCAGAGATTTTGAGACACAACGT
GGCTTTCCCCCCCCCCCCATTATTGAAGCATTTATCAGGGTTATTGTCTCATGAGCGGATACATA
TTTGAATGTATTTAGAAAAATAAACAAATAGGGGTTCCGCGCACATTTCCCCGAAAAGTGCCACC
TGACGTCTAAGAAACCATTATTATCATGACATTAACCTATAAAAATAGGCGTATCACGAGGCCCT
TTCGTCTCGCGCGTTTCGGTGATGACGGTGAAAACCTCTGACACATGCAGCTCCCGGAGACGGTC
ACAGCTTGTCTGTAAGCGGATGCCGGGAGCAGACAAGCCCGTCAGGGCGCGTCAGCGGGTGTTGG
CGGGTGTCGGGGCTGGCTTAACTATGCGGCATCAGAGCAGATTGTACTGAGAGTGCACCATATGC
GGTGTGAAATACCGCACAGATGCGTAAGGAGAAAATACCGCATCAGATTGGCTATTGG (5646)
protein:
M R K A A V S H W Q Q Q S Y L D S G H S G A T T T A A S L S
(CATE)
A S A G A (linker)
G V R N S V L S G K K A D E L E K R L R A N G K K K Y M L K
H V V W A A N E L D R F G L A E S L L E N K E G C Q K L S V
L A A L V A T G S E N L K S L Y N T V C V J W C H A E E K V
K H T E E A K Q V Q R H L V V E T G T T E T M A K T S R A T
A A S S G R G G N Y A V Q Q L G G N Y V H L A L S A R T L N
A W V K L E E K K F G A E V V A G F Q A L S E G C T A Y D N
Q M L N C V G D H Q A A M Q R D N E E A A D W D L Q H A Q A
A A Q Q G Q L R E A S G S D A G T T S S V D E Q Q W M Y R Q
Q N A A V G N Y R R W Q L G L Q K C V R M Y N A T N L D V K
Q G A K E A F Q S Y V D R F Y K S L R A E Q T D A A V K N W
M T Q T L L Q N A N A D C K L V L K G L G V N A T L E E M L
T A C Q G V G G A G Q K A R L M A E A L K E A L A A V A A F
A A A Q Q R G A R K A K C W N C G K E G H S A R Q C R A A R
R Q G C W K C G K M D H V M A K C A D R Q A G F L G L G A W
G K K A R N F A M A Q V H Q G L M A T A A A E D A A V D L L
K N Y M Q L G K Q Q R E K Q R E S R E K A Y K E V T E D L L
H L N S L F G G D Q • (p57gag)
pCMVMCA3p39gene: 758-2176
(1)CCTGGCCATTGCATACGTTGTATCCATATCATAATATGTACATTTATATTGGCTCATGTCCA
ACATTACCGCCATGTTGACATTGATTATTGACTAGTTATTAATAGTAATCAATTACGGGGTCATT
AGTTCATAGCCCATATATGGAGTTCCGCGTTACATAACTTACGGTAAATGGCCCGCCTGGCTGAC
CGCCCAACGACCCCCGCCCATTGACGTCAATAATGACGTATGTTCCCATAGTAACGCCAATAGGG
ACTTTCCATTGACGTCAATGGGTGGAGTATTTACGGTAAACTGCCCACTTGGCAGTACATCAAGT
GTATCATATGCCAAGTACGCCCCCTATTGACGTCAATGACGGTAAATGGCCCGCCTGGCATTATG
CCCAGTACATGACCTTATGGGACTTTCCTACTTGGCAGTACATCTACGTATTAGTCATCGCTATT
ACCATGGTGATGCGGTTTTGGCAGTACATCAATGGGCGTGGATAGCGGTTTGACTCACGGGGATT
TCCAAGTCTCCACCCCATTGACGTCAATGGGAGTTTGTTTTGGCACCAAAATCAACGGGACTTTC
CAAAATGTCGTAACAACTCCGCCCCATTGACGCAAATGGGCGGTAGGCGTGTACGGTGGGAGGTC
TATATAAGCAGAGCTCGTTTAGTGAACCGTCAGATCGCCTGGAGACGCCATCCACGCTGTTTTGA
CCTCCATAGAAGACACCGGGACCGATCCAGCCTCCGCGGGCGCGCATGAACCCAAGTGCTGCCGT
CATTTTCTGCCTCATCCTGCTGGGTCTGAGTGGGACTCAAGggatcctcgaCATGGCGCAACCGG
TAGGTATAAACACAAGCACAACCTGTTGCTATCGTTTCATAAATAAAAAGATACCGAAGCAACGT
CTGGAAAGCTATCGCCGTACCACTTCTAGCCACTGTCCGCGTGAAGCTGTTATATTCAAAACGAA
ACTGGATAAGGAGATCTGCGCCGACCCTACACAGAAATGGGTTCAGGACTTTATGAAGCACCTGG
ATAAAAAGACACAGACGCCGAAACTGGCTAGCGCAGGAGCAGGCGTGAGAAACTCCGTCTTGTCA
GGGAAGAAAGCAGATGAATTAGAAAAAATTAGGCTACGACCCAACGGAAAGAAAAAGTACATGTT
GAAGCATGTAGTATGGGCAGCAAATGAATTAGATAGATTTGGATTAGCAGAAAGCCTGTTGGAGA
ACAAAGAAGGATGTCAAAAAATACTTTCGGTCTTAGCTCCATTAGTGCCAACAGGCTCAGAAAAT
TTAAAAAGCCTTTATAATACTGTCTGCGTCATCTGGTGCATTCACGCAGAAGAGAAAGTGAAACA
CACTGAGGAAGCAAAACAGATAGTGCAGAGACACCTAGTGGTGGAAACAGGAACCACCGAAACCA
TGCCGAAGACCTCTCGACCAACAGCACCATCTAGCGGCAGAGGAGGAAACTACCCAGTACAGCAG
ATCGGTGGCAACTACGTCCACCTGCCACTGTCCCCGAGAACCCTGAACGCTTGGGTCAAGCTGAT
CGAGGAGAAGAAGTTCGGAGCAGAAGTAGTGCCAGGATTCCAGGCACTGTCAGAAGGTTGCACCC
CCTACGACATCAACCAGATGCTGAACTGCGTTGGAGACCATCAGGCGGCTATGCAGATCATCCGT
GACATCATCAACGAGGAGGCTGCAGATTGGGACTTGCAGCACCCACAACCAGCTCCACAACAAGG
ACAACTTAGGGAGCCGTCAGGATCAGACATCGCAGGAACCACCTCCTCAGTTGACGAACAGATCC
AGTGGATGTACCGTCAGCAGAACCCGATCCCAGTAGGCAACATCTACCGTCGATGGATCCAGCTG
GGTCTGCAGATTTGCGTCCGTATGTACAACCCGACCAACATTCTAGATGTAAAACAAGGGCCAAA
AGAGCCATTTCAGAGCTATGTAGACAGGTTCTACAAAAGTTTAAGAGCAGAACAGACAGATGCAG
CAGTAAAGAATTGGATGACTCAAACACTGCTGATTCAAAATGCTAACCCAGATTGCAAGCTAGTG
CTGAAGGGGCTGGGTGTGAATCCCACCCTAGAAGAAATGCTGACGGCTTGTCAAGGAGTAGGGGG
GCCGGGACAGAAGGCTAGATTAATGGAATTCTGATACGATCCaGATCTGCTGTGCCTTCTAGTTG
CCAGCCATCTGTTGTTTGCCCCTCCCCCGTGCCTTCCTTGACCCTGGAAGGTGCCACTCCCACTG
TCCTTTCCTAATAAAATGAGGAAATTGCATCGCATTGTCTGAGTAGGTGTCATTCTATTCTGGGG
GGTGGGGTGGGGCAGCACAGCAAGGGGGAGGATTGGGAAGACAATAGCAGGCATGCTGGGGATGC
GGTGGGCTCTATGGGTACCCAGGTGCTGAAGAATTGACCCGGTTCCTCCTGGGCCAGAAAGAAGC
AGGCACATCCCCTTCTCTGTGACACACCCTGTCCACGCCCCTGGTTCTTAGTTCCAGCCCCACTC
ATAGGACACTCATAGCTCAGGAGGGCTCCGCCTTCAATCCCACCCGCTAAAGTACTTGGAGCGGT
CTCTCCCTCCCTCATCAGCCCACCAAACCAAACCTAGCCTCCAAGAGTGGGAAGAAATTAAAGCA
AGATAGGCTATTAAGTGCAGAGGGAGAGAAAATGCCTCCAACATGTGAGGAAGTAATGAGAGAAA
TCATAGAATTTCTTCCGCTTCCTCGCTCACTGACTCGCTGCGCTCGGTCGTTCGGCTGCGGCGAG
CGGTATCAGCTCACTCAAAGGCGGTAATACGGTTATCCACAGAATCAGGGGATAACGCAGGAAAG
AACATGTGAGCAAAAGGCCAGCAAAAGGCCAGGAACCGTAAAAAGGCCGCGTTGCTGGCGTTTTT
CCATAGGCTCCGCCCCCCTGACGAGCATCACAAAAATCGACGCTCAAGTCAGAGGTGGCGAAACC
CGACAGGACTATAAAGATACCAGGCGTTTCCCCCTGGAAGCTCCCTCGTGCGCTCTCCTGTTCCG
ACCCTGCCGCTTACCGGATACCTGTCCGCCTTTCTCCCTTCGGGAAGCGTGGCGCTTTCTCAATG
CTCACGCTGTAGGTATCTCAGTTCGGTGTAGGTCGTTCGCTCCAAGCTGGGCTGTGTGCACGAAC
CCCCCGTTCAGCCCGACCGCTGCGCCTTATCCGGTAACTATCGTCTTGAGTCCAACCCGGTAAGA
CACGACTTATCGCCACTGGCAGCAGCCACTGGTAACAGGATTAGCAGAGCGAGGTATGTAGGCGG
TGCTACAGAGTTCTTGAAGTGGTGGCCTAACTACGGCTACACTAGAAGGACAGTATTTGGTATCT
GCGCTCTGCTGAAGCCAGTTACCTTCGGAAAAAGAGTTGGTAGCTCTTGATCCGGCAAACAAACC
ACCGCTGGTAGCGGTGGTTTTTTTGTTTGCAAGCAGCAGATTACGCGCAGAAAAAAAGGATCTCA
AGAAGATCCTTTGATCTTTTCTACGGGGTCTGACGCTCAGTGGAACGAAAACTCACGTTAAGGGA
TTTTGGTCATGAGATTATCAAAAAGGATCTTCACCTAGATCCTTTTAAATTAAAAATGAAGTTTT
AAATCAATCTAAAGTATATATGAGTAAACTTGGTCTGACAGTTACCAATGCTTAATCAGTGAGGC
ACCTATCTCAGCGATCTGTCTATTTCGTTCATCCATAGTTGCCTGACTCCGGGGGGGGGGGGCGC
TGAGGTCTGCCTCGTGAAGAAGGTGTTGCTGACTCATACCAGGCCTGAATCGCCCCATCATCCAG
CCAGAAAGTGAGGGAGCCACGGTTGATGAGAGCTTTGTTGTAGGTGGACCAGTTGGTGATTTTGA
ACTTTTGCTTTGCCACGGAACGGTCTGCGTTGTCGGGAAGATGCGTGATCTGATCCTTCAACTCA
GCAAAAGTTCGATTTATTCAACAAAGCCGCCGTCCCGTCAAGTCAGCGTAATGCTCTGCCAGTGT
TACAACCAATTAACCAATTCTGATTAGAAAAACTCATCGAGCATCAAATGAAACTGCAATTTATT
CATATCAGGATTATCAATACCATATTTTTGAAAAAGCCGTTTCTGTAATGAAGGAGAAAACTCAC
CGAGGCAGTTCCATAGGATGGCAAGATCCTGGTATCGGTCTGCGATTCCGACTCGTCCAACATCA
ATACAACCTATTAATTTCCCCTCGTCAAAAATAAGGTTATCAAGTGAGAAATCACCATGAGTGAC
GACTGAATCCGGTGAGAATGGCAAAAGCTTATGCATTTCTTTCCAGACTTGTTCAACAGGCCAGC
CATTACGCTCGTCATCAAAATCACTCGCATCAACCAAACCGTTATTCATTCGTGATTGCGCCTGA
GCGAGACGAAGCAGGAACACTGCCAGCGCATCAACAATATTTTCACCTGAATCAGGATATTCTTC
TAATACCTGGAATGCTGTTTTCCCGGGGATCGCAGTGGTGAGTAACCATGCATCATCAGGAGTAC
GGATAAAATGCTTGATGGTCGGAAGAGGCATAAATTCCGTCAGCCAGTTTAGTCTGACCATCTCA
TCTGTAACATCATTGGCAACGCTACCTTTGCCATGCCTGATTGCCCGACATTATCGCGAGCCCAT
TTATACCCATATAAAAATCAGCATCCATGTTGGAATTTAATCGCGGCCTCGAGC&AGACGTTTCC
CGTTGAATATGGCTCATAACACCCCTTGTATTACTGTTTATGTAAGCAGACAGTTTTATTGTTCA
TGATGATATATTTTTATCTTGTGCAJAATGTAACATCAGAGATTTTGAGACACAACGTGGCTTTC
CCCCCCCCCCCATTATTGAAGCATTTATCAGGGTTATTGTCTCATGAGCGGATACAAAAAGTGCC
ACCTGACGTCTAAGAACCATTATTATCATGACATTAACCTATAAAAATAGGCGTATCACGAGGCC
CTTTCGTCTCGCGCGTTTCGGTGATGACGGTGAAAACCTCTGACACATGCAGCTCCCGGAGACGG
TCACAGCTTGTCTGTAAGCGGATGCCGGGAGCAGACAAGCCCGTCAGGGCGCGTCAGCGGGTGTT
GGCGGGTGTCGGGGCTGGCTTAACTATGCGGCATCAGAGCAGATTGTACTGAGAGTGCACCATAT
GCGGTGTGAAATACCGCACAGATGCGTAAGGAGAAAAATACCGCATCAGATTGGCTATTGG
(5418)
protein:
M N A S A A V F C L L L G L S G T Q (IP10)
G L D (linker)
M A Q A V G N T S T T C C Y R F N K K A K Q R L E S Y R R T T S S
H C A R E A V F K T K L D K E C A D A T Q K W V Q D F M K H L D K
K T Q T A K L (MCP3)
A S A G A (linker)
G V R N S V L S G K K A D E L E K R L R A N G K K K Y M L K H V V
W A A N E L D R F G L A E S L L E N K E G C Q K L S V L A A L V A
T G S E N L K S L Y N T V C V W C H A E E K V K H T E E A K Q V Q
R H L V V E T G T T E T M A K T S R P T A P S S G R G G N Y A V Q
Q I G G N Y V H L P L S P R T L N A W V K L I E E K K F G A E V V
A G F Q A L S E G C T A Y D N Q M L N C V G D H Q A A M Q R D N E
E A A D W D L Q H A Q A A A Q Q G Q L R E A S G S D A G T T S S V
D E Q Q W M Y R Q Q N A A V G N Y R R W Q L G L Q K C V R M Y N A
T N L D V K Q G A K E A F Q S Y V D R F Y K S L R A E Q T D A A V
K N W M T Q T L L Q N A N A D C K L V L K G L G V N A T L E E M L
T A C Q G V G G A G Q K A R L M E F • (SIVp39gag)
pCMV SIV CATEpolNTV gene: 769-5655
(1)CCTGGCCATTGCATACGTTGTATCCATATCATAAATATGTACATTTATATTGGCTCATGTCC
AACATTACCGCCATGTTGACATTGATTATTGACTAGTTATTAATAGTAATCAATTACGGGGTCAT
TAGTTCATAGCCCATATATGGAGTTCCGCGTTACATJAACTTACGGTAAATGGCCCGCCTGGCTG
ACCGCCCAACGACCCCCGCCCATTGACGTATGGGTGGAGTATTTACGGTAAAACTGCCCACTTGG
CAGTACATCAAGTGTATCATATGCCAAGTACGCCCCCTATTGACGTCJAATGACGGTAAATGGCC
CGCCTGGCATTATGCCCAGTACATGACCTTATGGGACTTTCCTACTTGGCAGTACATCTACGTAT
TAGTCATCGCTATTACCATGGTGATGCGGTTTTGGCAGTACATCAATGGGCGTGGATAGCGGTTT
GACTCACGGGGATTTCCAAAAAAGTCTCCACCCCATTGACGTCAATGGGAGTTTGTTTTGGCACC
AAAATCAACGGGACTTTCCKAATGTCGTAACAACTCCGCCCCATTGACGCAAATGGGCGGTAGGC
GTGTACGGTGGGAGGTCTATATAAGCAGAGCTCGTTTAGTGAACCGTCAGATCGCCTGGAGACGC
CATCCACGCTGTTTTGACCTCCATAGAAGACACCGGGACCGATCCAGCCTCCGCGGGCGCGCGTC
GACAAGAAATGAGAAAAGCGGCTGTTAGTCACTGGCAGCAGCAGTCTTACCTGGACTCTGGAATC
CATTCTGGTGCCACTACCACAGCTCCTTCTCTGAGTGCTAGCGCAGGAGCATACCCCTACGACGT
GCCCGACTACGCCAGCCTGGGGGCCCATCGGGAGGCGTTGCAGGGGGGAGATCGGGGGTTCGCGG
CGCCGCAGTTCTCGCTGTGGCGGCGGCCGGTCGTCACTGCGCATATTGAGGGACAGCCGGTAGAG
GTATTGCTGGCGGCAGCGGCGGATGATTCGATTGTAACGGGAJLTAGAGTTGGGTCCGCATTATA
CCCCGAAGATAGTAGGGGGGATCGGGGGGTTTATCAATACGAAAGAGTAcAAJAATGTAGAGATA
GAGGTTTTGGGCAAACGGATTAAJAAGGGACGATCATGACAGGGGACACCCCGATTAACATCTTT
GGTCGGATAACTTGCTTATAACGGCGCTGGGGATGTCGCTTAAACTTTCCCATAGCGAAAGTAGA
GCCTGTAAAAGTCGCCTTGAAGCCGGGAAAAGGATGGACCGAAATTGAAGCAGTGGCCGTTGTCA
AAAGAGAAGATAGTTGCGTTGCGGGAGATCTGTGAGAAGATGGAGAAGGATGGACAGTTGGAGGA
GGCGCCCCCGACCAATCCATACAACACCCCCACATTCGCGATCAAGAAGAAGGATAAGAACAAGT
GGCGGATGCTGATAGACTTTCGGGAGTTGAATCGGGTCACGCAGGACTTTACGGAGGTCCAATTG
GGAATACCGCACCCGGCGGGACTAGCGAAACGGAAACGGATTACGGTACTGGATATAGGTGATGC
GTACTTCTCCATACCGCTTGATGAGGAGTTTCGGCAGTACACGGCCTTTACGCTTCCGTCAGTAA
ACAACGCGGAGCCGGGGAAGCGATACATATATAAGGTTCTGCCGCAGGGATGGAAGGGGTCGCCG
GCCATCTTCCAATACACGATGCGGCATGTGCTAGAGCCCTTCCGGAAGGCGAATCCGGATGTGAC
CTTGGTCCAGTATATGGCGGCGATCTTGATAGCGTCGGACCGGACGGACCTGGAGCATGACCGGG
TAGTTCTTCAGTCGAAGGAGCTCTTGAATAGCATAGGGTTTTCGACCCCGGAGGAGAAATTCCAA
AAAGATCCCCCGTTTCAATGGATGGGGTACGAGTTGTGGCCGACGAAATGGAAGTTGCAAAAGAT
AGAGTTGCCGCAACGGGAGACCTGGACAGTGAATGATATACAGAAGCTTGTAGGAGTACTTAATT
GGGCGGCTCAAATATATCCGGGTATAAAAACCAAACATCTCTGTCGGTTGATTCGGGGAAAAATG
ACGCTAACGGAGGAGGTTCAGTGGACGGAGATGGCGGAGGCAGAGTATGAGGAGAACAAGATCAT
CCTCTCGCAGGAGCAAGAGGGATGTTATTACCAAGAGGGCAAGCCGTTGGAGGCCACGGTAATCA
AGTCGCAGGACAATCAGTGGTCGTATAAGATCCACCAAGAGGACAAGATCCTGAAAGTAGGAAAG
TTCGCGAAGATCAAGAACACGCATACCAACGGAGTGCGGCTACTTGCGCATGTAATACAGAAAAT
AGGAAAGGAGGCGATAGTGATCTGGGGACAGGTCCCGAAATTCCACCTTCCGGTTGAGAAGGATG
TATGGGAGCAGTGGTGGACGGACTATTGGCAAGTAACCTGGATACCGGAGTGGGACTTTATCTCG
ACGCCGCCGCTAGTACGGCTTGTCTTCAATCTAGTGAAGGACCCGATAGAGGGAGAGGAGACCTA
TTATACGGATGGATCGTGTAACAAGCAGTCGAAAGAGGGGAAAGCGGGATATATCACGGATCGGG
GCAAAGACAAAGTAAAAGTGCTTGAGCAGACGACGAATCAACAAGCGGCGTTGGAGGCGTTTCTC
ATGGCGTTGACGGACTCGGGGCCAAAGGCGAACATCATCGTAGACTCGCAGTACGTCATGGGAAT
CATCACGGGATGCCCGACGGAGTCGGAGAGCCGGCTAGTCAACCAAATCATCGAGGAGATGATCA
AGAAGTCGGAGATATATGTAGCGTGGGTACCGGCGCACAAAGGTATAGGAGGAAACCAAGAGATA
GACCACCTAGTTTCGCAAGGGATTAGACAAGTTCTCTTCTTGGAGAAGATAGAGCCGGCGCAAGA
GGAGCATGATAAATACCATTCGAATGTAAAAGAGTTGGTATTCAAATTCGGACTTCCCCGGATAG
TGGCCCGGCAGATAGTAGACACCTGTGATAAATGTCATCAGAAAGGAGAGGCGATACATGGGCAG
GCGAACTCGGATCTAGGGACTTGGCAAATGGCGTGTACCCATCTAGAGGGAAAGATCATCATAGT
TGCGGTACATGTAGCGTCGGGATTCATAGAAGCGGAGGTAATTCCGCAAGAGACGGGACGGCAGA
CGGCGCTATTCCTGTTGAAATTGGCGGGCAGATGGCCTATTACGCATCTACACACGGCGAATGGT
GCGAACTTTGCGTCGCAAGAAGTAAAGATGGTTGCGTGGTGGGCGGGGATAGAGCACACCTTTGG
GGTACCGTACAATCCGCAGTCGCAGGGAGTAGTGGCGGCGATGAACCACCACCTGAAGAACCAAA
TCGATCGGATCAGGGAGCAAGCGAACTCAGTAGAGACCATAGTATTGATGGCGGTTCATTGCATG
AACTTCAAGCGGCGGGGAGGAATAGGGGATATGACGCCGGCGGAGCGGTTGATTAACATGATCAC
GACGGAGCAAGAGATCCAATTCCAACAATCGAAGAACTCGAAGTTCAAGAACTTTCGGGTCTATT
ACCGGGAGGGCCGGGATCAACTGTGGAAGGGACCCGGAGAGCTATTGTGGAAAGGGGAGGGAGCG
GTCATCTTGAAAGTAGGGACGGACATTAAGGTAGTACCCCGGCGGAAGGCGAAGATCATCAAGGA
TTATGGAGGAGGAAAAGAGGTGGATAGCTCGTCCCACATGGAGGATACCGGAGAGGCGCGGGAGG
TGGCACGCGTCGCGGCCGCGGCTATCTCCATGAGGCGGTCCAGGCCGTCTGGGGATCTGCGACAG
AGACTCTTGCGGGCGCGTGGGGAGACTTATGGGAGACTCTTAGGAGAGGTGGAAGATGGATACTC
GCAATCCCCAGGAGGATTAGACAAGGGCTTGAGCTCACTCTCGTGCGAGGGACAGAAGTACAACC
AGGGGCAGTACATGAACACTCCATGGAGAAACCCCGCTGAAGAGCGGGAGAAGTTGGCGTACCGG
AAGCAGAACATGGACGACATCGACGAGGAGGACGACGACTTAGTCGGGGTCTCAGTGCGGCCGAA
GGTCCCCCTACGGACGATGTCGTACAAGTTGGCGATAGACATGTCGCACTTCATCAAGGAGAAGG
GGGGACTGGAGGGGATCTACTACTCGGCGCGGCGGCACCGCATCCTCGACATCTACCTCGAGAAG
GAGGAGGGCATCATCCCGGACTGGCAGGACTACACCTCAGGACCAGGAATCAGATATCCAAAGAC
GTTCGGCTGGCTCTGGAAGCTCGTCCCTGTAAACGTCTCGGACGAGGCGCAGGAGGACGAGGAGC
ACTACCTCATGCATCCGGCGCAAACTTCCCAGTGGGATGACCCTTGGGGAGAGGTTCTAGCATGG
AAGTTTGATCCAACTCTGGCCTACACTTATGAGGCATATGTTAGATACCCAGAAGAGTTTGGAAG
CAAGTCAGGCCTGTCAGAGGAAGAGGTTAGAAGAAGGCTAACCGCAAGAGGCCTTCTTAACATGG
CTGACAAGAAGGAAACTCGCGGCGCCGAGACACCCTTGAGGGAGCAGGAGAACTCATTAGAATCC
TCCAACGAGCGCTCTTCATGCATTTCAGAGGCGGATGCATCCACTCCAGAATCGGCCAACCTGGG
GGAGGAAATCCTCTCTCAGCTATACCGCCCTCTCGAGGCGTGCTACAACACGTGCTACTGCAAGA
AGTGCTGCTACCACTGCCAGTTCTGCTTCCTTAAAAAGGGCCTGGGGATCTGCTACGAGCAGTCG
CGAAAGCGGCGGCGGACGCCGAAGAAGGCGAAGGCGAACACGTCGTCGGCGTCGAACAACAGACC
CATATCCAACAGGACCCGGCACTGCCAACCAGAGAAGGCAAAGAAAGAGACGGTGGAGAAGGCGG
TGGCAACAGCTCCTGGCCTTGGCAGAGGATCCGAGGAGGAAAAGAGGTGGATCGCAGTTCCCACG
TGGAGGATACCGGAGAGGCTAGAGAGGTGGCATAGCCTCATAAAGTACCTGAAGTACAAGACGAA
GGACCTCCAGAAGGTCTGCTATGTGCCCCACTTCAAAAGTCGGATGGGCATGGTGGACCTGCAGC
AGAGTCATCTTCCCCCTACAAGAOGGAAGCCACTTGGAGGTCCAGGGGTACTGGCACTTGACGCC
GGAGAAGGGGTGGCTCTCGACGTACGCGGTGCGGATCACCTGGTACTCGAAGAACTTCTGGACGG
ATGTCACGCCGAACTATGCGGACATCTTGCTGCATAGCACTTACTTCCCTTGCTTTACGGCGGGA
GAAGTGAGAAGGGCCATCAGGGGAGAGCAACTGCTGTCGTGCTGCCGGTTCCCGCGGGCGCACAA
GTACCAGGTACCGAGCCTACAGTACTTGGCGCTGAAGGTCGTCAGCGACGTCAGATCCCAGGGGG
AGAACCCCACCTGGAAGCAGTGGCGGCGGGACAACCGGAGAGGCCTTCGAATGGCGAAGCAGAAC
TCGCGGGGAGATAAGCAGCGGGGCGGTAAACCACCTACCAAGGGAGCGAACTTCCCGGGTTTGGC
AAAGGTCTTGGGAATACTGGCAGTTAACTGAGAATTCGATCCAGATCTGCTGTGCCTTCTAGTTG
CCAGCCATCTGTTGTTTGCCCCTCCCCCGTGCCTTCCTTGACCCTGGAAGGTGCCACTCCCACTG
TCCTTTCCTAATAAAATGAGGAAATTGCATCGCATTGTCTGAGTAGGTGTCATTCTATTCTGGGG
GGTGGGGTGGGGCAGCACAGCAAGGGGGAGGATTGGGAAGACAATAGCAGGCATGCTGGGGATGC
GGTGGGCTCTATGGGTACCCAGGTGCTGAAGAATTGACCCGGTTCCTCCTGGGCCAGAAAGAAGC
AGGCACATCCCCTTCTCTGTGACACACCCTGTCCACGCCCCTGGTTCTTAGTTCCAGCCCCACTC
ATAGGACACTCATAGCTCAGGAGGGCTCCGCCTTCAATCCCACCCGCTAAAGTACTTGGAGCGGT
CTCTCCCTCCCTCATCAGCCCACCAAACCAAAAACCTAGCCTCCAAGAGTGGGAAGAAATTAAAG
CAAGATAGGCTATTAAGTGCAGAGGGAGAGAAAATGCCTCCAACATGTGAGGAAGTAATGAGAGA
AATCATAGAATTTCTTCCGCTTCCTCGCTCACTGACTCGCTGCGCTCGGTCGTTCGGCTGCGGCG
AGCGGTATCAGCTCACTCAAAGGCGGTAATACGGTTATCCACAGAATCAGGGGATAACGCAGGAA
AGAACATGTGAGCAAAAGGCCAGCAAAAGGCCAGGAACCGTAAAAAGGCCGCGTTGCTGGCGTTT
TTCCATAGGCTCCGCCCCCCTGACGAGCATCACAAAAATCGACGCTCAAGTCAGAGGTGGCGAAA
CCCGACAGGACTATAAAGATACCAGGCGTTTCCCCCTGGAAGCTCCCTCGTGCGCTCTCCTGTTC
CGACCCTGCCGCTTACCGGATACCTGTCCGCCTTTCTCCCTTCGGGAAGCGTGGCGCTTTCTCAA
TGCTCACGCTGTAGGTATCTCAGTTCGGTGTAGGTCGTTCGCTCCAAGCTGGGCTGTGTGCACGA
ACCCCCCGTTCAGCCCGACCGCTGCGCCTTATCCGGTAACTATCGTCTTGAGTCCAACCCGGTAA
GACACGACTTATCGCCACTGGCAGCAGCCACTGGTAACAGGATTAGCAGAGCGAGGTATGTAGGC
GGTGCTACAGAGTTCTTGAAGTGGTGGCCTAACTACGGCTACACTAGAAGGACAGTATTTGGTAT
CTGCGCTCTGCTGAAGCCAGTTACCTTCGGAAAJAAAAAAGAGTTGGTAGCTCTTGATCCGGCAA
ACAAACCACCGCTGGTAGCGGTGGTTTTTTTGTTTGCAAGCAGCAGATTACGCGCAGAAGGATCT
CJAAGAAAGATCCTTTGATCTTTTCTACGGGGTCTGACGCTCAGTGGAACGAAAACTCACGTTAA
AGGGATTTTGCTTAATCAGTGAGGCACCTATCTCAGCGATCTGTCTATTTCGTTCATCCATAGTT
GCCTGACTCCGGGGGGGGGGGGCGCTGAGGTCTGCCTCGTGJAAGJAAGGTGTTGCTGACTCATA
CCAGGCCTGAATCGCCCCATCATCCAGCCAGAAAJAAAGTGAGGGAGCCACGGTTGATGAGAGCT
TTGTTGTAGGTGGACCAGTTGGTGATTTTGAACTTTTGCTTTGCCACGGAACGGTCTGCGTTGTC
GGGAAGATGCGTGATCTGATCCTTCAACTCAGCAAAAGTTCGATTTATTCAACAGCCGCCGTCCC
GTCAAGTCAGCGTAATGCTCTGCCAGTGTTACAACCAATTAACCAATTCTGATTAGAAAAAACTC
ATCGAGCATCAAATGAAACTGCAATTTATTCATATCAGGATTATCAATACCATATTTTTGAAAAA
GCCGTTTCTGTAATGAAGGAGAAAACTCACCGAGGCAGTTCCATAGGATGGTAATTTCCCCTCGT
CAATAAGGTTATCJAAGTGAGAAAATCACCATGAGTGACGCAGGCCAGCCATTACGCTCGTCATC
AAATCACTCGCATCAACCAAACCGTTATTCATTCGTGATTGCGCCTGAGCGAGACGATACGCGAT
CGCTGTTJAAAAAGGACAATTACAAACAGGAATCGATGCAACCGGCGCAGGAAACACTGCCAGCG
CATCAACGGATCGCAGTGGTGAGTAACCATGCATCATCAGGAGTACGGATAAAATGCTTGATGGT
CGGAAGAGGCATAAATTCCGTCAGCCAGTTTAGTCTGACCATCTCATCTGTAACATCATTGGCAA
CGCTACCTTTGCCATGTTTCAGAA&AAACAACTCTGGCGCATCGGGCTTCCCATACAATCGATAG
ATTGTCGCACCTGATTGCCCGACATTATCGCGAGGCAAGACGTTTCCCGTTGAATATGGCTCATA
ACACCCCTTGTATTACTGTTTATGTAAGCAGACAGTTTTATTGTTCATGATGATATATTTTTATC
TTGTGCAATGTJAACATCAGAGATTTTGAGACACAACGTGGCTTTCCCCCCCCCCCCATTATTGA
AGCATTTATCAGGGTTATTGTCTCATGAGCGGATACATATTTGAATGTATTTAGAATAAACAAAT
AGGGGTTCCGCGCACATTTCCCCGAAGTGCCACCTGACGTCTAAGAAACCATTATTATCATGACA
TTAACCTATAAA4ATAGGCGTATCACGAGGCCCTTTCGTCTCGCGCGTTTCGGTGATGACGGTGA
AAACCTCTGACACATGCAGCTCCCGGAGACGGTCACAGCTTGTCTGTAAGCGGATGCCGGGAGCA
GACAAGCCCGTCAGGGCGCGTCAGCGGGTGTTGGCGGGTGTCGGGGCTGGCTTAACTATGCGGCA
TCAGAGCAGATTGTACTGAGAGTGCACCATATGCGGTGTGAAATACCGCACAGATGCGTAAGGAG
AAAATACCGCATCAGATTGGCTATTGG (8900)
protein:
M R K A A V S H W Q Q Q S Y L D S G H S G A T T T A A S L S
(CATE)
A S A G A (linker)
Y A Y D V A D Y A S L (HA epitope)
G A R R E A L Q G G D R G F A A (pol ORF)
A Q F S L W R R A V V T A H E G Q A V E V L L A A A A D D S V T G
E L G A H Y T A K V G G G G F N T K E Y K N V E E V L G K R K G T
M T G D T A N F G R N L L T A L G M S L N F A J A K V E A V K V A
L K A G K D G A K L K Q W A L S K E K V A L R E C E K M E K D G Q
L E E A A A T N A Y N T A T F A K K K D K N K W R M L D F R E L N
R V T Q D F T E V Q L G J A H A A G L A K R K R T V L D G D A Y F
S A L D E E F R Q Y T A F T L A S V N N A E A G K R Y Y K V L A Q
G W K G S A A F Q Y T M R H V L E A F R K A N A D V T L V Q Y M A
A L A S D R T D L E H D R V V L Q S K E L L N S G F S T A E E K F
Q K D A A F Q W M G Y E L W A T K W K L Q K E L A Q R E T W T V N
D Q K L V G V L N W A A Q Y A G K T K H L C R L R G K M T L T E E
V Q W T E M A E A E Y E E N K J L S Q E Q E G C Y Y Q E G K A L E
A T V K S Q D N Q W S Y K H Q E D K L K V G K F A K J K N T H T N
G V R L L A H V Q K G K E A V W G Q V A K F H L A V E K D V W E Q
W W T D Y W Q V T W A E W D F S T A A L V R L V F N L V K D A E G
E E T Y Y T D G S C N K Q S K E G K A G Y T D R G K D K V K V L E
Q T T N Q Q A A L E A F L M A L T D S G A K A N V D S Q Y V M G I
I T G C A T E S E S R L V N Q E E M K K S E Y V A W V A A H K G G
G N Q E D H L V S Q G R Q V L F L E K E A A Q E E H D K Y H S N V
K E L V F K F G L A R V A R Q V D T C D K C H Q K G E A H G Q A N
S D L G T W Q M A C T H L E G K V A V H V A S G F E A E V A Q E T
G R Q T A L F L L K L A G R W A T H L H T A N G A N F A S Q E V K
M V A W W A G E H T F G V A Y N A Q S Q G V V A A M N H H L K N Q
D R R E Q A N S V E T V L M A V H C M N F K R R G G G D M T A A E
R L N M T T E Q E Q F Q Q S K N S K F K N F R V Y Y R E G R D Q L
W K G A G E L L W K G E G A V L K V G T D K V V A R R K A K K D Y
G G G K E V D S S S H M E D T G E A R E V A (pol)
R V A A A (linker)
A S M R R S R A S G D L R Q R L L R A R G E T Y G R L L G E V E D
G Y S Q S A G G L D K G L S S L S C E G Q K Y N Q G Q Y M N T A W
R N A A E E R E K L A Y R K Q N M D D D E E D D D L V G V S V R A
K V A L R T M S Y K L A D M S H F K E K G G L E G I Y Y S A R R H
R L D Y L E K E E G A D W Q D Y T S G P G I R Y A K T F G W L W K
L V A V N V S D E A Q E D E E H Y L M H P A Q T S Q W D D A W G E
V L A W K F D A T L A Y T Y E A Y V R Y A E E F G S K S G L S E E
E V R R R L T A R G L L N M A D K K E T R G A E T A L R E Q E N S
L E S S N E R S S C S E A D A S T P E S A N L G E E L S Q L Y R A
L E A C Y N T C Y C K K C C Y H C Q F C F L K K G L G C Y E Q S R
K R R R T A K K A K A N T S S A S N N R A S N R T R H C Q A E K A
K K E T V E K A V A T A P G L G R G S E E E K R W A V A T W R A E
R L E R W H S L K Y L K Y K T K D L Q K V C Y V A H F K V G W A W
W T C S R V F P L Q E G S H L E V Q G Y W H L T A E K G W L S T Y
A V R T W Y S K N F W T D V T A N Y A D L L H S T Y F A C F T A G
E V R R A I R G E Q L L S C C R F A R A H K Y Q V A S L Q Y L A L
K V V S D V R S Q G E N A T W K Q W R R D N R R G L R M A K Q N S
R G D K Q R G G K A A T K G A N F A G L A K V L G L A V N •
(NefTatVif)
Note:
pol has mutations to inactivate Protease, RT, Int

Comparison wildtype pol versus mutant pol (SIVmac239)

| | | | |
|---|---|---|---|
| Query: | 1 | PQFSLWRRPVVTAHIEGQPVEVLLDTGADDSIVTGIELGPHYTPKIVGGIGGFINTKEYK | 60 |
| | | PQFSLWRRPVVTAHIEGQPVEVLL ADDSIVTGIELGPHYTPKIVGGIGGFINTKEYK | |
| Sbjct: | 1 | PQFSLWRRPVVTAHIEGQPVEVLLAAAADDSIVTGIELGPHYTPKIVGGIGGFINTKEYK | 60 |
| Query: | 61 | NVEIEVLGKRIKGTIMTGDTPINIFGRNLLTALGMSLNFPIAKVEPVKVALKPGKDGPKL | 120 |
| | | NVEIEVLGKRIKGTIMTGDTPINIFGRNLLTALGMSLNFPIAKVEPVKVALKPGKDGPKL | |
| Sbjct: | 61 | NVEIEVLGKRIKGTIMTGDTPINIFGRNLLTALGMSLNFPIAKVEPVKVALKPGKDGPKL | 120 |
| Query: | 121 | KQWPLSKEKIVALREICEKMEKDGQLEEAPPTNPYNTPTFAIKKKDKNKWRMLIDFRELN | 180 |
| | | KQWPLSKEKIVALREICEKMEKDGQLEEAPPTNPYNTPTFAIKKKDKNKWRMLIDFRELN | |
| Sbjct: | 121 | KQWPLSKEKIVALREICEKMEKDGQLEEAPPTNPYNTPTFAIKKKDKNKWRMLIDFRELN | 180 |
| Query: | 181 | RVTQDFTEVQLGIPHPAGLAKRKRITVLDIGDAYFSIPLDEEFRQYTAFTLPSVNNAEPG | 240 |
| | | RVTQDFTEVQLGIPHPAGLAKRKRITVLDIGDAYFSIPLDEEFRQYTAFTLPSVNNAEPG | |
| Sbjct: | 181 | RVTQDFTEVQLGIPHPAGLAKRKRITVLDIGDAYFSIPLDEEFRQYTAFTLPSVNNAEPG | 240 |
| Query: | 241 | KRYIYKVLPQGWKGSPAIFQYTMRHVLEPFRKANPDVTLVQYMDDILIASDRTDLEHDRV | 300 |
| | | KRYIYKVLPQGWKGSPAIFQYTMRHVLEPFRKANPDVTLVQYM ILIASDRTDLEHDRV | |
| Sbjct: | 241 | KRYIYKVLPQGWKGSPAIFQYTMRHVLEPFRKANPDVTLVQYMAAILIASDRTDLEHDRV | 300 |
| Query: | 301 | VLQSKELLNSIGFSTPEEKFQKDPPFQWMGYELWPTKWKLQKIELPQRETWTVNDIQKLV | 360 |
| | | VLQSKELLNSIGFSTPEEKFQKDPPFQWMGYELWPTKWKLQKIELPQRETWTVNDIQKLV | |
| Sbjct: | 301 | VLQSKELLNSIGFSTPEEKFQKDPPFQWMGYELWPTKWKLQKIELPQRETWTVNDIQKLV | 360 |
| Query: | 361 | GVLNWAAQIYPGIKTKHLCRLIRGKMTLTEEVQWTEMAEAEYEENKIILSQEQEGCYYQE | 420 |
| | | GVLNWAAQIYPGIKTKHLCRLIRGKMTLTEEVQWTEMAEAEYEENKIILSQEQEGCYYQE | |
| Sbjct: | 361 | GVLNWAAQIYPGIKTKHLCRLIRGKMTLTEEVQWTEMAEAEYEENKIILSQEQEGCYYQE | 420 |
| Query: | 421 | GKPLEATVIKSQDNQWSYKIHQEDKILKVGKFAKIKNTHTNGVRLLAHVIQKIGKEAIVI | 480 |
| | | GKPLEATVIKSQDNQWSYKIHQEDKILKVGKFAKIKNTHTNGVRLLAHVIQKIGKEAIVI | |
| Sbjct: | 421 | GKPLEATVIKSQDNQWSYKIHQEDKILKVGKFAKIKNTHTNGVRLLAHVIQKIGKEAIVI | 480 |
| Query: | 481 | WGQVPKFHLPVEKDVWEQWWTDYWQVTWIPEWDFISTPPLVRLVFNLVKDPIEGEETYYT | 540 |
| | | WGQVPKFHLPVEKDVWEQWWTDYWQVTWIPEWDFISTPPLVRLVFNLVKDPIEGEETYYT | |
| Sbjct: | 481 | WGQVPKFHLPVEKDVWEQWWTDYWQVTWIPEWDFISTPPLVRLVFNLVKDPIEGEETYYT | 540 |
| Query: | 541 | DGSCNKQSKEGKAGYITDRGKDKVKVLEQTTNQQAELEAFLMALTDSGPKANIIVDSQYV | 600 |
| | | DGSCNKQSKEGKAGYITDRGKDKVKVLEQTTNQQAELEAFLMALTDSGPKANIIVDSQYV | |
| Sbjct: | 541 | DGSCNKQSKEGKAGYITDRGKDKVKVLEQTTNQQAELEAFLMALTDSGPKANIIVDSQYV | 600 |
| Query: | 601 | MGIITGCPTESESRLVNQIIEEMIKKSEIYVAWVPAHKGIGGNQEIDHLVSQGIRQVLFL | 660 |
| | | MGIITGCPTESESRLVNQIIEEMIKKSEIYVAWVPAHKGIGGNQEIDHLVSQGIRQVLFL | |
| Sbjct: | 601 | MGIITGCPTESESRLVNQIIEEMIKKSEIYVAWVPAHKGIGGNQEIDHLVSQGIRQVLFL | 660 |
| Query: | 661 | EKIEPAQEEHDKYHSNVKELVFKFGLPRIVARQIVDTCDKCHQKGEAIHGQANSDLGTWQ | 720 |
| | | EKIEPAQEEHDKYHSNVKELVFKFGLPRIVARQIVDTCDKCHQKGEAIHGQANSDLGTWQ | |
| Sbjct: | 661 | EKIEPAQEEHDKYHSNVKELVFKFGLPRIVARQIVDTCDKCHQKGEAIHGQANSDLGTWQ | 720 |
| Query: | 721 | MDCTHLEGKIIIVAVHVASGFIEAEVIPQETGRQTALFLLKLAGRWPITHLHTDNGANFA | 780 |
| | | M CTHLEGKIIIVAVHVASGFIEAEVIPQETGRQTALFLLKLAGRWPITHLHT NGANFA | |
| Sbjct: | 721 | MACTHLEGKIIIVAVHVASGFIEAEVIPQETGRQTALFLLKLAGRWPITHLHTANGANFA | 780 |
| Query: | 781 | SQEVKMVAWWAGIEHTFGVPYNPQSQGVVEAMNHHLKNQIDRIREQANSVETIVLMAVHC | 840 |
| | | SQEVKMVAWWAGIEHTFGVPYNPQSQGVVEAMNHHLKNQIDRIREQANSVETIVLMAVHC | |
| Sbjct: | 781 | SQEVKMVAWWAGIEHTFGVPYNPQSQGVVEAMNHHLKNQIDRIREQANSVETIVLMAVHC | 840 |
| Query: | 841 | MNFKRRGGIGDMTPAERLINMITTEQEIQFQQSKNSKFKNFRVYYREGRDQLWKGPGELL | 900 |
| | | MNFKRRGGIGDMTPAERLINMITTEQEIQFQQSKNSKFKNFRVYYREGRDQLWKGPGELL | |
| Sbjct: | 841 | MNFKRRGGIGDMTPAERLINMITTEQEIQFQQSKNSKFKNFRVYYREGRDQLWKGPGELL | 900 |
| Query: | 901 | WKGEGAVILKVGTDIKVVPRRKAKIIKDYGGGKEVDSSSHMEDTGEAREVA | 951 |
| | | WKGEGAVILKVGTDIKVVPRRKAKIIKDYGGGKEVDSSSHMEDTGEAREVA | |
| Sbjct: | 901 | WKGEGAVILKVGTDIKVVPRRKAKIIKDYGGGKEVDSSSHMEDTGEAREVA | 951 |

59S_CMV_CATESVenvi gene: 780-3452
CGATGATATCCATTGCATACGTTGTATCTATATCATAATATGTACATTTATATTGGCTCATGTCCA
ATATGACCGCCATGTTGACATTGATTATTGACTAGTTATTAATAGTAATCAATTACGGGGTCATTA
GTTCATAGCCCATATATGGAGTTCCGCGTTACATAACTTACGGTAAATGGCCCGCCTGGCTGACCG
CCCAACGACCCCCGCCCATTGACGTCAATAATGACGTATGTTCCCATAGTAACGCCAATAGGGACT
TTCCATTGACGTCAATGGGTGGAGTATTTACGGTAAACTGCCCACTTGGCAGTACATCAAGTGTAT
CATATGCCAAGTCCGCCCCCTATTGACGTCAATGACGGTAAATGGCCCGCCTGGCATTATGCCCAG
TACATGACCTTACGGGACTTTCCTACTTGGCAGTACATCTACGTATTAGTCATCGCTATTACCATG
GTGATGCGGTTTTGGCAGTACATCAATGGGCGTGGATAGCGGTTTGACTCACGGGGATTTCCAAGT
CTCCACCCCATTGACGTCAATGGGAGTTTGTTTTGGCACCAAAATCAACGGGACTTTCCAAAATGT
CGTAACAACTCCGCCCCATTGACGCAAATGGGCGGTAGGCGTGTACGGTGGGAGGTCTATATAAGC
AGAGCTCGTTTAGTGAACCGTCAGATCGCCTGGAGACGCCATCCACGCTGTTTTGACCTCCATAGA
AGACACCGGGACCCGATCCAGCCTCCGCGGGCGCGCGTCGAGGAATTCAAGAAATGAGAAAAGCGG
CTGTTAGTCACTGGCAGCAGCAGTCTTACCTGGACTCTGGAATCCATTCTGGTGCCACTACCACAG
CTCCTTCTCTGAGTATCTGCAGCCTGTACGTCACGGTCTTCTACGGCGTACCAGCTTGGAGGAATG
CGACAATTCCCCTCTTTTGTGCAACCAAGAATAGGGATACTTGGGGAACAACTCAGTGCCTACCGG
ACAACGGGGACTACTCGGAGGTGGCCCTGAACGTGACGGAGAGCTTCGACGCCTGGAACAACACGG
TCACGGAGCAGGCGATCGAGGACGTGTGGCAGCTGTTCGAGACCTCGATCAAGCCGTGCGTCAAGC
TGTCCCCGCTCTGCATCACGATGCGGTGCAACAAGAGCGAGACGGATCGGTGGGGGCTGACGAAGT
CGATCACGACGACGGCGTCGACCACGTCGACGACGGCGTCGGCGAAAGTGGACATGGTCAACGAGA
CCTCGTCGTGCATCGCCCAGGACAACTGCACGGGCCTGGAGCAGGAGCAGATGATCAGCTGCAAGT
TCAACATGACGGGGCTGAAGCGGGACAAGAAGAAGGAGTACAACGAGACGTGGTACTCGGCGGACC
TGGTGTGCGAGCAGGGGAACAACACGGGGAACGAGTCGCGGTGCTACATGAACCACTGCAACACGT
CGGTGATCCAGGAGTCGTGCGACAAGCACTACTGGGACGCGATCCGGTTCCGGTACTGCGCGCCGC
CGGGCTACGCGCTGCTGCGGTGCAACGACACGAACTACTCGGGCTTCATGCCGAAATGCTCGAAGG
TGGTGGTCTCGTCGTGCACGAGGATGATGGAGACGCAGACCTCGACGTGGTTCGGCTTCAACGGGA
CGCGGGCGGAGAACCGGACGTACATCTACTGGCACGGGCGGGACAACCGGACGATCATCTCGCTGA
ACAAGTACTACAACCTGACGATGAAGTGCCGGCGGCCGGGCAACAAGACGGTGCTCCCGGTCACCA
TCATGTCGGGGCTGGTGTTCCACTCGCAGCCGATCAACGACCGGCCGAAGCAGGCGTGGTGCTGGT
TCGGGGGGAAGTGGAAGGACGCGATCAAGGAGGTGAAGCAGACCATCGTCAAGCACCCCCGCTACA
CGGGGACGAACAACACGGACAAGATCAACCTGACGGCGCCGGGCGGGGGCGATCCGGAAGTTACCT
TCATGTGGACJAAJLTTGCAGAGGAGAGTTCCTCTACTGCAAGATGAACTGGTTCCTGAACTGGGT
GGAGGACAGGAACACGGCAGAACCAGAAGCCGAAGGAGCAGCACAAGCGGAACTACGTGCCGTGCC
ACATTCGGCAGATCATCAACACGTGGCACAAAGTGGGCAAGAACGTGTACCTGCCGCCGAGGGAGG
GCGACCTCACGTGCAACTCCACGGTGACCTCCCTCATCGCGAAAAACATCGACTGGATCGACGGCA
ACCAGACGAACATCACCATGTCGGCGGAGGTGGCGGAGCTGTACCGGCTGGAGCTGGGGGACTACA
AGCTGGTGGAGATCACGCCGATCGGCCTGGCCCCCACCGATGTGAAGCGCTACACGACCGGGGGGA
CGTCGCGGAACAAGCGGGGGGTCTTCGTCCTGGGGTTCCTGGGGTTCCTCGCGACGGCGGGGTCGG
CJAATGGGAGCCGCCAGCCTGACCCTCACGGCACAGTCCCGACTTTATTGGCTGGGATCGTCCAAC
AACAGCAGCAGCTGCTGGACGTGGTCAAGAGGCAGCAGGAGCTGCTGCGGCTGACCGTCTGGGGCA
CGAAGAACCTCCAGACGAGGGTCACGGCCATCGAGAAGTACCTGAAGGACCAGGCGCAGCTGAACG
CGTGGGGCTGTGCGTTTCGACAAGTCTGCCACACGACGGTCCCGTGGCCGAACGCGTCGCTGACGC
CGAAGTGGAACAACGAGACGTGGCAGGAGTGGGAGCGGAAGGTGGACTTCCTGGAGGAGAACATCA
CGGCCCTCCTGGAGGAGGCGCAGATCCAGCAGGAGAAGAACATGTACGAGCTGCJAAJAAGCTGAA
CAGCTGGGACGTGTTCGGCJAAJAACTGGTTCGACCTGGCGTCGTGGATCAAGTACATCCAGTACG
GCGTGTACATCGTGGTGGGGGTGATCCTGCTGCGGATCGTGATCTACATCGTCCAGATGCTGGCGA
AAGCTGCGGCAGGGCTATAGGCCAGTGTTCTCTTCCCCACCCTCTTATTTCCAACAAACCCATATC
CAAACAAGACCCGGCGCTGCCGACCCGGGAGGGCAAGGAGCGGGACGGCGGGGAGGGCGGCGGCAA
CAGCTCCTGGCCGTGGCAGATCGAGTACATCCACTTTCTTATTCGTCAGCTTATTAGACTCCTGAC
GTGGCTGTTCAGTAACTGTAGGACTCTGCTGTCGAGGGTGTACCAGATCCTCCAGCCGATCCTCCA
GCGGCTCTCGGCGACCCTCCAGAGGATTCGGGAGGTCCTCCGGACGGAGCTGACCTACCTCCAGTA
CGGGTGGAGCTATTTCCACGAGGCGGTCCAGGCCGTCTGGCGGTCGGCGACGGAGACGCTGGCGGG
CGCGTGGGGCGACCTGTGGGAGACGCTGCGGCGGGGCGGCCGGTGGATACTCGCGATCCCCCGGCG
GATCAGGCAGGGGCTGGAGCTCACGCTCCTGTGATAAGATATCGGATCCGCCCGGGCTAGAGCGGC
CACTCGAGAGGCGCGCCGAGCTCGCTGATCAGCCTCGACTGTGCCTTCTAGTTGCCAGCCATCTGT
TGTTTGCCCCTCCCCCGTGCCTTCCTTGACCCTGGAAGGTGCCACTCCCACTGTCCTTTCCTAATA
AATGAGGMAATTGCATCGCATTGTCTGAGTAGGTGTCATTCTATTCTGGGGGGTGGGGTGGGGCAG
GACAGCAAGGGGGAGGATGGGAAGACAATAGCAGGCATGCTGGGGAATTTAAATGGGGGCGCTGAG
GTCTGCCTCGTGAAGAAGGTGTTGCTGACTCATACCAGGCCTGAATCGCCCCATCATCCAGCCAGA
AAGTGAGGGAGCCACGGTTGATGAGAGCTTTGTTGTAGGTGGACCAGTTGGTGATTTTGAACTTTT
GCTTTGCCACGGAACGGTCTGCGTTGTCGGGAAGATGCGTGATCTGATCCTTCAACTCAGCAAAAG
TTCGATTTATTCAACAAAGCCGCCGTCCCGTCAAGTCAGCGTAATGCTCTGCCAGTGTTACAACCA
ATTAACCAATTCTGCGTTCAAAATGGTATGCGTTTTGACACATCCACTATATATCCGTGTCGTTCT
GTCCACTCCTGAATCCCATTCCAGAAATTCTCTAGCGATTCCAGAAGTTTCTCAGAGTCGGAAAGT
TGACCAGACATTACGAACTGGCACAGATGGTCATAACCTGAAGGAAGATCTGATTGCTTAACTGCT
TCAGTTAAGACCGACGCGCTCGTCGTATAACAGATGCGATGATGCAGACCAATCAACATGGCACCT
GCCATTGCTACCTGTACAGTCAAGGATGGTAGAAATGTTGTCGGTCCTTGCACACGAATATTACGC
CATTTGCCTGCATATTCAAACAGCTCTTCTACGATAAGGGCACAAATCGCATCGTGGAACGTTTGG
GCTTCTACCGATTTAGCAGTTTGATACACTTTCTCTAAGTATCCACCTGAATCATAAATCGGCAAA
ATAGAGAAAAATTGACCATGTGTAAGCGGCCAATCTGATTCCACCTGAGATGCATAATCTAGTAGA
ATCTCTTCGCTATCAAAATTCACTTCCACCTTCCACTCACCGGTTGTCCATTCATGGCTGAACTCT
GCTTCCTCTGTTGACATGACACACATCATCTCAATATCCGAATACGGACCATCAGTCTGACGACCA
AGAGAGCCATAAACACCAATAGCCTTAACATCATCCCCATATTTATCCAATATTCGTTCCTTAATT
TCATGAACAATCTTCATTCTTTCTTCTCTAGTCATTATTATTGGTCCGTTCATAACACCCCTTGTA
TTACTGTTTATGTAAGCAGACAGTTTTATTGTTCATGATGATATATTTTTATCTTGTGCAATGTAA
CATCAGAGATTTTGAGACACAACGTGGCTTTCCCCGGCCCATGACCAAAATCCCTTAACGTGAGTT
TTCGTTCCACTGAGCGTCAGACCCCGTAGAAAAGATCAAAGGATCTTCTTGAGATCCTTTTTTTCT
GCGCGTAATCTGCTGCTTGCAAACAAAAAAACCACCGCTACCAGCGGTGGTTTGTTTGCCGGATCA
AGAGCTACCAACTCTTTTTCCGAAGGTAACTGGCTTCAGCAGAGCGCAGATACCAAATACTGTCCT
TCTAGTGTAGCCGTAGTTAGGCCACCACTTCAAGAACTCTGTAGCACCGCCTACATACCTCGCTCT
GCTAATCCTGTTACCAGTGGCTGCTGCCAGTGGCGATAAGTCGTGTCTTACCGGGTTGGACTCAAG
ACGATAGTTACCGGATAAGGCGCAGCGGTCGGGCTGAACGGGGGGTTCGTGCACACAGCCCAGCTT
GGAGCGAACGACCTACACCGAACTGAGATACCTACAGCGTGAGCTATGAGAAAGCGCCACGCTTCC
CGAAGGGAGAAAGGCGGACAGGTATCCGGTAAGCGGCAGGGTCGGAACAGGAGAGCGCACGAGGGA
GCTTCCAGGGGGAAACGCCTGGTATCTTTATAGTCCTGTCGGGTTTCGCCACCTCTGACTTGAGCG
TCGATTTTTGTGATGCTCGTCAGGGGGGCGGAGCCTATGGAAAAACGCCAGCAACGCGGCCTTTTT
ACGGTTCCTGGCCTTTTGCTGGCCTTTTGCTCACATGTTCTTTCCTGCGTTATCCCCTGATTCTGT
GGATAACCGTATTACCGCCTTTGAGTGAGCTGATACCGCTCGCCGCAGCCGAACGACCGAGCGCAG
CGAGTCAGTGAGCGAGGAAGCGGAAGAGCGCCTGATGCGGTATTTTCTCCTTACGCATCTGTGCGG
TATTTCACACCGCATATGGTGCACTcTCAGTAcpAATCTGCTCTGATGCCGCATAGTTAAGCCAGT
ATCTGCTCCCTGCTTGTGTGTTGGAGGTCGCTGAGTAGTGCGCGAGCAAAATTTAAGCTACAACAA
GGCAAGGCTTGACCGACAATTGCATGAAGAATCTGCTTAGGGTTAGGCGTTTTGCGCTGCTTCGCG
ATGTACGGGCCAGATATAGCCGCGGCATCG (6022)
protein:
M R K A A V S H W Q Q Q S Y L D S G H S G A T T T A A S L S
(CATE)
I C S (linker)
L Y V T V F Y G V A A W R N A T A L F C A T K N R D T W G T T Q C
L A D N G D Y S E V A L N V T E S F D A W N N T V T E Q A E D V W
Q L F E T S K A C V K L S A L C T M R C N K S E T D R W G L T K S
T T T A S T T S T T A S A K V D M V N E T S S C A Q D N C T G L E
Q E Q M S C K F N M T G L K R D K K K E Y N E T W Y S A D L V C E
Q G N N T G N E S R C Y M N H C N T S V Q E S C D K H Y W D A R F
R Y C A A A G Y A L L R C N D T N Y S G F M A K C S K V V V S S C
T R M M E T Q T S T W F G F N G T R A E N R T Y Y W H G R D N R T
S L N K Y Y N L T M K C R R A G N K T V L A V T M S G L V F H S Q
A N D R A K Q A W C W F G G K W K D A K E V K Q T V K H A R Y T G
T N N T D K J N L T A A G G G D A E V T F M W T N C R G E F L Y C
K M N W F L N W V E D R N T A N Q K A K E Q H K R N Y V A C H R Q
N T W H K V G K N V Y L A A R E G D L T C N S T V T S L A N D W D
G N Q T N T M S A E V A E L Y R L E L G D Y K L V E T A G L A A T
D V K R Y T T G G T S R N K R G V F V L G F L G F L A T A G S A M
G A A S L T L T A Q S R T L L A G V Q Q Q Q Q L L D V V K R Q Q E
L L R L T V W G T K N L Q T R V T A E K Y L K D Q A Q L N A W G C
A F R Q V C H T T V A W A N A S L T A K W N N E T W Q E W E R K V
D F L E E N T A L L E E A Q Q Q E K N M Y E L Q K L N S W D V F G
N W F D L A S W K Y Q Y G V Y V V G V L L R V Y V Q M L A K L R Q
G Y R A V F S S A A S Y F Q Q T H Q Q D A A L A T R E G K E R D G
G E G G G N S S W A W Q E Y H F L R Q L R L L T W L F S N C R T L
L S R V Y Q L Q A L Q R L S A T L Q R R E V L R T E L T Y L Q Y G
W S Y F H E A V Q A V W R S A T E T L A G A W G D L W E T L R R G
G R W L A A R R R Q G L E L T L L • (env)
72S pCMV CATESIVenv CATE-env gene: 775-3447
(1)CCTGGCCATTGCATACGTTGTATCCATATCATAATATGTACATTTATATTGGCTCATGTCCAA
CATTACCGCCATGTTGACATTGATTATTGACTAGTTATTAATAGTAATCAATTACGGGGTCATTAG
TTCATAGCCCATATATGGAGTTCCGCGTTACATAACTTACGGTAAATGGCCCGCCTGGCTGACCGC
CCAACGACCCCCGCCCATTGACGTCAATAATGACGTATGTTCCCATAGTAACGCCAATAGGGACTT
TCCATTGACGTCAATGGGTGGAGTATTTACGGTAAACTGCCCACTTGGCAGTACATCAAGTGTATC
ATATGCCAAGTACGCCCCCTATTGACGTCAATGACGGTAAATGGCCCGCCTGGCATTATGCCCAGT
ACATGACCTTATGGGACTTTCCTACTTGGCAGTACATCTACGTATTAGTCATCGCTATTACCATGG
TGATGCGGTTTTGGCAGTACATCAATGGGCGTGGTAGCGGTTTGACTCACGGGGATTTCCAAGTCT
CCACCCCATTGACGTCAATGGGAGTTTGTTTTGGCACCAAAATCAACGGGACTTTCCAAAATGTCG
TAACAACTCCGCCCCATTGACGCAAATGGGCGGTAGGCGTGTACGGTGGGAGGTCTATATAAGCAG
AGCTCGTTTAGTGAACCGTCAGATCGCCTGGAGACGCCATCCACGCTGTTTTGACCTCCATAGAAG
ACACCGGGACCGATCCAGCCTCCGCGGGCGCGCGTCGAGGAATTCAAGAAATGAGAAAAGCGGCTG
TTAGTCACTGGCAGCAGCAGTCTTACCTGGACTCTGGAATCCATTCTGGTGCCACTACCACAGCTC
CTTCTCTGAGTATCTGCAGCCTGTACGTCACGGTCTTCTACGGCGTACCAGCTTGGAGGAATGCGA
CAATTCCCCTCTTTTGTGCAACCAAGAATAGGGATACTTGGGGAACAACTCAGTGCCTACCGGACA
ACGGGGACTACTCGGAGGTGGCCCTGAACGTGACGGAGAGCTTCGACGCCTGGAACAACACGGTCA
CGGAGCAGGCGATCGAGGACGTGTGGCAGCTGTTCGAGACCTCGATCAAGCCGTGCGTCAAGCTGT
CCCCGCTCTGCATCACGATGCGGTGCAACAAGAGCGAGACGGATCGGTGGGGGCTGACGAAGTCGA
TCACGACGACGGCGTCGACCACGTCGACGACGGCGTCGGCGAAAGTGGACATGGTCAACGAGACCT
CGTCGTGCATCGCCCAGGACAACTGCACGGGCCTGGAGCAGGAGCAGATGATCAGCTGCAAGTTCA
ACATGACGGGGCTGAAGCGGGACAAGAAGAJLGGAGTACAACGAGACGTGGTACTCGGCGGACCTG
GTGTGCGAGCAGGGGAACAACACGGGGAACGAGTCGCGGTGCTACATGAACCACTGCAACACGTCG
GTGATCCAGGAGTCGTGCGACAAGCACTACTGGGACGCGATCCGGTTCCGGTACTGCGCGCCGCCG
GGCTACGCGCTGCTGCGGTGCAACGACACGAACTACTCGGGCTTCATGCCGAAATGCTCGAAGGTG
GTGGTCTCGTCGTGCACGAGGATGATGGAGACGCAGACCTCGACGTGGTTCGGCTTCAACGGGACG
CGGGCGGAGAACCGGACGTACATCTACTGGCACGGGCGGGACAACCGGACGATCATCTCGCTGAAC
AAGTACTACAACCTGACGATGAAGTGCCGGCGGCCGGGCAACAAGACGGTGCTCCCGGTCACCATC
ATGTCGGGGCTGGTGTTCCACTCGCAGCCGATCAACGACCGGCCGAAGCAGGCGTGGTGCTGGTTC
GGGGGGAAGTGGAAGGACGCGATCAAGGAGGTGAAGCAGACCATCGTCAAGCACCCCCGCTACACG
GGGACGAACAACACGGACAAGATCAACCTGACGGCGCCGGGCGGGGGCGATCCGGAAGTTACCTTC
ATGTGGACAAATTGCAGAGGAGAGTTCCTCTACTGCAAGATGAACTGGTTCCTGAACTGGGTGGAG
GACAGGAACACGGCGAACCAGAAGCCGAAGGAGCAGCACAAGCGGAACTACGTGCCGTGCCACATT
CGGCAGATCATCAACACGTGGCACAAAGTGGGCAAGAACGTGTACCTGCCGCCGAGGGAGGGCGAC
CTCACGTGCAACTCCACGGTGACCTCCCTCATCGCGAACATCGACTGGATCGACGGCAACCAGACG
AACATCACCATGTCGGCGGAGGTGGCGGAGCTGTACCGGCTGGAGCTGGGGGACTACAAGCTGGTG
GAGATCACGCCGATCGGCCTGGCCCCCACCGATGTGAAGCGCTACACGACCGGGGGGACGTCGCGG
AACAAGCGGGGGGTCTTCGTCCTGGGGTTCCTGGGGTTCCTCGCGACGGCGGGGTCGGCAATGGGA
GCCGCCAGCCTGACCCTCACGGCACAGTCCCGAACTTTATTGGCTGGGATCGTCCAACAACAGCAG
CAGCTGCTGGACGTGGTCAAGAGGCAGCAGGAGCTGCTGCGGCTGACCGTCTGGGGCACGAAGAAC
CTCCAGACGAGGGTCACGGCCATCGAGAAGTACCTGAAGGACCAGGCGCAGCTGAACGCGTGGGGC
TGTGCGTTTCGACAAGTCTGCCACACGACGGTCCCGTGGCCGAACGCGTCGCTGACGCCGAAGTGG
AACAACGAGACGTGGCAGGAGTGGGAGCGGAAGGTGGACTTCCTGGAGGAGAACATCACGGCCCTC
CTGGAGGAGGCGCAGATCCAGCAGGAGAAGAACATGTACGAGCTGCAAAAGCTGAACAGCTGGGAC
GTGTTCGGCAACTGGTTCGACCTGGCGTCGTGGATCAAGTACATCCAGTACGGCGTGTACATCGTG
GTGGGGGTGATCCTGCTGCGGATCGTGATCTACATCGTCCAGATGCTGGCGAAGCTGCGGCAGGGC
TATAGGCCAGTGTTCTCTTCCCCACCCTCTTATTTCCAACAAACCCATATCCAACAAGACCCGGCG
CTGCCGACCCGGGAGGGCAAGGAGCGGGACGGCGGGGAGGGCGGCGGCAACAGCTCCTGGCCGTGG
CAGATCGAGTACATCCACTTTCTTATTCGTCAGCTTATTAGACTCCTGACGTGGCTGTTCAGTAAC
TGTAGGACTCTGCTGTCGAGGGTGTACCAGATCCTCCAGCCGATCCTCCAGCGGCTCTCGGCGACC
CTCCAGAGGATTCGGGAGGTCCTCCGGACGGAGCTGACCTACCTCCAGTACGGGTGGAGCTATTTC
CACGAGGCGGTCCAGGCCGTCTGGCGGTCGGCGACGGAGACGCTGGCGGGCGCGTGGGGCGACCTG
TGGGAGACGCTGCGGCGGGGCGGCCGGTGGATACTCGCGATCCCCCGGCGGATCAGGCAGGGGCTG
GAGCTCACGCTCCTGTGATAAGATATCGGATCTGCTGTGCCTTCTAGTTGCCAGCCATCTGTTGTT
TGCCCCTCCCCCGTGCCTTCCTTGACCCTGGAAGGTGCCACTCCCACTGTCCTTTCCTAATAAAAT
GAGGAAATTGCATCGCATTGTCTGAGTAGGTGTCATTCTATTCTGGGGGGTGGGGTGGGGCAGCAC
AGCAAGGGGGAGGATTGGGAAGACAATAGCAGGCATGCTGGGGATGCGGTGGGCTCTATGGGTACC
CAGGTGCTGAAGAATTGACCCGGTTCCTCCTGGGCCAGAAAGAAGCAGGCACATCCCCTTCTCTGT
GACACACCCTGTCCACGCCCCTGGTTCTTAGTTCCAGCCCCACTCATAGGACACTCATAGCTCAGG
AGGGCTCCGCCTTCAATCCCACCCGCTAAAGTACTTGGAGCGGTCTCTCCCTCCCTCATCAGCCCA
CCAAACCAAACCTAGCCTCCAAGAGTGGGAAGAAATTAAAGCAAGATAGGCTATTAAGTGCAGAGG
GAGAGAAAATGCCTCCAACATGTGAGGAAGTAATGAGAGAAATCATAGAATTTCTTCCGCTTCCTC
GCTCACTGACTCGCTGCGCTCGGTCGTTCGGCTGCGGCGAGCGGTATCAGCTCACTCAAAGGCGGT
AATACGGTTATCCACAGAATCAGGGGATAACGCAGGAAAGAACATGTGAGCAAAAGGCCAGCAAAA
GGCCAGGAACCGTAAAAAGGCCGCGTTGCTGGCGTTTTTCCATAGGCTCCGCCCCCCTGACGAGCA
TCACAAAAATCGACGCTCAAGTCAGAGGTGGCGAAACCCGACAGGACTATAAAGATACCAGGCGTT
TCCCCCTGGAAGCTCCCTCGTGCGCTCTCCTGTTCCGACCCTGCCGCTTACCGGATACCTGTCCGC
CTTTCTCCCTTCGGGAAGCGTGGCGCTTTCTCAATGCTCACGCTGTAGGTATCTCAGTTCGGTGTA
GGTCGTTCGCTCCAAGCTGGGCTGTGTGCACGAACCCCCCGTTCAGCCCGACCGCTGCGCCTTATC
CGGTAACTATCGTCTTGAGTCCAACCCGGTAAGACACGACTTATCGCCACTGGCAGCAGCCACTGG
TAACAGGATTAGCAGAGCGAGGTATGTAGGCGGTGCTACAGAGTTCTTGAAGTGGTGGCCTAACTA
CGGCTACACTAGAAGGACAGTATTTGGTATCTGCGCTCTGCTGAAGCCAGTTACCTTCGGAAAAAG
AGTTGGTAGCTCTTGATCCGGCAAACAAACCACCGCTGGTAGCGGTGGTTTTTTTGTTTGCAAGCA
GCAGATTACGCGCAGAAAAAAAGGATCTCAAGAAGATCCTTTGATCTTTTCTACGGGGTCTGACGC
TCAGTGGAACGAAAACTCACGTTAAGGGATTTTGGTCATGAGATTATCAAAAAGGATCTTCACCTA
GATCCTTTTAAATTAAAAATGAAGTTTTAAATCAATCTAAAGTATATATGAGTAAACTTGGTCTGA
CAGTTACCAATGCTTAATCAGTGAGGCACCTATCTCAGCGATCTGTCTATTTCGTTCATCCATAGT
TGCCTGACTCCGGGGGGGGGGGGCGCTGAGGTCTGCCTCGTGAAGAAGGTGTTGCTGACTCATACC
AGGCCTGAATCGCCCCATCATCCAGCCAGAAAGTGAGGGAGCCACGGTTGATGAGAGCTTTGTTGT
AGGTGGACCAGTTGGTGATTTTGAACTTTTGCTTTGCCACGGAACGGTCTGCGTTGTCGGGAAGAT
GCGTGATCTGATCCTTCAACTCAGCAAAAGTTCGATTTATTCAACAAAGCCGCCGTCCCGTCAAGT
CAGCGTAATGCTCTGCCAGTGTTACAACCAATTAACCAATTCTGATTAGAAAAACTCATCGAGCAT
CAAATGAAACTGCAATTTATTCATATCAGGATTATCAATACCATATTTTTGAAAAAGCCGTTTCTG
TAATGAAGGAGAAAACTCACCGAGGCAGTTCCATAGGATGGCAAGATCCTGGTATCGGTCTGCGAT
TCCGACTCGTCCAACATCAATACAACCTATTAATTTCCCCTCGTCAAAAATAAGGTTATCAAGTGA
GAAATCACCATGAGTGACGACTGAATCCGGTGAGAATGGCAAAAGCTTATGCATTTCTTTCCAGAC
TTGTTCAACAGGCCAGCCATTACGCTCGTCATCAAAATCACTCGCATCAACCAAACCGTTATTCAT
TCGTGATTGCGCCTGAGCGAGACGAAATACGCGATCGCTGTTAAAAGGACAATTACAAACAGGAAT
CGAATGCAACCGGCGCAGGAACACTGCCAGCGCATCAACAATATTTTCACCTGAATCAGGATATTC
TTCTAATACCTGGAATGCTGTTTTCCCGGGGATCGCAGTGGTGAGTAACCATGCATCATCAGGAGT
ACGGATAAAATGCTTGATGGTCGGAAGAGGCATAAATTCCGTCAGCCAGTTTAGTCTGACCATCTC
ATCTGTAACATCATTGGCAACGCTACCTTTGCCATGTTTCAGAAACAACTCTGGCGCATCGGGCTT
CCCATACAATCGATAGATTGTCGCACCTGATTGCCCGACATTATCGCGAGCCCATTTATACCCATA
TAAATCAGCATCCATGTTGGAATTTAATCGCGGCCTCGAGCAAGACGTTTCCCGTTGAATATGGCT
CATAACACCCCTTGTATTACTGTTTATGTAAGCAGACAGTTTTATTGTTCATGATGATATATTTTT
ATCTTGTGCAATGTAACATCAGAGATTTTGAGACACAACGTGGCTTTCCCCCCCCCCCCATTATTG
AAGCATTTATCAGGGTTATTGTCTCATGAGCGGATACATATTTGAATGTATTTAGAAAAATAAACA
AATAGGGGTTCCGCGCACATTTCCCCGAAAAGTGCCACCTGACGTCTAAGAAACCATTATTATCAT
GACATTAACCTATAAAAATAGGCGTATCACGAGGCCCTTTCGTCTCGCGCGTTTCGGTGATGACGG
TGAAAACCTCTGACACATGCAGCTCCCGGAGACGGTCACAGCTTGTCTGTAAGCGGATGCCGGGAG
CAGACAAGCCCGTCAGGGCGCGTCAGCGGGTGTTGGCGGGTGTCGGGGCTGGCTTAACTATGCGGC
ATCAGAGCAGATTGTACTGAGAGTGCACCATATGCGGTGTGAAATACCGCACAGATGCGTAAGGAG
AAAATACCGCATCAGATTGGCTATTGG (6690)
CATE-env protein:
M R K A A V S H W Q Q Q S Y L D S G H S G A T T T A A S L S
(CATE)
I C S (linker)
Env SIVmac239:
L Y V T V F Y G V A A W R N A T A L F C A T K N R D T W G T T Q C
L A D N G D Y S E V A L N V T E S F D A W N N T V T E Q A E D V W
Q L F E T S K A C V K L S A L C T M R C N K S E T D R W G L T K S
T T T A S T T S T T A S A K V D M V N E T S S C A Q D N C T G L E
Q E Q M S C K F N M T G L K R D K K K E Y N E T W Y S A D L V C E
Q G N N T G N E S R C Y M N H C N T S V Q E S C D K H Y W D A R F
R Y C A A A G Y A L L R C N D T N Y S G F M A K C S K V V V S S C
T R M M E T Q T S T W F G F N G T R A E N R T Y Y W H G R D N R T
S L N K Y Y N L T M K C R R A G N K T V L A V T M S G L V F H S Q
A N D R A K Q A W C W F G G K W K D A K E V K Q T V K H A R Y T G
T N N T D K N L T A A G G G D A E V T F M W T N C R G E F L Y C K
M N W F L N W V E D R N T A N Q K A K E Q H K R N Y V A C H R Q N
T W H K V G K N V Y L A A R E G D L T C N S T V T S L A N D W D G
N Q T N T M S A E V A E L Y R L E L G D Y K L V E T A G L A A T D
V K R Y T T G G T S R N K R G V F V L G F L G F L A T A G S A M G
A A S L T L T A Q S R T L L A G V Q Q Q Q Q L L D V V K R Q Q E L
L R L T V W G T K N L Q T R V T A E K Y L K D Q A Q L N A W G C A
F R Q V C H T T V A W A N A S L T A K W N N E T W Q E W E R K V D
F L E E N T A L L E E A Q Q Q E K N M Y E L Q K L N S W D V F G N
W F D L A S W K Y Q Y G V Y V V G V L L R V Y V Q M L A K L R Q G
Y R A V F S S A A S Y F Q Q T H Q Q D A A L A T R E G K E R D G G
E G G G N S S W A W Q E Y H F L R Q L R L L T W L F S N C R T L L
S R V Y Q L Q A L Q R L S A T L Q R R E V L R T E L T Y L Q Y G W
S Y F H E A V Q A V W R S A T E T L A G A W G D L W E T L R R G G
R W L A A R R R Q G L E L T L L
pCMV MCP3 SVenv gene: 775-3660
(1)CCTGGCCATTGCATACGTTGTATCCATATCATAATATGTACATTTATATTGGCTCATGTCCAA
CATTACCGCCATGTTGACATTGATTATTGACTAGTTATTAATAGTAATCAATTACGGGGTCATTAG
TTCATAGCCCATATATGGAGTTCCGCGTTACATAACTTACGGTAAATGGCCCGCCTGGCTGACCGC
CCAACGACCCCCGCCCATTGACGTCAATAATGACGTATGTTCCCATAGTAACGCCAATAGGGACTT
TCCATTGACGTCAATGGGTGGAGTATTTACGGTAAACTGCCCACTTGGCAGTACATCAAGTGTATC
ATATGCCAAGTACGCCCCCTATTGACGTCAATGACGGTAAATGGCCCGCCTGGCATTATGCCCAGT
ACATGACCTTATGGGACTTTCCTACTTGGCAGTACATCTACGTATTAGTCATCGCTATTACCATGG
TGATGCGGTTTTGGCAGTACATCAATGGGCGTGGATAGCGGTTTGACTCACGGGGATTTCCAAGTC
TCCACCCCATTGACGTCAATGGGAGTTTGTTTTGGCACCAAAATCAACGGGACTTTCCAAAATGTC
GTAACAACTCCGCCCCATTGACGCAAATGGGCGGTAGGCGTGTACGGTGGGAGGTCTATATAAGCA
GAGCTCGTTTAGTGAACCGTCAGATCGCCTGGAGACGCCATCCACGCTGTTTTGACCTCCATAGAA
GACACCGGGACCGATCCAGCCTCCGCGGGCGCGCGTCGAGGAATTCAAGAAATGAACCCAAGTGCT
GCCGTCATTTTCTGCCTCATCCTGCTGGGTCTGAGTGGGACTCAAGGGATCCTCGACATGGCGCAA
CCGGTAGGTATAAACACAAGCACAACCTGTTGCTATCGTTTCATAAATAAAAAGATACCGAAGCAA
CGTCTGGAAAGCTATCGCCGTACCACTTCTAGCCACTGTCCGCGTGAAGCTGTTATATTCAAAACG
AAACTGGATAAGGAGATCTGCGCCGACCCTACACAGAAATGGGTTCAGGACTTTATGAAGCACCTG
GATAAAAAGACACAGACGCCGAAACTGATCTGCAGCCTGTACGTCACGGTCTTCTACGGCGTACCA
GCTTGGAGGAATGCGACAATTCCCCTCTTTTGTGCAACCAAGAATAGGGATACTTGGGGAACAACT
CAGTGCCTACCGGACAACGGGGACTACTCGGAGGTGGCCCTGAACGTGACGGAGAGCTTCGACGCC
TGGAACAACACGGTCACGGAGCAGGCGATCGAGGACGTGTGGCAGCTGTTCGAGACCTCGATCAAG
CCGTGCGTCAAGCTGTCCCCGCTCTGCATCACGATGCGGTGCAACAAGAGCGAGACGGATCGGTGG
GGGCTGACGAAGTCGATCACGACGACGGCGTCGACCACGTCGACGACGGCGTCGGCGAAAGTGGAC
ATGGTCAACGAGACCTCGTCGTGCATCGCCCAGGACAACTGCACGGGCCTGGAGCAGGAGCAGATG
ATCAGCTGCAAGTTCAACATGACGGGGCTGAAGCGGGACAAGAAGAAGGAGTACAACGAGACGTGG
TACTCGGCGGACCTGGTGTGCGAGCAGGGGAACAACACGGGGAACGAGTCGCGGTGCTACATGAAC
CACTGCAACACGTCGGTGATCCAGGAGTCGTGCGACAAGCACTACTGGGACGCGATCCGGTTCCGG
TACTGCGCGCCGCCGGGCTACGCGCTGCTGCGGTGCAACGACACGAACTACTCGGGCTTCATGCCG
AAATGCTCGAAGGTGGTGGTCTCGTCGTGCACGAGGATGATGGAGACGCAGACCTCGACGTGGTTC
GGCTTCAACGGGACGCGGGCGGAGAACCGGACGTACATCTACTGGCACGGGCGGGACAACCGGACG
ATCATCTCGCTGAACAAGTACTACAACCTGACGATGAAGTGCCGGCGGCCGGGCAACAAGACGGTG
CTCCCGGTCACCATCATGTCGGGGCTGGTGTTCCACTCGCAGCCGATCAACGACCGGCCGAAGCAG
GCGTGGTGCTGGTTCGGGGGGAAGTGGAAGGACGCGATCAAGGAGGTGAAGCAGACCATCGTCAAG
CACCCCCGCTACACGGGGACGAACAACACGGACAAGATCAACCTGACGGCGCCGGGCGGGGGCGAT
CCGGAAGTTACCTTCATGTGGACAAATTGCAGAGGAGAGTTCCTCTACTGCAAGATGAACTGGTTC
CTGAACTGGGTGGAGGACAGGAACACGGCGAACCAGAAGCCGAAGGAGCAGCACAAGCGGAACTAC
GTGCCGTGCCACATTCGGCAGATCATCAACACGTGGCACAAAGTGGGCAAGAACGTGTACCTGCCG
CCGAGGGAGGGCGACCTCACGTGCAACTCCACGGTGACCTCCCTCATCGCGAACATCGACTGGATC
GACGGCAACCAGACGAACATCACCATGTCGGCGGAGGTGGCGGAGCTGTACCGGCTGGAGCTGGGG
GACTACAAGCTGGTGGAGATCACGCCGATCGGCCTGGCCCCCACCGATGTGAAGCGCTACACGACC
GGGGGGACGTCGCGGAACAAGCGGGGGGTCTTCGTCCTGGGGTTCCTGGGGTTCCTCGCGACGGCG
GGGTCGGCAATGGGAGCCGCCAGCCTGACCCTCACGGCACAGTCCCGAACTTTATTGGCTGGGATC
GTCCAACAACAGCAGCAGCTGCTGGACGTGGTCAAGAGGCAGCAGGAGCTGCTGCGGCTGACCGTC
TGGGGCACGAAGAACCTCCAGACGAGGGTCACGGCCATCGAGAAGTACCTGAAGGACCAGGCGCAG
CTGAACGCGTGGGGCTGTGCGTTTCGACAAGTCTGCCACACGACGGTCCCGTGGCCGAACGCGTCG
CTGACGCCGAAGTGGAACAACGAGACGTGGCAGGAGTGGGAGCGGAAGGTGGACTTCCTGGAGGAG
AACATCACGGCCCTCCTGGAGGAGGCGCAGATCCAGCAGGAGAAGAACATGTACGAGCTGCAAAAG
CTGAACAGCTGGGACGTGTTCGGCAACTGGTTCGACCTGGCGTCGTGGATCAAGTACATCCAGTAC
GGCGTGTACATCGTGGTGGGGGTGATCCTGCTGCGGATCGTGATCTACATCGTCCAGATGCTGGCG
AAGCTGCGGCAGGGCTATAGGCCAGTGTTCTCTTCCCCACCCTCTTATTTCCAACAAACCCATATC
CAACAAGACCCGGCGCTGCCGACCCGGGAGGGCAAGGAGCGGGACGGCGGGGAGGGCGGCGGCAAC
AGCTCCTGGCCGTGGCAGATCGAGTACATCCACTTTCTTATTCGTCAGCTTATTAGACTCCTGACG
TGGCTGTTCAGTAACTGTAGGACTCTGCTGTCGAGGGTGTACCAGATCCTCCAGCCGATCCTCCAG
CGGCTCTCGGCGACCCTCCAGAGGATTCGGGAGGTCCTCCGGACGGAGCTGACCTACCTCCAGTAC
GGGTGGAGCTATTTCCACGAGGCGGTCCAGGCCGTCTGGCGGTCGGCGACGGAGACGCTGGCGGGC
GCGTGGGGCGACCTGTGGGAGACGCTGCGGCGGGGCGGCCGGTGGATACTCGCGATCCCCCGGCGG
ATCAGGCAGGGGCTGGAGCTCACGCTCCTGTGATAAGATATCGGATCTGCTGTGCCTTCTAGTTGC
CAGCCATCTGTTGTTTGCCCCTCCCCCGTGCCTTCCTTGACCCTGGAAGGTGCCACTCCCACTGTC
CTTTCCTAATAAAATGAGGAAATTGCATCGCATTGTCTGAGTAGGTGTCATTCTATTCTGGGGGGT
GGGGTGGGGCAGCACAGCAAGGGGGAGGATTGGGAAGACAATAGCAGGCATGCTGGGGATGCGGTG
GGCTCTATGGGTACCCAGGTGCTGAAGAATTGACCCGGTTCCTCCTGGGCCAGAAAGAAGCAGGCA
CATCCCCTTCTCTGTGACACACCCTGTCCACGCCCCTGGTTCTTAGTTCCAGCCCCACTCATAGGA
CACTCATAGCTCAGGAGGGCTCCGCCTTCAATCCCACCCGCTAAAGTACTTGGAGCGGTCTCTCCC
TCCCTCATCAGCCCACCAAACCAAACCTAGCCTCCAAGAGTGGGAAGAAATTAAAGCAAGATAGGC
TATTAAGTGCAGAGGGAGAGAAAATGCCTCCAACATGTGAGGAAGTAATGAGAGAAATCATAGAAT
TTCTTCCGCTTCCTCGCTCACTGACTCGCTGCGCTCGGTCGTTCGGCTGCGGCGAGCGGTATCAGC
TCACTCAAAGGCGGTAATACGGTTATCCACAGAATCAGGGGATAACGCAGGAAAGAACATGTGAGC
AAAAGGCCAGCAAAAGGCCAGGAACCGTAAAAAGGCCGCGTTGCTGGCGTTTTTCCATAGGCTCCG
CCCCCCTGACGAGCATCACAAAAATCGACGCTCAAGTCAGAGGTGGCGAAACCCGACAGGACTATA
AAGATACCAGGCGTTTCCCCCTGGAAGCTCCCTCGTGCGCTCTCCTGTTCCGACCCTGCCGCTTAC
CGGATACCTGTCCGCCTTTCTCCCTTCGGGAAGCGTGGCGCTTTCTCAATGCTCACGCTGTAGGTA
TCTCAGTTCGGTGTAGGTCGTTCGCTCCAAGCTGGGCTGTGTGCACGAACCCCCCGTTCAGCCCGA
CCGCTGCGCCTTATCCGGTAACTATCGTCTTGAGTCCAJLCCCGGTAAGACACGACTTATCGCCAC
TGGCAGCAGCCACTGGTAACAGGATTAGCAGAGCGAGGTATGTAGGCGGTGCTACAGAGTTCTTGA
AGTGGTGGCCTAACTACGGCTACACTAGAAGGACAGTATTTGGTATCTGCGCTCTGCTGAAGCCAG
TTACCTTCGGAAGAGTTGGTAGCTCTTGATCCGGCAAACAAACCACCGCTGGTAGCGGTGGTTTTT
TTGTTTGCAAGCAGCAGATTACGCGCAGAAAAAAGGATCTCAAGAAGATCCTTTGATCTTTTCTAC
GGGGTCTGACGCTCAGTGGAACGAAAACTCACGTTAAGGGATTTTGGTCATGAGATTATCAAAAAG
GATCTTCACCTAGATCCTTTTAAATTAAAATGAAGTTTTAAATCAATCTAAAGTATATATGAGTAA
ACTTGGTCTGACAGTTACCAATGCTTAATCAGTGAGGCACCTATCTCAGCGATCTGTCTATTTCGT
TCATCCATAGTTGCCTGACTCCGGGGGGGGGGGGCGCTGAGGTCTGCCTCGTGAAGAAAGGTGTTG
CTGACTCATACCAGGCCTGAATCGCCCCATCATCCAGCCAGAAAGTGAGGGAGCCACGGTTGATGA
GAGCTTTGTTGTAGGTGGACCAGTTGGTGATTTTGAACTTTTGCTTTGCCACGGAACGGTCTGCGT
TGTCGGGAAGATGCGTGATCTGATCCTTCAACTCAGCAAAAGTTCGATTTATTCAACAAAGCCGCC
GTCCCGTCAAGTCAGCGTAATGCTCTGCCAGTGTTACAACCAATTAACCAATTCTGATTAGAAAAA
CTCATCGAGCATCAAATGAAACTGCAATTTATTCATATCAGGATTATCAATACCATATTTTTGAAA
AAGCCGTTTCTGTAATGAAGGAGAAAACTCACCGAGGCAGTTCCATAGGATGGCAAGATCCTGGTA
TCGGTCTGCGATTCCGACTCGTCCAACATCAATACAACCTATTAATTTCCCCTCGTCAAAAATAAG
GTTATCAAGTGAGAAATCACCATGAGTGACGACTGAATCCGGTGAGAATGGCAAAAGCTTATGCAT
TTCTTTCCAGACTTGTTCAACAGGCCAGCCATTACGCTCGTCATCAAAATCACTCGCATCAACCAA
ACCGTTATTCATTCGTGATTGCGCCTGAGCGAGACGAAATACGCGATCGCTGTTAAAAGGACAATT
ACAAACAGGAATCGAATGCAACCGGCGCAGGAACACTGCCAGCGCATCAACAATATTTTCACCTGA
ATCAGGATATTCTTCTAATACCTGGAATGCTGTTTTCCCGGGGATCGCAGTGGTGAGTAACCATGC
ATCATCAGGAGTACGGATAAAATGCTTGATGGTCGGAAGAGGCATAAATTCCGTCAGCCAGTTTAG
TCTGACCATCTCATCTGTAACATCATTGGCAACGCTACCTTTGCCATGTTTCAGAAACAACTCTGG
CGCATCGGGCTTCCCATACAATCGATAGATTGTCGCACCTGATTGCCCGACATTATCGCGAGCCCA
TTTATACCCATATAAATCAGCATCCATGTTGGAATTTAATCGCGGCCTCGAGCAAGACGTTTCCCG
TTGAATATGGCTCATAACACCCCTTGTATTACTGTTTATGTAAGCAGACAGTTTTATTGTTCATGA
TGATATATTTTTATCTTGTGCAATGTAACATCAGAGATTTTGAGACACAACGTGGCTTTCCCCCCC
CCCCCATTATTGAAGCATTTATCAGGGTTATTGTCTCATGAGCGGATACATATTTGAATGTATTTA
GAAAAATAAACAAATAGGGGTTCCGCGCACATTTCCCCGAAAAGTGCCACCTGACGTCTAAGAAAC
CATTATTATCATGACATTAACCTATAAAAATAGGCGTATCACGAGGCCCTTTCGTCTCGCGCGTTT
CGGTGATGACGGTGAAAACCTCTGACACATGCAGCTCCCGGAGACGGTCACAGCTTGTCTGTAAGC
GGATGCCGGGAGCAGACAAGCCCGTCAGGGCGCGTCAGCGGGTGTTGGCGGGTGTCGGGGCTGGCT
TAACTATGCGGCATCAGAGCAGATTGTACTGAGAGTGCACCATATGCGGTGTGAAATACCGCACAG
ATGCGTAAGGAGAAAATACCGCATCAGATTGGCTATTGG (6903)
protein:
M N A S A A V F C L L L G L S G T Q (IP10)
G I L D (linker)
M A Q A V G N T S T T C C Y R F N K K A K Q R L E S Y R R T T S S
H C A R E A V F K T K L D K E C A D A T Q K W V Q D F M K H L D K
K T Q T A K L (MCP3)
I C S (linker)
L Y V T V F Y G V A A W R N A T A L F C A T K N R D T W G T T Q C
L A D N G D Y S E V A L N V T E S F D A W N N T V T E Q A E D V W
Q L F E T S K A C V K L S A L C T M R C N K S E T D R W G L T K S
T T T A S T T S T T A S A K V D M V N E T S S C A Q D N C T G L E
Q E Q M S C K F N M T G L K R D K K K E Y N E T W Y S A D L V C E
Q G N N T G N E S R C Y M N H C N T S V Q E S C D K H Y W D A R F
R Y C A A A G Y A L L R C N D T N Y S G F M A K C S K V V V S S C
T R M M E T Q T S T W F G F N G T R A E N R T Y Y W H G R D N R T
L S L N K Y Y N L T M K C R R A G N K T V L A V T M S G L V F H S
Q A N D R A K Q A W C W F G G K W K D A K E V K Q T V K H A R Y T
G T N N T D K N L T A A G G G D A E V T F M W T N C R G E F L Y C
K M N W F L N W V E D R N T A N Q K A K E Q H K R N Y V A C H R Q
N T W H K V G K N V Y L A A R E G D L T C N S T V T S L A N D W D
G N Q T N T M S A E V A E L Y R L E L G D Y K L V E T A J G L A A
T D V K R Y T T G G T S R N K R G V F V L G F L G F L A T A G S A
M G A A S L T L T A Q S R T L L A G V Q Q Q Q Q L L D V V K R Q Q
E L L R L T V W G T K N L Q T R V T A E K Y L K D Q A Q L N A W G
C A F R Q V C H T T V A W A N A S L T A K W N N E T W Q E W E R K
V D F L E E N T A L L E E A Q Q Q E K N M Y E L Q K L N S W D V F
G N W F D L A S W K Y Q Y G V Y V V G V L L R V Y V Q M L A K L R
Q G Y R A V F S S A A S Y F Q Q T H Q Q D A A L A T R E G K E R D
G G E G G G N S S W A W Q E Y H F L R Q L R L L T W L F S N C R T
L L S R V Y Q L Q A L Q R L S A T L Q R R E V L R T E L T Y L Q Y
G W S Y F H E A V Q A V W R S A T E T L A G A W G D L W E T L R R
G G R W I L A I P R R I R Q G L E L T L L • (SIVmac239env)
Plasmid CMVtPAenvmac239
CCTGGCCATTGCATACGTTGTATCCATATCATAATATGTACATTTATATTGGCTCATGTCCAACAT
TACCGCCATGTTGACATTGATTATTGACTAGTTATTAATAGTAATCAATTACGGGGTCATTAGTTC
ATAGCCCATATATGGAGTTCCGCGTTACATAACTTACGGTAAATGGCCCGCCTGGCTGACCGCCCA
ACGACCCCCGCCCATTGACGTCAATAATGACGTATGTTCCCATAGTAACGCCAATAGGGACTTTCC
ATTGACGTCAATGGGTGGAGTATTTACGGTAAACTGCCCACTTGGCAGTACATCAAGTGTATCATA
TGCCAAGTACGCCCCCTATTGACGTCAATGACGGTAAATGGCCCGCCTGGCATTATGCCCAGTACA
TGACCTTATGGGACTTTCCTACTTGGCAGTACATCTACGTATTAGTCATCGCTATTACCATGGTGA
TGCGGTTTTGGCAGTACATCAATGGGCGTGGATAGCGGTTTGACTCACGGGGATTTCCAAGTCTCC
ACCCCATTGACGTCAATGGGAGTTTGTTTTGGCACCAAAATCAACGGGACTTTCCAAAATGTCGTA
ACAACTCCGCCCCATTGACGCAAATGGGCGGTAGGCGTGTACGGTGGGAGGTCTATATAAGCAGAG
CTCGTTTAGTGAACCGTCAGATCGCCTGGAGACGCCATCCACGCTGTTTTGACCTCCATAGAAGAC
ACCGGGACCGATCCAGCCTCCGCGGGCGCGCGTCGAGGAAAATTCAAGAAATGGATGCAATGAAGA
GAGGGCTCTGCTGTGTGCTGCTGCTGTGTGGAGCAGTCTTCGTTTCGCCCAGCCAGGAAATCCATG
CCCGATTCAGAAGAGGAGCCAGATCTATCTGCAGCCTGTACGTCACGGTCTTCTACGGCGTACCAG
CTTGGAGGAATGCGACAATTCCCCTCTTTTGTGCAACCAAGAATAGGGATACTTGGGGAACAAAAC
TCAGTGCCTACCGGACAACGGGGACTACTCGGAGGTGGCCCTGAACGTGACGGAGAGCTTCGACGC
CTGGAJLCAACACGGTCACGGAGCAGGCGATCGAGGACGTGTGGCAGCTGTTCGAGACCTCGATCA
AGCCGTGCGTCAAGCTGTCCCCGCTCTGCATCACGATGCGGTGCAACAAGAGCGAGACGGATCGGT
GGGGGCTGACGAAGTCGATCACGACGACGGCGTCGACCACGTCGACGACGGCGTCGGCGAAAGTGG
ACATGGTCAACGAGACCTCGTCGTGCATCGCCCAGGACAACTGCACGGGCCTGGAGCAGGAGCAGA
TGATCAGCTGCAAGTTCAACATGACGGGGTGAAGCGGGACAAGAAGAAGGAGTACAACGAGACGTG
GTACTCGGCGGACCTGGTGTGCGAGCAGGGGAACAAACACGGGGAACGAGTCGCGGTGCTACATGA
ACCACTGCAACACGTCGGTGATCCAGGAGTCGTGCGACAAGCACTACTGGGACGCGATCCGGTTCC
GGTACTGCGCGCCGCCGGGCTACGCGCTGCTGCGGTGCAACGACACGAACTACTCGGGCTTCATGC
CGAAATGCTCGAAGGTGGTGGTCTCGTCGTGCACGAGGATGATGGAGACGCAGACCTCGACGTGGT
TCGGCTTCAACGGGACGCGGGCGGAGAACCGGACGTACATCTACTGGCACGGGCGGGACAACCGGA
CGATCATCTCGCTGAACAAGTACTACAACCTGACGATGAAGTGCCGGCGGCCGGGCAACAAGACGG
TGCTCCCGGTCACCATCATGTCGGGGCTGGTGTTCCACTCGCAGCCGATCAACGACCGGCCGAAGC
AGGCGTGGTGCTGGTTCGGGGGGAAGTGGAAGGACGCGATCAAGGAGGTGAAGCAGACCATCGTCA
AGCACCCCCGCTACACGGGGACGAACAACACGGACAAGATCMAACCTGACGGCGCCGGGCGGGGGC
GATCCGGAAGTTACCTTCATGTGGACAAATTGCAGAGGAGAGTTCCTCTACTGCAAGATGAACTGG
TTCCTGAACTGGGTGGAGGACAGGAACACGGCGAACCAGAAGCCGAAGGAGCAGCACAAGCGGAAC
TACGTGCCGTGCCACATTCGGCAGATCATCAACACGTGGCACAAAGTGGGCAAGAACGTGTACCTG
CCGCCGAGGGAGGGCGACCTCACGTGCAACTCCACGGTGACCTCCCTCATCGCGJAACATCGACTG
GATCGACGGCAACCAGACGAACATCACCATGTCGGCGGAGGTGGCGGAGCTGTACCGGCTGGAGCT
GGGGGACTACAAGCTGGTGGAGATCACGCCGATCGGCCTGGCCCCCACCGATGTGAAGCGCTACAC
GACCGGGGGGACGTCGCGGAACAAGCGGGGGGTCTTCGTCCTGGGGTTCCTGGGGTTCCTCGCGAC
GGCGGGGTCGGCAATGGGAGCCGCCAGCCTGACCCTCACGGCACAGTCCCGAACTTTATTGGCTGG
GATCGTCCAACAACAGCAGCAGCTGCTGGACGTGGTCAAGAGGCAGCAGGAGCTGCTGCGGCTGAC
CGTCTGGGGCACGAAGAACCTCCAGACGAGGGTCACGGCCATCGAGAAGTACCTGAAGGACCAGGC
GCAGCTGAACGCGTGGGGCTGTGCGTTTCGACAAGTCTGCCACACGACGGTCCCGTGGCCGAACGC
GTCGCTGACGCCGAAGTGGAACAACGAGACGTGGCAGGAGTGGGAGCGGAAGGTGGACTTCCTGGA
GGAGAACATCACGGCCCTCCTGGAGGAGGCGCAGATCCAGCAGGAGAAGAACATGTACGAGCTGCA
AAAGCTGAACAGCTGGGACGTGTTCGGCAACTGGTTCGACCTGGCGTCGTGGATCAAGTACATCCA
GTACGGCGTGTACATCGTGGTGGGGGTGATCCTGCTGCGGATCGTGATCTACATCGTCCAGATGCT
GGCGAAGCTGCGGCAGGGCTATAGGCCAGTGTTCTCTTCCCCACCCTCTTATTTCCAACAAACCCA
TATCCAACAAGACCCGGCGCTGCCGACCCGGGAGGGCAAGGAGCGGGACGGCGGGGAGGGCGGCGG
CAACAGCTCCTGGCCGTGGCAGATCGAGTACATCCACTTTCTTATTCGTCAGCTTATTAGACTCCT
GACGTGGCTGTTCAGTAACTGTAGGACTCTGCTGTCGAGGGTGTACCAGATCCTCCAGCCGATCCT
CCAGCGGCTCTCGGCGACCCTCCAGAGGATTCGGGAGGTCCTCCGGACGGAGCTGACCTACCTCCA
GTACGGGTGGAGCTATTTCCACGAGGCGGTCCAGGCCGTCTGGCGGTCGGCGACGGAGACGCTGGC
GGGCGCGTGGGGCGACCTGTGGGAGACGCTGCGGCGGGGCGGCCGGTGGATACTCGCGATCCCCCG
GCGGATCAGGCAGGGGCTGGAGCTCACGCTCCTGTGATAAGATATCGGATCTGCTGTGCCTTCTAG
TTGCCAGCCATCTGTTGTTTGCCCCTCCCCCGTGCCTTCCTTGACCCTGGAAGGTGCCACTCCCAC
TGTCCTTTCCTAATAAAATGAGGAAATTGCATCGCATTGTCTGAGTAGGTGTCATTCTATTCTGGG
GGGTGGGGTGGGGCAGCACAGCAAGGGGGAGGATTGGGAAGACAATAGCAGGCATGCTGGGGATGC
GGTGGGCTCTATGGGTACCCAGGTGCTGAAGAATTGACCCGGTTCCTCCTGGGCCAGAAAGAAGCA
GGCACATCCCCTTCTCTGTGACACACCCTGTCCACGCCCCTGGTTCTTAGTTCCAGCCCCACTCAT
AGGACACTCATAGCTCAGGAGGGCTCCGCCTTCAATCCCACCCGCTAAAGTACTTGGAGCGGTCTC
TCCCTCCCTCATCAGCCCACCAAACCAAACCTAGCCTCCAAGAGTGGGAAGAAATTAAAGCAAGAT
AGGCTATTAAGTGCAGAGGGAGAGAAAATGCCTCCAACATGTGAGGAAGTAATGAGAGAAATCATA
GAATTTCTTCCGCTTCCTCGCTCACTGACTCGCTGCGCTCGGTCGTTCGGCTGCGGCGAGCGGTAT
CAGCTCACTCAAAGGCGGTAATACGGTTATCCACAGAATCAGGGGATAACGCAGGAAAGAACATGT
GAGCAAAAGGCCAGCAAAAGGCCAGGAACCGTAAAAAGGCCGCGTTGCTGGCGTTTTTCCATAGGC
TCCGCCCCCCTGACGAGCATCACAAAAATCGACGCTCAAGTCAGAGGTGGCGAAACCCGACAGGAC
TATAAAGATACCAGGCGTTTCCCCCTGGAAGCTCCCTCGTGCGCTCTCCTGTTCCGACCCTGCCGC
TTACCGGATACCTGTCCGCCTTTCTCCCTTCGGGAAGCGTGGCGCTTTCTCAATGCTCACGCTGTA
GGTATCTCAGTTCGGTGTAGGTCGTTCGCTCCAAGCTGGGCTGTGTGCACGAACCCCCCGTTCAGC
CCGACCGCTGCGCCTTATCCGGTAACTATCGTCTTGAGTCCAACCCGGTAAGACACGACTTATCGC
CACTGGCAGCAGCCACTGGTAACAGGATTAGCAGAGCGAGGTATGTAGGCGGTGCTACAGAGTTCT
TGAAGTGGTGGCCTAACTACGGCTACACTAGAAGGACAGTATTTGGTATCTGCGCTCTGCTGAAGC
CAGTTACCTTCGGAAAAAGAGTTGGTAGCTCTTGATCCGGCAAACAAACCACCGCTGGTAGCGGTG
GTTTTTTTGTTTGCAAGCAGCAGATTACGCGCAGAAAAAAAGGATCTCAAGAAGATCCTTTGATCT
TTTCTACGGGGTCTGACGCTCAGTGGAACGAAAACTCACGTTAAGGGATTTTGGTCATGAGATTAT
CAAAAAGGATCTTCACCTAGATCCTTTTAAATTAAAAATGAAGTTTTAAATCAATCTAJLAGTATA
TATGAGTAAACTTGGTCTGACAGTTACCAATGCTTAATCAGTGAGGCACCTATCTCAGCGATCTGT
CTATTTCGTTCATCCATAGTTGCCTGACTCCGGGGGGGGGGGGCGCTGAGGTCTGCCTCGTGAAGA
AGGTGTTGCTGACTCATACCAGGCCTGAATCGCCCCATCATCCAGCCAGAAAGTGAGGGAGCCACG
GTTGATGAGAGCTTTGTTGTAGGTGGACCAGTTGGTGATTTTGAACTTTTGCTTTGCCACGGAACG
GTCTGCGTTGTCGGGAAGATGCGTGATCTGATCCTTCAACTCAGCAAAAGTTCGATTTATTCAACA
AAGCCGCCGTCCCGTCAAGTCAGCGTAATGCTCTGCCAGTGTTACAACCAATTAACCAATTCTGAT
TAGAAAAACTCATCGAGCATCAAATGAAACTGCAATTTATTCATATCAGGATTATCAATACCATAT
TTTTGAAAAAGCCGTTTCTGTAATGAAGGAGAAAACTCACCGAGGCAGTTCCATAGGATGGCAAGA
TCCTGGTATCGGTCTGCGATTCCGACTCGTCCAACATCAATACAACCTATTAATTTCCCCTCGTCA
AAAATAAGGTTATCAAGTGAGAAATCACCATGAGTGACGACTGAATCCGGTGAGAATGGCAAAAGC
TTATGCATTTCTTTCCAGACTTGTTCAACAGGCCAGCCATTACGCTCGTCATCAAAATCACTCGCA
TCAACCAAACCGTTATTCATTCGTGATTGCGCCTGAGCGAGACGAAATACGCGATCGCTGTTAAAA
GGACAATTACAAACAGGAATCGAATGCAACCGGCGCAGGAACACTGCCAGCGCATCAACAATATTT
TCACCTGAATCAGGATATTCTTCTAATACCTGGAATGCTGTTTTCCCGGGGATCGCAGTGGTGAGT
AACCATGCATCATCAGGAGTACGGATAAAATGCTTGATGGTCGGAAGAGGCATAAATTCCGTCAGC
CAGTTTAGTCTGACCATCTCATCTGTAACATCATTGGCAACGCTACCTTTGCCATGTTTCAGAAAC
AACTCTGGCGCATCGGGCTTCCCATACAATCGATAGATTGTCGCACCTGATTGCCCGACATTATCG
CGAGCCCATTTATACCCATATAAATCAGCATCCATGTTGGAATTTAATCGCGGCCTCGAGCAAGAC
GTTTCCCGTTGAATATGGCTCATAACACCCCTTGTATTACTGTTTATGTAAGCAGACAGTTTTATT
GTTCATGATGATATATTTTTATCTTGTGCAATGTAACATCAGAGATTTTGAGACACAACGTGGCTT
TCCCCCCCCCCCCATTATTGAAGCATTTATCAGGGTTATTGTCTCATGAGCGGATACATATTTGAA
TGTATTTAGAAAAATAAACAAATAGGGGTTCCGCGCACATTTCCCCGAAAAGTGCCACCTGACGTC
TAAGAAACCATTATTATCATGACATTAACCTATAAAAATAGGCGTATCACGAGGCCCTTTCGTCTC
GCGCGTTTCGGTGATGACGGTGAAAACCTCTGACACATGCAGCTCCCGGAGACGGTCACAGCTTGT
CTGTAAGCGGATGCCGGGAGCAGACAAGCCCGTCAGGGCGCGTCAGCGGGTGTTGGCGGGTGTCGG
GGCTGGCTTAACTATGCGGCATCAGAGCAGATTGTACTGAGAGTGCACCATATGCGGTGTGAAATA
CCGCACAGATGCGTAAGGAGAAAATACCGCATCAGATTGGCTATTGG
tPA-env gene:
ATGGATGCAATGAAGAGAGGGCTCTGCTGTGTGCTGCTGCTGTGTGGAGCAGTCTTCGTTTCGCCC
AGCCAGGAAATCCATGCCCGATTCAGAAGAGGAGCCAGATCTATCTGCAGCCTGTACGTCACGGTC
TTCTACGGCGTACCAGCTTGGAGGAATGCGACAATTCCCCTCTTTTGTGCAACCAAGAATAGGGAT
ACTTGGGGAACAACTCAGTGCCTACCGGACAACGGGGACTACTCGGAGGTGGCCCTGAACGTGACG
GAGAGCTTCGACGCCTGGAACAACACGGTCACGGAGCAGGCGATCGAGGACGTGTGGCAGCTGTTC
GAGACCTCGATCAAGCCGTGCGTCAAGCTGTCCCCGCTCTGCATCACGATGCGGTGCAACAAGAGC
GAGACGGATCGGTGGGGGCTGACGAAGTCGATCACGACGACGGCGTCGACCACGTCGACGACGGCG
TCGGCGAAAGTGGACATGGTCAACGAGACCTCGTCGTGCATCGCCCAGGACAACTGCACGGGCCTG
GAGCAGGAGCAGATGATCAGCTGCAAGTTCAACATGACGGGGCTGAAGCGGGACAAGAAGAAGGAG
TACAACGAGACGTGGTACTCGGCGGACCTGGTGTGCGAGCAGGGGAACAACACGGGGAACGAGTCG
CGGTGCTACATGAACCACTGCAACACGTCGGTGATCCAGGAGTCGTGCGACAAGCACTACTGGGAC
GCGATCCGGTTCCGGTACTGCGCGCCGCCGGGCTACGCGCTGCTGCGGTGCAACGACACGAACTAC
TCGGGCTTCATGCCGAAAATGCTCGAAGGTGGTGGTCTCGTCGTGCACGAGGATGATGGAGACGCA
GACCTCGACGTGGTTCGGCTTCAACGGGACGCGGGCGGAGAACCGGACGTACATCTACTGGCACGG
GCGGGACAACCGGACGATCATCTCGCTGAACAAGTACTACAACCTGACGATGAAGTGCCGGCGGCC
GGGCAACAAGACGGTGCTCCCGGTCACCATCATGTCGGGGCTGGTGTTCCACTCGCAGCCGATCAA
CGACCGGCCGAAGCAGGCGTGGTGCTGGTTCGGGGGGAAGTGGAAGGACGCGATCAAGGAGGTGAA
GCAGACCATCGTCAAGCACCCCCGCTACACGGGGACGAACAACACGGACAAGATCAACCTGACGGC
GCCGGGCGGGGGCGATCCGGAAGTTACCTTCATGTGGACAAATTGCAGAGGAGAGTTCCTCTACTG
CAAGATGAACTGGTTCCTGAACTGGGTGGAGGACAGGAAAACACGGCGAACCAGAAGCCGAAGGAG
CAGCACAAGCGGAACTACGTGCCGTGCCACATTCGGCAGATCATCAACACGTGGCACAAAGTGGGC
AAGAACGTGTACCTGCCGCCGAGGGAGGGCGACCTCACGTGCAACTCCACGGTGACCTCCCTCATC
GCGAACATCGACTGGATCGACGGCAACCAGACGAACATGACCATGTCGGCGGAGGTGGCGGAGCTG
TACCGGCTGGAGCTGGGGGACTACAAGCTGGTGGAGATCACGCCGATCGGCCTGGCCCCCACCGAT
GTGAAGCGCTACACGACCGGGGGGACGTCGCGGAACAAAGCGGGGGGTCTTCGTCCTGGGGTTCCT
GGGGTTCCTCGCGACGGCGGGGTCGGCAATGGGAGCCGCCAGCCTGACCCTCACGGCACAGTCCCG
AACTTTATTGGCTGGGATCGTCCAACAACAGCAGCAGCTGCTGGACGTGGTCAAGAGGCAGCAGGA
GCTGCTGCGGCTGACCGTCTGGGGCACGAAGAACCTCCAGACGAGGGTCACGGCCATCGAGAAGTA
CCTGAAGGACCAGGCGCAGCTGAACGCGTGGGGCTGTGCGTTTCGACAAGTCTGCCACACGACGGT
CCCGTGGCCGAACGCGTCGCTGACGCCGAAGTGGAACAACGAGACGTGGCAGGAGTGGGAGCGGAA
GGTGGACTTCCTGGAGGAGAACATCACGGCCCTCCTGGAGGAGGCGCAGATCCAGCAGGAGAAGAA
CATGTACGAGCTGCAAAAGCTGAACAGCTGGGACGTGTTCGGCAACTGGTTCGACCTGGCGTCGTG
GATCAAGTACATCCAGTACGGCGTGTACATCGTGGTGGGGGTGATCCTGCTGCGGATCGTGATCTA
CATCGTCCAGATGCTGGCGAAGCTGCGGCAGGGCTATAGGCCAGTGTTCTCTTCCCCACCCTCTTA
TTTCCAACAAACCCATATCCAACAAGACCCGGCGCTGCCGACCCGGGAGGGCAAGGAGCGGGACGG
CGGGGAGGGCGGCGGCAACAGCTCCTGGCCGTGGCAGATCGAGTACATCCACTTTCTTATTCGTCA
GCTTATTAGACTCCTGACGTGGCTGTTCAGTAACTGTAGGACTCTGCTGTCGAGGGTGTACCAGAT
CCTCCAGCCGATCCTCCAGCGGCTCTCGGCGACCCTCCAGAGGATTCGGGAGGTCCTCCGGACGGA
GCTGACCTACCTCCAGTACGGGTGGAGCTATTTCCACGAGGCGGTCCAGGCCGTCTGGCGGTCGGC
GACGGAGACGCTGGCGGGCGCGTGGGGCGACCTGTGGGAGACGCTGCGGCGGGGCGGCCGGTGGAT
ACTCGCGATCCCCCGGCGGATCAGGCAGGGGCTGGAGCTCACGCTCCTGTGA
tPA-env protein
M D A M K R G L C C V L L L C G A V F V S A S Q E H A R F R R G A
R S (tPA)
C S (linker)
L Y V T V F Y G V A A W R N A T A L F C A T K N R D T W G T T Q C
L A D N G D Y S E V A L N V T E S F D A W N N T V T E Q A E D V W
Q L F E T S K A C V K L S A L C T M R C N K S E T D R W G L T K S
T T T A S T T S T T A S A K V D M V N E T S S C A Q D N C T G L E
Q E Q M S C K F N M T G L K R D K K K E Y N E T W Y S A D L V C E
Q G N N T G N E S R C Y M N H C N T S V Q E S C D K H Y W D A R F
R Y C A A A G Y A L L R C N D T N Y S G F M A K C S K V V V S S C
T R M M E T Q T S T W F G F N G T R A E N R T Y Y W H G R D N R T
S L N K Y Y N L T M K C R R A G N K T V L A V T M S G L V F H S Q
A N D R A K Q A W C W F G G K W K D A K E V K Q T V K H A R Y T G
T N N T D K N L T A A G G G D A E V T F M W T N C R G E F L Y C K
M N W F L N W V E D R N T A N Q K A K E Q H K R N Y V A C H R Q N
T W H K V G K N V Y L A A R E G D L T C N S T V T S L A N D W D G
N Q T N T M S A E V A E L Y R L E L G D Y K L V E L T A G L A A T
D V K R Y T T G G T S R N K R G V F V L G F L G F L A T A G S A M
G A A S L T L T A Q S R T L L A G V Q Q Q Q Q L L D V V K R Q Q E
L L R L T V W G T K N L Q T R V T A E K Y L K D Q A Q L N A W G C
A F R Q V C H T T V A W A N A S L T A K W N N E T W Q E W E R K V
D F L E E N T A L L E E A Q Q Q E K N M Y E L Q K L N S W D V F G
N W F D L A S W K Y Q Y G V Y V V G V L L R V Y V Q M L A K L R Q
G Y R A V F S S A A S Y F Q Q T H Q Q D A A L A T R E G K E R D G
G E G G G N S S W A W Q E Y H F L R Q L R L L T W L F S N C R T L
L S R V Y Q L Q A L Q R L S A T L Q R R E V L R T E L T Y L Q Y G
W S Y F H E A V Q A V W R S A T E T L A G A W G D L W E T L R R G
G R W L A A R R R Q G L E L T L L • (SIVmac239 env)
pCMV MCP3p39 (STY) gene: 769-2199
(1)CCTGGCCATTGCATACGTTGTATCCATATCATAATATGTACATTTATATTGGCTCATGTCCAA
CATTACCGCCATGTTGACATTGATTATTGACTAGTTATTAATAGTAATCAATTACGGGGTCATTAG
TTCATAGCCCATATATGGAGTTCCGCGTTACATAACTTACGGTAAATGGCCCGCCTGGCTGACCGC
CCAACGACCCCCGCCCATTGACGTCAATAATGACGTATGTTCCCATAGTAACGCCAATAGGGACTT
TCCATTGACGTCAATGGGTGGAGTATTTACGGTAAACTGCCCACTTGGCAGTACATCAAGTGTATC
ATATGCCAAGTACGCCCCCTATTGACGTCAATGACGGTAAATGGCCCGCCTGGCATTATGCCCAGT
ACATGACCTTATGGGACTTTCCTACTTGGCAGTACATCTACGTATTAGTCATCGCTATTACCATGG
TGATGCGGTTTTGGCAGTACATCAATGGGCGTGGATAGCGGTTTGACTCACGGGGATTTCCAAGTC
TCCACCCCATTGACGTCAATGGGAGTTTGTTTTGGCACCAAAATCAACGGGACTTTCCAAAATGTC
GTAACAACTCCGCCCCATTGACGCAAATGGGCGGTAGGCGTGTACGGTGGGAGGTCTATATAAGCA
GAGCTCGTTTAGTGAACCGTCAGATCGCCTGGAGACGCCATCCACGCTGTTTTGACCTCCATAGAA
GACACCGGGACCGATCCAGCCTCCGCGGGCGCGCGTCGACAAGAAATGAACCCAAGTGCTGCCGTC
ATTTTCTGCCTCATCCTGCTGGGTCTGAGTGGGACTCAAGGGATCCTCGACATGGCGCAACCGGTC
GGGATCAACACGAGCACGACCTGCTGCTACCGGTTCATCAACAAGAAGATCCCGAAGCAACGTCTG
GAAAGCTATCGCCGGACCACGTCGAGCCACTGCCCGCGGGAGGCGGTTATCTTCAAGACGAAGCTG
GACAAGGAGATCTGCGCCGACCCGACGCAGAAGTGGGTTCAGGACTTCATGAAGCACCTGGATAAG
AAGACGCAGACGCCGAAGCTGGCTAGCGCAGGAGCAGGCGTGCGGAACTCCGTCTTGTCGGGGAAG
AAAGCGGATGAGTTGGAGAAAATTCGGCTACGGCCCAACGGGAAGAAGAAGTACATGTTGAAGCAT
GTAGTATGGGCGGCGAATGAGTTGGATCGGTTTGGATTGGCGGAGAGCCTGTTGGAGAACAAAGAG
GGATGTCAGAAGATCCTTTCGGTCTTGGCGCCGTTGGTGCCGACGGGCTCGGAGAACTTGAAGAGC
CTCTACAACACGGTCTGCGTCATCTGGTGCATTCACGCGGAAGAGAAAGTGAAACACACGGAGGAA
GCGAAACAGATAGTGCAGCGGCACCTAGTGGTGGAAACGGGAACCACCGAAACCATGCCGAAGACC
TCGCGGCCGACGGCGCCGTCGAGCGGCAGGGGAGGAAACTACCCGGTACAGCAGATCGGTGGCAAC
TACGTCCACCTGCCGCTGTCCCCGCGGACCCTGAACGCGTGGGTCAAGCTGATCGAGGAGAAGAAG
TTCGGAGCGGAGGTAGTGCCGGGATTCCAGGCGCTGTCGGAAGGTTGCACCCCCTACGACATCAAC
CAGATGCTGAACTGCGTTGGAGACCATCAGGCGGCGATGCAGATCATCCGGGACATCATCAACGAG
GAGGCGGCGGATTGGGACTTGCAGCACCCGCAACCGGCGCCGCAACAAGGACAACTTCGGGAGCCG
TCGGGATCGGACATCGCGGGAACCACCTCCTCGGTTGACGAACAGATCCAGTGGATGTACCGGCAG
CAGAACCCGATCCCAGTAGGCAACATCTACCGGCGGTGGATCCAGCTGGGTCTGCAGAAATGCGTC
CGTATGTACAACCCGACCAACATTCTAGATGTAAAACAAGGGCCAAAGGAGCCGTTCCAGAGCTAC
GTCGACCGGTTCTACAAGTCGCTGCGGGCGGAGCAGACGGACGCGGCGGTCAAGAACTGGATGACG
CAGACGCTGCTGATCCAGAACGCGAACCCAGATTGCAAGCTAGTGCTGAAGGGGCTGGGTGTGAAT
CCCACCCTAGAAGAAATGCTGACGGCTTGTCAAGGAGTAGGGGGGCCGGGACAGAAGGCTAGATTA
ATGGGGGCCCATGCGGCCGCGTAGGAATTCGATCCAGATCTGCTGTGCCTTCTAGTTGCCAGCCAT
CTGTTGTTTGCCCCTCCCCCGTGCCTTCCTTGACCCTGGAAGGTGCCACTCCCACTGTCCTTTCCT
AATAAAATGAGGAAATTGCATCGCATTGTCTGAGTAGGTGTCATTCTATTCTGGGGGGTGGGGTGG
GGCAGCACAGCAAGGGGGAGGATTGGGAAGACAATAGCAGGCATGCTGGGGATGCGGTGGGCTCTA
TGGGTACCCAGGTGCTGAAGAATTGACCCGGTTCCTCCTGGGCCAGAAAGAAGCAGGCACATCCCC
TTCTCTGTGACACACCCTGTCCACGCCCCTGGTTCTTAGTTCCAGCCCCACTCATAGGACACTCAT
AGCTCAGGAGGGCTCCGCCTTCAATCCCACCCGCTAAAGTACTTGGAGCGGTCTCTCCCTCCCTCA
TCAGCCCACCAAACCAAACCTAGCCTCCAAGAGTGGGAAGAAATTAAAGCAAGATAGGCTATTAAG
TGCAGAGGGAGAGAAAATGCCTCCAACATGTGAGGAAGTAATGAGAGAAATCATAGAATTTCTTCC
GCTTCCTCGCTCACTGACTCGCTGCGCTCGGTCGTTCGGCTGCGGCGAGCGGTATCAGCTCACTCA
AAGGCGGTAATACGGTTATCCACAGAATCAGGGGATAACGCAGGKAAGAACATGTGAGCAAAAGGC
CAGCAAAAGGCCAGGAACCGTAAAAAGGCCGCGTTGCTGGCGTTTTTCCATAGGCTCCGCCCCCCT
GACGAGCATCACAAAAJAJJCGACGCTCAAGTCAGAGGTGGCGAAACCCGACAGGACTATAAAGAT
ACCAGGCGTTTCCCCCTGGAAGCTCCCTCGTGCGCTCTCCTGTTCCGACCCTGCCGCTTACCGGAT
ACCTGTCCGCCTTTCTCCCTTCGGGAAGCGTGGCGCTTTCTCAATGCTCACGCTGTAGGTATCTCA
GTTCGGTGTAGGTCGTTCGCTCCAAGCTGGGCTGTGTGCACGAACCCCCCGTTCAGCCCGACCGCT
GCGCCTTATCCGGTAACTATCGTCTTGAGTCCAACCCGGTAAGACACGACTTATCGCCACTGGCAG
CAGCCACTGGTAACAGGATTAGCAGAGCGAGGTATGTAGGCGGTGCTACAGAGTTCTTGAAGTGGT
GGCCTAACTACGGCTACACTAGAAGGACAGTATTTGGTATCTGCGCTCTGCTGAAGCCAGTTACCT
TCGGAAAAAGAGTTGGTAGCTCTTGATCCGGCAAACAAACCACCGCTGGTAGCGGTGGTTTTTTTG
TTTGCAAGCAGCAGATTACGCGCAGAAAAAAAGGATCTCAAGAAGATCCTTTGATCTTTTCTACGG
GGTCTGACGCTCAGTGGAACGAAAACTCACGTTAAGGGATTTTGGTCATGAGATTATCAAAAAGGA
TCTTCACCTAGATCCTTTTAAATTAAAAATGAAGTTTTAAATCAATCTAAAGTATATATGAGTAAA
CTTGGTCTGACAGTTACCAATGCTTAATCAGTGAGGCACCTATCTCAGCGATCTGTCTATTTCGTT
CATCCATAGTTGCCTGACTCCGGGGGGGGGGGGCGCTGAGGTCTGCCTCGTGAAGAAGGTGTTGCT
GACTCATACCAGGCCTGAATCGCCCCATCATCCAGCCAGAAAGTGAGGGAGCCACGGTTGATGAGA
GCTTTGTTGTAGGTGGACCAGTTGGTGATTTTGAACTTTTGCTTTGCCACGGAACGGTCTGCGTTG
TCGGGAAGATGCGTGATCTGATCCTTCAACTCAGCAAAAGTTCGATTTATTCAACAAAGCCGCCGT
CCCGTCAAGTCAGCGTAATGCTCTGCCAGTGTTACAACCAATTAACCAATTCTGATTAGAAAAACT
CATCGAGCATCAAATGAAACTGCAATTTATTCATATCAGGATTATCAATACCATATTTTTGAAAAA
GCCGTTTCTGTAATGAAGGAGAAAACTCACCGAGGCAGTTCCATAGGATGGCAAGATCCTGGTATC
GGTCTGCGATTCCGACTCGTCCAACATCAATACAACCTATTAATTTCCCCTCGTCAAAAATAAGGT
TATCAAGTGAGAAATCACCATGAGTGACGACTGAATCCGGTGAGAATGGCAAAAGCTTATGCATTT
CTTTCCAGACTTGTTCAACAGGCCAGCCATTACGCTCGTCATCAAAATCACTCGCATCAACCAAAC
CGTTATTCATTCGTGATTGCGCCTGAGCGAGACGAAATACGCGATCGCTGTTAAAAGGACAATTAC
AAACAGGAATCGAATGCAACCGGCGCAGGAACACTGCCAGCGCATCAACAATATTTTCACCTGAAT
CAGGATATTCTTCTAATACCTGGAATGCTGTTTTCCCGGGGATCGCAGTGGTGAGTAACCATGCAT
CATCAGGAGTACGGATAAAATGCTTGATGGTCGGAAGAGGCATAAATTCCGTCAGCCAGTTTAGTC
TGACCATCTCATCTGTAACATCATTGGCAACGCTACCTTTGCCATGTTTCAGAAACAACTCTGGCG
CATCGGGCTTCCCATACAATCGATAGATTGTCGCACCTGATTGCCCGACATTATCGCGAGCCCATT
TATACCCATATAAATCAGCATCCATGTTGGAATTTAATCGCGGCCTCGAGCAAGACGTTTCCCGTT
GAATATGGCTCATAACACCCCTTGTATTACTGTTTATGTAAGCAGACAGTTTTATTGTTCATGATG
ATATATTTTTATCTTGTGCAATGTAACATCAGAGATTTTGAGACACAACGTGGCTTTCCCCCCCCC
CCCATTATTGAAGCATTTATCAGGGTTATTGTCTCATGAGCGGATACATATTTGAATGTATTTAGA
AAAATAAACAAATAGGGGTTCCGCGCACATTTCCCCGAAAAGTGCCACCTGACGTCTAAGAAACCA
TTATTATCATGACATTAACCTATAAAAATAGGCGTATCACGAGGCCCTTTCGTCTCGCGCGTTTCG
GTGATGACGGTGAAAACCTCTGACACATGCAGCTCCCGGAGACGGTCACAGCTTGTCTGTAAGCGG
ATGCCGGGAGCAGACAAGCCCGTCAGGGCGCGTCAGCGGGTGTTGGCGGGTGTCGGGGCTGGCTTA
ACTATGCGGCATCAGAGCAGATTGTACTGAGAGTGCACCATATGCGGTGTGAAATACCGCACAGAT
GCGTAAGGAGAAAATACCGCATCAGATTGGCTATTGG (5444)
protein:
M N A S A A V F C L L L G L S G T Q (MCP3)
G L D (linker)
M A Q A V G N T S T T C C Y R F N K K A K Q R L E S Y R R T T S S
H C A R E A V F K T K L D K E C A D A T Q K W V Q D F M K H L D K
K T Q T A K L A S A G A G V R N S V L S G K K A D E L E K R L R A
N G K K K Y M L K H V V W A A N E L D R F G L A E S L L E N K E G
C Q K L S V L A A L V A T G S E N L K S L Y N T V C V W C H A E E
K V K H T E E A K Q V Q R H L V V E T G T T E T M A K T S R A T A
A S S G R G G N Y A V Q Q G G N Y V H L A L S A R T L N A W V K L
E E K K F G A E V V A G F Q A L S E G C T A Y D N Q M L N C V G D
H Q A A M Q R D N E E A A D W D L Q H A Q A A A Q Q G Q L R E A S
G S D A G T T S S V D E Q Q W M Y R Q Q N A A V G N Y R R W Q L G
L Q K C V R M Y N A T N J L D V K Q G A K E A F Q S Y V D R F Y K
S L R A E Q T D A A V K N W M T Q T L L Q N A N A D C K L V L K G
L G V N A T L E E M L T A C Q G V G G A G Q K A R L M G A H A A
A • (gag)

### Exemplary HIV Constructs:

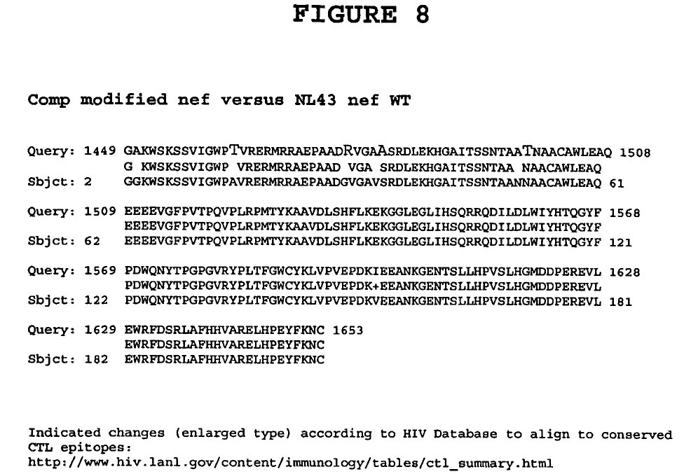

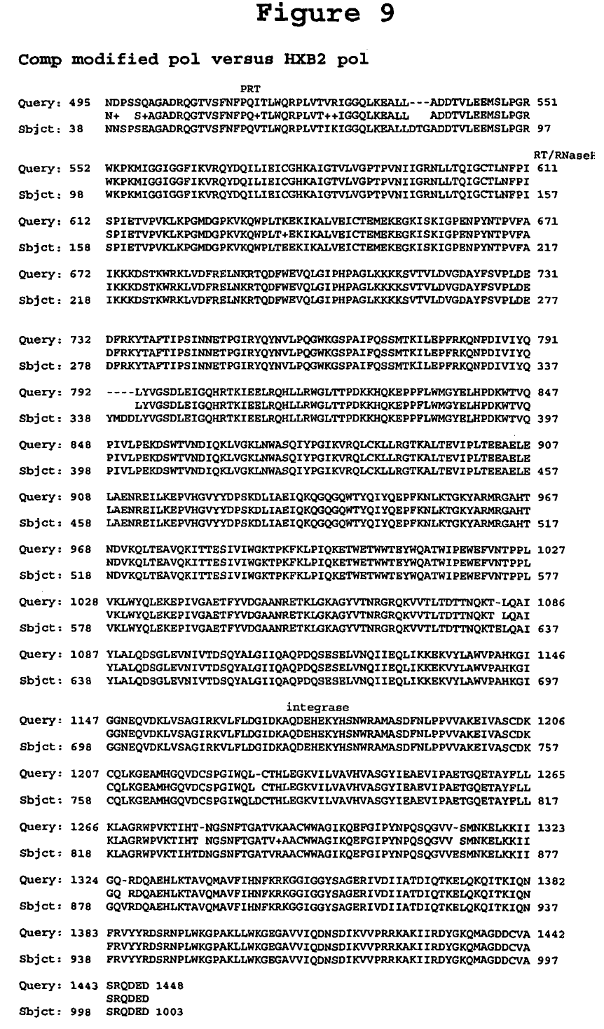

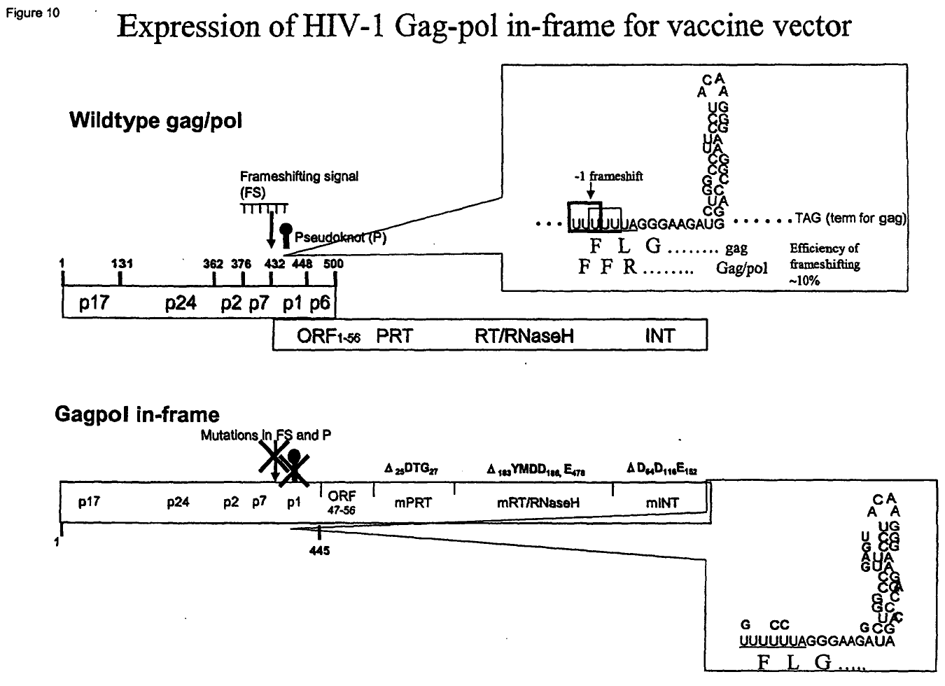

In some embodiments, the sequences are modified, e.g., to inactivate the protein or to align to conserved epitopes, such as CTL epitopes, to generate conserve epitopes. Exemplary modified HIV proteins are shown in FIGS. 8-11.

The following terminology is used with reference to the exemplary HIV constructs, the sequences of which are provided herein. All the genes are expressed from the CMV promoter and have BHG polyadenylation signal using the same or similar vectors as described for SIV.

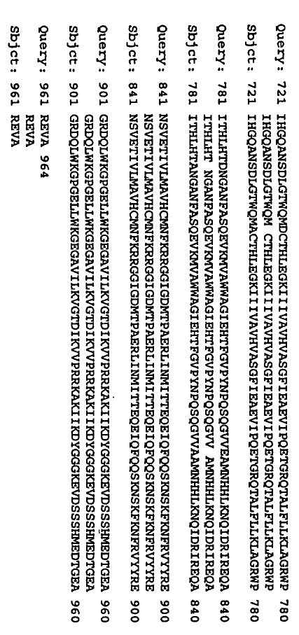

p37M1-10(gag) is the native N term portion of gag
CATEp37M1-10 is the CATE-p37gag fusion protein
MCP3p37M1-10 is the MCP3-p37gag fusion protein
CATEenv is the CATE-env fusion protein'
tPAenv is the tPA-env fusion
MCP3env is the MCP3env fusion
HIVgagpol is the gag-pol fusion protein
polNefTatVif is a fusion protein, all components are inactive—sequence comparisons for vif, tat, nef, and pol are shown in FIGS. 8-11. In some embodiments, these proteins are readily fused to CATE signals in recombinant fusion proteins. Schematics of changes in HIV-1 gagpol fusions and generation of Nef-tat-vif (NTV) fusion protein lacking nef/tat/vif function are shown in FIGS. 12 and 13. In FIG. 12, gagpol fusion protein or pol have the indicated mutations known to inactivate the function of protease, RT and integrase. In FIG. 13, Neftatvif has the mutations known to inactivate the individual proteins. All mutated constructs were tested for protein activity and shown to be inactive.

The following provides exemplary HIV gene and protein sequences used in vaccine constructs of the invention.

CATEp37gag(HIV)
ATGAGAAAAGCGGCTGTTAGTCACTGGCAGCAACAGTCTTACCTGGACTCTGGAATCCATTCTGG
TGCCACTACCACAGCTCCTTCTCTGAGTGTCGACAGAGAGATGGGTGCGAGAGCGTCAGTATTAA
GCGGGGGAGAATTAGATCGATGGGAAAAAATTCGGTTAAGGCCAGGGGGAAAGAAGAAGTACAAG
CTAAAGCACATCGTATGGGCAAGCAGGGAGCTAGAACGATTCGCAGTTAATCCTGGCCTGTTAGA
AACATCAGAAGGCTGTAGACAAATACTGGGACAGCTACAACCATCCCTTCAGACAGGATCAGAGG
AGCTTCGATCACTATACAACACAGTAGCAACCCTCTATTGTGTGCACCAGCGGATCGAGATCAAG
GACACCAAGGAAGCTTTAGACAAGATAGAGGAAGAGCAAAACAAGTCCAAGAAGAAGGCCCAGCA
GGCAGCAGCTGACACAGGACACAGCAATCAGGTCAGCCAAAATTACCCTATAGTGCAGAACATCC
AGGGGCAAATGGTACATCAGGCCATATCACCTAGAACTTTAAATGCATGGGTAAAAGTAGTAGAA
GAGAAGGCTTTCAGCCCAGAAGTGATACCCATGTTTTCAGCATTATCAGAAGGAGCCACCCCACA
GGACCTGAACACGATGTTGAACACCGTGGGGGGACATCAAGCAGCCATGCAAATGTTAAAAGAGA
CCATCAATGAGGAAGCTGCAGAATGGGATAGAGTGCATCCAGTGCATGCAGGGCCTATTGCACCA
GGCCAGATGAGAGAACCAAGGGGAAGTGACATAGCAGGAACTACTAGTACCCTTCAGGAACAAAT
AGGATGGATGACAAATAATCCACCTATCCCAGTAGGAGAGATCTACAAGAGGTGGATAATCCTGG
GATTGAACAAGATCGTGAGGATGTATAGCCCTACCAGCATTCTGGACATAAGACAAGGACCAAAG
GAACCCTTTAGAGACTATGTAGACCGGTTCTATAAAACTCTAAGAGCTGAGCAAGCTTCACAGGA
GGTAAAAAATTGGATGACAGAAACCTTGTTGGTCCAAAATGCGAACCCAGATTGTAAGACCATCC
TGAAGGCTCTCGGCCCAGCGGCTACACTAGAAGAAATGATGACAGCATGTCAGGGAGTAGGAGGA
CCCGGCCATAAGGCAAGAGTTTTGTAG
polypeptide:
M R K A A V S H W Q Q Q S Y L D S G I H S G A T T T A P S L S V D
R E M G A R A S V L S G G E L D R W E K I R L R P G G K K K Y K L
K H L V W A S R E L E R F A V N P G L L E T S E G C R Q I L G Q L
Q P S L Q T G S E E L R S L Y N T V A T L Y C V H Q R I E I K D T
K E A L D K I E E E Q N K S K K K A Q Q A A A D T G H S N Q V S Q
N Y P I V Q N I Q G Q M V H Q A I S P R T L N A W V K V V E E K A
F S P E V I P M F S A L S E G A T P Q D L N T M L N T V G G H Q A
A M Q M L K E T I N E E A A E W D R V H P V H A G P I A P G Q M R
E P R G S D I A G T T S T L Q E Q L G W M T N N P P I P V G E I Y
K R W I I L G L N K I V R M Y S P T S J L D I R Q G P K E P F R D
Y V D R F Y K T L R A E Q A S Q E V K N W M T E T L L V Q N A N P
D C K T I L K A L G P A A T L E E M M T A C Q G V G G P G H K A R
V L
PolNTV (HIV)
CCTCAGATCACGCTCTGGCAGCGGCCGCTCGTCACAATAAAGATCGGGGGGCAACTCAAGGAGGC
GCTGCTCGCGGACGACACGGTCTTGGAGGAGATGTCGTTGCCGGGGCGGTGGAAGCCGAAGATGA
TCGGGGGGATCGGGGGCTTCATCAAGGTGCGGCAGTACGACCAGATCCTCATCGAGATCTGCGGG
CACAAGGCGATCGGGACGGTCCTCGTCGGCCCGACGCCGGTCAACATCATCGGGCGGAACCTGTT
GACCCAGATCGGCTGCACCTTGAACTTCCCCATCAGCCCTATTGAGACGGTGCCCGTGAAGTTGA
AGCCGGGGATGGACGGCCCCAAGGTCAAGCAATGGCCATTGACGGAGGAGAAGATCAAGGCCTTA
GTCGAAATCTGTACAGAGATGGAGAAGGAAGGGAAGATCAGCAAGATCGGGCCTGAGAACCCCTA
CAACACTCCAGTCTTCGCAATCAAGAAGAAGGACAGTACCAAGTGGAGAAAGCTGGTGGACTTCA
GAGAGCTGAACAAGAGAACTCAGGACTTCTGGGAAGTTCAGCTGGGCATCCCACATCCCGCTGGG
TTGAAGAAGAAGAAGTCAGTGACAGTGCTGGATGTGGGTGATGCCTACTTCTCCGTTCCCTTGGA
CGAGGACTTCAGGAAGTACACTGCCTTCACGATACCTAGCATCAACAACGAGACACCAGGCATCC
GCTACCAGTACAACGTGCTGCCACAGGGATGGAAGGGATCACCAGCCATCTTTCAATCGTCGATG
ACCAAGATCCTGGAGCCCTTCCGCAAGGGAAAACCCAGACATCGTGATCTATCAGCTCTACGTAG
GAAGTGACCTGGAGATCGGGCAGCACAGGACCAAGATCGAGGAGCTGAGACAGCATCTGTTGAGG
TGGGGACTGACCACACCAGACAAGAAGCACCAGAAGGAACCTCCCTTCCTGTGGATGGGCTACGA
ACTGCATCCTGACAAGTGGACAGTGCAGCCCATCGTGCTGCCTGAGAAGGACAGCTGGACTGTGA
ACGACATACAGAAGCTCGTGGGCAAGTTGAACTGGGCAAGCCAGATCTACCCAGGCATCAAAGTT
AGGCAGCTGTGCAAGCTGCTTCGAGGAAACCAAAGGCACTGACAGAAGTGATCCCACTGACAGAG
GAAGCAGAGCTAGAACTGGCAGAGAACCGAGAGATCCTGAAGGAGCCAGTACATGGAGTGTACTA
CGACCCAAGCAAGGACCTGATCGCAGAGATCCAGAAGCAGGGGCAAGGCCAATGGACCTACCAAT
CTACCAGGAGCCCTTCAAGAACCTGAAGACAGGCAAGTACGCAAGGATGAGGGGTGCCCACACCA
ACGATGTGAAGCAGCTGACAGAGGCAGTGCAGAAGATCACCACAGAGAGCATCGTGATCTGGGGC
AAGACTCCCAAGTTCAAGCTGCCCATACAGAAGGAGACATGGGAGACATGGTGGACCGAGTACTG
GCAAGCCACCTGGATCCCTGAGTGGGAGTTCGTGAACACCCCTCCCTTGGTGAAAACTGTGGTAT
CAGCTGGAGAAGGAACCCATCGTGGGAGCAGAGACCTTCTACGTGGATGGGGCAGCCAACAGGGA
GACCAAGCTGGGCAAGGCAGGCTACGTGACCAACCGAGGACGACAGALAAGTGGTGACCCTGACT
GACACCACCAACCAGAAGACTCTGCAAGCCATCTACCTAGCTCTGCAAGACAGCGGACTGGAAGT
GAACATCGTGACAGACTCACAGTACGCACTGGGCATCATCCAAGCACAACCAGACCAATCCGAGT
CAGAGCTGGTGAACCAGATCATCGAGCAGCTGATCAAGAAGGAGAAAGTGTACCTGGCATGGGTC
CCGGCGCACAAGGGGATCGGGGGGAACGAGCAGGTCGACAAGTTGGTCTCGGCGGGGATCCGGAA
GGTGCTGTTCCTGGACGGGATCGATAAGGCCCAAGATGAACATGAGAAGTACCACTCCAACTGGC
GCGCTATGGCCAGCGACTTCAACCTGCCGCCGGTCGTCGCAAAAGAGATCGTCGCCAGCTGCGAC
AAGTGCCAGCTCAAGGGGGAGGCCATGCACGGGCAAGTCGACTGCAGTCCGGGGATCTGGCAGCT
GTGCACGCACCTGGAGGGGAGGTGATCCTGGTCGCGGTCCACGTCGCCAGCGGGTATATCGAGGC
GGAGGTCATCCCGGCTGAGACGGGGCAGGAGACGGCGTACTTCCTCTTGAAGCTCGCGGGGCGGT
GGCCGGTCAAGACGATCCACACGAACGGGAGCAACTTCACGGGGGCGACGGTCAAGGCCGCCTGT
TGGTGGGCGGGAATCAAGCAGGAATTTGGAATTCCCTACAATCCCCAATCGCAAGGAGTCGTGAG
CATGAACAJLGGAGCTGAAGAAGATCATCGGACAAAGGGATCAGGCTGAGCACCTGAAGACAGCA
GTGCAGATGGCAGTGTTCATCCACAACTTCAAAAGAAAAGGGGGGATTGGGGGGTACAGTGCGGG
GGAACGGATCGTGGACATCATCGCCACCGACATCCAAACCAAGGAGCTGCAGAAGCAGATCACCA
AGATCCAGAACTTCCGGGTGTACTACCGCGACAGCCGCAACCCACTGTGGAAGGGACCAGCAAAG
CTCCTCTGGAAGGGAGAGGGGGCAGTGGTGATCCAGGACAACAGTGACATCAAAGTGGTGCCAAG
GCGCAAGGCCAAGATCATCCGCGACTATGGAAAACAGATGGCAGGGGATGATTGTGTGGCAAGTA
GACAGGATGAGGATGGCGCCGCTAGCAAGTGGTCGAAGTCGTCGGTGATCGGGTGGCCGACTGTT
CGGGAGCGGATGCGGCGGGCGGAGCCGGCGGCGGATCGGGTGGGAGCGGCGTCGCGGGACCTTGA
GAAGCACGGGGCGATCACGTCGAGCAACACGGCGGCGACGAATGCGGCGTGTGCCTGGCTAGAGG
CGCAAGAGGAGGAGGAAGTGGGTTTTCCGGTCACGCCGCAGGTCCCGCTTCGGCCGATGACGTAC
AAGGCAGCGGTCGACCTCAGCCACTTCCTCAAGGAGAAGGGGGGACTGGAGGGGCTCATCCACTC
CCAGCGGCGGCAGGACATCCTTGACCTGTGGATCTACCACACACAAGGCTACTTCCCGGATTGGC
AGAACTACACGCCGGGGCCGGGGGTCCGGTATCCGCTGACCTTTGGATGGTGCTACAAGCTAGTA
CCGGTTGAGCCGGATAAGATCGAGGAGGCCAACAAGGGAGAGAACACCAGCTTGTTGCACCCTGT
GAGCCTGCATGGAATGGATGACCCGGAGCGGGAGGTGCTTGAGTGGCGGTTTGACAGCCGCCTAG
CGTTTCATCACGTGGCCCGAGAGCTGCATCCGGAGTACTTCAAGAACTGCGGATCCGAGCCAGTA
GATCCTAGACTAGAGCCCTGGAAGCATCCAGGATCGCAGCCGAAGACGGCGTGCACCAACTGCTA
CTGCAAGAAGTGCTTCCACCAGGTCTGCTTCATGACGAAGGCCTTGGGCATCTCCTATGGCCGGA
AGAAGCGGAGACAGCGACGAAGAGCTCATCAGAACTCGCAGACGCACCAGGCGTCGCTATCGAAG
CAACCCACCTCCCAATCCCGAGGGGACCCGACAGGCCCGAAGGAATCGAAGAAGGAGGTGGAGAG
AGAGACAGAGACAGATCCGTTCGACTGGTCTAGAGAGAACCGGTGGCAGGTGATGATTGTGTGGC
AGGTCGACCGGATGCGGATTCGGACGTGGAAGTCGCTTGTCAAGCACCACATGTACATCTCGGGG
AAGGCGAAGGGGTGGTTCTACCGGCACCACTATGAGTCGACGCACCCGCGGATCTCGTCGGAGGT
CCACATCCCGCTAGGGGACGCGAAGCTTGTCATCACGACGTACTGGGGTCTGCATACGGGAGAGC
GGGACTGGCATTTGGGTCAGGGAGTCTCCATAGAGTGGAGGAAAAAGCGGTATAGCACGCAAGTA
GACCCGGACCTAGCGGACCAGCTAATCCACCTGTACTACTTCGACTCGTTCTCGGAGTCGGCGAT
ACGGAATACCATCCTTGGGCGGATCGTTTCGCCGCGGAGTGAGTATCAAGCGGGGCACAACAAGG
TCGGGTCGCTACAGTACTTGGCGCTCGCGGCGTTGATCACGCCGAAGCAGATAAAGCCGCCGTTG
CCGTCGGTTACGAAACTGACGGAGGACCGGTGGAACAAGCCCCAGAAGACCAAGGGCCACCGGGG
GAGCCACACAATGAACGGGCACGTTAACTAG
protein:
M P Q I T L W Q R P L V T I K I G G Q L K E A L L A D D T V L E E
M S L P G R W K P K M I G G I G G F I K V R Q Y D Q I L I E I C G
H K A I G T V L V G P T P V N I I G R N L L T Q I G C T L N F P I
S P I E T V P V K L K P G M D G P K V K Q W P L T E E K I K A L V
E I C T E M E K E G K J S K I G P E N P Y N T P V F A I K K K D S
T K W R K L V D F R E L N K R T Q D F W E V Q L G I P H P A G L K
K K K S V T V L D V G D A Y F S V P L D E D F R K Y T A F T I P S
I N N E T P G I R Y Q Y N V L P Q G W K G S P A I F Q S S M T K I
L E P F R K Q N P D I V I Y Q L Y V G S D L E I G Q H R T K I E E
L R Q H L L R W G L T T P D K K H Q K E P P F L W M G Y E L H P D
K W T V Q P I V L P E K D S W T V N D I Q K L V G K L N W A S Q I
Y P G I K V R Q L C K L L R G T K A L T E V I P L T E E A E L E L
A E N R E I L K E P V H G V Y Y D P S K D L I A E I Q K Q G Q G Q
W T Y Q I Y Q E P F K N L K T G K Y A R M R G A H T N D V K Q L T
E A V Q K I T T E S I V I W G K T P K F K L P I Q K E T W E T W W
T E Y W Q A T W I P E W E F V N T P P L V K L W Y Q L E K E P I V
G A E T F Y V D G A A N R E T K L G K A G Y V T N R G R Q K V V T
L T D T T N Q K T L Q A I Y L A L Q D S G L E V N I V T D S Q Y A
L G I I Q A Q P D Q S E S E L V N Q I I E Q L I K K E K V Y L A W
V P A H K G I G G N E Q V D K L V S A G I R K V L F L D G I D K A
Q D E H E K Y H S N W R A M A S D F N L P P V V A K E I V A S C D
K C Q L K G E A M H G Q V D C S P G I W Q L C T H L E G K V J L V
A V H V A S G Y L E A E V I P A E T G Q E T A Y F L L K L A G R W
P V K T I H T N G S N F T G A T V K A A C W W A G I K Q E F G I P
Y N P Q S Q G V V S M N K E L K K I I G Q R D Q A E H L K T A V Q
M A V F I H N F K R K G G I G G Y S A G E R I V D I I A T D J Q T
K E L Q K Q I T K J Q N F R V Y Y R D S R N P L W K G P A K L L W
K G E G A V V I Q D N S D I K V V P R R K A K I I R D Y G K Q M A
G D D C V A S R Q D E D (pol)
G A A S (linker)
K W S K S S V I G W P T V R E R M R R A E P A A D R V G A A S R D
L E K H G A I T S S N T A A T N A A C A W L E A Q E E E E V G F P
V T P Q V P L R P M T Y K A A V D L S H F L K E K G G L E G L I H
S Q R R Q D I L D L W I Y H T Q G Y F P D W Q N Y T P G P G V R Y
P L T F G W C Y K L V P V E P D K I E E A N K G E N T S L L H P V
S L H G M D D P E R E V L E W R F D S R L A F H H V A R E L H P E
Y F K N C (nef)
G S (linker)
E P V D P R L E P W K H P G S Q P K T A C T N C Y C K K C F H Q V
C F M T K A L G I S Y G R K K R R Q R R R A H Q N S Q T H Q A S L
S K Q P T S Q S R G D P T G P K E S K K E V E R E T E T D P F D W
(tat)
S R (linker)
E N R W Q V M I V W Q V D R M R I R T W K S L V K H H M Y I S G K
A K G W F Y R H H Y E S T H P R I S S E V H I P L G D A K L V I T
T Y W G L H T G E R D W H L G Q G V S I E W R K K R Y S T Q V D P
D L A D Q L I H L Y Y F D S F S E S A I R N T I L G R I V S P R S
E Y Q A G H N K V G S L Q Y L A L A A L I T P K Q I K P P L P S V
T K L T E D R W N K P Q K T K G H R G S H T M N G H (vif)
V N • (linker)
tPAenv (HIV)
ATGGATGCAATGAAGAGAGGGCTCTGCTGTGTGCTGCTGCTGTGTGGAGCAGTCTTCGTTTCGCC
CAGCCAGGAAATCCATGCCCGATTCAGAAGAGGAGCCAGATCTATCTGCAGCGCCGAGGAGAAGC
TGTGGGTCACGGTCTATTATGGCGTGCCCGTGTGGAAAGAGGCAACCACCACGCTATTCTGCGCC
TCCGACGCCAAGGCACATCATGCAGAGGCGCACAACGTCTGGGCCACGCATGCCTGTGTACCCAC
GGACCCTAACCCCCAAGAGGTGATCCTGGAGAACGTGACCGAGAAGTACAACATGTGGAAAAATA
ACATGGTAGACCAGATGCATGAGGATATAATCAGTCTATGGGATCAAAGCCTAAAGCCATGTGTA
AAACTAACCCCCCTCTGCGTGACGCTGAATTGCACCAACGCGACGTATACGAATAGTGACAGTAA
GAATAGTACCAGTAATAGTAGTTTGGAGGACAGTGGGAAAGGAGACATGAACTGCTCGTTCGATG
TCACCACCAGCATCGACAAGAAGAAGAAGACGGAGTATGCCATCTTCGACAAGCTGGATGTAATG
AATATAGGAAATGGAAGATATACGCTATTGAATTGTAACACCAGTGTCATTACGCAGGCCTGTCC
AAAGATGTCCTTTGAGCCAATTCCCATACATTATTGTACCCCGGCCGGCTACGCGATCCTGAAGT
GCAACGACAATAAGTTCAATGGAACGGGACCATGTACGAATGTCAGCACGATACAATGTACGCAT
GGAATTAAGCCAGTAGTGTCGACGCAACTGCTGCTGAACGGCAGCCTGGCCGAGGGAGGAGAGGT
AATAATTCGGTCGGAGAACCTCACCGACAACGCCAAGACCATAATAGTACAGCTCAAGGAACCCG
TGGAGATCAACTGTACGAGACCCAACAACAACACCCGAAAGAGCATACATATGGGACCAGGAGCA
GCATTTTATGCAAGAGGAGAGGTAATAGGAGATATAAGACAAGCACATTGCAACATTAGTAGAGG
AAGATGGAATGACACTTTGAAACAGATAGCTAAAAAGCTGCGCGAGCAGTTTAACAAGACCATAA
GCCTTAACCAATCCTCGGGAGGGGACCTAGAGATTGTAATGCACACGTTTAATTGTGGAGGGGAG
TTTTTCTACTGTAACACGACCCAGCTGTTCAACAGCACCTGGAATGAGAATGATACGACCTGGAA
TAATACGGCAGGGTCGAATAACAATGAGACGATCACCCTGCCCTGTCGCATCAAGCAGATCATAA
ACAGGTGGCAGGAAGTAGGAAAAGCAATGTATGCCCCTCCCATCAGTGGCCCGATCAACTGCTTG
TCCAACATCACCGGGCTATTGTTGACGAGAGATGGTGGTGACAACAATAATACGATAGAGACCTT
CAGACCTGGAGGAGGAGATATGAGGGACAACTGGAGGAGCGAGCTGTACAAGTACAAGGTAGTGA
GGATCGAGCCATTGGGAATAGCACCCACCAAGGCAAAGAGAAGAGTGGTGCAAAGAGAGAAAAGA
GCAGTGGGAATAGGAGCTATGTTCCTTGGGTTCTTGGGAGCAGCAGGAAGCACTATGGGCGCAGC
GTCGGTGACCCTTACCGTGCAAGCTCGCCTGCTGCTGTCGGGTATAGTGCAACAGCAAAACAACC
TCCTCCGCGCAATCGAAGCCCAGCAGCATCTGTTGCAACTCACGGTCTGGGGCATCAAGCAGCTC
CAGGCTAGAGTCCTTGCCATGGAGCGTTATCTGAAAGACCAGCAACTTCTTGGGATTTGGGGTTG
CTCGGGAAAACTCATTTGCACCACGAATGTGCCTTGGAACGCCAGCTGGAGCAACAAGTCCCTGG
ACAAGATTTGGCATAACATGACCTGGATGGAGTGGGACCGCGAGATCGACAACTACACGAAATTG
ATATACACCCTGATCGAGGCGTCCCAGATCCAGCAGGAGAAGAATGAGCAAGAGTTGTTGGAGTT
GGATTCGTGGGCGTCGTTGTGGTCGTGGTTTGACATCTCGAAATGGCTGTGGTATATAGGAGTAT
TCATAATAGTAATAGGAGGTTTGGTAGGTTTGAAAATAGTTTTTGCTGTACTTTCGATAGTAAAT
CGAGTTAGGCAGGGATACTCGCCATTGTCATTTCAAACCCGCCTCCCAGCCCCGCGGGGACCCGA
CAGGCCCGAGGGCATCGAGGAGGGAGGCGGCGAGAGAGACAGAGACAGATCCGATCAATTGGTGA
CGGGATTCTTGGCACTCATCTGGGACGATCTGCGGAGCCTGTGCCTCTTCTCTTACCACCGCCTG
CGCGACCTGCTCCTGATCGTGGCGAGGATCGTGGAGCTTCTGGGACGCAGGGGGTGGGAGGCCCT
GAAGTACTGGTGGAACCTCCTGCAATATTGGATTCAGGAGCTGAAGAACAGCGCCGTTAGTCTGC
TGAACGCTACCGCTATCGCCGTGGCGGAAGGAACCGACAGGATTATAGAGGTAGTACAAAGGATT
GGTCGCGCCATCCTCCATATCCCCCGCCGCATCCGCCAGGGCTTGGAGAGGGCTTTGCTATAA
protein:
M D A M K R G L C C V L L L C G A V F V S P S Q E I H A R F R R G
A R S (tPA)
I C S (linker)
A E E K L W V T V Y Y G V P V W K E A T T T L F C A S D A K A H H
A E A H N V W A T H A C V P T D P N P Q E V I L E N V T E K Y N M
W K N N M V D Q M H E D I I S L W D Q S L K P C V K L T P L C V T
L N C T N A T Y T N S D S K N S T S N S S L E D S G K G D M N C S
F D V T T S I D K K K K T E Y A I F D K L D V M N I G N G R Y T L
L N C N T S V I T Q A C P K M S F E P I P I H Y C T P A G Y A I L
K C N D N K F N G T G P C T N V S T I Q C T H G I K P V V S T Q L
L L N G S L A E G G E V I I R S E N L T D N A K T I I V Q L K E P
V E I N C T R P N N N T R K S I H M G P G A A F Y A R G E V I G D
I R Q A H C N I S R G R W N D T L K Q I A K K L R E Q F N K T I S
L N Q S S G G D L E I V M H T F N C G G E F F Y C N T T Q L F N S
T W N E N D T T W N N T A G S N N N E T I T L P C R I K Q I I N R
W Q E V G K A M Y A P P I S G P I N C L S N I T G L L L T R D G G
D N N N T I E T F R P G G G D M R D N W R S E L Y K Y K V V R I E
P L G I A P T K A K R R V V Q R E K R A V G I G A M F L G F L G A
A G S T M G A A S V T L T V Q A R L L L S G I V Q Q Q N N L L R A
I E A Q Q H L L Q L T V W G I K Q L Q A R V L A M E R Y L K D Q Q
L L G I W G C S G K L I C T T N V P W N A S W S N K S L D K I W H
N M T W M E W D R E I D N Y T K L I Y T L I E A S Q I Q Q E K N E
Q E L L E L D S W A S L W S W F D I S K W L W Y I G V F I I V L G
G L V G L K I V F A V L S I V N R V R Q G Y S P L S F Q T R L P A
P R G P D R P E G I E E G G G E R D R D R S D Q L V T G F L A L I
W D D L R S L C L F S Y H R L R D L L L I V A R I V E L L G R R G
W E A L K Y W W N L L Q Y W I Q E L K N S A V S L L N A T A I A V
A E G T D R I I E V V Q R I G R A I L H I P R R I R Q G L E R A L
L • (env)
MCP3 HIVenv
ATGAACCCAAGTGCTGCCGTCATTTTCTGCCTCATCCTGCTGGGTCTGAGTGGGACTCAAGGGAT
CCTCGACATGGCGCAACCGGTAGGTATAAACACAAGCACAACCTGTTGCTATCGTTTCATAAATA
AAAAGATACCGAAGCAACGTCTGGAAAGCTATCGCCGTACCACTTCTAGCCACTGTCCGCGTGAA
GCTGTTATATTCAAAACGAAACTGGATAAGGAGATCTGCGCCGACCCTACACAGAAATGGGTTCA
GGACTTTATGAAGCACCTGGATAAAAAGACACAGACGCCGAAACTGATCTGCAGCGCCGAGGAGA
AGCTGTGGGTCACGGTCTATTATGGCGTGCCCGTGTGGAAAGAGGCAACCACCACGCTATTCTGC
GCCTCCGACGCCAAGGCACATCATGCAGAGGCGCACAACGTCTGGGCCACGCATGCCTGTGTACC
CACGGACCCTAACCCCCAAGAGGTGATCCTGGAGAACGTGACCGAGAAGTACAACATGTGGAAAA
ATAACATGGTAGACCAGATGCATGAGGATATAATCAGTCTATGGGATCAAAGCCTAAAGCCATGT
GTAAAACTAACCCCCCTCTGCGTGACGCTGAATTGCACCAACGCGACGTATACGAATAGTGACAG
TAAGAATAGTACCAGTAATAGTAGTTTGGAGGACAGTGGGAAAGGAGACATGAACTGCTCGTTCG
ATGTCACCACCAGCATCGACAAGAAGAAGAAGACGGAGTATGCCATCTTCGACAAGCTGGATGTA
ATGAATATAGGAAATGGAAGATATACGCTATTGAATTGTAACACCAGTGTCATTACGCAGGCCTG
TCCAAAGATGTCCTTTGAGCCAATTCCCATACATTATTGTACCCCGGCCGGCTACGCGATCCTGA
AGTGCAACGACAATAAGTTCAATGGAACGGGACCATGTACGAATGTCAGCACGATACAATGTACG
CATGGAATTAAGCCAGTAGTGTCGACGCAACTGCTGCTGAACGGCAGCCTGGCCGAGGGAGGAGA
GGTAATAATTCGGTCGGAGAACCTCACCGACAACGCCAAGACCATAATAGTACAGCTCAAGGAAC
CCGTGGAGATCAACTGTACGAGACCCAACAACAACACCCGAAAGAGCATACATATGGGACCAGGA
GCAGCATTTTATGCAAGAGGAGAGGTAATAGGAGATATAAGACAAGCACATTGCAACATTAGTAG
AGGAAGATGGAATGACACTTTGAAACAGATAGCTAAAAAGCTGCGCGAGCAGTTTAACAAGACCA
TAAGCCTTAACCAATCCTCGGGAGGGGACCTAGAGATTGTAATGCACACGTTTAATTGTGGAGGG
GAGTTTTTCTACTGTAACACGACCCAGCTGTTCAACAGCACCTGGAATGAGAATGATACGACCTG
GAATAATACGGCAGGGTCGAATAACAATGAGACGATCACCCTGCCCTGTCGCATCAAGCAGATCA
TAAACAGGTGGCAGGAAGTAGGAAAAGCAATGTATGCCCCTCCCATCAGTGGCCCGATCAACTGC
TTGTCCAACATCACCGGGCTATTGTTGACGAGAGATGGTGGTGACAACAATAATACGATAGAGAC
CTTCAGACCTGGAGGAGGAGATATGAGGGACAACTGGAGGAGCGAGCTGTACAAGTACAAGGTAG
TGAGGATCGAGCCATTGGGAATAGCACCCACCAAGGCAAAGAGAAGAGTGGTGCAAAGAGAGAAA
AGAGCAGTGGGAATAGGAGCTATGTTCCTTGGGTTCTTGGGAGCAGCAGGAAGCACTATGGGCGC
AGCGTCGGTGACCCTTACCGTGCAAGCTCGCCTGCTGCTGTCGGGTATAGTGCAACAGCAAAACA
ACCTCCTCCGCGCAATCGAAGCCCAGCAGCATCTGTTGCAACTCACGGTCTGGGGCATCAAGCAG
CTCCAGGCTAGAGTCCTTGCCATGGAGCGTTATCTGAAAGACCAGCAACTTCTTGGGATTTGGGG
TTGCTCGGGAAAACTCATTTGCACCACGAATGTGCCTTGGAACGCCAGCTGGAGCAACAAGTCCC
TGGACAAGATTTGGCATAACATGACCTGGATGGAGTGGGACCGCGAGATCGACAACTACACGAAA
TTGATATACACCCTGATCGAGGCGTCCCAGATCCAGCAGGAGAAGAATGAGCAAGAGTTGTTGGA
GTTGGATTCGTGGGCGTCGTTGTGGTCGTGGTTTGACATCTCGAAATGGCTGTGGTATATAGGAG
TATTCATAATAGTAATAGGAGGTTTGGTAGGTTTGAAAATAGTTTTTGCTGTACTTTCGATAGTA
AATCGAGTTAGGCAGGGATACTCGCCATTGTCATTTCAAACCCGCCTCCCAGCCCCGCGGGGACC
CGACAGGCCCGAGGGCATCGAGGAGGGAGGCGGCGAGAGAGACAGAGACAGATCCGATCAATTGG
TGACGGGATTCTTGGCACTCATCTGGGACGATCTGCGGAGCCTGTGCCTCTTCTCTTACCACCGC
CTGCGCGACCTGCTCCTGATCGTGGCGAGGATCGTGGAGCTTCTGGGACGCAGGGGGTGGGAGGC
CCTGAAGTACTGGTGGAACCTCCTGCAATATTGGATTCAGGAGCTGAAGAACAGCGCCGTTAGTC
TGCTGAACGCTACCGCTATCGCCGTGGCGGAAGGAACCGACAGGATTATAGAGGTAGTACAAAGG
ATTGGTCGCGCCATCCTCCATATCCCCCGCCGCATCCGCCAGGGCTTGGAGAGGGCTTTGCTATA
A
protein:
M N P S A A V I F C L I L L G L S G T Q G I L D M A Q P V G I N T
S T T C C Y R F I N K K I P K Q R L E S Y R R T T S S H C P R E A
V I F K T K L D K E I C A D P T Q K W V Q D F M K H L D K K T Q T
P K L I C S A E E K L W V T V Y Y G V P V W K E A T T T L F C A S
D A K A H H A E A H N V W A T H A C V P T D P N P Q E V I L E N V
T E K Y N M W K N N M V D Q M H E D I I S L W D Q S L K P C V K L
T P L C V T L N C T N A T Y T N S D S K N S T S N S S L E D S G K
G D M N C S F D V T T S I D K K K K T E Y A I F D K L D V M N I G
N G R Y T L L N C N T S V I T Q A C P K M S F E P I P I H Y C T P
A G Y A I L K C N D N K F N G T G P C T N V S T I Q C T H G I K P
V V S T Q L L L N G S L A E G G E V I I R S E N L T D N A K T I I
V Q L K E P V E I N C T R P N N N T R K S I H M G P G A A F Y A R
G E V I G D I R Q A H C N I S R G R W N D T L K Q I A K K L R E Q
F N K T I S L N Q S S G G D L E I V M H T F N C G G E F F Y C N T
T Q L F N S T W N E N D T T W N N T A G S N N N E T I T L P C R I
K Q I I N R W Q E V G K A M Y A P P I S G P I N C L S N I T G L L
L T R D G G D N N N T I E T F R P G G G D M R D N W R S E L Y K Y
K V V R I E P L G I A P T K A K R R V V Q R E K R A V G I G A M F
L G F L G A A G S T M G A A S V T L T V Q A R L L L S G I V Q Q Q
N N L L R A I E A Q Q H L L Q L T V W G I K Q L Q A R V L A M E R
Y L K D Q Q L L G I W G C S G K L I C T T N V P W N A S W S N K S
L D K I W H N M T W M E W D R E I D N Y T K L I Y T L I E A S Q I
Q Q E K N E Q E L L E L D S W A S L W S W F D I S K W L W Y I G V
F I I V I G G L V G L K I V F A V L S J V N R V R Q G Y S P L S F
Q T R L P A P R G P D R P E G I E E G G G E R D R D R S D Q L V T
G F L A L I W D D L R S L C L F S Y H R L R D L L L I V A R I V E
L L G R R G W E A L K Y W W N L L Q Y W I Q E L K N S A V S L L N
A T A I A V A E G T D R I I E V V Q R I G R A I L H I P R R I R Q
G L E R A L L •
CATEenv(HIV)
ATGAGAAAAGCGGCTGTTAGTCACTGGCAGCAGCAGTCTTACCTGGACTCTGGAATCCATTCTGG
TGCCACTACCACAGCTCCTTCTCTGAGTATCTGCAGCGCCGAGGAGAAGCTGTGGGTCACGGTCT
ATTATGGCGTGCCCGTGTGGAAAGAGGCAACCACCACGCTATTCTGCGCCTCCGACGCCAAGGCA
CATCATGCAGAGGCGCACAACGTCTGGGCCACGCATGCCTGTGTACCCACGGACCCTAACCCCCA
AGAGGTGATCCTGGAGAACGTGACCGAGAAGTACAACATGTGGAAAATAACATGGTAGACCAGAT
GCATGAGGATATAATCAGTCTATGGGATCAAAGCCTAAAGCCATGTGTMAACTAACCCCCCTCTG
CGTGACGCTGAATTGCACCAACGCGACGTATACGAATAGTGACAGTAAGAATAGTACCAGTAATA
GTAGTTTGGAGGACAGTGGGAAAGGAGACATGAACTGCTCGTTCGATGTCACCACCAGCATCGAC
AAAAGAAGAAGAAAGACGGAGTATGCCATCTTCGACAAGCTGGATGTAATGAATATAGGAAAAAT
GGAAGATATACGCTATTGAATTGTAACACCAGTGTCATTACGCAGGCCTGTCCAAAQATGTCCTT
TGAGCCAATTCCCATACATTATTGTACCCCGGCCGGCTACGCGATCCTGAAGTGCAACGACAATA
AGTTCAATGGAACGGGACCATGTACGAATGTCAGCACGATACAATGTACGCATGGAATTAAGCCA
GTAGTGTCGACGCAACTGCTGCTGAACGGCAGCCTGGCCGAGGGAGGAGAGGTAATAATTCGGTC
GGAGACCTCACCGACAACGCCAAGACCATAATAGTACAGCTCAAGGAACCCGTGGAGATCAACTG
TACGAGACCCAACAACAACACCCGAAAGAGCATACATATGGGACCAGGAGCAGCATTTTATGCAA
GAGGAGAGGTAATAGGAGATATAAGACAAGCACATTGCAACATTAGTAGAGGAAGATGGAATGAC
ACTTTGAAACAGATAGCTAAAAAGCTGCGCGAGCAGTTTAACAAGACCATAAGCCTTAACCAATC
CTCGGGAGGGGACCTAGAGATTGTPAAGCACACGTTTAATTGTGGAGGGGAGTTTTTCTACTGTA
ACACGACCCAGCTGTTCPCAGCACCTGGAATGAGAATGATACGACCTGGAATAATACGGCAGGGT
CGAATAACAATGAGACGATCACCCTGCCCTGTCGCATCAAGCAGATCATAAACAGGTGGCAGGAA
GTAGGAAAGCAATGTATGCCCCTCCCATCAGTGGCCCGATCAACTGCTTGTCCAACATCACCGGG
CTATTGTTGACGAGAGATGGTGGTGACAACAATAATACGATAGAGACCTTCAGACCTGGAGGAGG
AGATATGAGGGACAAAACTGGAGGAGCGAGCTGTACAAGTACAAGGTAGTGAGGATCGAGCCATT
GGGAATAGCACCCACCAAGGCAAAGAGAAGAGTGGTGCAAAGAGAGAAAAGAGCAGTGGGAATAG
GAGCTATGTTCCTTGGGTTCTTGGGAGCAGCAGGAAGCACTATGGGCGCAGCGTCGGTGACCCTT
ACCGTGCAAGCTCGCCTGCTGCTGTCGGGTATAGTGCAACAGCAAAACAACCTCCTCCGCGCAAT
CGAAGCCCAGCAGCATCTGTTGCAACTCACGGTCTGGGGCATCAAGCAGCTCCAGGCTAGAGTCC
TTGCCATGGAGCGTTATCTGAAAGACCAGCAATTTCTTGGGATTTGGGGTTGCTCGGGAACTCAT
TTGCACCACGAATGTGCCTTGGAACGCCAGCTGGAGCAACAAGTCCCTGGACAAGATTTGGCATA
ACATGACCTGGATGGAGTGGGACCGCGAGATCGACAACTACACGAAATTGATATACACCCTGATC
GAGGCGTCCCAGATCCAGCAGGAGAAGAATGAGCAAGAGTTGTTGGAGTTGGATTCGTGGGCGTC
GTTGTGGTCGTGGTTTGACATCTCGAAATGGCTGTGGTATATAGGAGTATTCATAATAGTAATAG
GAGGTTTGGTAGGTTTGMAATAGTTTTTGCTGTACTTTCGATAGTAAATCGAGTTAGGCAGGGAT
ACTCGCCATTGTCATTTCAAACCCGCCTCCCAGCCCCGCGGGGACCCGACAGGCCCGAGGGCATC
GAGGAGGGAGGCGGCGAGAGAGACAGAGACAGATCCGATCAATTGGTGACGGGATTCTTGGCACT
CATCTGGGACGATCTGCGGAGCCTGTGCCTCTTCTCTTACCACCGCCTGCGCGACCTGCTCCTGA
TCGTGGCGAGGATCGTGGAGCTTCTGGGACGCAGGGGGTGGGAGGCCCTGAAGTAGTCTGCTGAA
CGCTACCGCTATCGCCGTGGCGGAAGGAACCGACAGGATCGTTAGTCTGCTGAACGCTACCGCTA
TCGCCGTGGCGGAAAAGGAACCGACAGGATTATAGAGGTAGTACAAAGGATTGGTCGCGCCATCC
TCCATATCCCCCGCCGCATCCGCCAGGGCTTGGAGAGGGCTTTGCTATAA
protein:
M R K A A V S H W Q Q Q S Y L D S G I H S G A T T T A P S L S I C
S A E E K L W V T V Y Y G V P V W K E A T T T L F C A S D A K A H
H A E A H N V W A T H A C V P T D P N P Q E V I L E N V T E K Y N
M W K N N M V D Q M H E D I I S L W D Q S L K P C V K L T P L C V
T L N C T N A T Y T N S D S K N S T S N S S L E D S G K G D M N C
S F D V T T S I D K K K K T E Y A I F D K L D V M N I G N G R Y T
L L N C N T S V I T Q A C P K M S F E P I P I H Y C T P A G Y A I
L K C N D N K F N G T G P C T N V S T I Q C T H G I K P V V S T Q
L L L N G S L A E G G E V I I R S E N L T D N A K T I I V Q L K E
P V E I N C T R P N N N T R K S I H M G P G A A F Y A R G E V I G
D I R Q A H C N I S R G R W N D T L K Q I A K K L R E Q F N K T I
S L N Q S S G G D L E I V M H T F N C G G E F F Y C N T T Q L F N
S T W N E N D T T W N N T A G S N N N E T I T L P C R I K Q I I N
R W Q E V G K A M Y A P P I S G P I N C L S N I T G L L L T R D G
G D N N N T I E T F R P G G G D M R D N W R S E L Y K Y K V V R I
E P L G I A P T K A K R R V V Q R E K R A V G I G A M F L G F L G
A A G S T M G A A S V T L T V Q A R L L L S G I V Q Q Q N N L L R
A I E A Q Q H L L Q L T V W G I K Q L Q A R V L A M E R Y L K D Q
Q L L G I W G C S G K L I C T T N V P W N A S W S N K S L D K I W
H N M T W M E W D R E I D N Y T K L I Y T L I E A S Q I Q Q E K N
E Q E L L E L D S W A S L W S W F D I S K W L W Y I G V F I I V I
G G L V G L K I V F A V L S I V N R V R Q G Y S P L S F Q T R L P
A P R G P D R P E G I E E G G G E R D R D R S D Q L V T G F L A L
I W D D L R S L C L F S Y H R L R D L L L I V A R I V E L L G R R
G W E A L K Y W W N L L Q Y W I Q E L K N S A V S L L N A T A I A
V A E G T D R I I E V V Q R I G R A I L H I P R R I R Q G L E R A
L L •
PMCP3p37M1-10
ATGAACCCAAGTGCTGCCGTCATTTTCTGCCTCATCCTGCTGGGTCTGAGTGGGACTCAAGGGAT
CCTCGACATGGCGCAACCGGTAGGTATAAACACAAGCACAACCTGTTGCTATCGTTTCATAAATA
AAAAGATACCGAAGCAACGTCTGGAAAGCTATCGCCGTACCACTTCTAGCCACTGTCCGCGTGAA
GCTGTTATATTCAAAACGAAACTGGATAAGGAGATCTGCGCCGACCCTACACAGAAATGGGTTCA
GGACTTTATGAAGCACCTGGATAAAAAGACACAGACGCCGAAACTGGCTAGCGCAGGAGCAGGTG
CGAGAGCGTCAGTATTAAGCGGGGGAGAATTAGATCGATGGGAAAAAATTCGGTTAAGGCCAGGG
GGAAAGAAGAAGTACAAGCTAAAGCACATCGTATGGGCAAGCAGGGAGCTAGAACGATTCGCAGT
TAATCCTGGCCTGTTAGAAACATCAGAAGGCTGTAGACAAATACTGGGACAGCTACAACCATCCC
TTCAGACAGGATCAGAGGAGCTTCGATCACTATACAACACAGTAGCAACCCTCTATTGTGTGCAC
CAGCGGATCGAGATCAAGGACACCAAGGAAGCTTTAGACAAGATAGAGGAAGAGCAAAACAAGTC
CAAGAAGAAGGCCCAGCAGGCAGCAGCTGACACAGGACACAGCAATCAGGTCAGCCAAAATTACC
CTATAGTGCAGAACATCCAGGGGCAAATGGTACATCAGGCCATATCACCTAGAACTTTAAATGCA
TGGGTAAAAGTAGTAGAAGAGAAGGCTTTCAGCCCAGAAGTGATACCCATGTTTTCAGCATTATC
AGAAGGAGCCACCCCACAGGACCTGAACACGATGTTGAACACCGTGGGGGGACATCAAGCAGCCA
TGCAAATGTTAAAAGAGACCATCAATGAGGAAGCTGCAGAATGGGATAGAGTGCATCCAGTGCAT
GCAGGGCCTATTGCACCAGGCCAGATGAGAGAACCAAGGGGAAGTGACATAGCAGGAACTACTAG
TACCCTTCAGGAACAAATAGGATGGATGACAAATAATCCACCTATCCCAGTAGGAGAGATCTACA
AGAGGTGGATAATCCTGGGATTGAACAAGATCGTGAGGATGTATAGCCCTACCAGCATTCTGGAC
ATAAGACAAGGACCAAAGGAACCCTTTAGAGACTATGTAGACCGGTTCTATAAAACTCTAAGAGC
TGAGCAAGCTTCACAGGAGGTAAAAAATTGGATGACAGAAACCTTGTTGGTCCAAAATGCGAACC
CAGATTGTAAGACCATCCTGAAGGCTCTCGGCCCAGCGGCTACACTAGAAGAAATGATGACAGCA
TGTCAGGGAGTAGGAGGACCCGGCCATAAGGCAAGAGTTTTGGAATTCTGA
protein:
M N P S A A V I F C L I L L G L S G T Q (MCP3)
G I L D (linker)
M A Q P V G I N T S T T C C Y R F I N K K I P K Q R L E S Y R R T
T S S H C P R E A V I F K T K L D K E I C A D P T Q K W V Q D F M
K H L D K K T Q T P K L A S A G A G A R A S V L S G G E L D R W E
K I R L R P G G K K K Y K L K H I V W A S R E L E R F A V N P G L
L E T S E G C R Q I L G Q L Q P S L Q T G S E E L R S L Y N T V A
T L Y C V H Q R I E J K D T K E A L D K J E E E Q N K S K K K A Q
Q A A A D T G H S N Q V S Q N Y P I V Q N I Q G Q M V H Q A I S P
R T L N A W V K V V E E K A F S P E V I P M F S A L S E G A T P Q
D L N T M L N T V G G H Q A A M Q M L K E T I N E E A A E W D R V
H P V H A G P I A P G Q M R E P R G S D I A G T T S T L Q E Q I G
W M T N N P P I P V G E I Y K R W I I L G L N K I V R M Y S P T S
I L D I R Q G P K E P F R D Y V D R F Y K T L R A E Q A S Q E V K
N W M T E T L L V Q N A N P D C K T I L K A L G P A A T L E E M M
T A C Q G V G G P G H K A R V L E F • (p37gag)
p37M1-10 (HIV)
ATGGGTGCGAGAGCGTCAGTATTAAGCGGGGGAGAATTAGATCGATGGGAAAAAATTCGGTTAAG
GCCAGGGGGAAAGAAGAAGTACAAGCTAAAGCACATCGTATGGGCAAGCAGGGAGCTAGAACGAT
TCGCAGTTAATCCTGGCCTGTTAGAAACATCAGAAGGCTGTAGACAAATACTGGGACAGCTACAA
CCATCCCTTCAGACAGGATCAGAGGAGCTTCGATCACTATACAACACAGTAGCAACCCTCTATTG
TGTGCACCAGCGGATCGAGATCAAGGACACCAAGGAAGCTTTAGACAAGATAGAGGAAGAGCAAA
ACAAGTCCAAGAAGAAGGCCCAGCAGGCAGCAGCTGACACAGGACACAGCAAATCAGGTCAGCCA
AAATTACCCTATAGTGCAGAACATCCAGGGGCAAATGGTACATCAGGCCATATCACCTAGAACTT
TAAATGCATGGGTAAAAGTAGTAGAAGAGAAGGCTTTCAGCCCAGAAGTGATACCCATGTTTTCA
GCATTATCAGAAGGAGCCACCCCACAGGACCTGAACACGATGTTGAACACCGTGGGGGGACATCA
AGCAGCCATGCAAATGTTAAAAGAGACCATCAATGAGGAAGCTGCAGAATGGGATAGAGTGCATC
CAGTGCATGCAGGGCCTATTGCACCAGGCCAGATGAGAGAACCAAGGGGAAGTGACATAGCAGGA
ACTACTAGTACCCTTCAGGAACAAATAGGATGGATGACAAATAATCCACCTATCCCAGTAGGAGA
GATCTACAAGAGGTGGATAATCCTGGGATTGAACAAGATCGTGAGGATGTATAGCCCTACCAGCA
TTCTGGACATAAGACAAGGACCAAAGGAACCCTTTAGAGACTATGTAGACCGGTTCTATAAAACT
CTAAGAGCTGAGCAAGCTTCACAGGAGGTAAAAAATTGGATGACAGAAACCTTGTTGGTCCAAAA
ATGCGAACCCAGATTGTAAGACCATCCTGAAGGCTCTCGGCCCAGCGGCTACACTAGAAGAAATG
ATGACAGCATGTCAGGGAGTAGGAGGACCCGGCCATAAGGCAAGAGTTTTGTAG
protein:
M G A R A S V L S G G E L D R W E K I R L R P G G K K K Y K L K H
I V W A S R E L E R F A V N P G L L E T S E G C R Q I L G Q L Q P
S L Q T G S E E L R S L Y N T V A T L Y C V H Q R J E I K D T K E
A L D K I E E E Q N K S K K K A Q Q A A A D T G H S N Q V S Q N Y
P I V Q N I Q G Q M V H Q A I S P R T L N A W V K V V E E K A F S
P E V I P M F S A L S E G A T P Q D L N T M L N T V G G H Q A A M
Q M L K E T I N E E A A E W D R V H P V H A G P I A P G Q M R E P
R G S D I A G T T S T L Q E Q I G W M T N N P P I P V G E I Y K R
W I I L G L N K I V R M Y S P T S I L D I R Q G P K E P F R D Y V
D R F Y K T L R A E Q A S Q E V K N W M T E T L L V Q N A N P D C
K T I L K A L G P A A T L E E M M T A C Q G V G G P G H K A R V
L •
HIV gagpol
ATGGGTGCGAGAGCGTCAGTATTAAGCGGGGGAGAATTAGATCGATGGGAAAAAATTCGGTTAAG
GCCAGGGGGAAAGAAGAAGTACAAGCTAAAGCACATCGTATGGGCAAGCAGGGAGCTAGAACGAT
TCGCAGTTAATCCTGGCCTGTTAGAAACATCAGAAGGCTGTAGACAAATACTGGGACAGCTACAA
CCATCCCTTCAGACAGGATCAGAGGAGCTTCGATCACTATACAACACAGTAGCAACCCTCTATTG
TGTGCACCAGCGGATCGAGATCAAGGACACCAAGGAAGCTTTAGACAAGATAGAGGAAGAGCAAA
ACAAGTCCAAGAAGAAGGCCCAGCAGGCAGCAGCTGACACAGGACACAGCAATCAGGTCAGCCAA
AATTACCCTATAGTGCAGAACATCCAGGGGCAAATGGTACATCAGGCCATATCACCTAGAACTTT
AAATGCATGGGTAAAAGTAGTAGAAGAGAAGGCTTTCAGCCCAGAAGTGATACCCATGTTTTCAG
CATTATCAGAAGGAGCCACCCCACAGGACCTGAACACGATGTTGAACACCGTGGGGGGACATCAA
GCAGCCATGCAAATGTTAAAAGAGACCATCAATGAGGAAGCTGCAGAATGGGATAGAGTGCATCC
AGTGCATGCAGGGCCTATTGCACCAGGCCAGATGAGAGAACCAAGGGGAAGTGACATAGCAGGAA
CTACTAGTACCCTTCAGGAACAAATAGGATGGATGACAAATAATCCACCTATCCCAGTAGGAGAG
ATCTACAAGAGGTGGATAATCCTGGGATTGAACAAGATCGTGAGGATGTATAGCCCTACCAGCAT
TCTGGACATAAGAcALAGGACCAAAGGAACCCTTTAGAGACTATGTAGACCGGTTCTATAAAACT
CTAAGAGCTGAGCAAGCTTCACAGGAGGTAAAAATTGGATGACAGAAACCTTGTTGGTCCAAAAA
ATGCGAACCCAGATTGTAAGACCATCCTGAAGGCTCTCGGCCCAGCGGCTACACTAGAAGAAATG
ATGACAGCATGTCAGGGAGTAGGAGGACCCGGCCATAAAGGCAAAGAGTTTTGGCCGAGGCGATG
AGCCAGGTGACGAACTCGGCGACCATAATGATGCAGAGAGGCAACTTCCGGAACCAGCGGAAGAT
CGTCAAGTGCTTCAATTGTGGCAAAGAAGGGCACACCGCCAGGAACTGCCGGGCCCCCCGGAAGA
AGGGCTGCTGGAAGTGCGGGAAGGAGGGGCACCAGATGAAGGACTGCACGGAGCGGCAGGCGAAC
TTCCTGGGGAAGATATGGCCGAGTTACAAGGGAAGACCCGACCGGCAGGGGACGGTGTCGTTCAA
CTTCCCTCAGATCACGCTCTGGCAGCGGCCGCTCGTCACATAAAGATCGGGGGGCAACTCAAGGA
GGCGCTGCTCGCGGACGACACGGTCTTGGAGGAGATGTCGTTGCCGGGGCGGTGGAAGCCGAAGA
TGATCGGGGGGATCGGGGGCTTCATCAAGGTGCGGCAGTACGACCAGATCCTCATCGAGATCTGC
GGGCACAAGGCGATCGGGACGGTCCTCGTCGGCCCGACGCCGGTCAACATCATCGGGCGGAACCT
GTTGACCCAGATCGGCTGCACCTTGAACTTCCCCATCAGCCCTATTGAGACGGTGCCCGTGAAGT
TGAAGCCGGGGATGGACGGCCCCAAGGTCAAAGCAATGGCCATTGACGGAGGAGAAGATCAAGGC
CTTAGTCGAAATCTGTACAGAGATGGAGAAGGAAGGGAAGATCAGCAAGATCGGGCCTGAGAACC
CCTACAACACTCCAGTCTTCGCAATCAAGAAGAAGGACAGTACCAAGTGGAGAAAGCTGGTGGAC
TTCAGAGAGCTGAACAAGAGAACTCAGGACTTCTGGGAAGTTCAGCTGGGCATCCCACATCCCGC
TGGGTTGAAGAAGAAGAAGTCAGTGACAGTGCTGGATGTGGGTGATGCCTACTTCTCCGTTCCCT
TGGACGAGGACTTCAGGAAGTACACTGCCTTCACGATACCTAGCATCAACAACGAGACACCAGGC
ATCCGCTACCAGTACAACGTGCTGCCACAGGGATGGAAGGGATCACCAGCCATCTTTCAATCGTC
GATGACCAAGATCCTGGAGCCCTTCCGCAAGCAAAACCCAGACATCGTGATCTATCAGCTCTACG
TAGGAAGTGACCTGGAGATCGGGCAGCACAGGACCAAGATCGAGGAGCTGAGACAGCATCTGTTG
AGGTGGGGACTGACCACACCAGACAAGAAAGCACCAGAAGGACCTCCCTTCCTGTGGATGGGCTA
CGAACTGCATCCTGACAAGTGGACAGTGCAGCCCATCGTGCTGCCTGAGAAGGACAGCTGGACTG
TGAACGACATACAGAAGCTCGTGGGCAAGTTGAACTGGGCAAGCCAGATCTACCCAGGCATCAAA
GTTAGGCAGCTGTGCAAGCTGCTTCGAGGAACCAAGGCACTGACAGAAGTGATCCCACTGACAGA
GGAAGCAGAGCTAGAACTGGCAGAGAACCGAGAGATCCTGAAGGAGCCAGTACATGGAGTGTACT
ACGACCCAAGCAAGGACCTGATCGCAGAGATCCAGAAGCAGGGGCAAGGCCATGGACCTACCAAA
TCTACCAGGAGCCCTTCAAGAACCTGAAGACAGGCAAGTACGCAAGGATGAGGGGTGCCCACACC
AACGATGTGAAGCAGCTGACAGAGGCAGTGCAGAAGATCACCACAGAGAGCATCGTGATCTGGGG
CAAGACTCCCAAGTTCAAGCTGCCCATACAGAAGGAGACATGGGAGACATGGTGGACCGAGTACT
GGCAAGCCACCTGGATCCCTGAGTGGGAGTTCGTGAACACCCCTCCCTTGGTGAAACTGTGGTAT
CAGCTGGAGAAGGAACCCATCGTGGGAGCAGAGACCTTCTACGTGGATGGGGCAGCCAACAGGGA
GACCAAGCTGGGCAAGGCAGGCTACGTGACCAACCGAGGACGACAGAAAGTGGTGACCCTGACTG
ACACCACCAACCAGAAGACTCTGCAAGCCATCTACCTAGCTCTGCAAGACAGCGGACTGGAAGTG
AACATCGTGACAGACTCACAGTACGCACTGGGCATCATCCAAGCACAACCAGACCAATCCGAGTC
AGAGCTGGTGAACCAGATCATCGAGCAGCTGATCAAGAAGGAGAAAGTGTACCTGGCATGGGTCC
CGGCGCACAAGGGGATCGGGGGGAACGAGCAGGTCGACAAGTTGGTCTCGGCGGGGATCCGGAAG
GTGCTGTTCCTGGACGGGATCGATAAGGCCCAAGATGAACATGAGAAGTACCACTCCAACTGGCG
CGCTATGGCCAGCGACTTCAACCTGCCGCCGGTCGTCGCGAAGGAGATCGTCGCCAGCTGCGACA
AGTGCCAGCTCAAGGGGGAGGCCATGCACGGGCAAGTCGACTGCAGTCCGGGGATCTGGCAGCTG
TGCACGCACCTGGAGGGGAAGGTGATCCTGGTCGCGGTCCACGTCGCCAGCGGGTATATCGAGGC
GGAGGTCATCCCGGCTGAGACGGGGCAGGAGACGGCGTACTTCCTCTTGAAGCTCGCGGGGCGGT
GGCCGGTCAAGACGATCCACACGAACGGGAGCAACTTCACGGGGGCGACGGTCAAGGCCGCCTGT
TGGTGGGCGGGAATCAAGCAGGAATTTGGAATTCCCTACAATCCCCAATCGCAAGGAGTCGTGAG
CATGAACAAGGAGCTGAAGAAGATCATCGGACAAAGGGATCAGGCTGAGCACCTGAAGACAGCAG
TGCAGATGGCAGTGTTCATCCACAACTTCAAAAGAAAAGGGGGGATTGGGGGGTACAGTGCGGGG
GAACGGATCGTGGACATCATCGCCACCGACATCCAAACCAAGGAGCTGCAGAAGCAGATCACCAA
GATCCAGAACTTCCGGGTGTACTACCGCGACAGCCGCAACCCACTGTGGAAGGGACCAGCAAAGC
TCCTCTGGAAGGGAGAGGGGGCAGTGGTGATCCAGGACAACAGTGACATCAAAGTGGTGCCAAGG
CGCAAGGCCAAGATCATCCGCGACTATGGAAAACAGATGGCAGGGGATGATTGTGTGGCAAGTAG
ACAGGATGAGGATGGCGCCTAG
Protein:
M G A R A S V L S G G E L D R W E K I R L R P G G K K K Y K L K H
I V W A S R E L E R F A V N P G L L E T S E G C R Q I L G Q L Q P
S L Q T G S E E L R S L Y N T V A T L Y C V H Q R I E I K D T K E
A L D K I E E E Q N K S K K K A Q Q A A A D T G H S N Q V S Q N Y
P I V Q N I Q G Q M V H Q A I S P R T L N A W V K V V E E K A F S
P E V I P M F S A L S E G A T P Q D L N T M L N T V G G H Q A A M
Q M L K E T I N E E A A E W D R V H P V H A G P I A P G Q M R E P
R G S D I A G T T S T L Q E Q I G W M T N N P P I P V G E I Y K R
W I I L G L N K I V R M Y S P T S I L D I R Q G P K E P F R D Y V
D R F Y K T L R A E Q A S Q E V K N W M T E T L L V Q N A N P D C
K T I L K A L G P A A T L E E M M T A C Q G V G G P G H K A R V L
A E A M S Q V T N S A T I M M Q R G N F R N Q R K I V K C F N C G
K E G H T A R N C R A P R K K G C W K C G K E G H Q M K D C T E R
Q A N F L G K I W P S Y K G R P D R Q G T V S F N F P Q I T L W Q
R P L V T I K I G G Q L K E A L L A D D T V L E E M S L P G R W K
P K M I G G I G G F I K V R Q Y D Q I L I E I C G H K A I G T V L
V G P T P V N I I G R N L L T Q I G C T L N F P I S P I E T V P V
K L K P G M D G P K V K Q W P L T E E K I K A L V E I C T E M E K
E G K J S K I G P E N P Y N T P V F A I K K K D S T K W R K L V D
F R E L N K R T Q D F W E V Q L G I P H P A G L K K K K S V T V L
D V G D A Y F S V P L D E D F R K Y T A F T I P S I N N E T P G I
R Y Q Y N V L P Q G W K G S P A I F Q S S M T K I L E P F R K Q N
P D I V I Y Q L Y V G S D L E I G Q H R T K I E E L R Q H L L R W
G L T T P D K K H Q K E P P F L W M G Y E L H P D K W T V Q P I V
L P E K D S W T V N D I Q K L V G K L N W A S Q I Y P G I K V R Q
L C K L L R G T K A L T E V I P L T E E A E L E L A E N R E I L K
E P V H G V Y Y D P S K D L I A E I Q K Q G Q G Q W T Y Q I Y Q E
P F K N L K T G K Y A R M R G A H T N D V K Q L T E A V Q K I T T
E S I V I W G K T P K F K L P I Q K E T W E T W W T E Y W Q A T W
I P E W E F V N T P P L V K L W Y Q L E K E P I V G A E T F Y V D
G A A N R E T K L G K A G Y V T N R G R Q K V V T L T D T T N Q K
T L Q A I Y L A L Q D S G L E V N I V T D S Q Y A L G I I Q A Q P
D Q S E S E L V N Q I I E Q L I K K E K V Y L A W V P A H K G I G
G N E Q V D K L V S A G I R K V L F L D G I D K A Q D E H E K Y H
S N W R A M A S D F N L P P V V A K E I V A S C D K C Q L K G E A
M H G Q V D C S P G I W Q L C T H L E G K V I L V A V H V A S G Y
I E A E V I P A E T G Q E T A Y F L L K L A G R W P V K T I H T N
G S N F T G A T V K A A C W W A G I K Q E F G I P Y N P Q S Q G V
V S M N K E L K K I I G Q R D Q A E H L K T A V Q M A V F I H N F
K R K G G I G G Y S A G E R I V D I I A T D I Q T K E L Q K Q I T
K I Q N F R V Y Y R D S R N P L W K G P A K L L W K G E G A V V I
Q D N S D I K V V P R R K A K I I R D Y G K Q M A G D D C V A S R
Q D E D G A •
CATEp37gag(HIV)
ATGAGAAAAGCGGCTGTTAGTCACTGGCAGCAACAGTCTTACCTGGACTCTGGAATCCATTCTGG
TGCCACTACCACAGCTCCTTCTCTGAGTGTCGACAGAGAGATGGGTGCGAGAGCGTCAGTATTAA
GCGGGGGAGAATTAGATCGATGGGAAAAAATTCGGTTAAGGCCAGGGGGAAAGAAGAAGTACAAG
CTAAAGCACATCGTATGGGCAAGCAGGGAGCTAGAACGATTCGCAGTTAATCCTGGCCTGTTAGA
AACATCAGAAGGCTGTAGACAAATACTGGGACAGCTACAACCATCCCTTCAGACAGGATCAGAGG
AGCTTCGATCACTATACAACACAGTAGCAACCCTCTATTGTGTGCACCAGCGGATCGAGATCAAG
GACACCAAGGAAGCTTTAGACAAGATAGAGGAAGAGCAAAACAAGTCCAAGAAGAAGGCCCAGCA
GGCAGCAGCTGACACAGGACACAGCAATCAGGTCAGCCAAAATTACCCTATAGTGCAGAACATCC
AGGGGCAAATGGTACATCAGGCCATATCACCTAGAACTTTAAATGCATGGGTAAAAGTAGTAGAA
GAGAAGGCTTTCAGCCCAGAAGTGATACCCATGTTTTCAGCATTATCAGAAGGAGCCACCCCACA
GGACCTGAACACGATGTTGAACACCGTGGGGGGACATCAAGCAGCCATGCAAATGTTAAAAGAGA
CCATCAATGAGGAAGCTGCAGAATGGGATAGAGTGCATCCAGTGCATGCAGGGCCTATTGCACCA
GGCCAGATGAGAGAACCAAGGGGAAGTGACATAGCAGGAACTACTAGTACCCTTCAGGAACAAAT
AGGATGGATGACAAATAATCCACCTATCCCAGTAGGAGAGATCTACAAGAGGTGGATAATCCTGG
GATTGAACAAGATCGTGAGGATGTATAGCCCTACCAGCATTCTGGACATAAGACAAGGACCAAAG
GAACCCTTTAGAGACTATGTAGACCGGTTCTATAAAACTCTAAGAGCTGAGCAAGCTTCACAGGA
GGTAAAAAATTGGATGACAGAAACCTTGTTGGTCCAAAATGCGAACCCAGATTGTAAGACCATCC
TGAAGGCTCTCGGCCCAGCGGCTACACTAGAAGAAATGATGACAGCATGTCAGGGAGTAGGAGGA
CCCGGCCATAAGGCAAGAGTTTTGTAG
protein:
M R K A A V S H W Q Q Q S Y L D S G I H S G A T T T A P S L S V D
R E M G A R A S V L S G G E L D R W E K I R L R P G G K K K Y K L
K H L V W A S R E L E R F A V N P G L L E T S E G C R Q I L G Q L
Q P S L Q T G S E E L R S L Y N T V A T L Y C V H Q R I E I K D T
K E A L D K I E E E Q N K S K K K A Q Q A A A D T G H S N Q V S Q
N Y P I V Q N I Q G Q M V H Q A I S P R T L N A W V K V V E E K A
F S P E V I P M F S A L S E G A T P Q D L N T M L N T V G G H Q A
A M Q M L K E T I N E E A A E W D R V H P V H A G P I A P G Q M R
E P R G S D I A G T T S T L Q E Q I G W M T N N P P I P V G E I Y
K R W I I L G L N K I V R M Y S P T S I L D I R Q G P K E P F R D
Y V D R F Y K T L R A E Q A S Q E V K N W M T E T L L V Q N A N P
D C K T I L K A L G P A A T L E E M M T A C Q G V G G P G H K A R
V L •

The above examples are provided to illustrate the invention but not to limit its scope. Other variants of the invention will be readily apparent to one of ordinary skill in the art and are encompassed by the appended claims.

All publications, patents, accession numbers, and patent applications cited herein are hereby incorporated by reference for all purposes.

What is claimed is:

1. A method of treating an individual infected with a retrovirus, the method comprising:
administering a DNA vaccine comprising an expression vector selected from the group consisting of a) an expression vector encoding a fusion protein comprising a degradation polypeptide linked to an immunogenic retrovirus polypeptide or b) an expression vector encoding a secreted fusion protein comprising a secretory polypeptide linked to an immunogenic retrovirus polypeptide; and
administering antiretroviral therapy (ART);
wherein administration of the DNA vaccine results in control of viremia upon cessation of ART.

2. The method of claim 1, wherein the DNA vaccine is administered to the individual while the individual is undergoing ART.

3. The method of claim 1, wherein the expression vector encodes a fusion protein comprising a degradation polypeptide linked to an immunogenic retrovirus polypeptide.

4. The method of claim 3, further comprising a step of administering an expression vector that encodes a fusion protein comprising a secretory polypeptide.

5. The method of claim 4, wherein the fusion protein comprising a secretory polypeptide has an immunogenic polypeptide that is different from the immunogenic polypeptide included in the fusion protein comprising a degradation polypeptide linked to an immunogenic polypeptide.

6. The method of claim 4, wherein the expression vector that encodes the fusion protein comprising the secretory polypeptide is concurrent with the expression vector encoding a fusion protein comprising a degradation polypeptide

7. The method of claim 1, wherein the degradation polypeptide is selected from the group consisting of c-Mos aa1-35, cyclin B aa 10-95, β-catenin aa 19-44, and β-catenin aa 18-47.

8. The method of claim 7, wherein the degradation polypeptide is a degradation signal from β-catenin.

9. The method of claim 8, wherein the degradation signal from β-catenin is linked to a human immunodeficienty (HIV) gag polypeptide.

10. The method of claim 8, wherein the degradation signal from β-catenin is linked to an HIV env polypeptide.

11. The method of claim 1, wherein the immunogenic retrovirus polypeptide is an HIV antigen.

12. The method of claim 11, wherein the HIV antigen is selected from the group consisting of Gag, Env, Pol, Nef, Vpr, Vpu, Vif, Tat, and Rev.

13. The method of claim 12, wherein the HIV antigen comprises linked epitopes of HIV Gag, Env, Tat, Rev, and Nef, said epitopes linked in any order; or linked epitopes of Gag, Env, Pol, Tat, and Nef, said epitopes linked in any order.

14. The method of claim 13, wherein the HIV antigen is linked to a β-catenin degradation signal.

15. The method of claim 12, wherein the HIV antigen is linked to a secretory polypeptide.

16. The method of claim 12, wherein the HIV antigen comprises linked epitopes of gag, env, rev, tat, nef and vif; or linked epitopes of gag, env, pol, nef, tat, and vif.

17. The method of claim 16, wherein the HIV antigen is linked to a β-catenin degradation signal.

18. The method of claim 1, further comprising administering a nucleic acid sequence encoding an adjuvant.

19. The method of claim 18, wherein the adjuvant is IL-12 or IL-15.

20. The method of claim 1, wherein the expression vector is administered by intramuscular injection.

21. The method of claim 1, further comprising at least a second administration of the expression plasmid.

22. The method of claim 1, wherein the secretory polypeptide is MCP-3.

23. The method of claim 22, wherein the MCP-3 is joined to an immunogenic retroviral polypeptide that is an HIV antigen.

24. The method of claim 23, wherein the HIV antigen is selected from the group consisting of Gag, Env, Pol, Nef, Vpr, Vpu, Vif, Tat, and Rev.

25. The method of claim 24, wherein the HIV polypeptide is from gag.

26. A method of treating an individual undergoing antiretroviral therapy, the method comprising:
administering to the individual a DNA vaccine comprising an expression vector selected from the group consisting of a) an expression vector encoding a fusion protein comprising a degradation polypeptide linked to an immunogenic retrovirus polypeptide and b) an expression vector encoding a secreted fusion protein comprising a secretory polypeptide linked to an immunogenic retrovirus polypeptide; wherein administration of the DNA vaccine results in control of viremia upon cessation of ART.

27. The method of claim 26, wherein the immunogenic retrovirus polypeptide is an HIV antigen.

28. The method of claim 26, wherein the degradation polypeptide is selected from the group consisting of c-Mos aa1-35, cyclin B aa 10-95, β-catenin aa 19-44, and β-catenin aa 18-47.

29. The method of claim 26, wherein the secretory polypeptide is MCP-3 or the tissue plasminogen activator (tPA) signal peptide.

30. The method of claim 26, wherein the degradation polypeptide is β-catenin 18-47 and the secretory polypeptide is MCP-3 or the tPA signal peptide.

31. The method of claim 30, wherein the degradation polypeptide fusion protein comprises HIV Gag and HIV Pol.


SEQUENCE LISTING (95 sequences)

SEQ ID NO: 1 - protein, 191 aa, Human immunodeficiency virus type 1
  modified Vif (82H, 83H) positions 1-191
ENRWQVMIVWQVDRMRIRTWKSLVKHHMYISGKAKGWFYRHHYESTHPRISSEVHIPLGDAKLVITTYWGLHTGERDWHLGQGVSIEWRKKRYSTQVDPDLADQLIHLYYFDSFSESAIRNTILGRIVSPRSEYQAGHNKVGSLQYLALAALITPKQIKPPLPSVTKLTEDRWNKPQKTKGHRGSHTMNGH

SEQ ID NO: 2 - protein, 191 aa, Human immunodeficiency virus type 1
  NL43 vif positions 2-192
ENRWQVMIVWQVDRMRINTWKRLVKHHMYISRKAKDWFYRHHYESTNPKISSEVHIPLGDAKLVITTYWGLHTGERDWHLGQGVSIEWRKKRYSTQVDPDLADQLIHLHYFDCFSESAIRNTILGRIVSPRCEYQAGHNKVGSLQYLALAALIKPKQIKPPLPSVRKLTEDRWNKPQKTKGHRGSHTMNGH

SEQ ID NO: 3 - protein, 17 aa, Artificial Sequence
  Description of Artificial SequenceVif consensus sequence
ENRWQVMIVWQVDRMRI

SEQ ID NO: 4 - protein, 9 aa, Artificial Sequence
  Description of Artificial SequenceVif consensus sequence
LVKHHMYIS

SEQ ID NO: 5 - protein, 10 aa, Artificial Sequence
  Description of Artificial SequenceVif consensus sequence
WFYRHHYEST

SEQ ID NO: 6 - protein, 59 aa, Artificial Sequence
  Description of Artificial SequenceVif consensus sequence
ISSEVHIPLGDAKLVITTYWGLHTGERDWHLGQGVSIEWRKKRYSTQVDPDLADQLIHL

SEQ ID NO: 7 - protein, 18 aa, Artificial Sequence
  Description of Artificial SequenceVif consensus sequence
FSESAIRNTILGRIVSPR

SEQ ID NO: 8 - protein, 21 aa, Artificial Sequence
  Description of Artificial SequenceVif consensus sequence
EYQAGHNKVGSLQYLALAALI

SEQ ID NO: 9 - protein, 11 aa, Artificial Sequence
  Description of Artificial SequenceVif consensus sequence
PKQIKPPLPSV

SEQ ID NO: 10 - protein, 25 aa, Artificial Sequence
  Description of Artificial SequenceVif consensus sequence
KLTEDRWNKPQKTKGHRGSHTMNGH

SEQ ID NO: 11 - protein, 83 aa, Human immunodeficiency virus type 1
  modified Tat positions 1656-1738
EPVDPRLEPWKHPGSQPKTACTNCYCKKCFHQVCFMTKALGISYGRKKRRQRRRAHQNSQTHQASLSKQPTSQSRGDPTGPKE

SEQ ID NO: 12 - protein, 85 aa, Human immunodeficiency virus type 1
  NL43 tat wildtype positions 2-86
EPVDPRLEPWKHPGSQPKTACTNCYCKKCCFHCQVCFMTKALGISYGRKKRRQRRRAHQNSQTHQASLSKQPTSQSRGDPTGPKE

SEQ ID NO: 13 - protein, 29 aa, Artificial Sequence
  Description of Artificial SequenceTat consensus sequence
EPVDPRLEPWKHPGSQPKTACTNCYCKKC

SEQ ID NO: 14 - protein, 52 aa, Artificial Sequence
  Description of Artificial SequenceTat consensus sequence
QVCFMTKALGISYGRKKRRQRRRAHQNSQTHQASLSKQPTSQSRGDPTGPKE

SEQ ID NO: 15 - protein, 205 aa, Human immunodeficiency virus type 1
  modified nef positions 1449-1628
GAKWSKSSVIGWPTVRERMRRAEPAADRVGAASRDLEKHGAITSSNTAATNAACAWLEAQEEEEVGFPVTPQVPLRPMTYKAAVDLSHFLKEKGGLEGLIHSQRRQDILDLWIYHTQGYFPDWQNYTPGPGVRYPLTFGWCYKLVPVEPDKIEEANKGENTSLLHPVSLHGMDDPEREVLEWRFDSRLAFHHVARELHPEYFKNC

SEQ ID NO: 16 - protein, 205 aa, Human immunodeficiency virus type 1
  NL43 nef WT positions 2-206
GGKWSKSSVIGWPAVRERMRRAEPAADGVGAVSRDLEKHGAITSSNTAANNAACAWLEAQEEEEVGFPVTPQVPLRPMTYKAAVDLSHFLKEKGGLEGLIHSQRRQDILDLWIYHTQGYFPDWQNYTPGPGVRYPLTFGWCYKLVPVEPDKVEEANKGENTSLLHPVSLHGMDDPEREVLEWRFDSRLAFHHVARELHPEYFKNC

SEQ ID NO: 17 - protein, 11 aa, Artificial Sequence
  Description of Artificial SequenceNef consensus sequence
KWSKSSVIGWP

SEQ ID NO: 18 - protein, 13 aa, Artificial Sequence
  Description of Artificial SequenceNef consensus sequence
VRERMRRAEPAAD

SEQ ID NO: 19 - protein, 17 aa, Artificial Sequence
  Description of Artificial SequenceNef consensus sequence
SRDLEKHGAITSSNTAA

SEQ ID NO: 20 - protein, 101 aa, Artificial Sequence
  Description of Artificial SequenceNef consensus sequence
NAACAWLEAQEEEEVGFPVTPQVPLRPMTYKAAVDLSHFLKEKGGLEGLIHSQRRQDILDLWIYHTQGYFPDWQNYTPGPGVRYPLTFGWCYKLVPVEPDK

SEQ ID NO: 21 - protein, 53 aa, Artificial Sequence
  Description of Artificial SequenceNef consensus sequence
EEANKGENTSLLHPVSLHGMDDPEREVLEWRFDSRLAFHHVARELHPEYFKNC

SEQ ID NO: 22 - protein, 954 aa, Human immunodeficiency virus type 1
  modified pol positions 495-1448
NDPSSQAGADRQGTVSFNFPQITLWQRPLVTVRIGGQLKEALLADDTVLEEMSLPGRWKPKMIGGIGGFIKVRQYDQILIEICGHKAIGTVLVGPTPVNIIGRNLLTQIGCTLNFPISPIETVPVKLKPGMDGPKVKQWPLTKEKIKALVEICTEMEKEGKISKIGPENPYNTPVFAIKKKDSTKWRKLVDFRELNKRTQDFWEVQLGIPHPAGLKKKKSVTVLDVGDAYFSVPLDEDFRKYTAFTIPSINNETPGIRYQYNVLPQGWKGSPAIFQSSMTKILEPFRKQNPDIVIYQLYVGSDLEIGQHRTKIEELRQHLLRWGLTTPDKKHQKEPPFLWMGYELHPDKWTVQPIVLPEKDSWTVNDIQKLVGKLNWASQIYPGIKVRQLCKLLRGTKALTEVIPLTEEAELELAENREILKEPVHGVYYDPSKDLIAEIQKQGQGQWTYQIYQEPFKNLKTGKYARMRGAHTNDVKQLTEAVQKITTESIVIWGKTPKFKLPIQKETWETWWTEYWQATWIPEWEFVNTPPLVKLWYQLEKEPIVGAETFYVDGAANRETKLGKAGYVTNRGRQKVVTLTDTTNQKTLQAIYLALQDSGLEVNIVTDSQYALGIIQAQPDQSESELVNQIIEQLIKKEKVYLAWVPAHKGIGGNEQVDKLVSAGIRKVLFLDGIDKAQDEHEKYHSNWRAMASDFNLPPVVAKEIVASCDKCQLKGEAMHGQVDCSPGIWQLCTHLEGKVILVAVHVASGYIEAEVIPAETGQETAYFLLKLAGRWPVKTIHTNGSNFTGATVKAACWWAGIKQEFGIPYNPQSQGVVSMNKELKKIIGQRDQAEHLKTAVQMAVFIHNFKRKGGIGGYSAGERIVDIIATDIQTKELQKQITKIQNFRVYYRDSRNPLWKGPAKLLWKGEGAVVIQDNSDIKVVPRRKAKIIRDYGKQMAGDDCVASRQDED

SEQ ID NO: 23 - protein, 966 aa, Human immunodeficiency virus type 1
  HXB2 pol positions 38-1003
NNSPSEAGADRQGTVSFNFPQVTLWQRPLVTIKIGGQLKEALLDTGADDTVLEEMSLPGRWKPKMIGGIGGFIKVRQYDQILIEICGHKAIGTVLVGPTPVNIIGRNLLTQIGCTLNFPISPIETVPVKLKPGMDGPKVKQWPLTEEKIKALVEICTEMEKEGKISKIGPENPYNTPVFAIKKKDSTKWRKLVDFRELNKRTQDFWEVQLGIPHPAGLKKKKSVTVLDVGDAYFSVPLDEDFRKYTAFTIPSINNETPGIRYQYNVLPQGWKGSPAIFQSSMTKILEPFRKQNPDIVIYQYMDDLYVGSDLEIGQHRTKIEELRQHLLRWGLTTPDKKHQKEPPFLWMGYELHPDKWTVQPIVLPEKDSWTVNDIQKLVGKLNWASQIYPGIKVRQLCKLLRGTKALTEVIPLTEEAELELAENREILKEPVHGVYYDPSKDLIAEIQKQGQGQWTYQIYQEPFKNLKTGKYARMRGAHTNDVKQLTEAVQKITTESIVIWGKTPKFKLPIQKETWETWWTEYWQATWIPEWEFVNTPPLVKLWYQLEKEPIVGAETFYVDGAANRETKLGKAGYVTNRGRQKVVTLTDTTNQKTELQAIYLALQDSGLEVNIVTDSQYALGIIQAQPDQSESELVNQIIEQLIKKEKVYLAWVPAHKGIGGNEQVDKLVSAGIRKVLFLDGIDKAQDEHEKYHSNWRAMASDFNLPPVVAKEIVASCDKCQLKGEAMHGQVDCSPGIWQLDCTHLEGKVILVAVHVASGYIEAEVIPAETGQETAYFLLKLAGRWPVKTIHTDNGSNFTGATVRAACWWAGIKQEFGIPYNPQSQGVVESMNKELKKIIGQVRDQAEHLKTAVQMAVFIHNFKRKGGIGGYSAGERIVDIIATDIQTKELQKQITKIQNFRVYYRDSRNPLWKGPAKLLWKGEGAVVIQDNSDIKVVPRRKAKIIRDYGKQMAGDDCVASRQDED

SEQ ID NO: 24 - protein, 15 aa, Artificial Sequence
  Description of Artificial SequencePol consensus sequence
AGADRQGTVSFNFPQ

SEQ ID NO: 25 - protein, 9 aa, Artificial Sequence
  Description of Artificial SequencePol consensus sequence
TLWQRPLVT

SEQ ID NO: 26 - protein, 10 aa, Artificial Sequence
  Description of Artificial SequencePol consensus sequence
IGGQLKEALL

SEQ ID NO: 27 - protein, 99 aa, Artificial Sequence
  Description of Artificial SequencePol consensus sequence
ADDTVLEEMSLPGRWKPKMIGGIGGFIKVRQYDQILIEICGHKAIGTVLVGPTPVNIIGRNLLTQIGCTLNFPISPIETVPVKLKPGMDGPKVKQWPLT

SEQ ID NO: 28 - protein, 154 aa, Artificial Sequence
  Description of Artificial SequencePol consensus sequence
EKIKALVEICTEMEKEGKISKIGPENPYNTPVFAIKKKDSTKWRKLVDFRELNKRTQDFWEVQLGIPHPAGLKKKKSVTVLDVGDAYFSVPLDEDFRKYTAFTIPSINNETPGIRYQYNVLPQGWKGSPAIFQSSMTKILEPFRKQNPDIVIYQ

SEQ ID NO: 29 - protein, 291 aa, Artificial Sequence
  Description of Artificial SequencePol consensus sequence
LYVGSDLEIGQHRTKIEELRQHLLRWGLTTPDKKHQKEPPFLWMGYELHPDKWTVQPIVLPEKDSWTVNDIQKLVGKLNWASQIYPGIKVRQLCKLLRGTKALTEVIPLTEEAELELAENREILKEPVHGVYYDPSKDLIAEIQKQGQGQWTYQIYQEPFKNLKTGKYARMRGAHTNDVKQLTEAVQKITTESIVIWGKTPKFKLPIQKETWETWWTEYWQATWIPEWEFVNTPPLVKLWYQLEKEPIVGAETFYVDGAANRETKLGKAGYVTNRGRQKVVTLTDTTNQKT

SEQ ID NO: 30 - protein, 145 aa, Artificial Sequence
  Description of Artificial SequencePol consensus sequence
LQAIYLALQDSGLEVNIVTDSQYALGIIQAQPDQSESELVNQIIEQLIKKEKVYLAWVPAHKGIGGNEQVDKLVSAGIRKVLFLDGIDKAQDEHEKYHSNWRAMASDFNLPPVVAKEIVASCDKCQLKGEAMHGQVDCSPGIWQL

SEQ ID NO: 31 - protein, 51 aa, Artificial Sequence
  Description of Artificial SequencePol consensus sequence
CTHLEGKVILVAVHVASGYIEAEVIPAETGQETAYFLLKLAGRWPVKTIHT

SEQ ID NO: 32 - protein, 10 aa, Artificial Sequence
  Description of Artificial SequencePol consensus sequence
NGSNFTGATV

SEQ ID NO: 33 - protein, 24 aa, Artificial Sequence
  Description of Artificial SequencePol consensus sequence
AACWWAGIKQEFGIPYNPQSQGVV

SEQ ID NO: 34 - protein, 12 aa, Artificial Sequence
  Description of Artificial SequencePol consensus sequence
SMNKELKKIIGQ

SEQ ID NO: 35 - protein, 123 aa, Artificial Sequence
  Description of Artificial SequencePol consensus sequence
RDQAEHLKTAVQMAVFIHNFKRKGGIGGYSAGERIVDIIATDIQTKELQKQITKIQNFRVYYRDSRNPLWKGPAKLLWKGEGAVVIQDNSDIKVVPRRKAKIIRDYGKQMAGDDCVASRQDED

SEQ ID NO: 36 - RNA, 42 nt, Artificial Sequence
  Description of Artificial Sequencewildtype HIV-1 gag/pol
uuuuuuagggaagaucuggccuuccuacaagggaaggccagg

SEQ ID NO: 37 - RNA, 42 nt, Artificial Sequence
  Description of Artificial Sequenceexemplary HIV-1 Gag/pol in-frame with mutations in frameshifting signal (FS) and pseudoknot (P)
guuccuagggaagauguggccgaguuacaagggaagacccga

SEQ ID NO: 38 - protein, 964 aa, Simian immunodeficiency virus
  wildtype (wt) SIV pol
REALQGGDRGFAAPQFSLWRRPVVTAHIEGQPVEVLLDTGADDSIVTGIELGPHYTPKIVGGIGGFINTKEYKNVEIEVLGKRIKGTIMTGDTPINIFGRNLLTALGMSLNFPIAKVEPVKVALKPGKDGPKLKQWPLSKEKIVALREICEKMEKDGQLEEAPPTNPYNTPTFAIKKKDKNKWRMLIDFRELNRVTQDFTEVQLGIPHPAGLAKRKRITVLDIGDAYFSIPLDEEFRQYTAFTLPSVNNAEPGKRYIYKVLPQGWKGSPAIFQYTMRHVLEPFRKANPDVTLVQYMDDILIASDRTDLEHDRVVLQSKELLNSIGFSTPEEKFQKDPPFQWMGYELWPTKWKLQKIELPQRETWTVNDIQKLVGVLNWAAQIYPGIKTKHLCRLIRGKMTLTEEVQWTEMAEAEYEENKIILSQEQEGCYYQEGKPLEATVIKSQDNQWSYKIHQEDKILKVGKFAKIKNTHTNGVRLLAHVIQKIGKEAIVIWGQVPKFHLPVEKDVWEQWWTDYWQVTWIPEWDFISTPPLVRLVFNLVKDPIEGEETYYTDGSCNKQSKEGKAGYITDRGKDKVKVLEQTTNQQAELEAFLMALTDSGPKANIIVDSQYVMGIITGCPTESESRLVNQIIEEMIKKSEIYVAWVPAHKGIGGNQEIDHLVSQGIRQVLFLEKIEPAQEEHDKYHSNVKELVFKFGLPRIVARQIVDTCDKCHQKGEAIHGQANSDLGTWQMDCTHLEGKIIIVAVHVASGFIEAEVIPQETGRQTALFLLKLAGRWPITHLHTDNGANFASQEVKMVAWWAGIEHTFGVPYNPQSQGVVEAMNHHLKNQIDRIREQANSVETIVLMAVHCMNFKRRGGIGDMTPAERLINMITTEQEIQFQQSKNSKFKNFRVYYREGRDQLWKGPGELLWKGEGAVILKVGTDIKVVPRRKAKIIKDYGGGKEVDSSSHMEDTGEAREVA

SEQ ID NO: 39 - protein, 964 aa, Simian immunodeficiency virus
  modified SIV pol
REALQGGDRGFAAPQFSLWRRPVVTAHIEGQPVEVLLAAAADDSIVTGIELGPHYTPKIVGGIGGFINTKEYKNVEIEVLGKRIKGTIMTGDTPINIFGRNLLTALGMSLNFPIAKVEPVKVALKPGKDGPKLKQWPLSKEKIVALREICEKMEKDGQLEEAPPTNPYNTPTFAIKKKDKNKWRMLIDFRELNRVTQDFTEVQLGIPHPAGLAKRKRITVLDIGDAYFSIPLDEEFRQYTAFTLPSVNNAEPGKRYIYKVLPQGWKGSPAIFQYTMRHVLEPFRKANPDVTLVQYMAAILIASDRTDLEHDRVVLQSKELLNSIGFSTPEEKFQKDPPFQWMGYELWPTKWKLQKIELPQRETWTVNDIQKLVGVLNWAAQIYPGIKTKHLCRLIRGKMTLTEEVQWTEMAEAEYEENKIILSQEQEGCYYQEGKPLEATVIKSQDNQWSYKIHQEDKILKVGKFAKIKNTHTNGVRLLAHVIQKIGKEAIVIWGQVPKFHLPVEKDVWEQWWTDYWQVTWIPEWDFISTPPLVRLVFNLVKDPIEGEETYYTDGSCNKQSKEGKAGYITDRGKDKVKVLEQTTNQQAALEAFLMALTDSGPKANIIVDSQYVMGIITGCPTESESRLVNQIIEEMIKKSEIYVAWVPAHKGIGGNQEIDHLVSQGIRQVLFLEKIEPAQEEHDKYHSNVKELVFKFGLPRIVARQIVDTCDKCHQKGEAIHGQANSDLGTWQMACTHLEGKIIIVAVHVASGFIEAEVIPQETGRQTALFLLKLAGRWPITHLHTANGANFASQEVKMVAWWAGIEHTFGVPYNPQSQGVVAAMNHHLKNQIDRIREQANSVETIVLMAVHCMNFKRRGGIGDMTPAERLINMITTEQEIQFQQSKNSKFKNFRVYYREGRDQLWKGPGELLWKGEGAVILKVGTDIKVVPRRKAKIIKDYGGGKEVDSSSHMEDTGEAREVA

SEQ ID NO: 40 - protein, 37 aa, Artificial Sequence
  Description of Artificial SequenceSIV pol consensus sequence
REALQGGDRGFAAPQFSLWRRPVVTAHIEGQPVEVLL

SEQ ID NO: 41 - protein, 256 aa, Artificial Sequence
  Description of Artificial SequenceSIV pol consensus sequence
ADDSIVTGIELGPHYTPKIVGGIGGFINTKEYKNVEIEVLGKRIKGTIMTGDTPINIFGRNLLTALGMSLNFPIAKVEPVKVALKPGKDGPKLKQWPLSKEKIVALREICEKMEKDGQLEEAPPTNPYNTPTFAIKKKDKNKWRMLIDFRELNRVTQDFTEVQLGIPHPAGLAKRKRITVLDIGDAYFSIPLDEEFRQYTAFTLPSVNNAEPGKRYIYKVLPQGWKGSPAIFQYTMRHVLEPFRKANPDVTLVQYM

SEQ ID NO: 42 - protein, 290 aa, Artificial Sequence
  Description of Artificial SequenceSIV pol consensus sequence
ILIASDRTDLEHDRVVLQSKELLNSIGFSTPEEKFQKDPPFQWMGYELWPTKWKLQKIELPQRETWTVNDIQKLVGVLNWAAQIYPGIKTKHLCRLIRGKMTLTEEVQWTEMAEAEYEENKIILSQEQEGCYYQEGKPLEATVIKSQDNQWSYKIHQEDKILKVGKFAKIKNTHTNGVRLLAHVIQKIGKEAIVIWGQVPKFHLPVEKDVWEQWWTDYWQVTWIPEWDFISTPPLVRLVFNLVKDPIEGEETYYTDGSCNKQSKEGKAGYITDRGKDKVKVLEQTTNQQA

SEQ ID NO: 43 - protein, 145 aa, Artificial Sequence
  Description of Artificial SequenceSIV pol consensus sequence
LEAFLMALTDSGPKANIIVDSQYVMGIITGCPTESESRLVNQIIEEMIKKSEIYVAWVPAHKGIGGNQEIDHLVSQGIRQVLFLEKIEPAQEEHDKYHSNVKELVFKFGLPRIVARQIVDTCDKCHQKGEAIHGQANSDLGTWQM

SEQ ID NO: 44 - protein, 51 aa, Artificial Sequence
  Description of Artificial SequenceSIV pol consensus sequence
CTHLEGKIIIVAVHVASGFIEAEVIPQETGRQTALFLLKLAGRWPITHLHT

SEQ ID NO: 45 - protein, 35 aa, Artificial Sequence
  Description of Artificial SequenceSIV pol consensus sequence
NGANFASQEVKMVAWWAGIEHTFGVPYNPQSQGVV

SEQ ID NO: 46 - protein, 141 aa, Artificial Sequence
  Description of Artificial SequenceSIV pol consensus sequence
AMNHHLKNQIDRIREQANSVETIVLMAVHCMNFKRRGGIGDMTPAERLINMITTEQEIQFQQSKNSKFKNFRVYYREGRDQLWKGPGELLWKGEGAVILKVGTDIKVVPRRKAKIIKDYGGGKEVDSSSHMEDTGEAREVA

SEQ ID NO: 47 - protein, 30 aa, Artificial Sequence
  Description of Artificial Sequencebeta-catenin destabilization sequence (amino acids 18-47)
RKAAVSHWQQQSYLDSGIHSGATTTAPSLS

SEQ ID NO: 48 - protein, 31 aa, Artificial Sequence
  Description of Artificial Sequencebeta-catenin destabilization sequence (amino acids 18-47) added at N-terminus of HIV antigens with initiator Met
MRKAAVSHWQQQSYLDSGIHSGATTTAPSLS

SEQ ID NO: 49 - DNA, 5558 bp, Artificial Sequence
  Description of Artificial Sequenceplasmid pSIVgagDX
cctggccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccgcgggcgcgcgtcgacagagagatgggcgtgagaaactccgtcttgtcagggaagaaagcagatgaattagaaaaaattaggctacgacccaacggaaagaaaaagtacatgttgaagcatgtagtatgggcagcaaatgaattagatagatttggattagcagaaagcctgttggagaacaaagaaggatgtcaaaaaatactttcggtcttagctccattagtgccaacaggctcagaaaatttaaaaagcctttataatactgtctgcgtcatctggtgcattcacgcagaagagaaagtgaaacacactgaggaagcaaaacagatagtgcagagacacctagtggtggaaacaggaaccaccgaaaccatgccgaagacctctcgaccaacagcaccatctagcggcagaggaggaaactacccagtacagcagatcggtggcaactacgtccacctgccactgtccccgagaaccctgaacgcttgggtcaagctgatcgaggagaagaagttcggagcagaagtagtgccaggattccaggcactgtcagaaggttgcaccccctacgacatcaaccagatgctgaactgcgttggagaccatcaggcggctatgcagatcatccgtgacatcatcaacgaggaggctgcagattgggacttgcagcacccacaaccagctccacaacaaggacaacttagggagccgtcaggatcagacatcgcaggaaccacctcctcagttgacgaacagatccagtggatgtaccgtcagcagaacccgatcccagtaggcaacatctaccgtcgatggatccagctgggtctgcagaaatgcgtccgtatgtacaacccgaccaacattctagatgtaaaacaagggccaaaagagccatttcagagctatgtagacaggttctacaaaagtttaagagcagaacagacagatgcagcagtaaagaattggatgactcaaacactgctgattcaaaatgctaacccagattgcaagctagtgctgaaggggctgggtgtgaatcccaccctagaagaaatgctgacggcttgtcaaggagtaggggggccgggacagaaggctagattaatggcagaagccctgaaagaggccctcgcaccagtgccaatcccttttgcagcagcccaacagaggggaccaagaaagccaattaagtgttggaattgtgggaaagagggacactctgcaaggcaatgcagagccccaagaagacagggatgctggaaatgtggaaaaatggaccatgttatggccaaatgcccagacagacaggcgggttttttaggccttggtccatggggaaagaagccccgcaatttccccatggctcaagtgcatcaggggctgatgccaactgctcccccagaggacccagctgtggatctgctaaagaactacatgcagttgggcaagcagcagagagaaaagcagagagaaagcagagagaagccttacaaggaggtgacagaggatttgctgcacctcaattctctctttggaggagaccagtaggaattcgagctcggtacgatccagatctgctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcagcacagcaagggggaggattgggaagacaatagcaggcatgctggggatgcggtgggctctatgggtacccaggtgctgaagaattgacccggttcctcctgggccagaaagaagcaggcacatccccttctctgtgacacaccctgtccacgcccctggttcttagttccagccccactcataggacactcatagctcaggagggctccgccttcaatcccacccgctaaagtacttggagcggtctctccctccctcatcagcccaccaaaccaaacctagcctccaagagtgggaagaaattaaagcaagataggctattaagtgcagagggagagaaaatgcctccaacatgtgaggaagtaatgagagaaatcatagaatttcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccggggggggggggcgctgaggtctgcctcgtgaagaaggtgttgctgactcataccaggcctgaatcgccccatcatccagccagaaagtgagggagccacggttgatgagagctttgttgtaggtggaccagttggtgattttgaacttttgctttgccacggaacggtctgcgttgtcgggaagatgcgtgatctgatccttcaactcagcaaaagttcgatttattcaacaaagccgccgtcccgtcaagtcagcgtaatgctctgccagtgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacagttttattgttcatgatgatatatttttatcttgtgcaatgtaacatcagagattttgagacacaacgtggctttccccccccccccattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcagattggctattgg

SEQ ID NO: 50 - protein, 510 aa, Simian immunodeficiency virus
  SIV p57gag fusion protein
MGVRNSVLSGKKADELEKIRLRPNGKKKYMLKHVVWAANELDRFGLAESLLENKEGCQKILSVLAPLVPTGSENLKSLYNTVCVIWCIHAEEKVKHTEEAKQIVQRHLVVETGTTETMPKTSRPTAPSSGRGGNYPVQQIGGNYVHLPLSPRTLNAWVKLIEEKKFGAEVVPGFQALSEGCTPYDINQMLNCVGDHQAAMQIIRDIINEEAADWDLQHPQPAPQQGQLREPSGSDIAGTTSSVDEQIQWMYRQQNPIPVGNIYRRWIQLGLQKCVRMYNPTNILDVKQGPKEPFQSYVDRFYKSLRAEQTDAAVKNWMTQTLLIQNANPDCKLVLKGLGVNPTLEEMLTACQGVGGPGQKARLMAEALKEALAPVPIPFAAAQQRGPRKPIKCWNCGKEGHSARQCRAPRRQGCWKCGKMDHVMAKCPDRQAGFLGLGPWGKKPRNFPMAQVHQGLMPTAPPEDPAVDLLKNYMQLGKQQREKQRESREKPYKEVTEDLLHLNSLFGGDQ

SEQ ID NO: 51 - DNA, 5646 bp, Artificial Sequence
  Description of Artificial Sequence pCATESIVgagDX
cctggccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccgcgggcgcgcatgagaaaagcggctgttagtcactggcagcagcagtcttacctggactctggaatccattctggtgccactaccacagctccttctctgagtgctagcgcaggagcaggcgtgagaaactccgtcttgtcagggaagaaagcagatgaattagaaaaaattaggctacgacccaacggaaagaaaaagtacatgttgaagcatgtagtatgggcagcaaatgaattagatagatttggattagcagaaagcctgttggagaacaaagaaggatgtcaaaaaatactttcggtcttagctccattagtgccaacaggctcagaaaatttaaaaagcctttataatactgtctgcgtcatctggtgcattcacgcagaagagaaagtgaaacacactgaggaagcaaaacagatagtgcagagacacctagtggtggaaacaggaaccaccgaaaccatgccgaagacctctcgaccaacagcaccatctagcggcagaggaggaaactacccagtacagcagatcggtggcaactacgtccacctgccactgtccccgagaaccctgaacgcttgggtcaagctgatcgaggagaagaagttcggagcagaagtagtgccaggattccaggcactgtcagaaggttgcaccccctacgacatcaaccagatgctgaactgcgttggagaccatcaggcggctatgcagatcatccgtgacatcatcaacgaggaggctgcagattgggacttgcagcacccacaaccagctccacaacaaggacaacttagggagccgtcaggatcagacatcgcaggaaccacctcctcagttgacgaacagatccagtggatgtaccgtcagcagaacccgatcccagtaggcaacatctaccgtcgatggatccagctgggtctgcagaaatgcgtccgtatgtacaacccgaccaacattctagatgtaaaacaagggccaaaagagccatttcagagctatgtagacaggttctacaaaagtttaagagcagaacagacagatgcagcagtaaagaattggatgactcaaacactgctgattcaaaatgctaacccagattgcaagctagtgctgaaggggctgggtgtgaatcccaccctagaagaaatgctgacggcttgtcaaggagtaggggggccgggacagaaggctagattaatggcagaagccctgaaagaggccctcgcaccagtgccaatcccttttgcagcagcccaacagaggggaccaagaaagccaattaagtgttggaattgtgggaaagagggacactctgcaaggcaatgcagagccccaagaagacagggatgctggaaatgtggaaaaatggaccatgttatggccaaatgcccagacagacaggcgggttttttaggccttggtccatggggaaagaagccccgcaatttccccatggctcaagtgcatcaggggctgatgccaactgctcccccagaggacccagctgtggatctgctaaagaactacatgcagttgggcaagcagcagagagaaaagcagagagaaagcagagagaagccttacaaggaggtgacagaggatttgctgcacctcaattctctctttggaggagaccagtaggaattctgatacgatccagatctgctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcagcacagcaagggggaggattgggaagacaatagcaggcatgctggggatgcggtgggctctatgggtacccaggtgctgaagaattgacccggttcctcctgggccagaaagaagcaggcacatccccttctctgtgacacaccctgtccacgcccctggttcttagttccagccccactcataggacactcatagctcaggagggctccgccttcaatcccacccgctaaagtacttggagcggtctctccctccctcatcagcccaccaaaccaaacctagcctccaagagtgggaagaaattaaagcaagataggctattaagtgcagagggagagaaaatgcctccaacatgtgaggaagtaatgagagaaatcatagaatttcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccggggggggggggcgctgaggtctgcctcgtgaagaaggtgttgctgactcataccaggcctgaatcgccccatcatccagccagaaagtgagggagccacggttgatgagagctttgttgtaggtggaccagttggtgattttgaacttttgctttgccacggaacggtctgcgttgtcgggaagatgcgtgatctgatccttcaactcagcaaaagttcgatttattcaacaaagccgccgtcccgtcaagtcagcgtaatgctctgccagtgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacagttttattgttcatgatgatatatttttatcttgtgcaatgtaacatcagagattttgagacacaacgtggctttccccccccccccattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcagattggctattgg

SEQ ID NO: 52 - protein, 545 aa, Artificial Sequence
  Description of Artificial Sequence CATESIVp57gag fusion protein
MRKAAVSHWQQQSYLDSGIHSGATTTAPSLSASAGAGVRNSVLSGKKADELEKIRLRPNGKKKYMLKHVVWAANELDRFGLAESLLENKEGCQKILSVLAPLVPTGSENLKSLYNTVCVIWCIHAEEKVKHTEEAKQIVQRHLVVETGTTETMPKTSRPTAPSSGRGGNYPVQQIGGNYVHLPLSPRTLNAWVKLIEEKKFGAEVVPGFQALSEGCTPYDINQMLNCVGDHQAAMQIIRDIINEEAADWDLQHPQPAPQQGQLREPSGSDIAGTTSSVDEQIQWMYRQQNPIPVGNIYRRWIQLGLQKCVRMYNPTNILDVKQGPKEPFQSYVDRFYKSLRAEQTDAAVKNWMTQTLLIQNANPDCKLVLKGLGVNPTLEEMLTACQGVGGPGQKARLMAEALKEALAPVPIPFAAAQQRGPRKPIKCWNCGKEGHSARQCRAPRRQGCWKCGKMDHVMAKCPDRQAGFLGLGPWGKKPRNFPMAQVHQGLMPTAPPEDPAVDLLKNYMQLGKQQREKQRESREKPYKEVTEDLLHLNSLFGGDQ

SEQ ID NO: 53 - DNA, 5418 bp, Artificial Sequence
  Description of Artificial SequencepCMVMCP3p39
cctggccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccgcgggcgcgcatgaacccaagtgctgccgtcattttctgcctcatcctgctgggtctgagtgggactcaagggatcctcgacatggcgcaaccggtaggtataaacacaagcacaacctgttgctatcgtttcataaataaaaagataccgaagcaacgtctggaaagctatcgccgtaccacttctagccactgtccgcgtgaagctgttatattcaaaacgaaactggataaggagatctgcgccgaccctacacagaaatgggttcaggactttatgaagcacctggataaaaagacacagacgccgaaactggctagcgcaggagcaggcgtgagaaactccgtcttgtcagggaagaaagcagatgaattagaaaaaattaggctacgacccaacggaaagaaaaagtacatgttgaagcatgtagtatgggcagcaaatgaattagatagatttggattagcagaaagcctgttggagaacaaagaaggatgtcaaaaaatactttcggtcttagctccattagtgccaacaggctcagaaaatttaaaaagcctttataatactgtctgcgtcatctggtgcattcacgcagaagagaaagtgaaacacactgaggaagcaaaacagatagtgcagagacacctagtggtggaaacaggaaccaccgaaaccatgccgaagacctctcgaccaacagcaccatctagcggcagaggaggaaactacccagtacagcagatcggtggcaactacgtccacctgccactgtccccgagaaccctgaacgcttgggtcaagctgatcgaggagaagaagttcggagcagaagtagtgccaggattccaggcactgtcagaaggttgcaccccctacgacatcaaccagatgctgaactgcgttggagaccatcaggcggctatgcagatcatccgtgacatcatcaacgaggaggctgcagattgggacttgcagcacccacaaccagctccacaacaaggacaacttagggagccgtcaggatcagacatcgcaggaaccacctcctcagttgacgaacagatccagtggatgtaccgtcagcagaacccgatcccagtaggcaacatctaccgtcgatggatccagctgggtctgcagaaatgcgtccgtatgtacaacccgaccaacattctagatgtaaaacaagggccaaaagagccatttcagagctatgtagacaggttctacaaaagtttaagagcagaacagacagatgcagcagtaaagaattggatgactcaaacactgctgattcaaaatgctaacccagattgcaagctagtgctgaaggggctgggtgtgaatcccaccctagaagaaatgctgacggcttgtcaaggagtaggggggccgggacagaaggctagattaatggaattctgatacgatccagatctgctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcagcacagcaagggggaggattgggaagacaatagcaggcatgctggggatgcggtgggctctatgggtacccaggtgctgaagaattgacccggttcctcctgggccagaaagaagcaggcacatccccttctctgtgacacaccctgtccacgcccctggttcttagttccagccccactcataggacactcatagctcaggagggctccgccttcaatcccacccgctaaagtacttggagcggtctctccctccctcatcagcccaccaaaccaaacctagcctccaagagtgggaagaaattaaagcaagataggctattaagtgcagagggagagaaaatgcctccaacatgtgaggaagtaatgagagaaatcatagaatttcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccggggggggggggcgctgaggtctgcctcgtgaagaaggtgttgctgactcataccaggcctgaatcgccccatcatccagccagaaagtgagggagccacggttgatgagagctttgttgtaggtggaccagttggtgattttgaacttttgctttgccacggaacggtctgcgttgtcgggaagatgcgtgatctgatccttcaactcagcaaaagttcgatttattcaacaaagccgccgtcccgtcaagtcagcgtaatgctctgccagtgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacagttttattgttcatgatgatatatttttatcttgtgcaatgtaacatcagagattttgagacacaacgtggctttccccccccccccattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcagattggctattgg

SEQ ID NO: 54 - protein, 472 aa, Artificial Sequence
  Description of Artificial SequenceIP10-MCP3-SIVp39gag fusion protein
MNPSAAVIFCLILLGLSGTQGILDMAQPVGINTSTTCCYRFINKKIPKQRLESYRRTTSSHCPREAVIFKTKLDKEICADPTQKWVQDFMKHLDKKTQTPKLASAGAGVRNSVLSGKKADELEKIRLRPNGKKKYMLKHVVWAANELDRFGLAESLLENKEGCQKILSVLAPLVPTGSENLKSLYNTVCVIWCIHAEEKVKHTEEAKQIVQRHLVVETGTTETMPKTSRPTAPSSGRGGNYPVQQIGGNYVHLPLSPRTLNAWVKLIEEKKFGAEVVPGFQALSEGCTPYDINQMLNCVGDHQAAMQIIRDIINEEAADWDLQHPQPAPQQGQLREPSGSDIAGTTSSVDEQIQWMYRQQNPIPVGNIYRRWIQLGLQKCVRMYNPTNILDVKQGPKEPFQSYVDRFYKSLRAEQTDAAVKNWMTQTLLIQNANPDCKLVLKGLGVNPTLEEMLTACQGVGGPGQKARLMEF

SEQ ID NO: 55 - DNA, 8900 bp, Artificial Sequence
  Description of Artificial SequencepCMV SIV CATEpolNTV
cctggccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccgcgggcgcgcgtcgacaagaaatgagaaaagcggctgttagtcactggcagcagcagtcttacctggactctggaatccattctggtgccactaccacagctccttctctgagtgctagcgcaggagcatacccctacgacgtgcccgactacgccagcctgggggcccatcgggaggcgttgcaggggggagatcgggggttcgcggcgccgcagttctcgctgtggcggcggccggtcgtcactgcgcatattgagggacagccggtagaggtattgctggcggcagcggcggatgattcgattgtaacgggaatagagttgggtccgcattataccccgaagatagtaggggggatcggggggtttatcaatacgaaagagtacaaaaatgtagagatagaggttttgggcaaacggattaaagggacgatcatgacaggggacaccccgattaacatctttggtcggaacttgctaacggcgctggggatgtcgctaaactttcccatagcgaaagtagagcctgtaaaagtcgccttgaagccgggaaaggatggaccgaaattgaagcagtggccgttgtcaaaagagaagatagttgcgttgcgggagatctgtgagaagatggagaaggatggacagttggaggaggcgcccccgaccaatccatacaacacccccacattcgcgatcaagaagaaggataagaacaagtggcggatgctgatagactttcgggagttgaatcgggtcacgcaggactttacggaggtccaattgggaataccgcacccggcgggactagcgaaacggaaacggattacggtactggatataggtgatgcgtacttctccataccgcttgatgaggagtttcggcagtacacggcctttacgcttccgtcagtaaacaacgcggagccggggaagcgatacatatataaggttctgccgcagggatggaaggggtcgccggccatcttccaatacacgatgcggcatgtgctagagcccttccggaaggcgaatccggatgtgaccttggtccagtatatggcggcgatcttgatagcgtcggaccggacggacctggagcatgaccgggtagttcttcagtcgaaggagctcttgaatagcatagggttttcgaccccggaggagaaattccaaaaagatcccccgtttcaatggatggggtacgagttgtggccgacgaaatggaagttgcaaaagatagagttgccgcaacgggagacctggacagtgaatgatatacagaagcttgtaggagtacttaattgggcggctcaaatatatccgggtataaaaaccaaacatctctgtcggttgattcggggaaaaatgacgctaacggaggaggttcagtggacggagatggcggaggcagagtatgaggagaacaagatcatcctctcgcaggagcaagagggatgttattaccaagagggcaagccgttggaggccacggtaatcaagtcgcaggacaatcagtggtcgtataagatccaccaagaggacaagatcctgaaagtaggaaagttcgcgaagatcaagaacacgcataccaacggagtgcggctacttgcgcatgtaatacagaaaataggaaaggaggcgatagtgatctggggacaggtcccgaaattccaccttccggttgagaaggatgtatgggagcagtggtggacggactattggcaagtaacctggataccggagtgggactttatctcgacgccgccgctagtacggcttgtcttcaatctagtgaaggacccgatagagggagaggagacctattatacggatggatcgtgtaacaagcagtcgaaagaggggaaagcgggatatatcacggatcggggcaaagacaaagtaaaagtgcttgagcagacgacgaatcaacaagcggcgttggaggcgtttctcatggcgttgacggactcggggccaaaggcgaacatcatcgtagactcgcagtacgtcatgggaatcatcacgggatgcccgacggagtcggagagccggctagtcaaccaaatcatcgaggagatgatcaagaagtcggagatatatgtagcgtgggtaccggcgcacaaaggtataggaggaaaccaagagatagaccacctagtttcgcaagggattagacaagttctcttcttggagaagatagagccggcgcaagaggagcatgataaataccattcgaatgtaaaagagttggtattcaaattcggacttccccggatagtggcccggcagatagtagacacctgtgataaatgtcatcagaaaggagaggcgatacatgggcaggcgaactcggatctagggacttggcaaatggcgtgtacccatctagagggaaagatcatcatagttgcggtacatgtagcgtcgggattcatagaagcggaggtaattccgcaagagacgggacggcagacggcgctattcctgttgaaattggcgggcagatggcctattacgcatctacacacggcgaatggtgcgaactttgcgtcgcaagaagtaaagatggttgcgtggtgggcggggatagagcacacctttggggtaccgtacaatccgcagtcgcagggagtagtggcggcgatgaaccaccacctgaagaaccaaatcgatcggatcagggagcaagcgaactcagtagagaccatagtattgatggcggttcattgcatgaacttcaagcggcggggaggaataggggatatgacgccggcggagcggttgattaacatgatcacgacggagcaagagatccaattccaacaatcgaagaactcgaagttcaagaactttcgggtctattaccgggagggccgggatcaactgtggaagggacccggagagctattgtggaaaggggagggagcggtcatcttgaaagtagggacggacattaaggtagtaccccggcggaaggcgaagatcatcaaggattatggaggaggaaaagaggtggatagctcgtcccacatggaggataccggagaggcgcgggaggtggcacgcgtcgcggccgcggctatctccatgaggcggtccaggccgtctggggatctgcgacagagactcttgcgggcgcgtggggagacttatgggagactcttaggagaggtggaagatggatactcgcaatccccaggaggattagacaagggcttgagctcactctcgtgcgagggacagaagtacaaccaggggcagtacatgaacactccatggagaaaccccgctgaagagcgggagaagttggcgtaccggaagcagaacatggacgacatcgacgaggaggacgacgacttagtcggggtctcagtgcggccgaaggtccccctacggacgatgtcgtacaagttggcgatagacatgtcgcacttcatcaaggagaaggggggactggaggggatctactactcggcgcggcggcaccgcatcctcgacatctacctcgagaaggaggagggcatcatcccggactggcaggactacacctcaggaccaggaatcagatatccaaagacgttcggctggctctggaagctcgtccctgtaaacgtctcggacgaggcgcaggaggacgaggagcactacctcatgcatccggcgcaaacttcccagtgggatgacccttggggagaggttctagcatggaagtttgatccaactctggcctacacttatgaggcatatgttagatacccagaagagtttggaagcaagtcaggcctgtcagaggaagaggttagaagaaggctaaccgcaagaggccttcttaacatggctgacaagaaggaaactcgcggcgccgagacacccttgagggagcaggagaactcattagaatcctccaacgagcgctcttcatgcatttcagaggcggatgcatccactccagaatcggccaacctgggggaggaaatcctctctcagctataccgccctctcgaggcgtgctacaacacgtgctactgcaagaagtgctgctaccactgccagttctgcttccttaaaaagggcctggggatctgctacgagcagtcgcgaaagcggcggcggacgccgaagaaggcgaaggcgaacacgtcgtcggcgtcgaacaacagacccatatccaacaggacccggcactgccaaccagagaaggcaaagaaagagacggtggagaaggcggtggcaacagctcctggccttggcagaggatccgaggaggaaaagaggtggatcgcagttcccacgtggaggataccggagaggctagagaggtggcatagcctcataaagtacctgaagtacaagacgaaggacctccagaaggtctgctatgtgccccacttcaaagtcggatgggcatggtggacctgcagcagagtcatcttccccctacaagagggaagccacttggaggtccaggggtactggcacttgacgccggagaaggggtggctctcgacgtacgcggtgcggatcacctggtactcgaagaacttctggacggatgtcacgccgaactatgcggacatcttgctgcatagcacttacttcccttgctttacggcgggagaagtgagaagggccatcaggggagagcaactgctgtcgtgctgccggttcccgcgggcgcacaagtaccaggtaccgagcctacagtacttggcgctgaaggtcgtcagcgacgtcagatcccagggggagaaccccacctggaagcagtggcggcgggacaaccggagaggccttcgaatggcgaagcagaactcgcggggagataagcagcggggcggtaaaccacctaccaagggagcgaacttcccgggtttggcaaaggtcttgggaatactggcagttaactgagaattcgatccagatctgctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcagcacagcaagggggaggattgggaagacaatagcaggcatgctggggatgcggtgggctctatgggtacccaggtgctgaagaattgacccggttcctcctgggccagaaagaagcaggcacatccccttctctgtgacacaccctgtccacgcccctggttcttagttccagccccactcataggacactcatagctcaggagggctccgccttcaatcccacccgctaaagtacttggagcggtctctccctccctcatcagcccaccaaaccaaacctagcctccaagagtgggaagaaattaaagcaagataggctattaagtgcagagggagagaaaatgcctccaacatgtgaggaagtaatgagagaaatcatagaatttcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccggggggggggggcgctgaggtctgcctcgtgaagaaggtgttgctgactcataccaggcctgaatcgccccatcatccagccagaaagtgagggagccacggttgatgagagctttgttgtaggtggaccagttggtgattttgaacttttgctttgccacggaacggtctgcgttgtcgggaagatgcgtgatctgatccttcaactcagcaaaagttcgatttattcaacaaagccgccgtcccgtcaagtcagcgtaatgctctgccagtgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacagttttattgttcatgatgatatatttttatcttgtgcaatgtaacatcagagattttgagacacaacgtggctttccccccccccccattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcagattggctattgg

SEQ ID NO: 56 - protein, 1628 aa, Artificial Sequence
  Description of Artificial SequenceCATE-HA epitope-pol ORF-pol-NefTatVif (NTV) fusion protein
MRKAAVSHWQQQSYLDSGIHSGATTTAPSLSASAGAYPYDVPDYASLGAHREALQGGDRGFAAPQFSLWRRPVVTAHIEGQPVEVLLAAAADDSIVTGIELGPHYTPKIVGGIGGFINTKEYKNVEIEVLGKRIKGTIMTGDTPINIFGRNLLTALGMSLNFPIAKVEPVKVALKPGKDGPKLKQWPLSKEKIVALREICEKMEKDGQLEEAPPTNPYNTPTFAIKKKDKNKWRMLIDFRELNRVTQDFTEVQLGIPHPAGLAKRKRITVLDIGDAYFSIPLDEEFRQYTAFTLPSVNNAEPGKRYIYKVLPQGWKGSPAIFQYTMRHVLEPFRKANPDVTLVQYMAAILIASDRTDLEHDRVVLQSKELLNSIGFSTPEEKFQKDPPFQWMGYELWPTKWKLQKIELPQRETWTVNDIQKLVGVLNWAAQIYPGIKTKHLCRLIRGKMTLTEEVQWTEMAEAEYEENKIILSQEQEGCYYQEGKPLEATVIKSQDNQWSYKIHQEDKILKVGKFAKIKNTHTNGVRLLAHVIQKIGKEAIVIWGQVPKFHLPVEKDVWEQWWTDYWQVTWIPEWDFISTPPLVRLVFNLVKDPIEGEETYYTDGSCNKQSKEGKAGYITDRGKDKVKVLEQTTNQQAALEAFLMALTDSGPKANIIVDSQYVMGIITGCPTESESRLVNQIIEEMIKKSEIYVAWVPAHKGIGGNQEIDHLVSQGIRQVLFLEKIEPAQEEHDKYHSNVKELVFKFGLPRIVARQIVDTCDKCHQKGEAIHGQANSDLGTWQMACTHLEGKIIIVAVHVASGFIEAEVIPQETGRQTALFLLKLAGRWPITHLHTANGANFASQEVKMVAWWAGIEHTFGVPYNPQSQGVVAAMNHHLKNQIDRIREQANSVETIVLMAVHCMNFKRRGGIGDMTPAERLINMITTEQEIQFQQSKNSKFKNFRVYYREGRDQLWKGPGELLWKGEGAVILKVGTDIKVVPRRKAKIIKDYGGGKEVDSSSHMEDTGEAREVARVAAAAISMRRSRPSGDLRQRLLRARGETYGRLLGEVEDGYSQSPGGLDKGLSSLSCEGQKYNQGQYMNTPWRNPAEEREKLAYRKQNMDDIDEEDDDLVGVSVRPKVPLRTMSYKLAIDMSHFIKEKGGLEGIYYSARRHRILDIYLEKEEGIIPDWQDYTSGPGIRYPKTFGWLWKLVPVNVSDEAQEDEEHYLMHPAQTSQWDDPWGEVLAWKFDPTLAYTYEAYVRYPEEFGSKSGLSEEEVRRRLTARGLLNMADKKETRGAETPLREQENSLESSNERSSCISEADASTPESANLGEEILSQLYRPLEACYNTCYCKKCCYHCQFCFLKKGLGICYEQSRKRRRTPKKAKANTSSASNNRPISNRTRHCQPEKAKKETVEKAVATAPGLGRGSEEEKRWIAVPTWRIPERLERWHSLIKYLKYKTKDLQKVCYVPHFKVGWAWWTCSRVIFPLQEGSHLEVQGYWHLTPEKGWLSTYAVRITWYSKNFWTDVTPNYADILLHSTYFPCFTAGEVRRAIRGEQLLSCCRFPRAHKYQVPSLQYLALKVVSDVRSQGENPTWKQWRRDNRRGLRMAKQNSRGDKQRGGKPPTKGANFPGLAKVLGILAVN

SEQ ID NO: 57 - protein, 951 aa, Simian immunodeficiency virus
  wildtype SIV pol
PQFSLWRRPVVTAHIEGQPVEVLLDTGADDSIVTGIELGPHYTPKIVGGIGGFINTKEYKNVEIEVLGKRIKGTIMTGDTPINIFGRNLLTALGMSLNFPIAKVEPVKVALKPGKDGPKLKQWPLSKEKIVALREICEKMEKDGQLEEAPPTNPYNTPTFAIKKKDKNKWRMLIDFRELNRVTQDFTEVQLGIPHPAGLAKRKRITVLDIGDAYFSIPLDEEFRQYTAFTLPSVNNAEPGKRYIYKVLPQGWKGSPAIFQYTMRHVLEPFRKANPDVTLVQYMDDILIASDRTDLEHDRVVLQSKELLNSIGFSTPEEKFQKDPPFQWMGYELWPTKWKLQKIELPQRETWTVNDIQKLVGVLNWAAQIYPGIKTKHLCRLIRGKMTLTEEVQWTEMAEAEYEENKIILSQEQEGCYYQEGKPLEATVIKSQDNQWSYKIHQEDKILKVGKFAKIKNTHTNGVRLLAHVIQKIGKEAIVIWGQVPKFHLPVEKDVWEQWWTDYWQVTWIPEWDFISTPPLVRLVFNLVKDPIEGEETYYTDGSCNKQSKEGKAGYITDRGKDKVKVLEQTTNQQAELEAFLMALTDSGPKANIIVDSQYVMGIITGCPTESESRLVNQIIEEMIKKSEIYVAWVPAHKGIGGNQEIDHLVSQGIRQVLFLEKIEPAQEEHDKYHSNVKELVFKFGLPRIVARQIVDTCDKCHQKGEAIHGQANSDLGTWQMDCTHLEGKIIIVAVHVASGFIEAEVIPQETGRQTALFLLKLAGRWPITHLHTDNGANFASQEVKMVAWWAGIEHTFGVPYNPQSQGVVEAMNHHLKNQIDRIREQANSVETIVLMAVHCMNFKRRGGIGDMTPAERLINMITTEQEIQFQQSKNSKFKNFRVYYREGRDQLWKGPGELLWKGEGAVILKVGTDIKVVPRRKAKIIKDYGGGKEVDSSSHMEDTGEAREVA

SEQ ID NO: 58 - protein, 951 aa, Simian immunodeficiency virus
  mutant SIV pol (SIVmac239)
PQFSLWRRPVVTAHIEGQPVEVLLAAAADDSIVTGIELGPHYTPKIVGGIGGFINTKEYKNVEIEVLGKRIKGTIMTGDTPINIFGRNLLTALGMSLNFPIAKVEPVKVALKPGKDGPKLKQWPLSKEKIVALREICEKMEKDGQLEEAPPTNPYNTPTFAIKKKDKNKWRMLIDFRELNRVTQDFTEVQLGIPHPAGLAKRKRITVLDIGDAYFSIPLDEEFRQYTAFTLPSVNNAEPGKRYIYKVLPQGWKGSPAIFQYTMRHVLEPFRKANPDVTLVQYMAAILIASDRTDLEHDRVVLQSKELLNSIGFSTPEEKFQKDPPFQWMGYELWPTKWKLQKIELPQRETWTVNDIQKLVGVLNWAAQIYPGIKTKHLCRLIRGKMTLTEEVQWTEMAEAEYEENKIILSQEQEGCYYQEGKPLEATVIKSQDNQWSYKIHQEDKILKVGKFAKIKNTHTNGVRLLAHVIQKIGKEAIVIWGQVPKFHLPVEKDVWEQWWTDYWQVTWIPEWDFISTPPLVRLVFNLVKDPIEGEETYYTDGSCNKQSKEGKAGYITDRGKDKVKVLEQTTNQQAALEAFLMALTDSGPKANIIVDSQYVMGIITGCPTESESRLVNQIIEEMIKKSEIYVAWVPAHKGIGGNQEIDHLVSQGIRQVLFLEKIEPAQEEHDKYHSNVKELVFKFGLPRIVARQIVDTCDKCHQKGEAIHGQANSDLGTWQMACTHLEGKIIIVAVHVASGFIEAEVIPQETGRQTALFLLKLAGRWPITHLHTANGANFASQEVKMVAWWAGIEHTFGVPYNPQSQGVVAAMNHHLKNQIDRIREQANSVETIVLMAVHCMNFKRRGGIGDMTPAERLINMITTEQEIQFQQSKNSKFKNFRVYYREGRDQLWKGPGELLWKGEGAVILKVGTDIKVVPRRKAKIIKDYGGGKEVDSSSHMEDTGEAREVA

SEQ ID NO: 59 - protein, 24 aa, Artificial Sequence
  Description of Artificial SequenceSIV pol consensus sequence
PQFSLWRRPVVTAHIEGQPVEVLL

SEQ ID NO: 60 - protein, 256 aa, Artificial Sequence; identical to SEQ ID NO: 41
  Description of Artificial SequenceSIV pol consensus sequence

SEQ ID NO: 61 - protein, 290 aa, Artificial Sequence; identical to SEQ ID NO: 42
  Description of Artificial SequenceSIV pol consensus sequence

SEQ ID NO: 62 - protein, 145 aa, Artificial Sequence; identical to SEQ ID NO: 43
  Description of Artificial SequenceSIV pol consensus sequence

SEQ ID NO: 63 - protein, 51 aa, Artificial Sequence; identical to SEQ ID NO: 44
  Description of Artificial SequenceSIV pol consensus sequence

SEQ ID NO: 64 - protein, 35 aa, Artificial Sequence; identical to SEQ ID NO: 45
  Description of Artificial SequenceSIV pol consensus sequence

SEQ ID NO: 65 - protein, 141 aa, Artificial Sequence; identical to SEQ ID NO: 46
  Description of Artificial SequenceSIV pol consensus sequence

SEQ ID NO: 66 - DNA, 6023 bp, Artificial Sequence
  Description of Artificial Sequence59S_CMV_CATESIVenv1
cgatgatatccattgcatacgttgtatctatatcataatatgtacatttatattggctcatgtccaatatgaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggacccgatccagcctccgcgggcgcgcgtcgaggaattcaagaaatgagaaaagcggctgttagtcactggcagcagcagtcttacctggactctggaatccattctggtgccactaccacagctccttctctgagtatctgcagcctgtacgtcacggtcttctacggcgtaccagcttggaggaatgcgacaattcccctcttttgtgcaaccaagaatagggatacttggggaacaactcagtgcctaccggacaacggggactactcggaggtggccctgaacgtgacggagagcttcgacgcctggaacaacacggtcacggagcaggcgatcgaggacgtgtggcagctgttcgagacctcgatcaagccgtgcgtcaagctgtccccgctctgcatcacgatgcggtgcaacaagagcgagacggatcggtgggggctgacgaagtcgatcacgacgacggcgtcgaccacgtcgacgacggcgtcggcgaaagtggacatggtcaacgagacctcgtcgtgcatcgcccaggacaactgcacgggcctggagcaggagcagatgatcagctgcaagttcaacatgacggggctgaagcgggacaagaagaaggagtacaacgagacgtggtactcggcggacctggtgtgcgagcaggggaacaacacggggaacgagtcgcggtgctacatgaaccactgcaacacgtcggtgatccaggagtcgtgcgacaagcactactgggacgcgatccggttccggtactgcgcgccgccgggctacgcgctgctgcggtgcaacgacacgaactactcgggcttcatgccgaaatgctcgaaggtggtggtctcgtcgtgcacgaggatgatggagacgcagacctcgacgtggttcggcttcaacgggacgcgggcggagaaccggacgtacatctactggcacgggcgggacaaccggacgatcatctcgctgaacaagtactacaacctgacgatgaagtgccggcggccgggcaacaagacggtgctcccggtcaccatcatgtcggggctggtgttccactcgcagccgatcaacgaccggccgaagcaggcgtggtgctggttcggggggaagtggaaggacgcgatcaaggaggtgaagcagaccatcgtcaagcacccccgctacacggggacgaacaacacggacaagatcaacctgacggcgccgggcgggggcgatccggaagttaccttcatgtggacaaattgcagaggagagttcctctactgcaagatgaactggttcctgaactgggtggaggacaggaacacggcgaaccagaagccgaaggagcagcacaagcggaactacgtgccgtgccacattcggcagatcatcaacacgtggcacaaagtgggcaagaacgtgtacctgccgccgagggagggcgacctcacgtgcaactccacggtgacctccctcatcgcgaacatcgactggatcgacggcaaccagacgaacatcaccatgtcggcggaggtggcggagctgtaccggctggagctgggggactacaagctggtggagatcacgccgatcggcctggcccccaccgatgtgaagcgctacacgaccggggggacgtcgcggaacaagcggggggtcttcgtcctggggttcctggggttcctcgcgacggcggggtcggcaatgggagccgccagcctgaccctcacggcacagtcccgaactttattggctgggatcgtccaacaacagcagcagctgctggacgtggtcaagaggcagcaggagctgctgcggctgaccgtctggggcacgaagaacctccagacgagggtcacggccatcgagaagtacctgaaggaccaggcgcagctgaacgcgtggggctgtgcgtttcgacaagtctgccacacgacggtcccgtggccgaacgcgtcgctgacgccgaagtggaacaacgagacgtggcaggagtgggagcggaaggtggacttcctggaggagaacatcacggccctcctggaggaggcgcagatccagcaggagaagaacatgtacgagctgcaaaagctgaacagctgggacgtgttcggcaactggttcgacctggcgtcgtggatcaagtacatccagtacggcgtgtacatcgtggtgggggtgatcctgctgcggatcgtgatctacatcgtccagatgctggcgaagctgcggcagggctataggccagtgttctcttccccaccctcttatttccaacaaacccatatccaacaagacccggcgctgccgacccgggagggcaaggagcgggacggcggggagggcggcggcaacagctcctggccgtggcagatcgagtacatccactttcttattcgtcagcttattagactcctgacgtggctgttcagtaactgtaggactctgctgtcgagggtgtaccagatcctccagccgatcctccagcggctctcggcgaccctccagaggattcgggaggtcctccggacggagctgacctacctccagtacgggtggagctatttccacgaggcggtccaggccgtctggcggtcggcgacggagacgctggcgggcgcgtggggcgacctgtgggagacgctgcggcggggcggccggtggatactcgcgatcccccggcggatcaggcaggggctggagctcacgctcctgtgataagatatcggatccgcccgggctagagcggccactcgagaggcgcgccgagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggaatttaaatgggggcgctgaggtctgcctcgtgaagaaggtgttgctgactcataccaggcctgaatcgccccatcatccagccagaaagtgagggagccacggttgatgagagctttgttgtaggtggaccagttggtgattttgaacttttgctttgccacggaacggtctgcgttgtcgggaagatgcgtgatctgatccttcaactcagcaaaagttcgatttattcaacaaagccgccgtcccgtcaagtcagcgtaatgctctgccagtgttacaaccaattaaccaattctgcgttcaaaatggtatgcgttttgacacatccactatatatccgtgtcgttctgtccactcctgaatcccattccagaaattctctagcgattccagaagtttctcagagtcggaaagttgaccagacattacgaactggcacagatggtcataacctgaaggaagatctgattgcttaactgcttcagttaagaccgacgcgctcgtcgtataacagatgcgatgatgcagaccaatcaacatggcacctgccattgctacctgtacagtcaaggatggtagaaatgttgtcggtccttgcacacgaatattacgccatttgcctgcatattcaaacagctcttctacgataagggcacaaatcgcatcgtggaacgtttgggcttctaccgatttagcagtttgatacactttctctaagtatccacctgaatcataaatcggcaaaatagagaaaaattgaccatgtgtaagcggccaatctgattccacctgagatgcataatctagtagaatctcttcgctatcaaaattcacttccaccttccactcaccggttgtccattcatggctgaactctgcttcctctgttgacatgacacacatcatctcaatatccgaatacggaccatcagtctgacgaccaagagagccataaacaccaatagccttaacatcatccccatatttatccaatattcgttccttaatttcatgaacaatcttcattctttcttctctagtcattattattggtccgttcataacaccccttgtattactgtttatgtaagcagacagttttattgttcatgatgatatatttttatcttgtgcaatgtaacatcagagattttgagacacaacgtggctttccccggcccatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatagccgcggcatcg

SEQ ID NO: 67 - protein, 890 aa, Artificial Sequence
  Description of Artificial SequenceCATE-env fusion protein
MRKAAVSHWQQQSYLDSGIHSGATTTAPSLSICSLYVTVFYGVPAWRNATIPLFCATKNRDTWGTTQCLPDNGDYSEVALNVTESFDAWNNTVTEQAIEDVWQLFETSIKPCVKLSPLCITMRCNKSETDRWGLTKSITTTASTTSTTASAKVDMVNETSSCIAQDNCTGLEQEQMISCKFNMTGLKRDKKKEYNETWYSADLVCEQGNNTGNESRCYMNHCNTSVIQESCDKHYWDAIRFRYCAPPGYALLRCNDTNYSGFMPKCSKVVVSSCTRMMETQTSTWFGFNGTRAENRTYIYWHGRDNRTIISLNKYYNLTMKCRRPGNKTVLPVTIMSGLVFHSQPINDRPKQAWCWFGGKWKDAIKEVKQTIVKHPRYTGTNNTDKINLTAPGGGDPEVTFMWTNCRGEFLYCKMNWFLNWVEDRNTANQKPKEQHKRNYVPCHIRQIINTWHKVGKNVYLPPREGDLTCNSTVTSLIANIDWIDGNQTNITMSAEVAELYRLELGDYKLVEITPIGLAPTDVKRYTTGGTSRNKRGVFVLGFLGFLATAGSAMGAASLTLTAQSRTLLAGIVQQQQQLLDVVKRQQELLRLTVWGTKNLQTRVTAIEKYLKDQAQLNAWGCAFRQVCHTTVPWPNASLTPKWNNETWQEWERKVDFLEENITALLEEAQIQQEKNMYELQKLNSWDVFGNWFDLASWIKYIQYGVYIVVGVILLRIVIYIVQMLAKLRQGYRPVFSSPPSYFQQTHIQQDPALPTREGKERDGGEGGGNSSWPWQIEYIHFLIRQLIRLLTWLFSNCRTLLSRVYQILQPILQRLSATLQRIREVLRTELTYLQYGWSYFHEAVQAVWRSATETLAGAWGDLWETLRRGGRWILAIPRRIRQGLELTLL

SEQ ID NO: 68 - DNA, 6690 bp, Artificial Sequence
  Description of Artificial Sequence72S pCMV CATESIVenv
cctggccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccgcgggcgcgcgtcgaggaattcaagaaatgagaaaagcggctgttagtcactggcagcagcagtcttacctggactctggaatccattctggtgccactaccacagctccttctctgagtatctgcagcctgtacgtcacggtcttctacggcgtaccagcttggaggaatgcgacaattcccctcttttgtgcaaccaagaatagggatacttggggaacaactcagtgcctaccggacaacggggactactcggaggtggccctgaacgtgacggagagcttcgacgcctggaacaacacggtcacggagcaggcgatcgaggacgtgtggcagctgttcgagacctcgatcaagccgtgcgtcaagctgtccccgctctgcatcacgatgcggtgcaacaagagcgagacggatcggtgggggctgacgaagtcgatcacgacgacggcgtcgaccacgtcgacgacggcgtcggcgaaagtggacatggtcaacgagacctcgtcgtgcatcgcccaggacaactgcacgggcctggagcaggagcagatgatcagctgcaagttcaacatgacggggctgaagcgggacaagaagaaggagtacaacgagacgtggtactcggcggacctggtgtgcgagcaggggaacaacacggggaacgagtcgcggtgctacatgaaccactgcaacacgtcggtgatccaggagtcgtgcgacaagcactactgggacgcgatccggttccggtactgcgcgccgccgggctacgcgctgctgcggtgcaacgacacgaactactcgggcttcatgccgaaatgctcgaaggtggtggtctcgtcgtgcacgaggatgatggagacgcagacctcgacgtggttcggcttcaacgggacgcgggcggagaaccggacgtacatctactggcacgggcgggacaaccggacgatcatctcgctgaacaagtactacaacctgacgatgaagtgccggcggccgggcaacaagacggtgctcccggtcaccatcatgtcggggctggtgttccactcgcagccgatcaacgaccggccgaagcaggcgtggtgctggttcggggggaagtggaaggacgcgatcaaggaggtgaagcagaccatcgtcaagcacccccgctacacggggacgaacaacacggacaagatcaacctgacggcgccgggcgggggcgatccggaagttaccttcatgtggacaaattgcagaggagagttcctctactgcaagatgaactggttcctgaactgggtggaggacaggaacacggcgaaccagaagccgaaggagcagcacaagcggaactacgtgccgtgccacattcggcagatcatcaacacgtggcacaaagtgggcaagaacgtgtacctgccgccgagggagggcgacctcacgtgcaactccacggtgacctccctcatcgcgaacatcgactggatcgacggcaaccagacgaacatcaccatgtcggcggaggtggcggagctgtaccggctggagctgggggactacaagctggtggagatcacgccgatcggcctggcccccaccgatgtgaagcgctacacgaccggggggacgtcgcggaacaagcggggggtcttcgtcctggggttcctggggttcctcgcgacggcggggtcggcaatgggagccgccagcctgaccctcacggcacagtcccgaactttattggctgggatcgtccaacaacagcagcagctgctggacgtggtcaagaggcagcaggagctgctgcggctgaccgtctggggcacgaagaacctccagacgagggtcacggccatcgagaagtacctgaaggaccaggcgcagctgaacgcgtggggctgtgcgtttcgacaagtctgccacacgacggtcccgtggccgaacgcgtcgctgacgccgaagtggaacaacgagacgtggcaggagtgggagcggaaggtggacttcctggaggagaacatcacggccctcctggaggaggcgcagatccagcaggagaagaacatgtacgagctgcaaaagctgaacagctgggacgtgttcggcaactggttcgacctggcgtcgtggatcaagtacatccagtacggcgtgtacatcgtggtgggggtgatcctgctgcggatcgtgatctacatcgtccagatgctggcgaagctgcggcagggctataggccagtgttctcttccccaccctcttatttccaacaaacccatatccaacaagacccggcgctgccgacccgggagggcaaggagcgggacggcggggagggcggcggcaacagctcctggccgtggcagatcgagtacatccactttcttattcgtcagcttattagactcctgacgtggctgttcagtaactgtaggactctgctgtcgagggtgtaccagatcctccagccgatcctccagcggctctcggcgaccctccagaggattcgggaggtcctccggacggagctgacctacctccagtacgggtggagctatttccacgaggcggtccaggccgtctggcggtcggcgacggagacgctggcgggcgcgtggggcgacctgtgggagacgctgcggcggggcggccggtggatactcgcgatcccccggcggatcaggcaggggctggagctcacgctcctgtgataagatatcggatctgctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcagcacagcaagggggaggattgggaagacaatagcaggcatgctggggatgcggtgggctctatgggtacccaggtgctgaagaattgacccggttcctcctgggccagaaagaagcaggcacatccccttctctgtgacacaccctgtccacgcccctggttcttagttccagccccactcataggacactcatagctcaggagggctccgccttcaatcccacccgctaaagtacttggagcggtctctccctccctcatcagcccaccaaaccaaacctagcctccaagagtgggaagaaattaaagcaagataggctattaagtgcagagggagagaaaatgcctccaacatgtgaggaagtaatgagagaaatcatagaatttcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccggggggggggggcgctgaggtctgcctcgtgaagaaggtgttgctgactcataccaggcctgaatcgccccatcatccagccagaaagtgagggagccacggttgatgagagctttgttgtaggtggaccagttggtgattttgaacttttgctttgccacggaacggtctgcgttgtcgggaagatgcgtgatctgatccttcaactcagcaaaagttcgatttattcaacaaagccgccgtcccgtcaagtcagcgtaatgctctgccagtgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacagttttattgttcatgatgatatatttttatcttgtgcaatgtaacatcagagattttgagacacaacgtggctttccccccccccccattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcagattggctattgg

SEQ ID NO: 69 - protein, 890 aa, Artificial Sequence; identical to SEQ ID NO: 67
  Description of Artificial SequenceCATE-env (CATE-Env SIVmac239) fusion protein

SEQ ID NO: 70 - DNA, 6903 bp, Artificial Sequence
  Description of Artificial SequencepCMV MCP3 SIVenv
cctggccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccgcgggcgcgcgtcgaggaattcaagaaatgaacccaagtgctgccgtcattttctgcctcatcctgctgggtctgagtgggactcaagggatcctcgacatggcgcaaccggtaggtataaacacaagcacaacctgttgctatcgtttcataaataaaaagataccgaagcaacgtctggaaagctatcgccgtaccacttctagccactgtccgcgtgaagctgttatattcaaaacgaaactggataaggagatctgcgccgaccctacacagaaatgggttcaggactttatgaagcacctggataaaaagacacagacgccgaaactgatctgcagcctgtacgtcacggtcttctacggcgtaccagcttggaggaatgcgacaattcccctcttttgtgcaaccaagaatagggatacttggggaacaactcagtgcctaccggacaacggggactactcggaggtggccctgaacgtgacggagagcttcgacgcctggaacaacacggtcacggagcaggcgatcgaggacgtgtggcagctgttcgagacctcgatcaagccgtgcgtcaagctgtccccgctctgcatcacgatgcggtgcaacaagagcgagacggatcggtgggggctgacgaagtcgatcacgacgacggcgtcgaccacgtcgacgacggcgtcggcgaaagtggacatggtcaacgagacctcgtcgtgcatcgcccaggacaactgcacgggcctggagcaggagcagatgatcagctgcaagttcaacatgacggggctgaagcgggacaagaagaaggagtacaacgagacgtggtactcggcggacctggtgtgcgagcaggggaacaacacggggaacgagtcgcggtgctacatgaaccactgcaacacgtcggtgatccaggagtcgtgcgacaagcactactgggacgcgatccggttccggtactgcgcgccgccgggctacgcgctgctgcggtgcaacgacacgaactactcgggcttcatgccgaaatgctcgaaggtggtggtctcgtcgtgcacgaggatgatggagacgcagacctcgacgtggttcggcttcaacgggacgcgggcggagaaccggacgtacatctactggcacgggcgggacaaccggacgatcatctcgctgaacaagtactacaacctgacgatgaagtgccggcggccgggcaacaagacggtgctcccggtcaccatcatgtcggggctggtgttccactcgcagccgatcaacgaccggccgaagcaggcgtggtgctggttcggggggaagtggaaggacgcgatcaaggaggtgaagcagaccatcgtcaagcacccccgctacacggggacgaacaacacggacaagatcaacctgacggcgccgggcgggggcgatccggaagttaccttcatgtggacaaattgcagaggagagttcctctactgcaagatgaactggttcctgaactgggtggaggacaggaacacggcgaaccagaagccgaaggagcagcacaagcggaactacgtgccgtgccacattcggcagatcatcaacacgtggcacaaagtgggcaagaacgtgtacctgccgccgagggagggcgacctcacgtgcaactccacggtgacctccctcatcgcgaacatcgactggatcgacggcaaccagacgaacatcaccatgtcggcggaggtggcggagctgtaccggctggagctgggggactacaagctggtggagatcacgccgatcggcctggcccccaccgatgtgaagcgctacacgaccggggggacgtcgcggaacaagcggggggtcttcgtcctggggttcctggggttcctcgcgacggcggggtcggcaatgggagccgccagcctgaccctcacggcacagtcccgaactttattggctgggatcgtccaacaacagcagcagctgctggacgtggtcaagaggcagcaggagctgctgcggctgaccgtctggggcacgaagaacctccagacgagggtcacggccatcgagaagtacctgaaggaccaggcgcagctgaacgcgtggggctgtgcgtttcgacaagtctgccacacgacggtcccgtggccgaacgcgtcgctgacgccgaagtggaacaacgagacgtggcaggagtgggagcggaaggtggacttcctggaggagaacatcacggccctcctggaggaggcgcagatccagcaggagaagaacatgtacgagctgcaaaagctgaacagctgggacgtgttcggcaactggttcgacctggcgtcgtggatcaagtacatccagtacggcgtgtacatcgtggtgggggtgatcctgctgcggatcgtgatctacatcgtccagatgctggcgaagctgcggcagggctataggccagtgttctcttccccaccctcttatttccaacaaacccatatccaacaagacccggcgctgccgacccgggagggcaaggagcgggacggcggggagggcggcggcaacagctcctggccgtggcagatcgagtacatccactttcttattcgtcagcttattagactcctgacgtggctgttcagtaactgtaggactctgctgtcgagggtgtaccagatcctccagccgatcctccagcggctctcggcgaccctccagaggattcgggaggtcctccggacggagctgacctacctccagtacgggtggagctatttccacgaggcggtccaggccgtctggcggtcggcgacggagacgctggcgggcgcgtggggcgacctgtgggagacgctgcggcggggcggccggtggatactcgcgatcccccggcggatcaggcaggggctggagctcacgctcctgtgataagatatcggatctgctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcagcacagcaagggggaggattgggaagacaatagcaggcatgctggggatgcggtgggctctatgggtacccaggtgctgaagaattgacccggttcctcctgggccagaaagaagcaggcacatccccttctctgtgacacaccctgtccacgcccctggttcttagttccagccccactcataggacactcatagctcaggagggctccgccttcaatcccacccgctaaagtacttggagcggtctctccctccctcatcagcccaccaaaccaaacctagcctccaagagtgggaagaaattaaagcaagataggctattaagtgcagagggagagaaaatgcctccaacatgtgaggaagtaatgagagaaatcatagaatttcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccggggggggggggcgctgaggtctgcctcgtgaagaaggtgttgctgactcataccaggcctgaatcgccccatcatccagccagaaagtgagggagccacggttgatgagagctttgttgtaggtggaccagttggtgattttgaacttttgctttgccacggaacggtctgcgttgtcgggaagatgcgtgatctgatccttcaactcagcaaaagttcgatttattcaacaaagccgccgtcccgtcaagtcagcgtaatgctctgccagtgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacagttttattgttcatgatgatatatttttatcttgtgcaatgtaacatcagagattttgagacacaacgtggctttccccccccccccattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcagattggctattgg

SEQ ID NO: 71 - protein, 961 aa, Artificial Sequence
  Description of Artificial SequenceIP10-MCP3-SIVmac239 env fusion protein
MNPSAAVIFCLILLGLSGTQGILDMAQPVGINTSTTCCYRFINKKIPKQRLESYRRTTSSHCPREAVIFKTKLDKEICADPTQKWVQDFMKHLDKKTQTPKLICSLYVTVFYGVPAWRNATIPLFCATKNRDTWGTTQCLPDNGDYSEVALNVTESFDAWNNTVTEQAIEDVWQLFETSIKPCVKLSPLCITMRCNKSETDRWGLTKSITTTASTTSTTASAKVDMVNETSSCIAQDNCTGLEQEQMISCKFNMTGLKRDKKKEYNETWYSADLVCEQGNNTGNESRCYMNHCNTSVIQESCDKHYWDAIRFRYCAPPGYALLRCNDTNYSGFMPKCSKVVVSSCTRMMETQTSTWFGFNGTRAENRTYIYWHGRDNRTIISLNKYYNLTMKCRRPGNKTVLPVTIMSGLVFHSQPINDRPKQAWCWFGGKWKDAIKEVKQTIVKHPRYTGTNNTDKINLTAPGGGDPEVTFMWTNCRGEFLYCKMNWFLNWVEDRNTANQKPKEQHKRNYVPCHIRQIINTWHKVGKNVYLPPREGDLTCNSTVTSLIANIDWIDGNQTNITMSAEVAELYRLELGDYKLVEITPIGLAPTDVKRYTTGGTSRNKRGVFVLGFLGFLATAGSAMGAASLTLTAQSRTLLAGIVQQQQQLLDVVKRQQELLRLTVWGTKNLQTRVTAIEKYLKDQAQLNAWGCAFRQVCHTTVPWPNASLTPKWNNETWQEWERKVDFLEENITALLEEAQIQQEKNMYELQKLNSWDVFGNWFDLASWIKYIQYGVYIVVGVILLRIVIYIVQMLAKLRQGYRPVFSSPPSYFQQTHIQQDPALPTREGKERDGGEGGGNSSWPWQIEYIHFLIRQLIRLLTWLFSNCRTLLSRVYQILQPILQRLSATLQRIREVLRTELTYLQYGWSYFHEAVQAVWRSATETLAGAWGDLWETLRRGGRWILAIPRRIRQGLELTLL

SEQ ID NO: 72 - DNA, 6705 bp, Artificial Sequence
  Description of Artificial Sequenceplasmid CMVtPAenvmac239
cctggccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccgcgggcgcgcgtcgaggaattcaagaaatggatgcaatgaagagagggctctgctgtgtgctgctgctgtgtggagcagtcttcgtttcgcccagccaggaaatccatgcccgattcagaagaggagccagatctatctgcagcctgtacgtcacggtcttctacggcgtaccagcttggaggaatgcgacaattcccctcttttgtgcaaccaagaatagggatacttggggaacaactcagtgcctaccggacaacggggactactcggaggtggccctgaacgtgacggagagcttcgacgcctggaacaacacggtcacggagcaggcgatcgaggacgtgtggcagctgttcgagacctcgatcaagccgtgcgtcaagctgtccccgctctgcatcacgatgcggtgcaacaagagcgagacggatcggtgggggctgacgaagtcgatcacgacgacggcgtcgaccacgtcgacgacggcgtcggcgaaagtggacatggtcaacgagacctcgtcgtgcatcgcccaggacaactgcacgggcctggagcaggagcagatgatcagctgcaagttcaacatgacggggctgaagcgggacaagaagaaggagtacaacgagacgtggtactcggcggacctggtgtgcgagcaggggaacaacacggggaacgagtcgcggtgctacatgaaccactgcaacacgtcggtgatccaggagtcgtgcgacaagcactactgggacgcgatccggttccggtactgcgcgccgccgggctacgcgctgctgcggtgcaacgacacgaactactcgggcttcatgccgaaatgctcgaaggtggtggtctcgtcgtgcacgaggatgatggagacgcagacctcgacgtggttcggcttcaacgggacgcgggcggagaaccggacgtacatctactggcacgggcgggacaaccggacgatcatctcgctgaacaagtactacaacctgacgatgaagtgccggcggccgggcaacaagacggtgctcccggtcaccatcatgtcggggctggtgttccactcgcagccgatcaacgaccggccgaagcaggcgtggtgctggttcggggggaagtggaaggacgcgatcaaggaggtgaagcagaccatcgtcaagcacccccgctacacggggacgaacaacacggacaagatcaacctgacggcgccgggcgggggcgatccggaagttaccttcatgtggacaaattgcagaggagagttcctctactgcaagatgaactggttcctgaactgggtggaggacaggaacacggcgaaccagaagccgaaggagcagcacaagcggaactacgtgccgtgccacattcggcagatcatcaacacgtggcacaaagtgggcaagaacgtgtacctgccgccgagggagggcgacctcacgtgcaactccacggtgacctccctcatcgcgaacatcgactggatcgacggcaaccagacgaacatcaccatgtcggcggaggtggcggagctgtaccggctggagctgggggactacaagctggtggagatcacgccgatcggcctggcccccaccgatgtgaagcgctacacgaccggggggacgtcgcggaacaagcggggggtcttcgtcctggggttcctggggttcctcgcgacggcggggtcggcaatgggagccgccagcctgaccctcacggcacagtcccgaactttattggctgggatcgtccaacaacagcagcagctgctggacgtggtcaagaggcagcaggagctgctgcggctgaccgtctggggcacgaagaacctccagacgagggtcacggccatcgagaagtacctgaaggaccaggcgcagctgaacgcgtggggctgtgcgtttcgacaagtctgccacacgacggtcccgtggccgaacgcgtcgctgacgccgaagtggaacaacgagacgtggcaggagtgggagcggaaggtggacttcctggaggagaacatcacggccctcctggaggaggcgcagatccagcaggagaagaacatgtacgagctgcaaaagctgaacagctgggacgtgttcggcaactggttcgacctggcgtcgtggatcaagtacatccagtacggcgtgtacatcgtggtgggggtgatcctgctgcggatcgtgatctacatcgtccagatgctggcgaagctgcggcagggctataggccagtgttctcttccccaccctcttatttccaacaaacccatatccaacaagacccggcgctgccgacccgggagggcaaggagcgggacggcggggagggcggcggcaacagctcctggccgtggcagatcgagtacatccactttcttattcgtcagcttattagactcctgacgtggctgttcagtaactgtaggactctgctgtcgagggtgtaccagatcctccagccgatcctccagcggctctcggcgaccctccagaggattcgggaggtcctccggacggagctgacctacctccagtacgggtggagctatttccacgaggcggtccaggccgtctggcggtcggcgacggagacgctggcgggcgcgtggggcgacctgtgggagacgctgcggcggggcggccggtggatactcgcgatcccccggcggatcaggcaggggctggagctcacgctcctgtgataagatatcggatctgctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcagcacagcaagggggaggattgggaagacaatagcaggcatgctggggatgcggtgggctctatgggtacccaggtgctgaagaattgacccggttcctcctgggccagaaagaagcaggcacatccccttctctgtgacacaccctgtccacgcccctggttcttagttccagccccactcataggacactcatagctcaggagggctccgccttcaatcccacccgctaaagtacttggagcggtctctccctccctcatcagcccaccaaaccaaacctagcctccaagagtgggaagaaattaaagcaagataggctattaagtgcagagggagagaaaatgcctccaacatgtgaggaagtaatgagagaaatcatagaatttcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccggggggggggggcgctgaggtctgcctcgtgaagaaggtgttgctgactcataccaggcctgaatcgccccatcatccagccagaaagtgagggagccacggttgatgagagctttgttgtaggtggaccagttggtgattttgaacttttgctttgccacggaacggtctgcgttgtcgggaagatgcgtgatctgatccttcaactcagcaaaagttcgatttattcaacaaagccgccgtcccgtcaagtcagcgtaatgctctgccagtgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacagttttattgttcatgatgatatatttttatcttgtgcaatgtaacatcagagattttgagacacaacgtggctttccccccccccccattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcagattggctattgg

SEQ ID NO: 73 - DNA, 2688 bp, Artificial Sequence
  Description of Artificial SequencetPA-env gene
atggatgcaatgaagagagggctctgctgtgtgctgctgctgtgtggagcagtcttcgtttcgcccagccaggaaatccatgcccgattcagaagaggagccagatctatctgcagcctgtacgtcacggtcttctacggcgtaccagcttggaggaatgcgacaattcccctcttttgtgcaaccaagaatagggatacttggggaacaactcagtgcctaccggacaacggggactactcggaggtggccctgaacgtgacggagagcttcgacgcctggaacaacacggtcacggagcaggcgatcgaggacgtgtggcagctgttcgagacctcgatcaagccgtgcgtcaagctgtccccgctctgcatcacgatgcggtgcaacaagagcgagacggatcggtgggggctgacgaagtcgatcacgacgacggcgtcgaccacgtcgacgacggcgtcggcgaaagtggacatggtcaacgagacctcgtcgtgcatcgcccaggacaactgcacgggcctggagcaggagcagatgatcagctgcaagttcaacatgacggggctgaagcgggacaagaagaaggagtacaacgagacgtggtactcggcggacctggtgtgcgagcaggggaacaacacggggaacgagtcgcggtgctacatgaaccactgcaacacgtcggtgatccaggagtcgtgcgacaagcactactgggacgcgatccggttccggtactgcgcgccgccgggctacgcgctgctgcggtgcaacgacacgaactactcgggcttcatgccgaaatgctcgaaggtggtggtctcgtcgtgcacgaggatgatggagacgcagacctcgacgtggttcggcttcaacgggacgcgggcggagaaccggacgtacatctactggcacgggcgggacaaccggacgatcatctcgctgaacaagtactacaacctgacgatgaagtgccggcggccgggcaacaagacggtgctcccggtcaccatcatgtcggggctggtgttccactcgcagccgatcaacgaccggccgaagcaggcgtggtgctggttcggggggaagtggaaggacgcgatcaaggaggtgaagcagaccatcgtcaagcacccccgctacacggggacgaacaacacggacaagatcaacctgacggcgccgggcgggggcgatccggaagttaccttcatgtggacaaattgcagaggagagttcctctactgcaagatgaactggttcctgaactgggtggaggacaggaacacggcgaaccagaagccgaaggagcagcacaagcggaactacgtgccgtgccacattcggcagatcatcaacacgtggcacaaagtgggcaagaacgtgtacctgccgccgagggagggcgacctcacgtgcaactccacggtgacctccctcatcgcgaacatcgactggatcgacggcaaccagacgaacatcaccatgtcggcggaggtggcggagctgtaccggctggagctgggggactacaagctggtggagatcacgccgatcggcctggcccccaccgatgtgaagcgctacacgaccggggggacgtcgcggaacaagcggggggtcttcgtcctggggttcctggggttcctcgcgacggcggggtcggcaatgggagccgccagcctgaccctcacggcacagtcccgaactttattggctgggatcgtccaacaacagcagcagctgctggacgtggtcaagaggcagcaggagctgctgcggctgaccgtctggggcacgaagaacctccagacgagggtcacggccatcgagaagtacctgaaggaccaggcgcagctgaacgcgtggggctgtgcgtttcgacaagtctgccacacgacggtcccgtggccgaacgcgtcgctgacgccgaagtggaacaacgagacgtggcaggagtgggagcggaaggtggacttcctggaggagaacatcacggccctcctggaggaggcgcagatccagcaggagaagaacatgtacgagctgcaaaagctgaacagctgggacgtgttcggcaactggttcgacctggcgtcgtggatcaagtacatccagtacggcgtgtacatcgtggtgggggtgatcctgctgcggatcgtgatctacatcgtccagatgctggcgaagctgcggcagggctataggccagtgttctcttccccaccctcttatttccaacaaacccatatccaacaagacccggcgctgccgacccgggagggcaaggagcgggacggcggggagggcggcggcaacagctcctggccgtggcagatcgagtacatccactttcttattcgtcagcttattagactcctgacgtggctgttcagtaactgtaggactctgctgtcgagggtgtaccagatcctccagccgatcctccagcggctctcggcgaccctccagaggattcgggaggtcctccggacggagctgacctacctccagtacgggtggagctatttccacgaggcggtccaggccgtctggcggtcggcgacggagacgctggcgggcgcgtggggcgacctgtgggagacgctgcggcggggcggccggtggatactcgcgatcccccggcggatcaggcaggggctggagctcacgctcctgtga

SEQ ID NO: 74 - protein, 895 aa, Artificial Sequence
  Description of Artificial SequencetPA-env fusion protein
MDAMKRGLCCVLLLCGAVFVSPSQEIHARFRRGARSICSLYVTVFYGVPAWRNATIPLFCATKNRDTWGTTQCLPDNGDYSEVALNVTESFDAWNNTVTEQAIEDVWQLFETSIKPCVKLSPLCITMRCNKSETDRWGLTKSITTTASTTSTTASAKVDMVNETSSCIAQDNCTGLEQEQMISCKFNMTGLKRDKKKEYNETWYSADLVCEQGNNTGNESRCYMNHCNTSVIQESCDKHYWDAIRFRYCAPPGYALLRCNDTNYSGFMPKCSKVVVSSCTRMMETQTSTWFGFNGTRAENRTYIYWHGRDNRTIISLNKYYNLTMKCRRPGNKTVLPVTIMSGLVFHSQPINDRPKQAWCWFGGKWKDAIKEVKQTIVKHPRYTGTNNTDKINLTAPGGGDPEVTFMWTNCRGEFLYCKMNWFLNWVEDRNTANQKPKEQHKRNYVPCHIRQIINTWHKVGKNVYLPPREGDLTCNSTVTSLIANIDWIDGNQTNITMSAEVAELYRLELGDYKLVEITPIGLAPTDVKRYTTGGTSRNKRGVFVLGFLGFLATAGSAMGAASLTLTAQSRTLLAGIVQQQQQLLDVVKRQQELLRLTVWGTKNLQTRVTAIEKYLKDQAQLNAWGCAFRQVCHTTVPWPNASLTPKWNNETWQEWERKVDFLEENITALLEEAQIQQEKNMYELQKLNSWDVFGNWFDLASWIKYIQYGVYIVVGVILLRIVIYIVQMLAKLRQGYRPVFSSPPSYFQQTHIQQDPALPTREGKERDGGEGGGNSSWPWQIEYIHFLIRQLIRLLTWLFSNCRTLLSRVYQILQPILQRLSATLQRIREVLRTELTYLQYGWSYFHEAVQAVWRSATETLAGAWGDLWETLRRGGRWILAIPRRIRQGLELTLL

SEQ ID NO: 75 - DNA, 5444 bp, Artificial Sequence
  Description of Artificial SequencepCMV MCP3p39 (SIV)
cctggccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccgcgggcgcgcgtcgacaagaaatgaacccaagtgctgccgtcattttctgcctcatcctgctgggtctgagtgggactcaagggatcctcgacatggcgcaaccggtcgggatcaacacgagcacgacctgctgctaccggttcatcaacaagaagatcccgaagcaacgtctggaaagctatcgccggaccacgtcgagccactgcccgcgggaggcggttatcttcaagacgaagctggacaaggagatctgcgccgacccgacgcagaagtgggttcaggacttcatgaagcacctggataagaagacgcagacgccgaagctggctagcgcaggagcaggcgtgcggaactccgtcttgtcggggaagaaagcggatgagttggagaaaattcggctacggcccaacgggaagaagaagtacatgttgaagcatgtagtatgggcggcgaatgagttggatcggtttggattggcggagagcctgttggagaacaaagagggatgtcagaagatcctttcggtcttggcgccgttggtgccgacgggctcggagaacttgaagagcctctacaacacggtctgcgtcatctggtgcattcacgcggaagagaaagtgaaacacacggaggaagcgaaacagatagtgcagcggcacctagtggtggaaacgggaaccaccgaaaccatgccgaagacctcgcggccgacggcgccgtcgagcggcaggggaggaaactacccggtacagcagatcggtggcaactacgtccacctgccgctgtccccgcggaccctgaacgcgtgggtcaagctgatcgaggagaagaagttcggagcggaggtagtgccgggattccaggcgctgtcggaaggttgcaccccctacgacatcaaccagatgctgaactgcgttggagaccatcaggcggcgatgcagatcatccgggacatcatcaacgaggaggcggcggattgggacttgcagcacccgcaaccggcgccgcaacaaggacaacttcgggagccgtcgggatcggacatcgcgggaaccacctcctcggttgacgaacagatccagtggatgtaccggcagcagaacccgatcccagtaggcaacatctaccggcggtggatccagctgggtctgcagaaatgcgtccgtatgtacaacccgaccaacattctagatgtaaaacaagggccaaaggagccgttccagagctacgtcgaccggttctacaagtcgctgcgggcggagcagacggacgcggcggtcaagaactggatgacgcagacgctgctgatccagaacgcgaacccagattgcaagctagtgctgaaggggctgggtgtgaatcccaccctagaagaaatgctgacggcttgtcaaggagtaggggggccgggacagaaggctagattaatgggggcccatgcggccgcgtaggaattcgatccagatctgctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcagcacagcaagggggaggattgggaagacaatagcaggcatgctggggatgcggtgggctctatgggtacccaggtgctgaagaattgacccggttcctcctgggccagaaagaagcaggcacatccccttctctgtgacacaccctgtccacgcccctggttcttagttccagccccactcataggacactcatagctcaggagggctccgccttcaatcccacccgctaaagtacttggagcggtctctccctccctcatcagcccaccaaaccaaacctagcctccaagagtgggaagaaattaaagcaagataggctattaagtgcagagggagagaaaatgcctccaacatgtgaggaagtaatgagagaaatcatagaatttcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccggggggggggggcgctgaggtctgcctcgtgaagaaggtgttgctgactcataccaggcctgaatcgccccatcatccagccagaaagtgagggagccacggttgatgagagctttgttgtaggtggaccagttggtgattttgaacttttgctttgccacggaacggtctgcgttgtcgggaagatgcgtgatctgatccttcaactcagcaaaagttcgatttattcaacaaagccgccgtcccgtcaagtcagcgtaatgctctgccagtgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacagttttattgttcatgatgatatatttttatcttgtgcaatgtaacatcagagattttgagacacaacgtggctttccccccccccccattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcagattggctattgg

SEQ ID NO: 76 - protein, 476 aa, Artificial Sequence
  Description of Artificial Sequence MCP3-p39SIVgag fusion protein
MNPSAAVIFCLILLGLSGTQGILDMAQPVGINTSTTCCYRFINKKIPKQRLESYRRTTSSHCPREAVIFKTKLDKEICADPTQKWVQDFMKHLDKKTQTPKLASAGAGVRNSVLSGKKADELEKIRLRPNGKKKYMLKHVVWAANELDRFGLAESLLENKEGCQKILSVLAPLVPTGSENLKSLYNTVCVIWCIHAEEKVKHTEEAKQIVQRHLVVETGTTETMPKTSRPTAPSSGRGGNYPVQQIGGNYVHLPLSPRTLNAWVKLIEEKKFGAEVVPGFQALSEGCTPYDINQMLNCVGDHQAAMQIIRDIINEEAADWDLQHPQPAPQQGQLREPSGSDIAGTTSSVDEQIQWMYRQQNPIPVGNIYRRWIQLGLQKCVRMYNPTNILDVKQGPKEPFQSYVDRFYKSLRAEQTDAAVKNWMTQTLLIQNANPDCKLVLKGLGVNPTLEEMLTACQGVGGPGQKARLMGAHAAA

SEQ ID NO: 77 - DNA, 1197 bp, Artificial Sequence
  Description of Artificial Sequence CATEp37gag(HIV)
atgagaaaagcggctgttagtcactggcagcaacagtcttacctggactctggaatccattctggtgccactaccacagctccttctctgagtgtcgacagagagatgggtgcgagagcgtcagtattaagcgggggagaattagatcgatgggaaaaaattcggttaaggccagggggaaagaagaagtacaagctaaagcacatcgtatgggcaagcagggagctagaacgattcgcagttaatcctggcctgttagaaacatcagaaggctgtagacaaatactgggacagctacaaccatcccttcagacaggatcagaggagcttcgatcactatacaacacagtagcaaccctctattgtgtgcaccagcggatcgagatcaaggacaccaaggaagctttagacaagatagaggaagagcaaaacaagtccaagaagaaggcccagcaggcagcagctgacacaggacacagcaatcaggtcagccaaaattaccctatagtgcagaacatccaggggcaaatggtacatcaggccatatcacctagaactttaaatgcatgggtaaaagtagtagaagagaaggctttcagcccagaagtgatacccatgttttcagcattatcagaaggagccaccccacaggacctgaacacgatgttgaacaccgtggggggacatcaagcagccatgcaaatgttaaaagagaccatcaatgaggaagctgcagaatgggatagagtgcatccagtgcatgcagggcctattgcaccaggccagatgagagaaccaaggggaagtgacatagcaggaactactagtacccttcaggaacaaataggatggatgacaaataatccacctatcccagtaggagagatctacaagaggtggataatcctgggattgaacaagatcgtgaggatgtatagccctaccagcattctggacataagacaaggaccaaaggaaccctttagagactatgtagaccggttctataaaactctaagagctgagcaagcttcacaggaggtaaaaaattggatgacagaaaccttgttggtccaaaatgcgaacccagattgtaagaccatcctgaaggctctcggcccagcggctacactagaagaaatgatgacagcatgtcagggagtaggaggacccggccataaggcaagagttttgtag

SEQ ID NO: 78 - protein, 398 aa, Artificial Sequence
  Description of Artificial Sequence CATEp37gag(HIV) fusion protein
MRKAAVSHWQQQSYLDSGIHSGATTTAPSLSVDREMGARASVLSGGELDRWEKIRLRPGGKKKYKLKHIVWASRELERFAVNPGLLETSEGCRQILGQLQPSLQTGSEELRSLYNTVATLYCVHQRIEIKDTKEALDKIEEEQNKSKKKAQQAAADTGHSNQVSQNYPIVQNIQGQMVHQAISPRTLNAWVKVVEEKAFSPEVIPMFSALSEGATPQDLNTMLNTVGGHQAAMQMLKETINEEAAEWDRVHPVHAGPIAPGQMREPRGSDIAGTTSTLQEQIGWMTNNPPIPVGEIYKRWIILGLNKIVRMYSPTSILDIRQGPKEPFRDYVDRFYKTLRAEQASQEVKNWMTETLLVQNANPDCKTILKALGPAATLEEMMTACQGVGGPGHKARVL

SEQ ID NO: 79 - DNA, 4317 bp, Artificial Sequence
  Description of Artificial SequencePolNTV (HIV)
cctcagatcacgctctggcagcggccgctcgtcacaataaagatcggggggcaactcaaggaggcgctgctcgcggacgacacggtcttggaggagatgtcgttgccggggcggtggaagccgaagatgatcggggggatcgggggcttcatcaaggtgcggcagtacgaccagatcctcatcgagatctgcgggcacaaggcgatcgggacggtcctcgtcggcccgacgccggtcaacatcatcgggcggaacctgttgacccagatcggctgcaccttgaacttccccatcagccctattgagacggtgcccgtgaagttgaagccggggatggacggccccaaggtcaagcaatggccattgacggaggagaagatcaaggccttagtcgaaatctgtacagagatggagaaggaagggaagatcagcaagatcgggcctgagaacccctacaacactccagtcttcgcaatcaagaagaaggacagtaccaagtggagaaagctggtggacttcagagagctgaacaagagaactcaggacttctgggaagttcagctgggcatcccacatcccgctgggttgaagaagaagaagtcagtgacagtgctggatgtgggtgatgcctacttctccgttcccttggacgaggacttcaggaagtacactgccttcacgatacctagcatcaacaacgagacaccaggcatccgctaccagtacaacgtgctgccacagggatggaagggatcaccagccatctttcaatcgtcgatgaccaagatcctggagcccttccgcaagcaaaacccagacatcgtgatctatcagctctacgtaggaagtgacctggagatcgggcagcacaggaccaagatcgaggagctgagacagcatctgttgaggtggggactgaccacaccagacaagaagcaccagaaggaacctcccttcctgtggatgggctacgaactgcatcctgacaagtggacagtgcagcccatcgtgctgcctgagaaggacagctggactgtgaacgacatacagaagctcgtgggcaagttgaactgggcaagccagatctacccaggcatcaaagttaggcagctgtgcaagctgcttcgaggaaccaaggcactgacagaagtgatcccactgacagaggaagcagagctagaactggcagagaaccgagagatcctgaaggagccagtacatggagtgtactacgacccaagcaaggacctgatcgcagagatccagaagcaggggcaaggccaatggacctaccaaatctaccaggagcccttcaagaacctgaagacaggcaagtacgcaaggatgaggggtgcccacaccaacgatgtgaagcagctgacagaggcagtgcagaagatcaccacagagagcatcgtgatctggggcaagactcccaagttcaagctgcccatacagaaggagacatgggagacatggtggaccgagtactggcaagccacctggatccctgagtgggagttcgtgaacacccctcccttggtgaaactgtggtatcagctggagaaggaacccatcgtgggagcagagaccttctacgtggatggggcagccaacagggagaccaagctgggcaaggcaggctacgtgaccaaccgaggacgacagaaagtggtgaccctgactgacaccaccaaccagaagactctgcaagccatctacctagctctgcaagacagcggactggaagtgaacatcgtgacagactcacagtacgcactgggcatcatccaagcacaaccagaccaatccgagtcagagctggtgaaccagatcatcgagcagctgatcaagaaggagaaagtgtacctggcatgggtcccggcgcacaaggggatcggggggaacgagcaggtcgacaagttggtctcggcggggatccggaaggtgctgttcctggacgggatcgataaggcccaagatgaacatgagaagtaccactccaactggcgcgctatggccagcgacttcaacctgccgccggtcgtcgcgaaggagatcgtcgccagctgcgacaagtgccagctcaagggggaggccatgcacgggcaagtcgactgcagtccggggatctggcagctgtgcacgcacctggaggggaaggtgatcctggtcgcggtccacgtcgccagcgggtatatcgaggcggaggtcatcccggctgagacggggcaggagacggcgtacttcctcttgaagctcgcggggcggtggccggtcaagacgatccacacgaacgggagcaacttcacgggggcgacggtcaaggccgcctgttggtgggcgggaatcaagcaggaatttggaattccctacaatccccaatcgcaaggagtcgtgagcatgaacaaggagctgaagaagatcatcggacaaagggatcaggctgagcacctgaagacagcagtgcagatggcagtgttcatccacaacttcaaaagaaaaggggggattggggggtacagtgcgggggaacggatcgtggacatcatcgccaccgacatccaaaccaaggagctgcagaagcagatcaccaagatccagaacttccgggtgtactaccgcgacagccgcaacccactgtggaagggaccagcaaagctcctctggaagggagagggggcagtggtgatccaggacaacagtgacatcaaagtggtgccaaggcgcaaggccaagatcatccgcgactatggaaaacagatggcaggggatgattgtgtggcaagtagacaggatgaggatggcgccgctagcaagtggtcgaagtcgtcggtgatcgggtggccgactgttcgggagcggatgcggcgggcggagccggcggcggatcgggtgggagcggcgtcgcgggaccttgagaagcacggggcgatcacgtcgagcaacacggcggcgacgaatgcggcgtgtgcctggctagaggcgcaagaggaggaggaagtgggttttccggtcacgccgcaggtcccgcttcggccgatgacgtacaaggcagcggtcgacctcagccacttcctcaaggagaaggggggactggaggggctcatccactcccagcggcggcaggacatccttgacctgtggatctaccacacacaaggctacttcccggattggcagaactacacgccggggccgggggtccggtatccgctgacctttggatggtgctacaagctagtaccggttgagccggataagatcgaggaggccaacaagggagagaacaccagcttgttgcaccctgtgagcctgcatggaatggatgacccggagcgggaggtgcttgagtggcggtttgacagccgcctagcgtttcatcacgtggcccgagagctgcatccggagtacttcaagaactgcggatccgagccagtagatcctagactagagccctggaagcatccaggatcgcagccgaagacggcgtgcaccaactgctactgcaagaagtgcttccaccaggtctgcttcatgacgaaggccttgggcatctcctatggccggaagaagcggagacagcgacgaagagctcatcagaactcgcagacgcaccaggcgtcgctatcgaagcaacccacctcccaatcccgaggggacccgacaggcccgaaggaatcgaagaaggaggtggagagagagacagagacagatccgttcgactggtctagagagaaccggtggcaggtgatgattgtgtggcaggtcgaccggatgcggattcggacgtggaagtcgcttgtcaagcaccacatgtacatctcggggaaggcgaaggggtggttctaccggcaccactatgagtcgacgcacccgcggatctcgtcggaggtccacatcccgctaggggacgcgaagcttgtcatcacgacgtactggggtctgcatacgggagagcgggactggcatttgggtcagggagtctccatagagtggaggaaaaagcggtatagcacgcaagtagacccggacctagcggaccagctaatccacctgtactacttcgactcgttctcggagtcggcgatacggaataccatccttgggcggatcgtttcgccgcggagtgagtatcaagcggggcacaacaaggtcgggtcgctacagtacttggcgctcgcggcgttgatcacgccgaagcagataaagccgccgttgccgtcggttacgaaactgacggaggaccggtggaacaagccccagaagaccaagggccaccgggggagccacacaatgaacgggcacgttaactag

SEQ ID NO: 80 - protein, 1439 aa, Artificial Sequence
  Description of Artificial SequencePolNTV (HIV) (pol-nef-tat-vif) fusion protein
MPQITLWQRPLVTIKIGGQLKEALLADDTVLEEMSLPGRWKPKMIGGIGGFIKVRQYDQILIEICGHKAIGTVLVGPTPVNIIGRNLLTQIGCTLNFPISPIETVPVKLKPGMDGPKVKQWPLTEEKIKALVEICTEMEKEGKISKIGPENPYNTPVFAIKKKDSTKWRKLVDFRELNKRTQDFWEVQLGIPHPAGLKKKKSVTVLDVGDAYFSVPLDEDFRKYTAFTIPSINNETPGIRYQYNVLPQGWKGSPAIFQSSMTKILEPFRKQNPDIVIYQLYVGSDLEIGQHRTKIEELRQHLLRWGLTTPDKKHQKEPPFLWMGYELHPDKWTVQPIVLPEKDSWTVNDIQKLVGKLNWASQIYPGIKVRQLCKLLRGTKALTEVIPLTEEAELELAENREILKEPVHGVYYDPSKDLIAEIQKQGQGQWTYQIYQEPFKNLKTGKYARMRGAHTNDVKQLTEAVQKITTESIVIWGKTPKFKLPIQKETWETWWTEYWQATWIPEWEFVNTPPLVKLWYQLEKEPIVGAETFYVDGAANRETKLGKAGYVTNRGRQKVVTLTDTTNQKTLQAIYLALQDSGLEVNIVTDSQYALGIIQAQPDQSESELVNQIIEQLIKKEKVYLAWVPAHKGIGGNEQVDKLVSAGIRKVLFLDGIDKAQDEHEKYHSNWRAMASDFNLPPVVAKEIVASCDKCQLKGEAMHGQVDCSPGIWQLCTHLEGKVILVAVHVASGYIEAEVIPAETGQETAYFLLKLAGRWPVKTIHTNGSNFTGATVKAACWWAGIKQEFGIPYNPQSQGVVSMNKELKKIIGQRDQAEHLKTAVQMAVFIHNFKRKGGIGGYSAGERIVDIIATDIQTKELQKQITKIQNFRVYYRDSRNPLWKGPAKLLWKGEGAVVIQDNSDIKVVPRRKAKIIRDYGKQMAGDDCVASRQDEDGAASKWSKSSVIGWPTVRERMRRAEPAADRVGAASRDLEKHGAITSSNTAATNAACAWLEAQEEEEVGFPVTPQVPLRPMTYKAAVDLSHFLKEKGGLEGLIHSQRRQDILDLWIYHTQGYFPDWQNYTPGPGVRYPLTFGWCYKLVPVEPDKIEEANKGENTSLLHPVSLHGMDDPEREVLEWRFDSRLAFHHVARELHPEYFKNCGSEPVDPRLEPWKHPGSQPKTACTNCYCKKCFHQVCFMTKALGISYGRKKRRQRRRAHQNSQTHQASLSKQPTSQSRGDPTGPKESKKEVERETETDPFDWSRENRWQVMIVWQVDRMRIRTWKSLVKHHMYISGKAKGWFYRHHYESTHPRISSEVHIPLGDAKLVITTYWGLHTGERDWHLGQGVSIEWRKKRYSTQVDPDLADQLIHLYYFDSFSESAIRNTILGRIVSPRSEYQAGHNKVGSLQYLALAALITPKQIKPPLPSVTKLTEDRWNKPQKTKGHRGSHTMNGHVN

SEQ ID NO: 81 - DNA, 2598 bp, Artificial Sequence
  Description of Artificial SequencetPAenv (HIV)
atggatgcaatgaagagagggctctgctgtgtgctgctgctgtgtggagcagtcttcgtttcgcccagccaggaaatccatgcccgattcagaagaggagccagatctatctgcagcgccgaggagaagctgtgggtcacggtctattatggcgtgcccgtgtggaaagaggcaaccaccacgctattctgcgcctccgacgccaaggcacatcatgcagaggcgcacaacgtctgggccacgcatgcctgtgtacccacggaccctaacccccaagaggtgatcctggagaacgtgaccgagaagtacaacatgtggaaaaataacatggtagaccagatgcatgaggatataatcagtctatgggatcaaagcctaaagccatgtgtaaaactaacccccctctgcgtgacgctgaattgcaccaacgcgacgtatacgaatagtgacagtaagaatagtaccagtaatagtagtttggaggacagtgggaaaggagacatgaactgctcgttcgatgtcaccaccagcatcgacaagaagaagaagacggagtatgccatcttcgacaagctggatgtaatgaatataggaaatggaagatatacgctattgaattgtaacaccagtgtcattacgcaggcctgtccaaagatgtcctttgagccaattcccatacattattgtaccccggccggctacgcgatcctgaagtgcaacgacaataagttcaatggaacgggaccatgtacgaatgtcagcacgatacaatgtacgcatggaattaagccagtagtgtcgacgcaactgctgctgaacggcagcctggccgagggaggagaggtaataattcggtcggagaacctcaccgacaacgccaagaccataatagtacagctcaaggaacccgtggagatcaactgtacgagacccaacaacaacacccgaaagagcatacatatgggaccaggagcagcattttatgcaagaggagaggtaataggagatataagacaagcacattgcaacattagtagaggaagatggaatgacactttgaaacagatagctaaaaagctgcgcgagcagtttaacaagaccataagccttaaccaatcctcgggaggggacctagagattgtaatgcacacgtttaattgtggaggggagtttttctactgtaacacgacccagctgttcaacagcacctggaatgagaatgatacgacctggaataatacggcagggtcgaataacaatgagacgatcaccctgccctgtcgcatcaagcagatcataaacaggtggcaggaagtaggaaaagcaatgtatgcccctcccatcagtggcccgatcaactgcttgtccaacatcaccgggctattgttgacgagagatggtggtgacaacaataatacgatagagaccttcagacctggaggaggagatatgagggacaactggaggagcgagctgtacaagtacaaggtagtgaggatcgagccattgggaatagcacccaccaaggcaaagagaagagtggtgcaaagagagaaaagagcagtgggaataggagctatgttccttgggttcttgggagcagcaggaagcactatgggcgcagcgtcggtgacccttaccgtgcaagctcgcctgctgctgtcgggtatagtgcaacagcaaaacaacctcctccgcgcaatcgaagcccagcagcatctgttgcaactcacggtctggggcatcaagcagctccaggctagagtccttgccatggagcgttatctgaaagaccagcaacttcttgggatttggggttgctcgggaaaactcatttgcaccacgaatgtgccttggaacgccagctggagcaacaagtccctggacaagatttggcataacatgacctggatggagtgggaccgcgagatcgacaactacacgaaattgatatacaccctgatcgaggcgtcccagatccagcaggagaagaatgagcaagagttgttggagttggattcgtgggcgtcgttgtggtcgtggtttgacatctcgaaatggctgtggtatataggagtattcataatagtaataggaggtttggtaggtttgaaaatagtttttgctgtactttcgatagtaaatcgagttaggcagggatactcgccattgtcatttcaaacccgcctcccagccccgcggggacccgacaggcccgagggcatcgaggagggaggcggcgagagagacagagacagatccgatcaattggtgacgggattcttggcactcatctgggacgatctgcggagcctgtgcctcttctcttaccaccgcctgcgcgacctgctcctgatcgtggcgaggatcgtggagcttctgggacgcagggggtgggaggccctgaagtactggtggaacctcctgcaatattggattcaggagctgaagaacagcgccgttagtctgctgaacgctaccgctatcgccgtggcggaaggaaccgacaggattatagaggtagtacaaaggattggtcgcgccatcctccatatcccccgccgcatccgccagggcttggagagggctttgctataa

SEQ ID NO: 82 - protein, 865 aa, Artificial Sequence
  Description of Artificial SequencetPAenv (HIV) fusion protein
MDAMKRGLCCVLLLCGAVFVSPSQEIHARFRRGARSICSAEEKLWVTVYYGVPVWKEATTTLFCASDAKAHHAEAHNVWATHACVPTDPNPQEVILENVTEKYNMWKNNMVDQMHEDIISLWDQSLKPCVKLTPLCVTLNCTNATYTNSDSKNSTSNSSLEDSGKGDMNCSFDVTTSIDKKKKTEYAIFDKLDVMNIGNGRYTLLNCNTSVITQACPKMSFEPIPIHYCTPAGYAILKCNDNKFNGTGPCTNVSTIQCTHGIKPVVSTQLLLNGSLAEGGEVIIRSENLTDNAKTIIVQLKEPVEINCTRPNNNTRKSIHMGPGAAFYARGEVIGDIRQAHCNISRGRWNDTLKQIAKKLREQFNKTISLNQSSGGDLEIVMHTFNCGGEFFYCNTTQLFNSTWNENDTTWNNTAGSNNNETITLPCRIKQIINRWQEVGKAMYAPPISGPINCLSNITGLLLTRDGGDNNNTIETFRPGGGDMRDNWRSELYKYKVVRIEPLGIAPTKAKRRVVQREKRAVGIGAMFLGFLGAAGSTMGAASVTLTVQARLLLSGIVQQQNNLLRAIEAQQHLLQLTVWGIKQLQARVLAMERYLKDQQLLGIWGCSGKLICTTNVPWNASWSNKSLDKIWHNMTWMEWDREIDNYTKLIYTLIEASQIQQEKNEQELLELDSWASLWSWFDISKWLWYIGVFIIVIGGLVGLKIVFAVLSIVNRVRQGYSPLSFQTRLPAPRGPDRPEGIEEGGGERDRDRSDQLVTGFLALIWDDLRSLCLFSYHRLRDLLLIVARIVELLGRRGWEALKYWWNLLQYWIQELKNSAVSLLNATAIAVAEGTDRIIEVVQRIGRAILHIPRRIRQGLERALL

SEQ ID NO: 83 - DNA, 2796 bp, Artificial Sequence
  Description of Artificial SequenceMCP3 HIVenv
atgaacccaagtgctgccgtcattttctgcctcatcctgctgggtctgagtgggactcaagggatcctcgacatggcgcaaccggtaggtataaacacaagcacaacctgttgctatcgtttcataaataaaaagataccgaagcaacgtctggaaagctatcgccgtaccacttctagccactgtccgcgtgaagctgttatattcaaaacgaaactggataaggagatctgcgccgaccctacacagaaatgggttcaggactttatgaagcacctggataaaaagacacagacgccgaaactgatctgcagcgccgaggagaagctgtgggtcacggtctattatggcgtgcccgtgtggaaagaggcaaccaccacgctattctgcgcctccgacgccaaggcacatcatgcagaggcgcacaacgtctgggccacgcatgcctgtgtacccacggaccctaacccccaagaggtgatcctggagaacgtgaccgagaagtacaacatgtggaaaaataacatggtagaccagatgcatgaggatataatcagtctatgggatcaaagcctaaagccatgtgtaaaactaacccccctctgcgtgacgctgaattgcaccaacgcgacgtatacgaatagtgacagtaagaatagtaccagtaatagtagtttggaggacagtgggaaaggagacatgaactgctcgttcgatgtcaccaccagcatcgacaagaagaagaagacggagtatgccatcttcgacaagctggatgtaatgaatataggaaatggaagatatacgctattgaattgtaacaccagtgtcattacgcaggcctgtccaaagatgtcctttgagccaattcccatacattattgtaccccggccggctacgcgatcctgaagtgcaacgacaataagttcaatggaacgggaccatgtacgaatgtcagcacgatacaatgtacgcatggaattaagccagtagtgtcgacgcaactgctgctgaacggcagcctggccgagggaggagaggtaataattcggtcggagaacctcaccgacaacgccaagaccataatagtacagctcaaggaacccgtggagatcaactgtacgagacccaacaacaacacccgaaagagcatacatatgggaccaggagcagcattttatgcaagaggagaggtaataggagatataagacaagcacattgcaacattagtagaggaagatggaatgacactttgaaacagatagctaaaaagctgcgcgagcagtttaacaagaccataagccttaaccaatcctcgggaggggacctagagattgtaatgcacacgtttaattgtggaggggagtttttctactgtaacacgacccagctgttcaacagcacctggaatgagaatgatacgacctggaataatacggcagggtcgaataacaatgagacgatcaccctgccctgtcgcatcaagcagatcataaacaggtggcaggaagtaggaaaagcaatgtatgcccctcccatcagtggcccgatcaactgcttgtccaacatcaccgggctattgttgacgagagatggtggtgacaacaataatacgatagagaccttcagacctggaggaggagatatgagggacaactggaggagcgagctgtacaagtacaaggtagtgaggatcgagccattgggaatagcacccaccaaggcaaagagaagagtggtgcaaagagagaaaagagcagtgggaataggagctatgttccttgggttcttgggagcagcaggaagcactatgggcgcagcgtcggtgacccttaccgtgcaagctcgcctgctgctgtcgggtatagtgcaacagcaaaacaacctcctccgcgcaatcgaagcccagcagcatctgttgcaactcacggtctggggcatcaagcagctccaggctagagtccttgccatggagcgttatctgaaagaccagcaacttcttgggatttggggttgctcgggaaaactcatttgcaccacgaatgtgccttggaacgccagctggagcaacaagtccctggacaagatttggcataacatgacctggatggagtgggaccgcgagatcgacaactacacgaaattgatatacaccctgatcgaggcgtcccagatccagcaggagaagaatgagcaagagttgttggagttggattcgtgggcgtcgttgtggtcgtggtttgacatctcgaaatggctgtggtatataggagtattcataatagtaataggaggtttggtaggtttgaaaatagtttttgctgtactttcgatagtaaatcgagttaggcagggatactcgccattgtcatttcaaacccgcctcccagccccgcggggacccgacaggcccgagggcatcgaggagggaggcggcgagagagacagagacagatccgatcaattggtgacgggattcttggcactcatctgggacgatctgcggagcctgtgcctcttctcttaccaccgcctgcgcgacctgctcctgatcgtggcgaggatcgtggagcttctgggacgcagggggtgggaggccctgaagtactggtggaacctcctgcaatattggattcaggagctgaagaacagcgccgttagtctgctgaacgctaccgctatcgccgtggcggaaggaaccgacaggattatagaggtagtacaaaggattggtcgcgccatcctccatatcccccgccgcatccgccagggcttggagagggctttgctataa

SEQ ID NO: 84 - protein, 931 aa, Artificial Sequence
  Description of Artificial SequenceMCP3 HIVenv fusion protein
MNPSAAVIFCLILLGLSGTQGILDMAQPVGINTSTTCCYRFINKKIPKQRLESYRRTTSSHCPREAVIFKTKLDKEICADPTQKWVQDFMKHLDKKTQTPKLICSAEEKLWVTVYYGVPVWKEATTTLFCASDAKAHHAEAHNVWATHACVPTDPNPQEVILENVTEKYNMWKNNMVDQMHEDIISLWDQSLKPCVKLTPLCVTLNCTNATYTNSDSKNSTSNSSLEDSGKGDMNCSFDVTTSIDKKKKTEYAIFDKLDVMNIGNGRYTLLNCNTSVITQACPKMSFEPIPIHYCTPAGYAILKCNDNKFNGTGPCTNVSTIQCTHGIKPVVSTQLLLNGSLAEGGEVIIRSENLTDNAKTIIVQLKEPVEINCTRPNNNTRKSIHMGPGAAFYARGEVIGDIRQAHCNISRGRWNDTLKQIAKKLREQFNKTISLNQSSGGDLEIVMHTFNCGGEFFYCNTTQLFNSTWNENDTTWNNTAGSNNNETITLPCRIKQIINRWQEVGKAMYAPPISGPINCLSNITGLLLTRDGGDNNNTIETFRPGGGDMRDNWRSELYKYKVVRIEPLGIAPTKAKRRVVQREKRAVGIGAMFLGFLGAAGSTMGAASVTLTVQARLLLSGIVQQQNNLLRAIEAQQHLLQLTVWGIKQLQARVLAMERYLKDQQLLGIWGCSGKLICTTNVPWNASWSNKSLDKIWHNMTWMEWDREIDNYTKLIYTLIEASQIQQEKNEQELLELDSWASLWSWFDISKWLWYIGVFIIVIGGLVGLKIVFAVLSIVNRVRQGYSPLSFQTRLPAPRGPDRPEGIEEGGGERDRDRSDQLVTGFLALIWDDLRSLCLFSYHRLRDLLLIVARIVELLGRRGWEALKYWWNLLQYWIQELKNSAVSLLNATAIAVAEGTDRIIEVVQRIGRAILHIPRRIRQGLERALL

SEQ ID NO: 85 - DNA, 2583 bp, Artificial Sequence
  Description of Artificial SequenceCATEenv(HIV)
atgagaaaagcggctgttagtcactggcagcagcagtcttacctggactctggaatccattctggtgccactaccacagctccttctctgagtatctgcagcgccgaggagaagctgtgggtcacggtctattatggcgtgcccgtgtggaaagaggcaaccaccacgctattctgcgcctccgacgccaaggcacatcatgcagaggcgcacaacgtctgggccacgcatgcctgtgtacccacggaccctaacccccaagaggtgatcctggagaacgtgaccgagaagtacaacatgtggaaaaataacatggtagaccagatgcatgaggatataatcagtctatgggatcaaagcctaaagccatgtgtaaaactaacccccctctgcgtgacgctgaattgcaccaacgcgacgtatacgaatagtgacagtaagaatagtaccagtaatagtagtttggaggacagtgggaaaggagacatgaactgctcgttcgatgtcaccaccagcatcgacaagaagaagaagacggagtatgccatcttcgacaagctggatgtaatgaatataggaaatggaagatatacgctattgaattgtaacaccagtgtcattacgcaggcctgtccaaagatgtcctttgagccaattcccatacattattgtaccccggccggctacgcgatcctgaagtgcaacgacaataagttcaatggaacgggaccatgtacgaatgtcagcacgatacaatgtacgcatggaattaagccagtagtgtcgacgcaactgctgctgaacggcagcctggccgagggaggagaggtaataattcggtcggagaacctcaccgacaacgccaagaccataatagtacagctcaaggaacccgtggagatcaactgtacgagacccaacaacaacacccgaaagagcatacatatgggaccaggagcagcattttatgcaagaggagaggtaataggagatataagacaagcacattgcaacattagtagaggaagatggaatgacactttgaaacagatagctaaaaagctgcgcgagcagtttaacaagaccataagccttaaccaatcctcgggaggggacctagagattgtaatgcacacgtttaattgtggaggggagtttttctactgtaacacgacccagctgttcaacagcacctggaatgagaatgatacgacctggaataatacggcagggtcgaataacaatgagacgatcaccctgccctgtcgcatcaagcagatcataaacaggtggcaggaagtaggaaaagcaatgtatgcccctcccatcagtggcccgatcaactgcttgtccaacatcaccgggctattgttgacgagagatggtggtgacaacaataatacgatagagaccttcagacctggaggaggagatatgagggacaactggaggagcgagctgtacaagtacaaggtagtgaggatcgagccattgggaatagcacccaccaaggcaaagagaagagtggtgcaaagagagaaaagagcagtgggaataggagctatgttccttgggttcttgggagcagcaggaagcactatgggcgcagcgtcggtgacccttaccgtgcaagctcgcctgctgctgtcgggtatagtgcaacagcaaaacaacctcctccgcgcaatcgaagcccagcagcatctgttgcaactcacggtctggggcatcaagcagctccaggctagagtccttgccatggagcgttatctgaaagaccagcaacttcttgggatttggggttgctcgggaaaactcatttgcaccacgaatgtgccttggaacgccagctggagcaacaagtccctggacaagatttggcataacatgacctggatggagtgggaccgcgagatcgacaactacacgaaattgatatacaccctgatcgaggcgtcccagatccagcaggagaagaatgagcaagagttgttggagttggattcgtgggcgtcgttgtggtcgtggtttgacatctcgaaatggctgtggtatataggagtattcataatagtaataggaggtttggtaggtttgaaaatagtttttgctgtactttcgatagtaaatcgagttaggcagggatactcgccattgtcatttcaaacccgcctcccagccccgcggggacccgacaggcccgagggcatcgaggagggaggcggcgagagagacagagacagatccgatcaattggtgacgggattcttggcactcatctgggacgatctgcggagcctgtgcctcttctcttaccaccgcctgcgcgacctgctcctgatcgtggcgaggatcgtggagcttctgggacgcagggggtgggaggccctgaagtactggtggaacctcctgcaatattggattcaggagctgaagaacagcgccgttagtctgctgaacgctaccgctatcgccgtggcggaaggaaccgacaggattatagaggtagtacaaaggattggtcgcgccatcctccatatcccccgccgcatccgccagggcttggagagggctttgctataa

SEQ ID NO: 86 - protein, 860 aa, Artificial Sequence
  Description of Artificial SequenceCATEenv(HIV) fusion protein
MRKAAVSHWQQQSYLDSGIHSGATTTAPSLSICSAEEKLWVTVYYGVPVWKEATTTLFCASDAKAHHAEAHNVWATHACVPTDPNPQEVILENVTEKYNMWKNNMVDQMHEDIISLWDQSLKPCVKLTPLCVTLNCTNATYTNSDSKNSTSNSSLEDSGKGDMNCSFDVTTSIDKKKKTEYAIFDKLDVMNIGNGRYTLLNCNTSVITQACPKMSFEPIPIHYCTPAGYAILKCNDNKFNGTGPCTNVSTIQCTHGIKPVVSTQLLLNGSLAEGGEVIIRSENLTDNAKTIIVQLKEPVEINCTRPNNNTRKSIHMGPGAAFYARGEVIGDIRQAHCNISRGRWNDTLKQIAKKLREQFNKTISLNQSSGGDLEIVMHTFNCGGEFFYCNTTQLFNSTWNENDTTWNNTAGSNNNETITLPCRIKQIINRWQEVGKAMYAPPISGPINCLSNITGLLLTRDGGDNNNTIETFRPGGGDMRDNWRSELYKYKVVRIEPLGIAPTKAKRRVVQREKRAVGIGAMFLGFLGAAGSTMGAASVTLTVQARLLLSGIVQQQNNLLRAIEAQQHLLQLTVWGIKQLQARVLAMERYLKDQQLLGIWGCSGKLICTTNVPWNASWSNKSLDKIWHNMTWMEWDREIDNYTKLIYTLIEASQIQQEKNEQELLELDSWASLWSWFDISKWLWYIGVFIIVIGGLVGLKIVFAVLSIVNRVRQGYSPLSFQTRLPAPRGPDRPEGIEEGGGERDRDRSDQLVTGFLALIWDDLRSLCLFSYHRLRDLLLIVARIVELLGRRGWEALKYWWNLLQYWIQELKNSAVSLLNATAIAVAEGTDRIIEVVQRIGRAILHIPRRIRQGLERALL

SEQ ID NO: 87 - DNA, 1416 bp, Artificial Sequence
  Description of Artificial Sequence PMCP3p37M1-10
atgaacccaagtgctgccgtcattttctgcctcatcctgctgggtctgagtgggactcaagggatcctcgacatggcgcaaccggtaggtataaacacaagcacaacctgttgctatcgtttcataaataaaaagataccgaagcaacgtctggaaagctatcgccgtaccacttctagccactgtccgcgtgaagctgttatattcaaaacgaaactggataaggagatctgcgccgaccctacacagaaatgggttcaggactttatgaagcacctggataaaaagacacagacgccgaaactggctagcgcaggagcaggtgcgagagcgtcagtattaagcgggggagaattagatcgatgggaaaaaattcggttaaggccagggggaaagaagaagtacaagctaaagcacatcgtatgggcaagcagggagctagaacgattcgcagttaatcctggcctgttagaaacatcagaaggctgtagacaaatactgggacagctacaaccatcccttcagacaggatcagaggagcttcgatcactatacaacacagtagcaaccctctattgtgtgcaccagcggatcgagatcaaggacaccaaggaagctttagacaagatagaggaagagcaaaacaagtccaagaagaaggcccagcaggcagcagctgacacaggacacagcaatcaggtcagccaaaattaccctatagtgcagaacatccaggggcaaatggtacatcaggccatatcacctagaactttaaatgcatgggtaaaagtagtagaagagaaggctttcagcccagaagtgatacccatgttttcagcattatcagaaggagccaccccacaggacctgaacacgatgttgaacaccgtggggggacatcaagcagccatgcaaatgttaaaagagaccatcaatgaggaagctgcagaatgggatagagtgcatccagtgcatgcagggcctattgcaccaggccagatgagagaaccaaggggaagtgacatagcaggaactactagtacccttcaggaacaaataggatggatgacaaataatccacctatcccagtaggagagatctacaagaggtggataatcctgggattgaacaagatcgtgaggatgtatagccctaccagcattctggacataagacaaggaccaaaggaaccctttagagactatgtagaccggttctataaaactctaagagctgagcaagcttcacaggaggtaaaaaattggatgacagaaaccttgttggtccaaaatgcgaacccagattgtaagaccatcctgaaggctctcggcccagcggctacactagaagaaatgatgacagcatgtcagggagtaggaggacccggccataaggcaagagttttggaattctga

SEQ ID NO: 88 - protein, 471 aa, Artificial Sequence
  Description of Artificial Sequence PMCP3p37M1-10 fusion protein, MCP3-p37gag fusion protein
MNPSAAVIFCLILLGLSGTQGILDMAQPVGINTSTTCCYRFINKKIPKQRLESYRRTTSSHCPREAVIFKTKLDKEICADPTQKWVQDFMKHLDKKTQTPKLASAGAGARASVLSGGELDRWEKIRLRPGGKKKYKLKHIVWASRELERFAVNPGLLETSEGCRQILGQLQPSLQTGSEELRSLYNTVATLYCVHQRIEIKDTKEALDKIEEEQNKSKKKAQQAAADTGHSNQVSQNYPIVQNIQGQMVHQAISPRTLNAWVKVVEEKAFSPEVIPMFSALSEGATPQDLNTMLNTVGGHQAAMQMLKETINEEAAEWDRVHPVHAGPIAPGQMREPRGSDIAGTTSTLQEQIGWMTNNPPIPVGEIYKRWIILGLNKIVRMYSPTSILDIRQGPKEPFRDYVDRFYKTLRAEQASQEVKNWMTETLLVQNANPDCKTILKALGPAATLEEMMTACQGVGGPGHKARVLEF

SEQ ID NO: 89 - DNA, 1092 bp, Artificial Sequence
  Description of Artificial Sequencep37M1-10 (HIV)
atgggtgcgagagcgtcagtattaagcgggggagaattagatcgatgggaaaaaattcggttaaggccagggggaaagaagaagtacaagctaaagcacatcgtatgggcaagcagggagctagaacgattcgcagttaatcctggcctgttagaaacatcagaaggctgtagacaaatactgggacagctacaaccatcccttcagacaggatcagaggagcttcgatcactatacaacacagtagcaaccctctattgtgtgcaccagcggatcgagatcaaggacaccaaggaagctttagacaagatagaggaagagcaaaacaagtccaagaagaaggcccagcaggcagcagctgacacaggacacagcaatcaggtcagccaaaattaccctatagtgcagaacatccaggggcaaatggtacatcaggccatatcacctagaactttaaatgcatgggtaaaagtagtagaagagaaggctttcagcccagaagtgatacccatgttttcagcattatcagaaggagccaccccacaggacctgaacacgatgttgaacaccgtggggggacatcaagcagccatgcaaatgttaaaagagaccatcaatgaggaagctgcagaatgggatagagtgcatccagtgcatgcagggcctattgcaccaggccagatgagagaaccaaggggaagtgacatagcaggaactactagtacccttcaggaacaaataggatggatgacaaataatccacctatcccagtaggagagatctacaagaggtggataatcctgggattgaacaagatcgtgaggatgtatagccctaccagcattctggacataagacaaggaccaaaggaaccctttagagactatgtagaccggttctataaaactctaagagctgagcaagcttcacaggaggtaaaaaattggatgacagaaaccttgttggtccaaaatgcgaacccagattgtaagaccatcctgaaggctctcggcccagcggctacactagaagaaatgatgacagcatgtcagggagtaggaggacccggccataaggcaagagttttgtag

SEQ ID NO: 90 - protein, 363 aa, Artificial Sequence
  Description of Artificial Sequencep37M1-10 (HIV) fusion protein
MGARASVLSGGELDRWEKIRLRPGGKKKYKLKHIVWASRELERFAVNPGLLETSEGCRQILGQLQPSLQTGSEELRSLYNTVATLYCVHQRIEIKDTKEALDKIEEEQNKSKKKAQQAAADTGHSNQVSQNYPIVQNIQGQMVHQAISPRTLNAWVKVVEEKAFSPEVIPMFSALSEGATPQDLNTMLNTVGGHQAAMQMLKETINEEAAEWDRVHPVHAGPIAPGQMREPRGSDIAGTTSTLQEQIGWMTNNPPIPVGEIYKRWIILGLNKIVRMYSPTSILDIRQGPKEPFRDYVDRFYKTLRAEQASQEVKNWMTETLLVQNANPDCKTILKALGPAATLEEMMTACQGVGGPGHKARVL

SEQ ID NO: 91 - DNA, 4179 bp, Artificial Sequence
  Description of Artificial SequenceHIV gagpol
atgggtgcgagagcgtcagtattaagcgggggagaattagatcgatgggaaaaaattcggttaaggccagggggaaagaagaagtacaagctaaagcacatcgtatgggcaagcagggagctagaacgattcgcagttaatcctggcctgttagaaacatcagaaggctgtagacaaatactgggacagctacaaccatcccttcagacaggatcagaggagcttcgatcactatacaacacagtagcaaccctctattgtgtgcaccagcggatcgagatcaaggacaccaaggaagctttagacaagatagaggaagagcaaaacaagtccaagaagaaggcccagcaggcagcagctgacacaggacacagcaatcaggtcagccaaaattaccctatagtgcagaacatccaggggcaaatggtacatcaggccatatcacctagaactttaaatgcatgggtaaaagtagtagaagagaaggctttcagcccagaagtgatacccatgttttcagcattatcagaaggagccaccccacaggacctgaacacgatgttgaacaccgtggggggacatcaagcagccatgcaaatgttaaaagagaccatcaatgaggaagctgcagaatgggatagagtgcatccagtgcatgcagggcctattgcaccaggccagatgagagaaccaaggggaagtgacatagcaggaactactagtacccttcaggaacaaataggatggatgacaaataatccacctatcccagtaggagagatctacaagaggtggataatcctgggattgaacaagatcgtgaggatgtatagccctaccagcattctggacataagacaaggaccaaaggaaccctttagagactatgtagaccggttctataaaactctaagagctgagcaagcttcacaggaggtaaaaaattggatgacagaaaccttgttggtccaaaatgcgaacccagattgtaagaccatcctgaaggctctcggcccagcggctacactagaagaaatgatgacagcatgtcagggagtaggaggacccggccataaggcaagagttttggccgaggcgatgagccaggtgacgaactcggcgaccataatgatgcagagaggcaacttccggaaccagcggaagatcgtcaagtgcttcaattgtggcaaagaagggcacaccgccaggaactgccgggccccccggaagaagggctgctggaagtgcgggaaggaggggcaccagatgaaggactgcacggagcggcaggcgaacttcctggggaagatatggccgagttacaagggaagacccgaccggcaggggacggtgtcgttcaacttccctcagatcacgctctggcagcggccgctcgtcacaataaagatcggggggcaactcaaggaggcgctgctcgcggacgacacggtcttggaggagatgtcgttgccggggcggtggaagccgaagatgatcggggggatcgggggcttcatcaaggtgcggcagtacgaccagatcctcatcgagatctgcgggcacaaggcgatcgggacggtcctcgtcggcccgacgccggtcaacatcatcgggcggaacctgttgacccagatcggctgcaccttgaacttccccatcagccctattgagacggtgcccgtgaagttgaagccggggatggacggccccaaggtcaagcaatggccattgacggaggagaagatcaaggccttagtcgaaatctgtacagagatggagaaggaagggaagatcagcaagatcgggcctgagaacccctacaacactccagtcttcgcaatcaagaagaaggacagtaccaagtggagaaagctggtggacttcagagagctgaacaagagaactcaggacttctgggaagttcagctgggcatcccacatcccgctgggttgaagaagaagaagtcagtgacagtgctggatgtgggtgatgcctacttctccgttcccttggacgaggacttcaggaagtacactgccttcacgatacctagcatcaacaacgagacaccaggcatccgctaccagtacaacgtgctgccacagggatggaagggatcaccagccatctttcaatcgtcgatgaccaagatcctggagcccttccgcaagcaaaacccagacatcgtgatctatcagctctacgtaggaagtgacctggagatcgggcagcacaggaccaagatcgaggagctgagacagcatctgttgaggtggggactgaccacaccagacaagaagcaccagaaggaacctcccttcctgtggatgggctacgaactgcatcctgacaagtggacagtgcagcccatcgtgctgcctgagaaggacagctggactgtgaacgacatacagaagctcgtgggcaagttgaactgggcaagccagatctacccaggcatcaaagttaggcagctgtgcaagctgcttcgaggaaccaaggcactgacagaagtgatcccactgacagaggaagcagagctagaactggcagagaaccgagagatcctgaaggagccagtacatggagtgtactacgacccaagcaaggacctgatcgcagagatccagaagcaggggcaaggccaatggacctaccaaatctaccaggagcccttcaagaacctgaagacaggcaagtacgcaaggatgaggggtgcccacaccaacgatgtgaagcagctgacagaggcagtgcagaagatcaccacagagagcatcgtgatctggggcaagactcccaagttcaagctgcccatacagaaggagacatgggagacatggtggaccgagtactggcaagccacctggatccctgagtgggagttcgtgaacacccctcccttggtgaaactgtggtatcagctggagaaggaacccatcgtgggagcagagaccttctacgtggatggggcagccaacagggagaccaagctgggcaaggcaggctacgtgaccaaccgaggacgacagaaagtggtgaccctgactgacaccaccaaccagaagactctgcaagccatctacctagctctgcaagacagcggactggaagtgaacatcgtgacagactcacagtacgcactgggcatcatccaagcacaaccagaccaatccgagtcagagctggtgaaccagatcatcgagcagctgatcaagaaggagaaagtgtacctggcatgggtcccggcgcacaaggggatcggggggaacgagcaggtcgacaagttggtctcggcggggatccggaaggtgctgttcctggacgggatcgataaggcccaagatgaacatgagaagtaccactccaactggcgcgctatggccagcgacttcaacctgccgccggtcgtcgcgaaggagatcgtcgccagctgcgacaagtgccagctcaagggggaggccatgcacgggcaagtcgactgcagtccggggatctggcagctgtgcacgcacctggaggggaaggtgatcctggtcgcggtccacgtcgccagcgggtatatcgaggcggaggtcatcccggctgagacggggcaggagacggcgtacttcctcttgaagctcgcggggcggtggccggtcaagacgatccacacgaacgggagcaacttcacgggggcgacggtcaaggccgcctgttggtgggcgggaatcaagcaggaatttggaattccctacaatccccaatcgcaaggagtcgtgagcatgaacaaggagctgaagaagatcatcggacaaagggatcaggctgagcacctgaagacagcagtgcagatggcagtgttcatccacaacttcaaaagaaaaggggggattggggggtacagtgcgggggaacggatcgtggacatcatcgccaccgacatccaaaccaaggagctgcagaagcagatcaccaagatccagaacttccgggtgtactaccgcgacagccgcaacccactgtggaagggaccagcaaagctcctctggaagggagagggggcagtggtgatccaggacaacagtgacatcaaagtggtgccaaggcgcaaggccaagatcatccgcgactatggaaaacagatggcaggggatgattgtgtggcaagtagacaggatgaggatggcgcctag

SEQ ID NO: 92 - protein, 1392 aa, Artificial Sequence
  Description of Artificial SequenceHIV gagpol fusion protein
MGARASVLSGGELDRWEKIRLRPGGKKKYKLKHIVWASRELERFAVNPGLLETSEGCRQILGQLQPSLQTGSEELRSLYNTVATLYCVHQRIEIKDTKEALDKIEEEQNKSKKKAQQAAADTGHSNQVSQNYPIVQNIQGQMVHQAISPRTLNAWVKVVEEKAFSPEVIPMFSALSEGATPQDLNTMLNTVGGHQAAMQMLKETINEEAAEWDRVHPVHAGPIAPGQMREPRGSDIAGTTSTLQEQIGWMTNNPPIPVGEIYKRWIILGLNKIVRMYSPTSILDIRQGPKEPFRDYVDRFYKTLRAEQASQEVKNWMTETLLVQNANPDCKTILKALGPAATLEEMMTACQGVGGPGHKARVLAEAMSQVTNSATIMMQRGNFRNQRKIVKCFNCGKEGHTARNCRAPRKKGCWKCGKEGHQMKDCTERQANFLGKIWPSYKGRPDRQGTVSFNFPQITLWQRPLVTIKIGGQLKEALLADDTVLEEMSLPGRWKPKMIGGIGGFIKVRQYDQILIEICGHKAIGTVLVGPTPVNIIGRNLLTQIGCTLNFPISPIETVPVKLKPGMDGPKVKQWPLTEEKIKALVEICTEMEKEGKISKIGPENPYNTPVFAIKKKDSTKWRKLVDFRELNKRTQDFWEVQLGIPHPAGLKKKKSVTVLDVGDAYFSVPLDEDFRKYTAFTIPSINNETPGIRYQYNVLPQGWKGSPAIFQSSMTKILEPFRKQNPDIVIYQLYVGSDLEIGQHRTKIEELRQHLLRWGLTTPDKKHQKEPPFLWMGYELHPDKWTVQPIVLPEKDSWTVNDIQKLVGKLNWASQIYPGIKVRQLCKLLRGTKALTEVIPLTEEAELELAENREILKEPVHGVYYDPSKDLIAEIQKQGQGQWTYQIYQEPFKNLKTGKYARMRGAHTNDVKQLTEAVQKITTESIVIWGKTPKFKLPIQKETWETWWTEYWQATWIPEWEFVNTPPLVKLWYQLEKEPIVGAETFYVDGAANRETKLGKAGYVTNRGRQKVVTLTDTTNQKTLQAIYLALQDSGLEVNIVTDSQYALGIIQAQPDQSESELVNQIIEQLIKKEKVYLAWVPAHKGIGGNEQVDKLVSAGIRKVLFLDGIDKAQDEHEKYHSNWRAMASDFNLPPVVAKEIVASCDKCQLKGEAMHGQVDCSPGIWQLCTHLEGKVILVAVHVASGYIEAEVIPAETGQETAYFLLKLAGRWPVKTIHTNGSNFTGATVKAACWWAGIKQEFGIPYNPQSQGVVSMNKELKKIIGQRDQAEHLKTAVQMAVFIHNFKRKGGIGGYSAGERIVDIIATDIQTKELQKQITKIQNFRVYYRDSRNPLWKGPAKLLWKGEGAVVIQDNSDIKVVPRRKAKIIRDYGKQMAGDDCVASRQDEDGA

SEQ ID NO: 93 - DNA, 1197 bp, Artificial Sequence; identical to SEQ ID NO: 77
  Description of Artificial Sequence CATEp37gag(HIV)

SEQ ID NO: 94 - protein, 398 aa, Artificial Sequence; identical to SEQ ID NO: 78
  Description of Artificial Sequence CATEp37gag(HIV) fusion protein

SEQ ID NO: 95 - protein, 4 aa, Artificial Sequence
  Description of Artificial Sequence183YMDD186 from Gagpol in-frame
YMDD